(19) United States
(12) Patent Application Publication
(10) Pub. No.: US 2026/0297595 A1
(43) Pub. Date: Oct. 1, 2026

(54) TRANSCRIPTIONAL RECORDING BY CRISPR SPACER ACQUISITION FROM RNA

(71) Applicant: ETH Zurich, Zurich (CH)

(72) Inventors: Randall Platt, Zurich (CH); Florian Schmidt, Zurich (CH); Alexander Klotz, Zurich (CH); Ricardo Ricaurte Rodriguez, Zurich (CH); Tarek Farouni, Zurich (CH)

(73) Assignee: ETH Zurich, Zurich (CH)

(21) Appl. No.: 19/639,325

(22) Filed: Apr. 6, 2026

Related U.S. Application Data
(63) Continuation-in-part of application No. 17/274,443, filed on Sep. 11, 2019, now Pat. No. 12,595,468.

(30) Foreign Application Priority Data
Sep. 11, 2018 (EP) 18193881.2

(51) Int. Cl. C12N 15/70; C12N 9/12; C12N 9/22; C12N 15/11

(52) U.S. Cl. CPC C12N 15/70; C12N 9/1276; C12N 9/224; C12N 15/11; C12Y 207/07049; C12N 2310/20; C12N 2830/002

(57) ABSTRACT

The present invention relates to an expression vector and a cell comprising an RT-Cas1 and Cas2 and a corresponding CRISPR array from different species.

### CROSS-REFERENCE TO RELATED APPLICATIONS

This is a Continuation-in-Part of U.S. patent application Ser. No. 17/274,443, filed Mar. 9, 2021, which is the US National Stage of International Patent Application No. PCT/EP2019/074267, filed Sep. 11, 2019, which in turn claims priority to European Patent Application No. 18193881.2, filed Sep. 11, 2018. The contents of the foregoing applications are incorporated by reference herein in their entirety.

### BRIEF DESCRIPTION OF THE SEQUENCE LISTING

The nucleotide and amino acid sequences provided herewith are shown using standard letter abbreviations for nucleotide bases and amino acids as defined in 37 CFR 1.831 through 37 CFR 1.835. Only one strand of each nucleic acid sequence is shown, but the complementary strand is understood as included by any reference to the displayed strand. The Sequence Listing is submitted as an XML file named 95083_315_2801 revseq, approximately 519,000 bytes, created Jun. 15, 2026, the contents of which are incorporated by reference herein in their entirety.

### FIELD

The present invention relates to an expression vector and a cell comprising an RT-Cas1 and Cas2 and a corresponding CRISPR array from different species.

### BACKGROUND

A central challenge in biology is to understand how the molecular components of a cell function and integrate to enable complex cell behaviors. This challenge has fueled the creation of increasingly sophisticated technologies facilitating detailed intracellular observations at the level of DNA, RNA, protein, and metabolites. In particular, RNA sequencing technologies enable transcriptome quantification within multiple or single cells, revealing the molecular signatures of cell behaviors, states, and types with unprecedented detail. Despite the power of these technologies, they require destructive methods and therefore observations are limited to a few snapshots in time or select asynchronous cellular processes. One provocative solution to this is to introduce synthetic memory devices within cells that enable encoding, storage, and retrieval of transcriptional information.

The bacterial adaptive immune system CRISPR-Cas embodies the ideal molecular recorder. Molecular memories of plasmid or viral infections are stored within CRISPR arrays in the form of short nucleic acid segments (spacers) separated by direct repeats (DRs). New memories are acquired via the action of Cas1 and Cas2, which as a complex integrate new spacers ahead (next to the leader sequence or proximal to the leader sequence) of old spacers within the CRISPR array, thereby providing a temporal memory of molecular events. The prototype Type I-E CRISPR acquisition system from E. coli was recently leveraged to store arbitrary information and quantifiable records of defined stimuli within bacterial populations (Shipman et al, Science, vol. 353(6298), (2016), aaf1175; Shipman et al, Nature, vol. 547, (2017), 346-349; and Sheth et al, Science, 10.1126/science.aao0958, (2017)). These systems elegantly demonstrate the potential of using CRISPR spacer acquisition as a molecular recorder, but they are currently limited by the need to electroporate chemically synthesized nucleotides or, analogous to prior technologies, the availability of inducible promoters. Moreover, these systems acquire spacers derived from DNA but not RNA, and therefore do not globally reflect the transcriptional history of a cell.

Based on this background is the objective of the present invention to provide a method and means for recording changes in the expression pattern of RNAs within the living cell without destroying the cell. This objective is attained by the subject matter of the claims of the present specification.

### Terms and Definitions

The term CRISPR is an abbreviation for clustered regularly interspaced short palindromic repeats.

In the context of the present specification, the term spacer relates to polynucleotides that are inserted into a CRISPR array. The complex of Cas1 and Cas2 cuts the DNA inside the CRISPR array and integrates spacers at that position. Spacers are integrated upstream of a direct repeat sequence.

In the context of the present specification, the term CRISPR array refers to a nucleic acid sequence, in which acquired spacers are inserted or integrated by a Cas1-Cas2 complex.

In the context of the present specification, the term protospacer relates to the precursor of a spacer before being integrated into the CRISPR array as spacer. If the protospacer is a single-stranded RNA, the RNA is first integrated into the CRISPR array and then reverse-transcribed into DNA.

In the context of the present specification, the term transgene or transgeneic relates to a gene or coding sequence, partially or fully originating from a different organism than the host organism, in relation to which the sequence is a transgene sequence.

In the context of the present specification, the term codon-optimized relates a change of nucleotide sequence without changing the amino acid sequence it encodes. Every organism has a certain codon usage and by optimizing the codons with respect to the host organism, the efficiency of expression may be increased.

In the context of the present specification, the term overexpression relates to the expression of an artificially introduced gene, which is higher than the expression of a constitutively expressed gene such as a household gene of the host organisms, particularly two-fold higher, more particular 5-fold higher, even more particular 10-fold higher.

In the context of the present specification, the term transcriptome relates to the set of all RNAs inside the host or test cell, particularly the set of all mRNAs inside the host or test cell.

In context of the present specification, the term leader sequence relates to a nucleic acid sequence that is located immediately before or after the first or last CRISPR direct repeat sequence of a CRISPR array or locus.

In the context of the present specification, the terms sequence identity and percentage of sequence identity refer to a single quantitative parameter representing the result of a sequence comparison determined by comparing two aligned sequences position by position. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

### DETAILED DESCRIPTION OF THE INVENTION

A first aspect of the invention relates to an expression vector comprising the following sequence elements:
a first transgene nucleic acid sequence encoding a first amino acid sequence of a fusion protein of a reverse transcriptase and a Cas1 polypeptide, and a second transgene nucleic acid sequence encoding a second amino acid sequence of a Cas2 polypeptide, wherein said first transgene nucleic acid sequence and said second transgene nucleic acid sequence are under transcriptional control of an inducible promoter sequence, and
a CRISPR array sequence comprising a CRISPR direct repeat (DR) sequence, wherein said CRISPR direct repeat sequence is specifically recognizable by a RT-Cas1-Cas2 complex formed by the expression products of said first nucleic acid sequence and said second nucleic acid sequence;
wherein said first amino acid sequence consists of a sequence being ≥90%, or ≥91%%, or ≥92%%, or ≥93%%, or ≥94%%, or ≥95%%, or ≥96%%, or ≥97%%, or ≥98%%, or ≥98.2%%, or ≥98.4%%, or ≥98.6%%, or ≥98.8%%, or 99%, or ≥99.1%%, or ≥99.2%%, or ≥99.3%%, or ≥99.4%, or ≥99.5%, or ≥99.6%, or ≥99.7%, or ≥99.8%, or ≥99.9%, or 100% identical to a sequence selected from the group consisting of SEQ ID NO: 1-18;
and wherein said second amino acid sequence consists of a sequence being 90%, or 91%%, or ≥92%%, or ≥93%%, or ≥94%%, or ≥95%%, or ≥96%%, or ≥97%%, or ≥98%%, or ≥98.2%%, or ≥98.4%%, or ≥98.6%%, or ≥98.8%%, or ≥99%, or ≥99.1%%, or ≥99.2%%, or ≥99.3%%, or ≥99.4%, or ≥99.5%, or ≥99.6%, or ≥99.7%, or 99.8%, or ≥99.9%, or 100% identical to a sequence selected from the group consisting of SEQ ID NO: 21-38;
and wherein said CRISPR array sequence consists of a sequence being ≥98% or ≥98.5%, or ≥99%, or ≥299.5% or 100% identical to a sequence selected from the group consisting of SEQ ID NO: 40-106.

A second aspect of the invention relates to a bacterial cell (“test cell”) comprising the first transgene nucleic acid sequence, the second transgene nucleic acid sequence and the CRISPR array sequence of the first aspect,
wherein said first transgene nucleic acid sequence, said second transgene nucleic acid sequence and said CRISPR array sequence are
comprised in an expression vector of the first aspect, or
integrated into the genome of said cell.

In certain embodiments, the RT-Cas1 protein and the Cas2 protein are employed together with their respective array, as grouped by species in the sequence section.

In certain embodiments, said first amino acid sequence being ≥90% identical to a sequence selected from the group consisting of SEQ ID NO: 1-18 comprises only substitutions in relation to SEQ ID NO: 1-18, and no insertions or deletions.

In certain embodiments, said second amino acid sequence being ≥90% identical to a sequence selected from the group consisting of SEQ ID NO: 21-38 comprises only substitutions in relation to SEQ ID NO: 21-38, and no insertions or deletions.

In certain embodiments, said first amino acid sequence comprises 0 to 20 (0, 1, 2, 3, 4, 5, 6, 7, 8, 9, 10, 11, 12, 13, 14, 15, 16, 17, 18, 19, 20) substitutions with respect to SEQ ID NO: 1, wherein following fixed amino acid positions with respect to SEQ ID NO: 1 are not substituted:
Highly conserved amino acid positions of Fs.RT-Cas1 (SEQ ID NO: 001) based on the DMS data (no other amino acids can be inserted at these positions):
M1, F2, I4, E6, N12, A16, F17, H19, F20, K23, D25, G28, D30, G31, M32, S35, E36, L37, Y40, W41, N44, 148, D51, L52, E56, Y57, Q58, P59, G60, E66, K70, G72, K73, R74, R75, N76, 177, A78, L80, N81, V82, I83, D84, R85, F86, 187, T88, R89, L90, Q93, L95, L99, F103, S107, Y108, A109, Y110, Q111, K114, G115, A119, K124, Y126, G130, D138, L139, Y142, F143, D144, I146, L148, L151, I152, P153, I155, V164, L167, I168, K169, Y171, L172, C174, D175, G180, I182, R184, G188, I189, G192, N193, A194, I195, S196, P197, L199, S200, N201, L202, Y203, L204, F207, D208, W218, R220, Y221, D223, N224, Y228, L236, L251, N254, K257, S258, G259, F261, R266, L269, G270, Y271, D272, I273, D281, R283, H285, Y287, Y293, W296, L301, I304, N305, G306, R307, Y308, H309, I310, S312, D313, G314, I315, R318, D320, F321, L323, L324, F325, E326, N327, 1334, P335, E337, V338, D340, N343, N347, V348, L350, V354, L355, I363, V365, F367, F368, D369, K370, Y371, G372, L374, 1375, G376, F378, L379, P380, E381, A387, I389, L391, Q393, Y397, R403, A407, R408, M410, A413, L415, H416, N417, I418, R419, A420, N421, L422, R423, Y424, Y425, K427, K428, F433, V437, D438, I440, I444, L447, S452, V453, M456, L458, E460, A461, A463, R464, Y467, Y468, F471, N472, 1474, L475, F480, F482, R485, T486, P489, P490, D492, A493, I494, N495, A496, I498, S499, F500, G501, N502, T503, L504, L505, Y506, N507, F509, I512, I513, W514, K516, G517, L518, D519, P520, R521, F522, G523, V524, H526, R531, S534, L535, N536, L537, D538, F539, A540, D541, I542, F543, K544, P545, I546, V547, D549, R550, I552, F553, T554, M555, I556, N557, K558, L561, F567, G573, V574, Y575, L576, S577, G580, K581, F584, L585, E589, K591, L592, K593, T597, K599, G600, M603, S604, Y605, L608, L609, E610, E612, V613, Q614, Y616, K617, N618, I620, L621, G623, Y626, K627, P628, Y629, K630, Y631, Y632

and wherein following conserved amino acid positions with respect to SEQ ID NO: 1 can only be substituted according to substitution rules given below:
Conservative exchangeable amino acid positions of Fs.RT-Cas1 (SEQ ID NO: 001) based on the DMS data (10-30% of possible amino acids can be inserted at these positions):
T3, D5, M7, L8, A21, G26, H33, V34, E38, M43, Q47, K53, I61, I62, I64, R65, M68, S79, L91, A100, V116, V120, K122, A123, R132, V134, I137, N141, E156, Y158, I159, D161, L165, H166, F173, F178, E179, K185, V190, N205, D206, L211, I219, A222, I225, Y226, I227, Y232, E233, A235, Y239, L246, V253, E256, V260, V263, I268, L274, K278, K279, V280, V282, K284, I286, H297, D298, S299, T311, L316, N317, G322, E328, K330, K331, Y333, S339, Q341, L342, 1344, Y345, G346, T349, A351, S352, N353, Q356, S357, F358, S359, S366, R373, T383, K384, K385, S386, 1390, L398, N399, D401, M404, D405, T406, R409, E411, G414, D426, H429, K430, G431, K434, E435, S441, Y443, A446, A450, D455, M457, L459, K462, Q465, L466, C470, Q473, D479, K487, R488, K491, C497, N511, K515, V525, A527, S528, N529, I548, I551, K559, M560, D566, E568, R578, Y588, S594, R595, I596, I598, Y606, Q607, N615, F619, E624;
or wherein 0-20 (0, 1, 2, 3, 4, 5, 6, 7, 8, 9, 10, 11, 12, 13, 14, 15, 16, 17, 18, 19, 20) amino acid positions of each sequence of SEQ ID NO: 2-17 are substituted, wherein amino acids homologous to the fixed amino acids of SEQ ID NO: 1 (according to the alignment of FIG. 7) cannot be substituted, and wherein amino acids homologous to conserved amino acids of SEQ ID NO: 1 (according to the alignment of FIG. 7) can only be substituted according to the substitution rules.

This means that the following flexible amino acid positions with respect to SEQ ID NO: 1 can freely be substituted (by any other of the remaining 19 proteinogenic amino acids):
Interchangeable amino acid positions of Fs.RT-Cas1 (SEQ ID NO: 001) based on the DMS data (≥30% of possible amino acids can be inserted at these positions):
S9, K10, N11, Q13, R14, L15, E18, T22, N24, C27, P29, K39, R42, H45, D46, 149, S50, N54, Q55, L63, H67, N69, T71, S92, K94, N96, R97, Y98, P101, I102, C104, E105, N106, D112, S113, M117, P118, L121, E125, V127, E128, L129, M131, H133, I135, E136, K140, T145, P147, E149, N150, E154, R157, T160, E162, A163, Q170, I176, S177, K181, S183, T186, Q187, Q191, I198, K209, E210, D212, E213, S214, K215, L216, C217, M229, D230, S231, K234, L237, V238, S240, E241, L242, T243, E244, R245, E247, R248, R249, K250, T252, K255, D262, S264, T265, S267, I275, R276, N277, K288, S289, V290, N291, Q292, S294, N295, R300, E302, F303, Q319, Q329, H332, V336, N360, R361, E362, K364, S377, K382, E388, V392, S394, K395, N396, E400, V402, 1412, D432, K436, A439, G442, D445, N448, R449, P451, N454, T469, E476, T477, S478, Q481, E483, K484, L508, V510, K530, N532, Q533, T562, L563, L564, T565, T569, S570, N571, Q572, E579, N582, I583, Q586, M587, E590, K601, E602, S611, T622, T625.

In certain embodiments, said second amino acid sequence comprises 1 to 8 (1, 2, 3, 4, 5, 6, 7, 8) substitutions with respect to SEQ ID NO: 21, wherein following fixed amino acid positions with respect to SEQ ID NO: 21 are not substituted:
Highly conserved amino acid positions of Fs.Cas2 (SEQ ID NO: 021) based on the DMS data (no other amino acids can be inserted at these positions):
M1, Y2, I4, L5, Y7, D8, K12, R13, R22, Y24, L25, Q29, K30, S31, F33, G35, E39, K41, L45, E48, L49, D54, D58, Y63, L65, S67, K69, Y70, K73, G77, 178, S81, I86
and wherein following conserved amino acid positions with respect to SEQ ID NO: 21 can only be substituted according to substitution rules given below:
Conservative exchangeable amino acid positions of Fs.Cas2 (SEQ ID NO: 021) based on the DMS data (10-30% of possible amino acids can be inserted at these positions):
V3, V6, I9, V14, K16, A17, L18, I20, C21, K23, I28, V32, I37, T38, K46, I53, Q56, M57, V60, I61, I62, V68, T71, K72, T82, S83, V85
or wherein 0-8 (0, 1, 2, 3, 4, 5, 6, 7, 8) amino acid positions of each sequence of SEQ ID NO: 22-37 are substituted, wherein amino acids homologous to the fixed amino acids of SEQ ID NO: 21 (according to the alignment of FIG. 7) cannot be substituted, and wherein amino acids homologous to conserved amino acids of SEQ ID NO: 21 (according to the alignment of FIG. 7) can only be substituted according to the substitution rules.

This means that the following flexible amino acid positions with respect to SEQ ID NO: 21 can freely be substituted (by any other of the remaining 19 proteinogenic amino acids):
Interchangeable amino acid positions of Fs.Cas2 (SEQ ID NO: 021) based on the DMS data (≥30% of possible amino acids can be inserted at these positions):
H10, Q11, G15, K19, I26, H27, E34, N36, S40, L42, K43, A44, E47, G50, H51, L52, T55, S59, H64, D66, E74, Q75, I76, V79, Q80, N84.

### Substitution Rules:

glycine (G) and alanine (A) are interchangeable; valine (V), leucine (L), and isoleucine (I) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) and histidine (H) are interchangeable;
W, F, Y, A, I, L, and V are interchangeable.

The idea of the substitution possibilities is that the inventors performed a deep mutational screen to identify the amino acid position in RT-Cas1 and in Cas2, which may be exchanged or may be conservatively exchanged, or must be fixed. This is shown in FIGS. 4 and 5. Then, the inventors aligned the SEQ ID NO: 1 and SEQ ID NO: 21 to their homologs from other species (FIG. 7) and found that most of the fixed positions are present in all homologs and also most of the conservatively exchangeable positions only show conservative exchanges. Thus, the inventors claim that the homologs of SEQ ID NO: 1 and SEQ ID NO: 21 can be mutated at the same positions as the parent sequence. Fixed positions may occur outside of highly conserved regions of the alignment, representing regions of importance relative to SEQ ID NO: 1 and SEQ ID NO: 21, such as positions involved in functional or structural interactions. The possibility of adding several mutations to the same protein was tested, results are shown in Table 3.

In certain embodiments, said CRISPR array sequence further comprises a CRISPR leader sequence, wherein said CRISPR leader sequence and said CRISPR direct repeat sequence are separated by 10 to 0 bp.

In certain embodiments, said CRISPR array sequence does not comprise any further CRISPR repeat sequence specifically recognizable by said RT-Cas1-Cas2 complex.

In certain embodiments, the expression vector is further comprising an endonuclease recognition site sequence downstream or within of said CRISPR direct repeat, wherein said endonuclease recognition site sequence is specifically recognizable by a site-specific endonuclease, and said CRISPR direct repeat and said restriction site sequence are separated by 10 bps to 0 bps.

In certain embodiments, said site-specific endonuclease is a Type IIS or Type IIG restriction endonuclease.

In certain embodiments, said inducible promoter sequence is operable in E. coli and is selected from T7 promoter, lac promoter, tac promoter, Pₜₑₜ promoter, P_{C} promoter and P_{BAD} promoter.

In certain embodiments, said first transgene nucleic acid sequence and said second transgene nucleic acid sequence are codon-optimized for E. coli.

Acquisition of protospacers is performed by RT-Cas1 and Cas2 forming a complex which associates itself with nucleic acid molecules, particularly with RNA molecules. RT-Cas1 and Cas2 encoded by the first and second transgene nucleic acid sequence form a stable, functional complex that is able to acquire protospacers, particularly from RNA, integrate them into CRISPR arrays and reverse-transcribe them. Thus, protospacers are transformed into spacers, which are pieces of DNA inside the CRISPR array. These spacers can be isolated and sequenced to elucidate the sequence of the protospacers, which are derived from the transcriptome.

Alternatively, the first and second transgene nucleic acid sequence may be under transcriptional control of a constitutive promoter or a promoter expressed under auxotrophic conditions such as hypoxic or anaerobic conditions.

In certain embodiments, said test cell additionally comprises
a fourth transgene nucleic acid sequence encoding a sensor, wherein said sensor will be activated when contacted with an analyte molecule yielding an activated sensor, wherein said activated sensor will induce the expression of a record gene inside the cell;
and wherein in said exposure step, if said analyte molecule is present, said activated sensor induces the expression of a record gene inside the cell and RNA derived from said record gene is acquired as a spacer.

Thus, in certain embodiments, the host cell further comprises a fourth transgene nucleic acid sequence under transcriptional control of an inducible promoter sequence or a constitutive promoter sequence. The inducible or constitutive promoter sequence may be equal to or different from the inducible or constitutive promoter sequence, which controls the expression of the first and second transgene nucleic acid sequence. Preferably, the fourth transgene nucleic acid sequence is under transcriptional control of a synthetic promoter sequence.

Advantageously, specific arbitrary sequences may be expressed and acquired as protospacers that are indicative of a specific stimulus (e.g. the inducing compound). For example, an E. coli cell is engineered to express a specific receptor for a biomarker of a human disease present in the gastrointestinal tract. The recording E. coli by the method of the invention records the downstream intracellular events enacted by the sensor (such as the expression of an arbitrary sequence like a transgene). This allows to equip the recording E. coli cells with multiple diagnostic sensors. Adding transcriptional recording on top of the sensors will aid in further distinguishing disease types or states. Non-limiting examples for suitable biomarkers include sfGFP, Rluc, Fluc. Additionally, non-limiting examples for suitable biomarkers include arbitrary sequences, that is any composition of DNA nucleotides that are for example optimized to be preferentially integrated by the RT-Cas1-Cas2 complex, that are uniquely paired to the biomarker.

In certain embodiments, the third transgene nucleic acid sequence further comprises a CRISPR leader sequence, wherein the CRISPR leader sequence is specifically recognizable by the RT-Cas1-Cas2 complex formed by the expression products of the first transgene nucleic acid sequence and the second transgene nucleic acid sequence. Particularly, the CRISPR direct repeat sequence and the CRISPR leader sequence are in immediate vicinity to each other, e.g. separated by not more than 10 to 0 bp.

Direct repeat sequences and leader sequences may appear in both possible orientations. Accordingly, the third transgene nucleic acid sequence comprising the direct repeat sequence and optionally the leader sequence may be on the sense or anti-sense strand of the DNA of the host organism, irrespective whether the third transgene nucleic acid is integrated in the genome of test cell or the third transgene nucleic acid is comprised within a vector.

In certain embodiments, the third transgene nucleic acid sequence does not comprise any further CRISPR direct repeat sequence.

In certain embodiments, the bacterial cell is an E. coli cell. In certain embodiments, the bacterial cell is an E. coli K12 strain or an E. coli B strain. In certain embodiments, the bacterial cell is an E. coli strain selected from the list of BL21(DE3), BL21AI, NovaBlue(DE3), BW25113, Stbl3, MG1655, JM83, Top10, Nissle 1917, and NGF-1.

In certain embodiments, the conditions, under which expression of the first transgene nucleic acid sequence and the second transgene nucleic acid sequence is induced, result in an overexpression of the first transgene nucleic acid sequence and the second transgene nucleic acid sequence.

In certain embodiments, the conditions, under which expression of the first transgene nucleic acid sequence and the second transgene nucleic acid sequence is induced,
comprise contacting the test cell with an inducer compound and said inducible promoter is a promoter inducible by the inducer compound; or
comprise anaerobic conditions and said inducible promoter is an anaerobically inducible promoter.

In certain embodiments, the inducer compound is IPTG, lactose, arabinose, rhamnose or anhydrotetracycline.

When a promoter is used that is only active in the oxygen poor (anaerobic) environment of the gut, and not the oxygen rich environment outside of the body, the promoter is called anaerobically inducible promoter.

Alternatively, the inducible promoter may be induced by changes in the environment surrounding the test cell or by a changed environment, such as for example temperature, pH value, inflammation, micronutrients, macronutrients, or occurring hypoxic or anaerobic conditions.

In certain embodiments, the third transgene nucleic acid sequence comprises an endonuclease recognition site sequence downstream or within the CRISPR direct repeat, wherein the endonuclease recognition site sequence is specifically recognizable by a site-specific endonuclease, particularly a site-specific restriction endonuclease. In certain embodiments, the CRISPR direct repeat and the restriction site sequence are separated by 10 bps to 0 bps. In certain embodiments, the site-specific endonuclease is a Type IIS restriction endonuclease, particularly FaqI, BsmFI, BsIFI, FinI, or BpuSI.

In certain embodiments, the isolated modified third transgene nucleic acid sequence is contacted with the specific endonuclease before sequencing, wherein the full length CRISPR direct repeat adjacent to said endonuclease site is cleaved into a truncated CRISPR direct repeat sequence.

Advantageously, the site-specific restriction endonuclease truncates the direct repeat sequence most distant to the leader sequence. As the direct repeat sequence is duplicated upon spacer acquisition, modified third transgene nucleic acid sequences comprising at least one acquired spacer will still comprise a full length CRISPR direct repeat after digestion with the above named site-specific endonuclease, while unmodified third transgene nucleic acids (without acquired spacer) will comprise only a truncated CRISPR direct repeat sequence after digestions with the site-specific endonuclease.

In certain embodiments, the sequencing comprises the use of a PCR primer, wherein the PCR primer comprises a nucleic acid sequence being essentially complementary to a full length CRISPR direct repeat sequence within the modified third nucleic acid sequence, wherein the full length CRISPR direct repeat sequence results from or is formed by at least one spacer acquisition event, particularly the portion of said restriction site sequence that is cleaved away upon digestion with said site-specific restriction endonuclease.

The above-mentioned preferred PCR primer binds this region, but not to the truncated CRISPR direct repeat within an unmodified third transgene nucleic acid sequence without acquired spacer. Thus, arrays with only a truncated single DR (i.e. no newly acquired spacers) have no primer binding sequence and are therefore not exponentially amplified. Thus, the site-specific restriction endonuclease site and the preferred primer advantageously enable preferentially amplifying arrays with a new spacer.

In certain embodiments, the expression vector does not comprise any further CRISPR direct repeat sequences recognizable by the RT-Cas1-Cas2 complex encoded by the first and second transgene nucleic acid sequence.

In certain embodiments, the expression vector further comprises a CRISPR leader sequence, wherein the CRISPR leader sequence is specifically recognizable by the RT-Cas1-Cas2 complex formed by the expression products of the first nucleic acid sequence and the second nucleic acid sequence, and wherein particularly the CRISPR leader sequence and the CRISPR direct repeat sequence are separated by 10 to 0 bp.

In certain embodiments, the expression vector further comprises an endonuclease recognition site sequence downstream or within of said CRISPR direct repeat. In certain embodiments, the endonuclease recognition site sequence is specifically recognizable by a site-specific endonuclease, particularly a site-specific restriction endonuclease. In certain embodiments, said CRISPR direct repeat and said restriction site sequence are separated by 10 bps to 0 bps.

In certain embodiments, said site-specific endonuclease is a Type IIS restriction endonuclease, particularly FaqI, BsmFI, BsIFI, FinI, or BpuSI.

In certain embodiments, the CRISPR leader sequence and/or the CRISPR direct repeat sequence are specifically recognizable by a RT-Cas1-Cas2 complex of F. saccharivorans, Candidatus accumlibacter (particularly sp. BA-91 or sp. SK-02), Eubacterium saburreum (particularly DSM 3986), Bacteroides fragiles (particularly strain S14), Camplyobacter fetus (particularly subspecies Fetus), Teredinibacter turnerae (particularly T8412), Woodsholea maritima, Desulfaculus baarsii (particularly DSM 2075), Azospirillum lipoferum (particularly 4B), Cellulomonospora bogoriensis (particularly 69B4), Micromonospora rosaria, Tolypothirx camplyonemoides, Oscillatoriales cyanobacterium, or Rivularia sp. (particularly PCC 7116), or a RT-Cas1-Cas2 complex originating thereof.

In certain embodiments, said inducible promoter sequence is operable in E. coli and is particularly selected from T7 promoter, lac promoter, tac promoter, Pₜₑₜ promoter, P_{C} promoter or P_{BAD} promoter.

In certain embodiments, the first and second transgene nucleic acid sequence are codon-optimized for expression in E. coli.

In certain embodiments, the bacterial cell additionally comprises
a fourth transgene nucleic acid sequence encoding a fourth transgene product, particularly a polypeptide sensor or a nucleic acid sensor, wherein said fourth transgene product is capable of modulating [directly or indirectly] the expression of a record gene inside the cell, and wherein such modulating the expression of said record gene is dependent on the presence or absence of an analyte molecule;
wherein said molecule of interest is selected from any molecule in the environment or inside of said cell, particularly a small molecule,
and wherein said record gene is not expressed under conditions in which no activated sensor is present.

A small molecule in the context of the invention is a molecule with a molecular weight of below 800 Da.

In certain embodiments, said fourth transgene product is a sensor which will be activated when contacted with a molecule of interest yielding an activated sensor, wherein said activated sensor will induce [directly or indirectly] the expression of a record gene inside the cell.

Direct modulation of gene expression is achieved when the fourth transgene product is a transcription factor, which is able to induce expression directly.

Indirect modulation of gene expression is achieved when the fourth transgene product is a receptor, which, when activated, starts a signal cascade leading to a modulation of gene expression.

### BRIEF DESCRIPTION OF THE FIGURES

FIG. 1 shows (A) Schematic representations of plasmids pFS_1595 and pAK0044, illustrating the arrangement of constitutive and regulated promoters (P_{cat}, P_{TetA}, P_{bla(trunc)}, and P_{KanR}) alongside the tetracycline-inducible repressor (TetR) and Kanamycin resistance cassette (KanR) coding sequences as well as terminators depicted as dark octagons. The constructs feature a series of operator sites and repressor binding regions designed for precise transcriptional control and vector counter-selection. Strategic placement of Type IIs restriction sites, specifically BbsI and BsaI, enables the modular modification of the vector backbone and the directional insertion of genetic parts via Golden Gate assembly. (B) Record-seq readout of RT-Cas1 orthologs and CRISPR array directionalities. Acquisition efficiency for forward (fw) and reverse complement (rc) directionality of each array are plotted in blue and orange, respectively. Values are genome-aligning spacers per million sequencing reads, n=1 biological sample.

FIG. 2 shows bar plot (mean±SEM, n=6) illustrating either (A) unique genome-aligning spacer counts, or (B) plasmid-aligning spacer counts for the indicated RT-Cas1:Cas2 constructs in terrific broth displayed on a log₁₀ scale. The constructs correspond to the expression of an RT-Cas1:Cas2 complex from the following organisms (from left to right): Psychrobacter lutiphocae, Blautia sp. Array1, Blautia sp. Array2, and Fusicatenibacter saccharivorans Array 2.

FIG. 3 shows histograms of the distribution of length (left column, 1 bp bins) and GC content (right column, 3% bins) for spacers acquired by the indicated RT-Cas1:Cas2 constructs characterizing the architectural preferences of each system, specifically Psychrobacter lutiphocae, Blautia sp. Array1, Blautia sp. Array2, and Fusicatenibacter saccharivorans Array 2. Data are presented as counts of unique spacers across the host genome.

FIG. 4 shows a heat map of deep mutational scanning results showing the effects of single amino-acid substitutions across FsRT-Cas1 on spacer acquisition efficiency relative to wild-type. Columns correspond to residue positions and rows correspond to substituted amino acids. Each cell reports the log₂ fold-change in spacer acquisition efficiency for the indicated substitution relative to wild-type. The wild-type residue at each position is indicated by an outlined cell. The cell shading represents the log₂ fold-change from lower values (lighter) to higher values (darker).

FIG. 5 shows a heat map of deep mutational scanning results showing the effects of single amino-acid substitutions across FsCas2 on spacer acquisition efficiency relative to wild-type. Columns correspond to residue positions and rows correspond to substituted amino acids. Each cell reports the log₂ fold-change in spacer acquisition efficiency for the indicated substitution relative to wild-type. The wild-type residue at each position is indicated by an outlined cell. The cell shading represents the log₂ fold-change from lower values (lighter) to higher values (darker).

FIG. 6 shows grouped box plot of validation results for engineered FsRT-Cas1-Cas2 variants designed from deep mutational scanning (DMS) data. Candidate substitutions from the DMS screen were combined to generate variants at increasing edit distance from wild type (x-axis), shown in two panels: A, combinatorial variants within domain (edit distance 1-4); B, further higher-order combinatorial variants (≥4 edits). The y-axis shows log₂ fold-change improvement in spacer acquisition efficiency relative to wild type. Each point represents an individual variant, with shading indicating the domain/type containing substituted residues (RT, Cas1, Cas2, or RT-Cas1), as in the legend. Boxplots summarize the distribution within each group, and the dashed horizontal line at log₂ fold-change=0 marks wild type-level efficiency.

FIG. 7 Illustrates multiple sequence alignments of RT-Cas1 and Cas2 amino acid sequences from diverse microbial species, showing conserved residue patterns across the functional domains. Conservation levels are indicated by shading as follows: black represents 100% identity; dark grey represents 80-100% conservation; light grey represents 60-80% conservation; and white represents <60% conservation. Sites that have been identified as essential are marked with * and those that can only be changed conservatively are marked with a #.

The alignments include sequences from the following organisms: Azospirillum lipoferum 4B; Bacteroides fragilis S14; Blautia sp. AM42-2; Campylobacter fetus subsp. fetus; Candidatus Accumulibacter sp. BA-91; Candidatus Accumulibacter sp. SK-02; Cellulomonas bogoriensis 69B4; Desulfarculus baarsii DSM 2075; Eubacterium saburreum DSM 3986; Fusicatenibacter saccharivorans; Micromonospora rosaria; Oscillatoriales cyanobacterium; Psychrobacter lutiphocae DSM 21542; Ruminococcus sp. TF08-4; Teredinibacter turnerae T8412; Tolypothrix campylonemoides; Vibrio sinaloensis strain T08; and Woodsholea maritima.

### EXAMPLES

The inventors hypothesized that direct CRISPR spacer acquisition from RNA could be leveraged to store transcriptional records in CRISPR arrays within living cells. Therefore, several orthologous RT-Cas1-containing CRISPR-Cas systems were characterized. The inventors identified one from Fusicatenibacter saccharivorans to be capable of acquiring RNA spacers heterologously in E. coli. Leveraging F. saccharivorans RT-Cas1 and Cas2 (FsRT-Cas1-Cas2) and developed Record-seq, a method enabling transcriptome-scale molecular recordings into populations of cells. Transcriptional events are recorded according to RNA abundance, stored in CRISPR arrays within DNA, and can be leveraged to describe continuous as well as transient complex cellular behaviors.

### CRISPR Spacer Acquisition by FsRT-Cas1-Cas2

The inventors set out to identify an RT-Cas1-Cas2 CRISPR acquisition complex with the ability to acquire spacers directly from RNA upon heterologous expression in E. coli. The inventors identified 121 RT-Cas1 orthologs (Table 1), and selected 14 representatives for functional characterization. The inventors overexpressed corresponding RT-Cas1 and Cas2 proteins from a plasmid additionally containing their predicted CRISPR array. Using a previously established spacer acquisition assay, the inventors discovered that the ortholog of F. saccharivorans actively acquired new spacers. The endogenous F. saccharivorans locus contains two CRISPR arrays and the inventors observed novel spacers derived from the overexpression plasmid as well as the E. coli genome were acquired into either.

### Selective Amplification of Expanded CRISPR Arrays

Using the previously established spacer acquisition assay, the inventors obtained approximately 1300 newly acquired spacers per 1 million deep sequencing reads for FsRT-Cas1-Cas2. To improve detection of novel spacers, the inventors developed Selective amplification of expanded CRISPR arrays (SENECA), a method to selectively amplify CRISPR arrays that acquired new spacers. A typical SENECA-assisted Record-seq experiment uses an input of ~180 ng of plasmid DNA extracted from an overnight culture of E. coli overexpressing FsRT-Cas1-Cas2, and yields 950,000 total spacers aligning to the plasmid or host genome for every 1 million sequencing reads. This marks an improvement of several thousand-fold compared to recent reports. Using Record-seq, the inventors readily demonstrated in vivo activity of FsRT-Cas1-Cas2 in various E. coli strains and throughout growth phases.

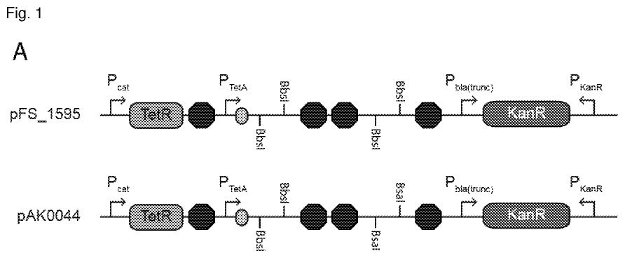

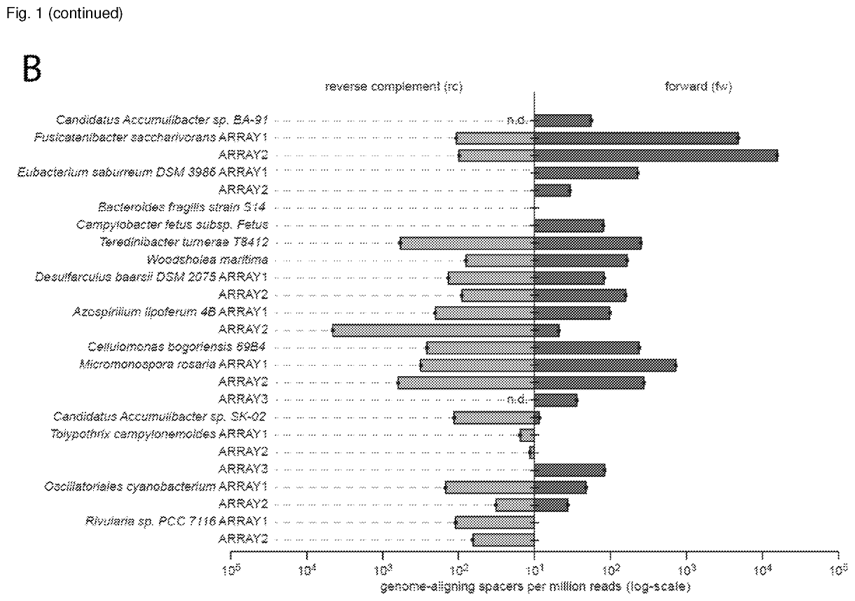

The inventors then employed Record-seq to rescreen their initial selection of RT-Cas1 orthologs. Furthermore, the inventors included all potential CRISPR arrays present in their endogenous loci in both possible directionalities in order to overcome the challenges associated with predicting these apriori. Due to the improved sensitivity of Record-seq compared to the classic readout, the inventors readily detected newly acquired spacers for the majority of orthologs upon RT-Cas1-Cas2 expression (FIG. 1, panel B). Only a few orthologs exhibited a preferred directionality of the CRISPR array (i.e., specificity for an upstream leader sequence). Consistent with the classic readout, FsRT-Cas1-Cas2 outperformed all other orthologs in terms of spacer acquisition efficiency and was chosen for further characterization. The concepts employed by Record-seq may also be applied to characterize spacer acquisition in other CRISPR-Cas systems that have been intractable due to low spacer acquisition efficiencies.

### Characteristics of FsRT-Cas1-Cas2 Spacer Acquisition

In order to better understand the properties of FsRT-Cas1-Cas2, the inventors extensively characterized newly acquired spacers by performing Record-seq on populations of E. coli overexpressing FsRT-Cas1-Cas2. The inventors observed that genome-aligning spacers were preferentially acquired with a specific ‘antisense’ orientation, whereby spacers were complementary to the originating RNA. The median spacer length was 39 bp, with a distribution biased towards longer lengths. The median GC content was 36%, showing a strong bias towards AT-rich spacers. In line with previously described Type III CRISPR systems, the inventors did not find a sequence preference within or adjacent to newly adapted spacers acquired from either plasmid or genome, implying that the FsRT-Cas1-Cas2 complex exhibits no protospacer adjacent motif (PAM). While observing spacer alignments to the E. coli genome the inventors noted that many coverage peaks were located near the termini of genes. Consistent with this observation, the inventors found that at the genome-wide level, most spacers were derived from the 5′, and to a lesser extent, 3′ ends of genes. This finding raised the possibility that the apparent bias towards AT-rich spacers might be caused by the AT-richness of RNA ends in E. coli, however the bias towards AT-rich spacers persisted when only considering spacers derived from within the gene body (FIG. 1, panel B). The inventors directly compared SENECA with the classic spacer readout to determine whether SENECA introduces additional biases but found no major differences. Taken together, these results reflect a process by which FsRT-Cas1-Cas2 selects AT-rich spacers based sequences related to the beginning or end of a gene, such as the ends of an RNA molecule.

FsRT-Cas1-Cas2 Acquires Spacers Directly from RNA

To determine whether FsRT-Cas1-Cas2 acquires spacers directly from RNA, the inventors utilized a self-splicing td group I intron. This intron is a functional ribozyme, catalyzing its own excision from the pre-mRNA, resulting in a characteristic splice junction that is not present at the DNA-level. The inventors constructed three intron-interrupted constructs based on genes that were highly sampled by spacers, namely cspA, rpoS and argR. Upon expression of these constructs followed by Record-seq the inventors observed unique spacers spanning the splice junctions. To exclude the possibility that splice junction-containing spacers were acquired from extended complementary DNA copies generated through unspecific RT activity in E. coli, the inventors performed targeted deep sequencing on genomic DNA extracted from td intron construct-expressing cultures showing that the splice junction was absent at the DNA-level. Importantly, these results do not exclude the possibility of spacer acquisition from DNA. Taken together, FsRT-Cas1-Cas2 facilitates CRISPR spacer acquisition from RNA heterologously in E. coli.

To further validate this finding, the inventors utilized the Enterobacteria phage MS2. MS2 phages exist as both sense and antisense single-stranded RNAs during their lifecycle but have no DNA intermediates. Given that MS2 phages require the F pilus for cell entry, which is missing in E. coli BL21(DE3) cells, the inventors turned to the E. coli K12 strain NovaBlue(DE3). Upon infection of FsRT-Cas1-Cas2 expressing cells with MS2 phage, the inventors could readily observe novel MS2-aligning spacers sampled from throughout the MS2 genome. The MS2-aligning spacers shared no sequence similarity with the plasmid or host genome, confirming their specificity. In sum, FsRT-Cas1-Cas2 enables spacer acquisition directly from a foreign RNA, thereby providing a molecular memory of an invading virus.

### Recording of Arbitrary Transcripts Using Record-Seq

To assess the potential of FsRT-Cas1-Cas2 for quantitatively recording transcriptional events, the inventors utilized an inducible expression system to directly determine whether spacers were being acquired according to RNA abundance. The corresponding constructs contained super-folder GFP (sfGFP) or Renilla luciferase (Rluc) genes under transcriptional control of the anhydrotetracycline (aTc)-inducible P_{tetA} promoter. The inventors introduced these into E. coli cultured in increasing levels of aTc and subsequently harvested both total RNA and plasmid DNA for qRT-PCR and Record-seq, respectively. The inventors observed that upon increasing induction of sfGFPor Rluc there was a concordant dose-dependent increase in the coverage of spacers aligning to the respective coding sequence. The inventors quantified this response and observed a linear relationship (R² value of 0.97) between spacer counts and absolute mRNA copy number as well as aTc concentration in the media. Furthermore, sfGFP-aligning spacers were readily detected against the backdrop of genome-aligning spacers by almost an order of magnitude, which is in line with using a strong synthetic inducible promoter such at P_{tetA}. Importantly, spacers aligning to the constitutively expressed KanR gene were not dependent on the aTc concentration.

To further generalize these findings, the inventors evaluated a second inducible expression system, placing the firefly luciferase (Fluc) gene downstream of the 3-oxohexanoyl-homoserine lactone (30C6-HSL)-inducible P_{LuxR} promoter. Induction led to a 4-fold increase in F/uc-aligning spacers. Furthermore, combining both the aTc-inducible P_{tetA} and the 30C6-HSL-inducible P_{LuxR} transcription system enabled orthogonal recording of two independent stimuli in parallel. This suggests that Record-seq is compatible with seemingly any inducible expression system, thereby enabling recording of multiple orthogonal sets of defined stimuli within a population of living cells. Taken together, these results show that CRISPR spacer acquisition from RNA can generate a quantifiable record of cumulative transcript abundance, and also that the transcriptional records are efficiently retrieved using standard molecular and sequencing methods.

### Record-Seq Shows Cumulatively Highly Expressed Genesa

Considering that FsRT-Cas1-Cas2 acquired spacers directly from RNA in an abundance-dependent manner, the inventors investigated whether this could enable quantification of the cumulative cellular transcriptome. The inventors harvested both plasmid DNA for Record-seq and total RNA for RNA-seq E. coli cultures overexpressing FsRT-Cas1-Cas2. First, the inventors confirmed the reproducibility of Record-seq between biological replicates (Pearson Correlation=0.996 to 0.999 and R²=0.560 to 0.618), and then assessed the influence of gene expression on spacer acquisition. The FsRT-Cas1-Cas2 spacers showed a strong bias towards highly transcribed genes and correlated with RNA-seq-based gene expression values transcriptome-wide at various growth stages. While certain CRISPR-Cas subtypes possess active mechanisms for preferentially acquiring plasmid-derived spacers, the inventors did not observe the same after accounting for the high expression level of these genes. Taken together, spacers are systematically acquired from highly transcribed genes, and represent cumulative transcript expression.

### Transcriptome-Scale Recording Reveals Cell Behaviors

To determine whether Record-seq could be used to record and describe complex cellular behaviors, the inventors turned to the well-studied oxidative stress and acid stress responses in E. coli. The inventors performed Record-seq on oxidative and acid stress stimulated FsRT-Cas1-Cas2 expressing cultures and analyzed cumulative expression counts using unsupervised hierarchical clustering as well as principal component analysis (PCA). Both approaches were successful in distinguishing treatment conditions, suggesting that Record-seq captured the differential molecular histories. To identify the cumulatively differentially expressed genes the inventors leveraged standard differential expression (DE) analysis tools developed for RNA sequencing. To overcome specific biases and assumptions of individual tools, the inventors utilized three complementary tools, namely DESeq2, edgeR, and baySeq. After identifying DE genes with each tool, the inventors generated a set of signature genes for each stimulus based on the union of the top 20 DE genes from each analysis, which the inventors hierarchically clustered and plotted along with their expression values. Among the signature genes the inventors identified several that were expected to dominate the cellular responses for each stimulus. The inventors investigated the minimum number of cells required for assessing complex cellular behaviors by Record-seq, finding that 8.8×10⁶ cells are sufficient to appropriately classify treatment conditions. In sum, these data support the notion that the RNA-derived spacers stored within CRISPR arrays can be utilized to reconstruct the transcriptional response underlying a complex cellular behavior.

### Vector Architecture and Expression System

The present invention utilizes a modular plasmid architecture for the expression and characterization of CRISPR-associated proteins, including RT-Cas1, RT, Cas1, and Cas2 orthologs, alongside their corresponding leader and direct repeat (DR) sequences. As illustrated in FIG. 1, the protein coding sequences were integrated into a dual-BbsI restriction site locus positioned downstream of an anhydrotetracycline (aTc)-inducible promoter. To evaluate functional activity, the corresponding leader:DR arrays were integrated into BbsI or BsaI sites located between two transcriptional terminators downstream of the protein expression cassette. This configuration ensures tightly regulated expression for assessing spacer acquisition efficiency in Escherichia coli.

### Identification of Novel Functional Ortholoqs

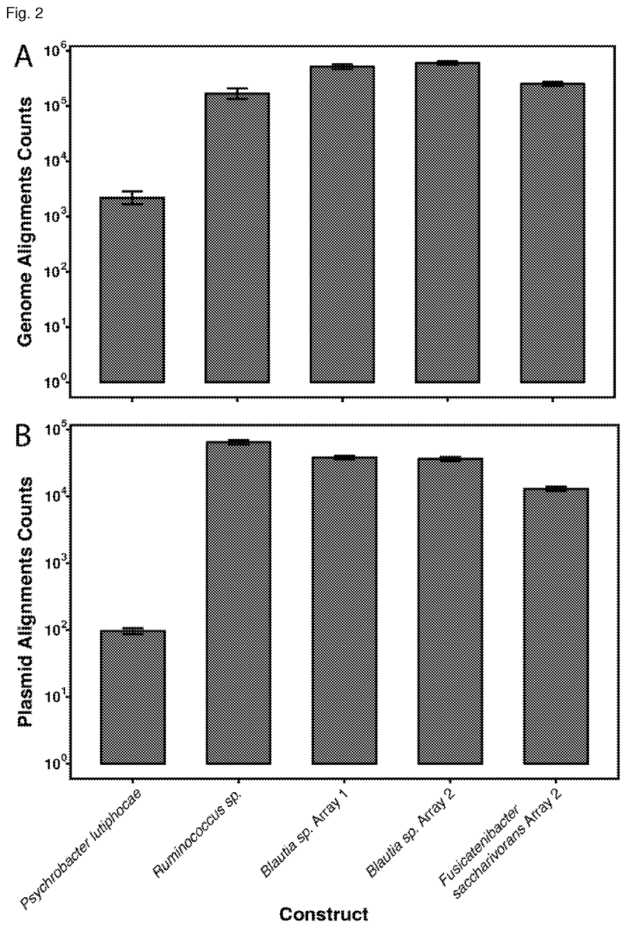

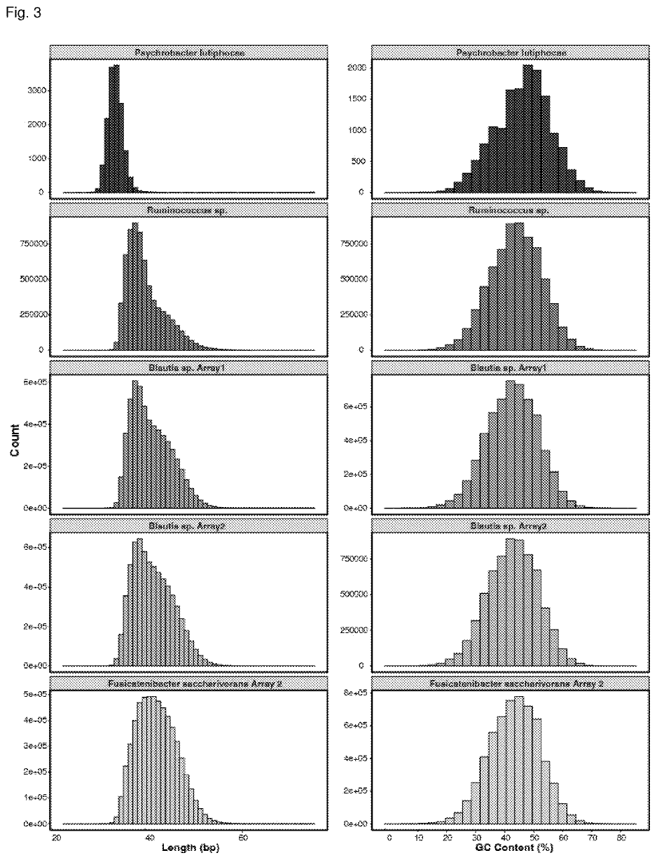

Using the SENECA screening platform, we evaluated the efficiency of spacer acquisition across a library of previously characterized and newly identified protein variants. This screen identified three novel orthologs that exhibit robust activity, characterized by a significant frequency of spacer acquisition events originating from the E. coli genome and subsequent integration into the cognate leader:DR array. As shown in FIG. 2, these systems demonstrate high-efficiency acquisition in Terrific Broth. Furthermore, analysis of the acquired spacers reveals distinct biophysical profiles; specifically, certain orthologs exhibit variations in spacer length and GC content distribution compared to the Fusicatenibacter saccharivorans reference system (FIG. 3).

### Deep Mutational Scanning (DMS) and Functional Mapping

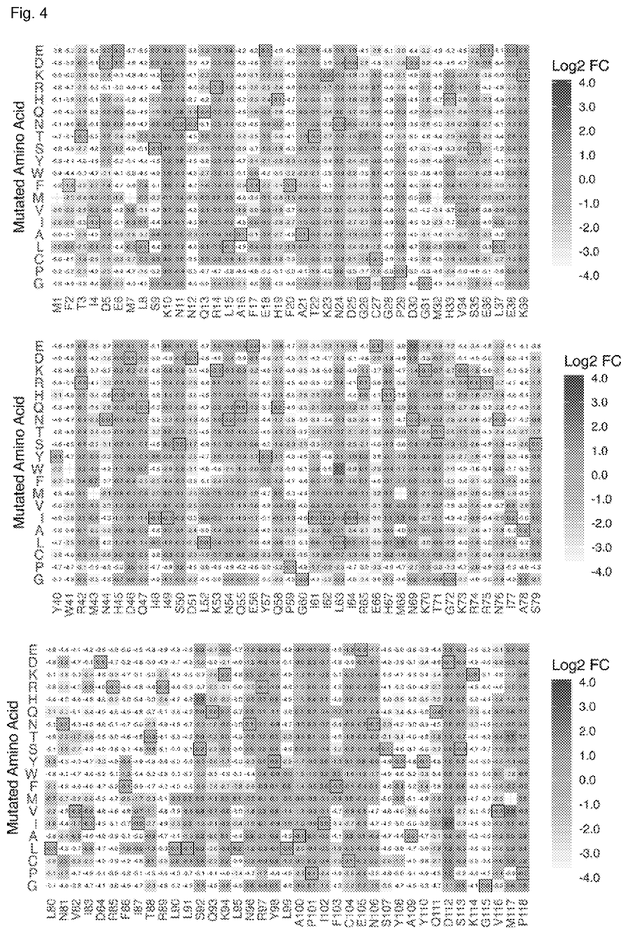

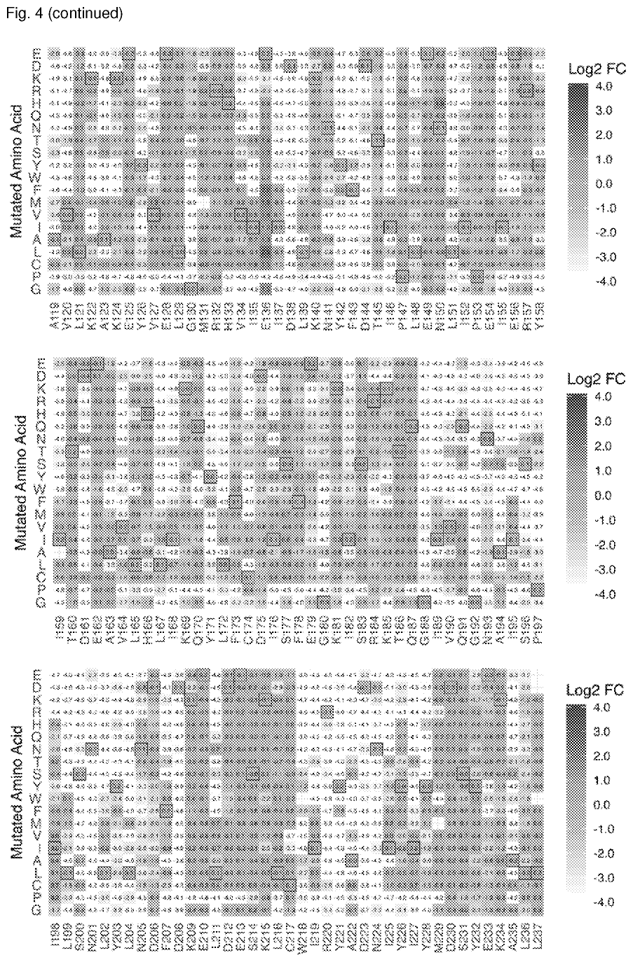

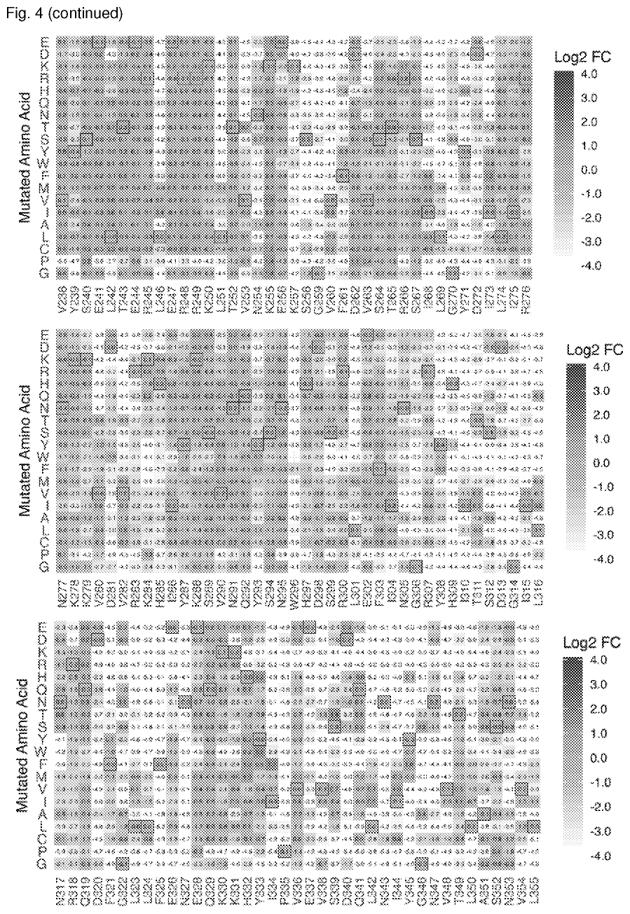

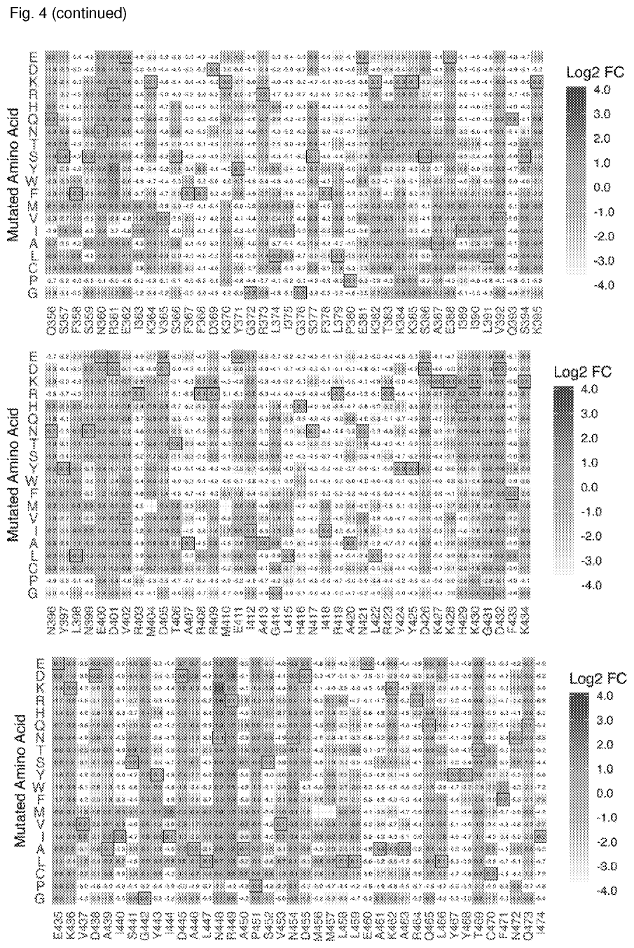

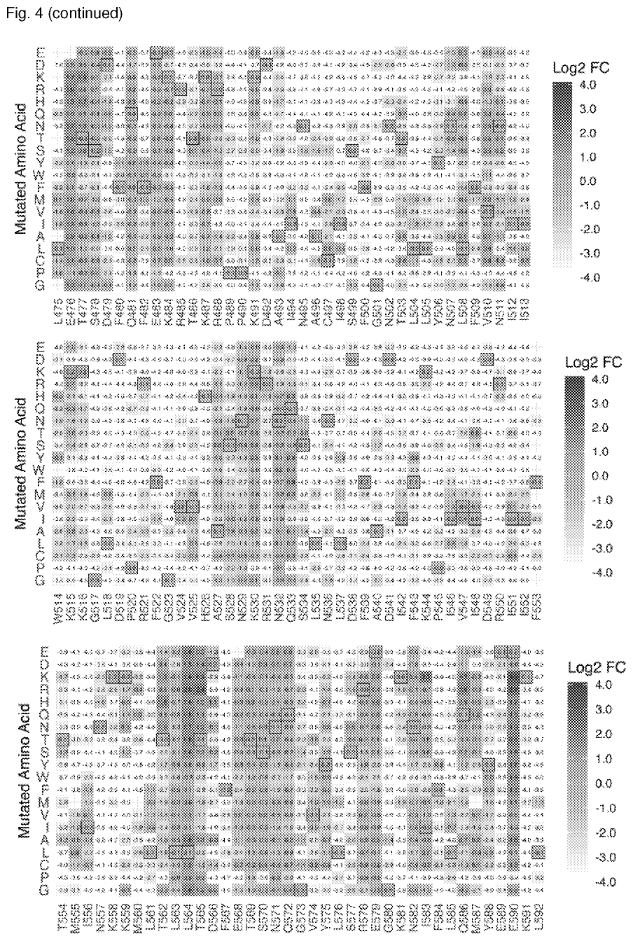

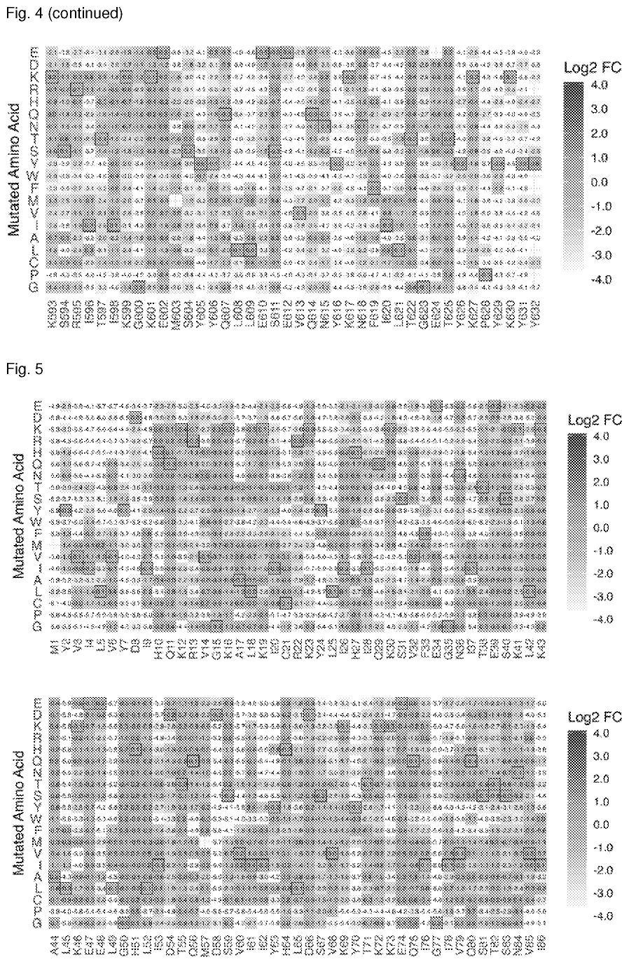

To define the sequence-function relationship of the Fusicatenibacter saccharivorans RT-Cas1 and Cas2 proteins, we performed a comprehensive Deep Mutational Scan (DMS) targeting single amino acid substitutions. The resulting functional maps, depicted in FIG. 4 and FIG. 5, identify “mutational hotspots” where residues are either essential for catalytic activity or highly tolerant to substitution. Quantitative analysis indicates that approximately 50% of the amino acid positions are interchangeable—allowing for substitution without a deleterious effect on functionality—as detailed in Table 2. These DMS-derived findings were validated through arrayed-format testing of select variants, with results summarized in Table 3.

### Combinatorial Engineering and Enhanced Efficiency

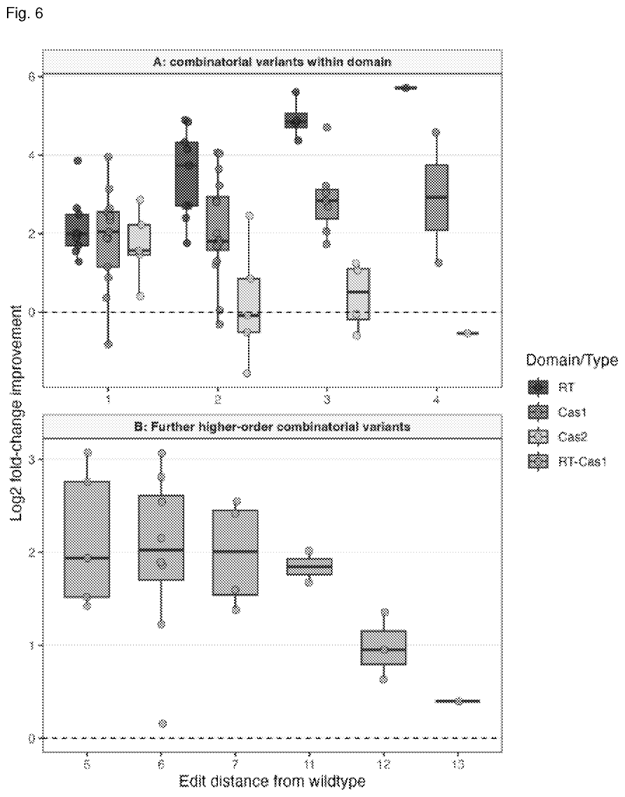

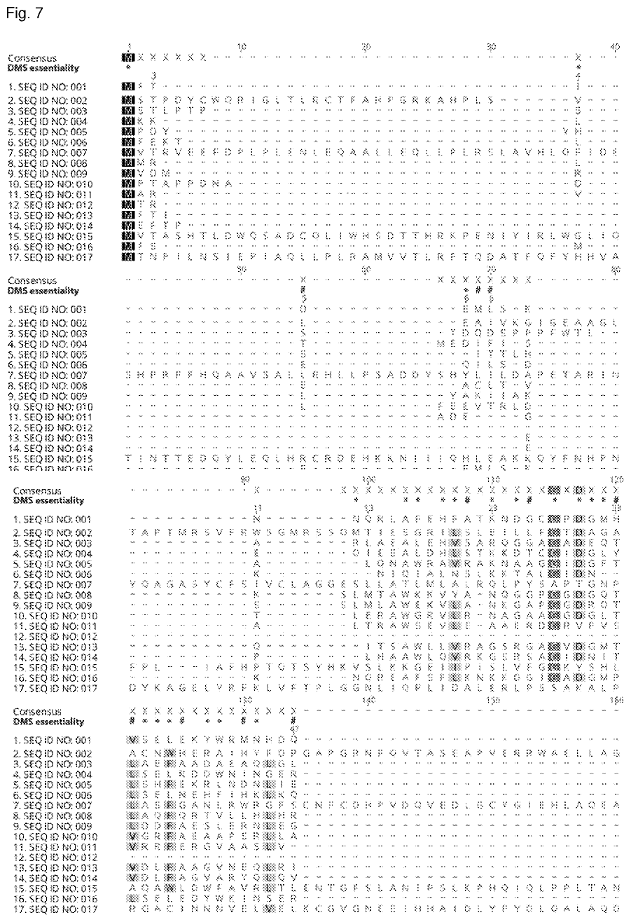

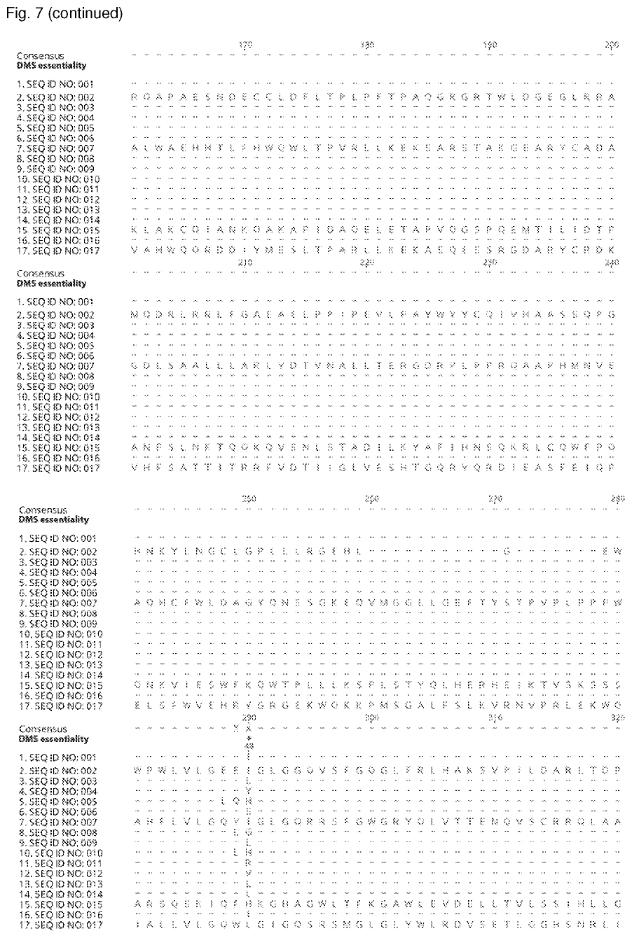

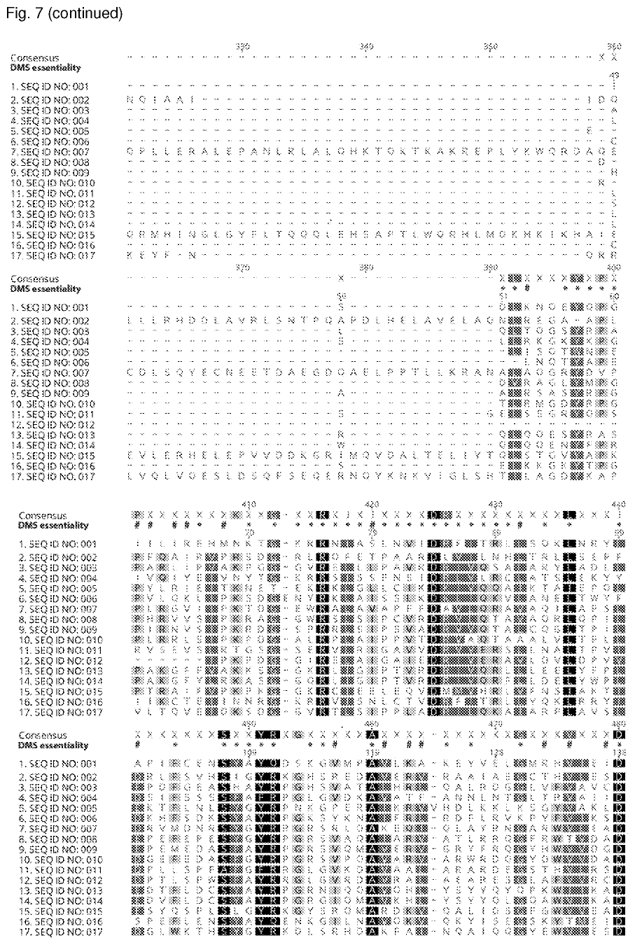

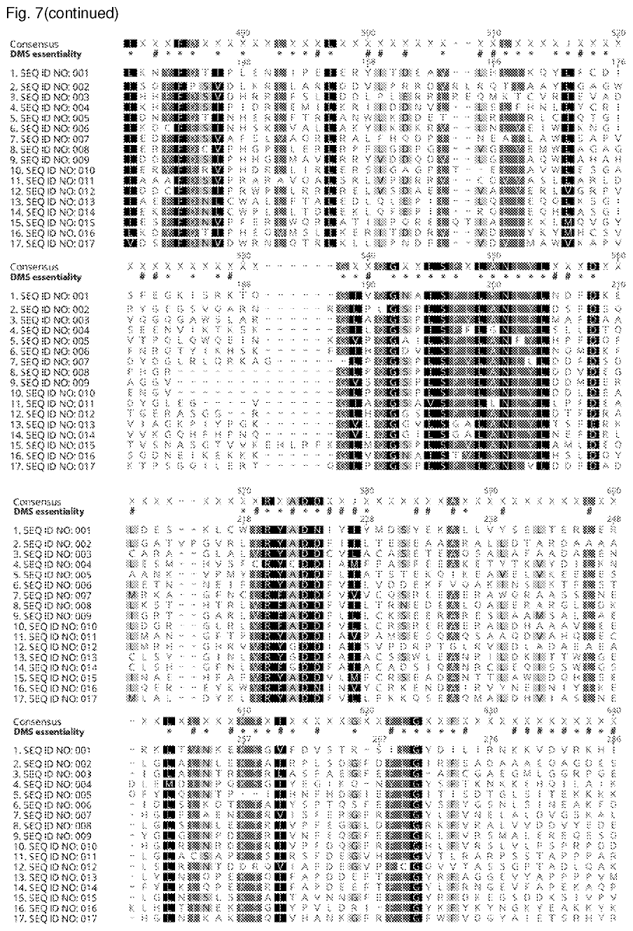

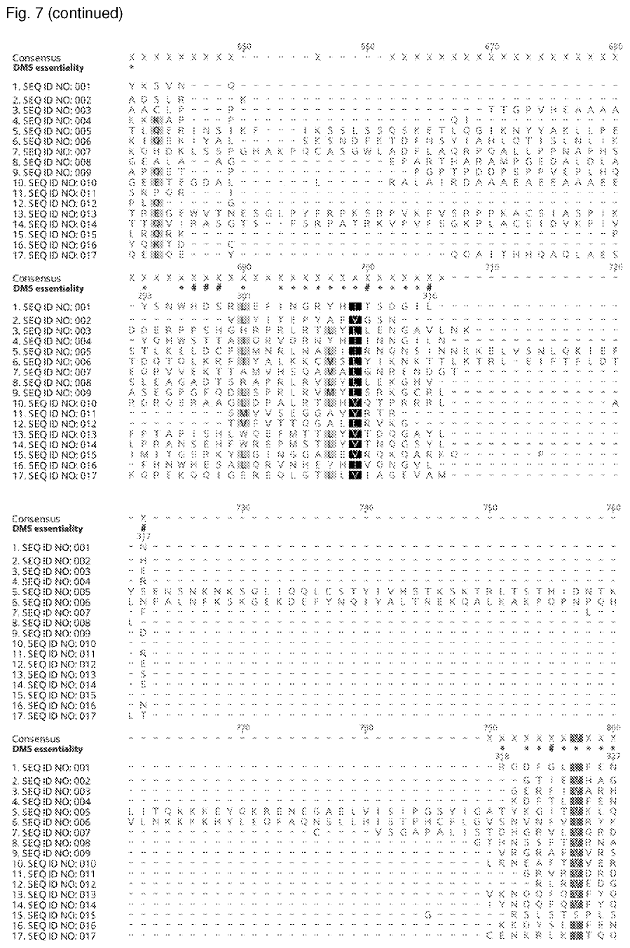

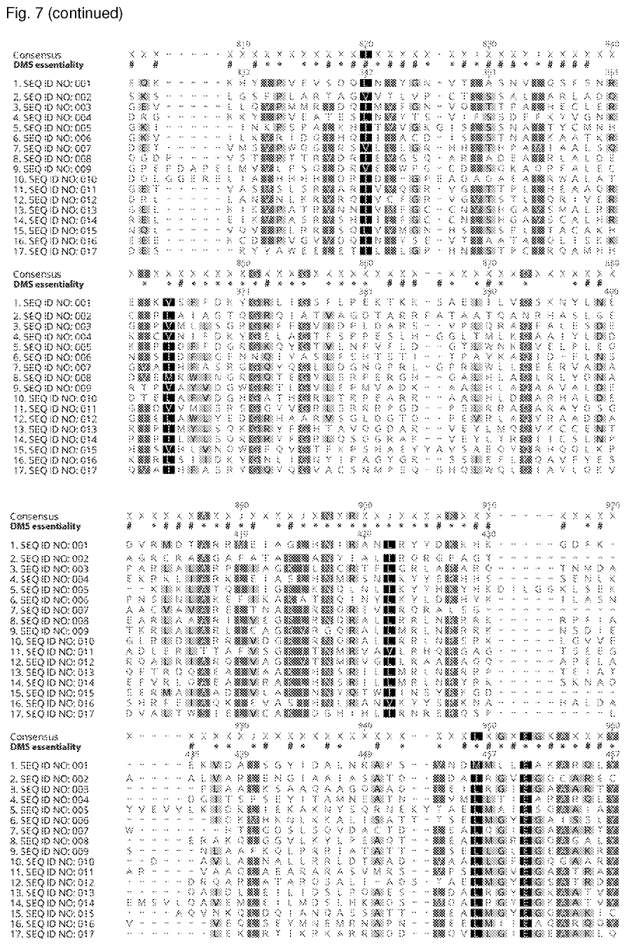

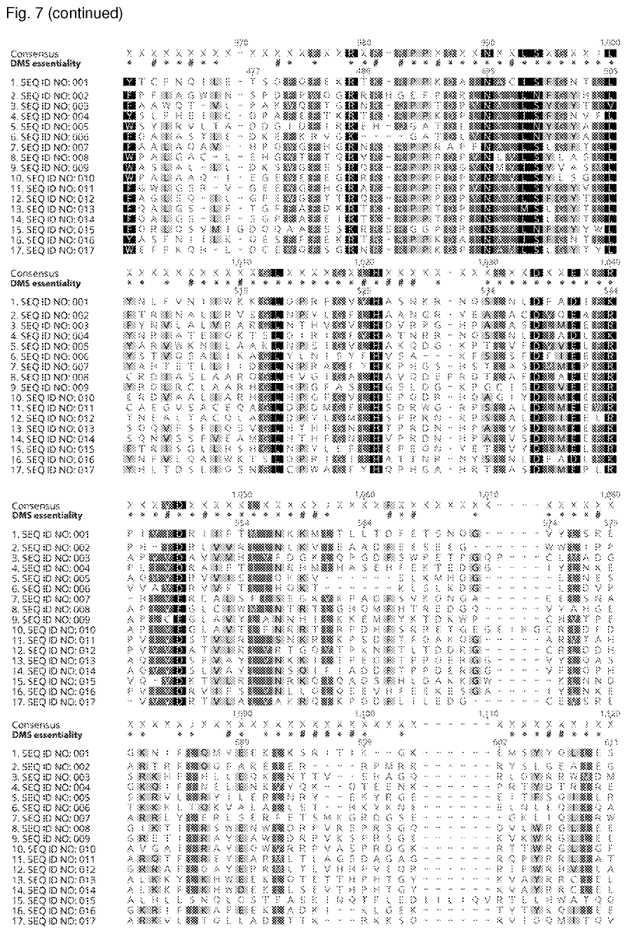

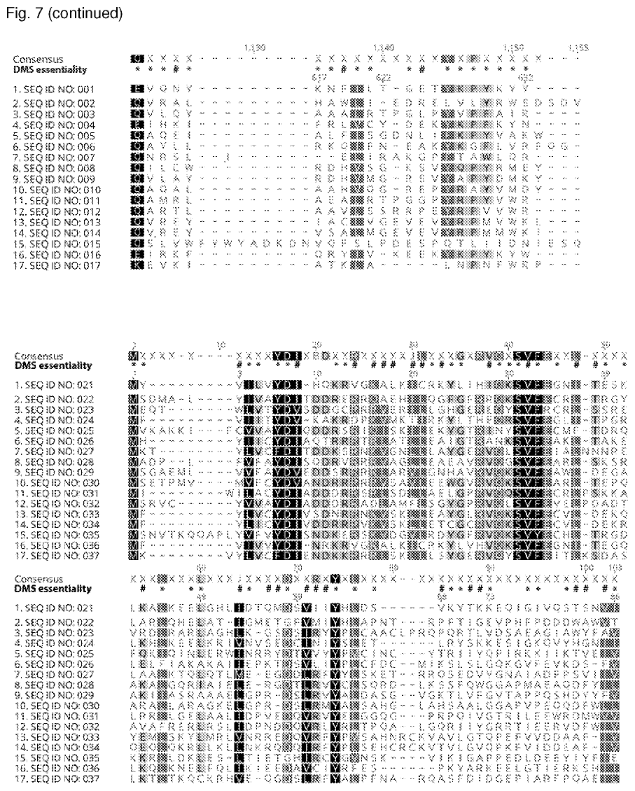

Building upon the DMS data, we designed multi-mutant variants by combining substitutions that independently correlated with increased spacer acquisition counts. Validation experiments demonstrate that the accumulation of beneficial or neutral mutations does not obstruct enzymatic function. Notably, combinatorial variants containing up to 12 distinct mutations maintained or improved acquisition efficiency relative to the wild-type enzyme (FIG. 6). These results confirm that utilizing amino acids identified as non-deleterious in the DMS allows for extensive protein engineering without compromising system performance.

### Discussion

Here, the inventors describe Record-seq, a technology to encode transcriptome-scale events into DNA and assess the cumulative gene expression of populations of cells. The inventors demonstrate its potential by recording specific and complex transcriptional information. First, to improve upon existing spacer readout methods the inventors developed SENECA, resulting in a several thousand-fold improvement of spacer detection efficiency compared to recent reports, thereby enabling in-depth characterization of FsRT-Cas1-Cas2 and its application as a molecular recorder. The inventors' results suggest that RNA-derived spacers are preferentially acquired from the ends of abundant transcripts from AT-rich regions with no PAM, and are broadly sampled at transcriptome-scale, enabling the parallelized quantification of cumulative transcript expression.

In a set of experiments, the inventors show that upon increasing induction of arbitrary sequences, spacers are acquired in an orthogonal, dose-dependent manner and highly correlate with the absolute mRNA copy number in the cell, thus demonstrating that the molecular record faithfully recapitulates the initial stimulus in a predictable way. This also paves the way for increasingly multiplexed and orthogonal molecular recording devices. Upon inducing complex cellular behaviors, Record-seq provides a meaningful transcriptome-scale record of molecular events, which exceeds the capabilities of current molecular recording technologies that only record specific stimuli. Finally, the inventors use Record-seq to elucidate dose-dependent features of the complex cellular response to the bacteriostatic herbicide paraquat, and demonstrate that Record-seq, but not RNA-seq, is capable of recording transient paraquat stimulation.

Although additional work will greatly improve the capacity of Record-seq to encode richer and more dynamic expression and lineage information within fewer cells, the inventors' proof-of-principle experiments introduce a powerful tool to record transcriptome-scale events permanently in DNA for later reconstructing complex molecular histories from populations of cells. The inventors show that the recorded transcriptional histories reflect the underlying gene expression changes and could therefore be used to interrogate biological or disease processes. In the long term, the inventors envision that CRISPR spacer acquisition components could be introduced into other cell types to record the molecular sequence of events, and lineage path, that gives rise to particular cell behaviors, cell states and types.

### Methods

### Ortholog Discovery Pipeline

The protein sequence of Arthrospira platensis RT-Cas1 (WP_006620498) was used as a seed sequence, and a JACKHMMER search was run against all NCBI Non-redundant protein sequences using HMMER v3.1b2 (E-value cutoff of 1E-05). Proteins with both Cas1 and RT domains were subsequently identified using HMMSCAN (E-value cutoff of 1E-05). Genome sequence information for the candidate proteins were retrieved and further inspected for the presence of RT-Cas1, Cas2, and a CRISPR array using CRISPRdetect v2.0, CRISPRone, and HMMSCAN. From 121 candidate proteins, 14 CRISPR loci were selected and subsequently aligned using MUSCLE v3.8.31 to identify candidate domains and catalytic residues. Genetic distances were computed using the Jukes-Cantor method and a phylogenetic tree was built using the Nearest-Neighbour method.

### Bacterial Strains and Culture Conditions

Escherichia coli strains used in this study were Stbl3 (Thermo Fisher Scientific) for cloning purposes as well as BL21(DE3) Gold (Agilent Technologies), BL21AI (Invitrogen) and NovaBlue(DE3) (EMD Millipore) as a K12 strain for acquisition assays. All strains were made competent using the Mix & Go E. coli Transformation Kit & Buffer Set (Zymo Research) following the manufacturer's protocol with growth in ZymoBroth at 19° C. directly from fresh colonies. After transformation, cells were grown at 37° C. on lysogenic broth (LB) (Difco) 1.5% agar plates containing 50 μg/mL kanamycin and 1% glucose (w/v) to reduce background expression from the T7lac system. Liquid cultures for plasmid isolation were grown in TB media (24 g/L yeast extract, 20 g/L tryptone, 4 mL/L glycerol, 17 mM KH₂PO₄, 72 mM K₂HPO₄) containing 1% glucose (w/v).

Generation of Golden Gate Compatible pET30 Overexpression Vector

All standard PCRs for cloning were performed using Phusion Flash High-Fidelity PCR Master Mix (Thermo Scientific) or KAPA HiFi HotStart ReadyMix (Roche), oligonucleotides and gBlocks were ordered from Integrated DNA technologies. pET30b(+) (kind gift from Markus Jeschek) was PCR amplified as five fragments using primers FS_151/FS_152, FS_153/FS_154, FS_155/FS_156, FS_157/FS_158, FS_159/FS_160, respectively in order to remove the five undesired BbsI restriction sites present in the backbone. The resulting PCR fragments were assembled using 2×HiFi DNA Assembly Mastermix (NEB), yielding pFS_0012. Subsequently, oligos FS_380 and FS_381 were annealed to generate a double stranded DNA (dsDNA) fragment encoding the T7 terminator and cloned into pFS_0012 using XhoI/CsiI, yielding pFS_0013—a pET30 derived overexpression vector harboring two Golden Gate cloning sites and thus facilitating parallel cloning of RT-Cas1, Cas2 as well as a corresponding CRISPR array. Nucleotide sequences of all RT-Cas1 and Cas2 orthologs tested in this study along with their corresponding CRISPR arrays are listed under Sequences.

### Golden Gate Assembly of RT-Cas1-Cas2 Overexpression Vectors for Ortholog Screen

RT-Cas1, Cas2 and CRISPR array sequences were ordered from Twist Biosciences and Genscript. Putative CRISPR arrays were ordered as sequences consisting of the leader sequence followed, by DR-nativespacer1-DR-nativespacer2-DR. Furthermore, each fragment was flanked by BbsI restriction sites generating overhangs facilitating Golden Gate Assembly into pFS_0013. Briefly, 40 fmol per fragment (RT-Cas1, Cas2, corresponding CRISPR array, pFS_0013 acceptor vector), 1 μL ATP/DTT mix (10 mM each), 0.25 μL T7 DNA Ligase (Enzymatics), 0.75 μL BpiI (Thermo Scientific), 1 μL buffer green up to 10 μL with PCR grade H₂O were subjected to 99 cycles of 37° C. for 3 min, 16° C. for 5 min, followed by 80° C. for 10 min. Subsequently, 5 μL of this mixture were transformed into 50 μL Stbl3 cells and recovered in SOC media for 30 min at 37° C., 1000 rpm before spreading on plates.

### Spacer Acquisition

Acquisition assays were performed at 37° C., 300 rpm in bacterial culture tubes containing 3 mL of TB media supplied with 100 μM isopropyl-β-D-thiogalactopyranoside (IPTG) (Sigma Aldrich) and for BL21(DE3) Gold and NovaBlue(DE3). For E. coli BL21AI, L-(+)-arabinose (Sigma Aldrich) was additionally added to 0.2% (w/v). Each culture was inoculated with 2 colonies of bacteria stored no longer than 14 days at 4° C. upon transformation and overnight growth at 37° C. When cultures reached saturation (typically 12-14 h post inoculation), 2 mL of bacterial culture were harvested and plasmids containing CRISPR arrays were isolated by standard plasmid Mini-Prep procedures to serve as a template for preparation of deep sequencing libraries.

### Amplification of CRISPR Arrays for Classical Acquisition Readout by Deep Sequencing

Leader proximal spacers were PCR amplified from 3 ng of plasmid DNA per μL of PCR reaction using NEBNext High-Fidelity 2×PCR Master Mix (NEB) with a forward primer binding in the leader sequence of the respective CRISPR array and a reverse primer binding in the first native spacer. For each biological replicate, 12 individual PCR reactions of 10 μL were performed with an extension time of 15 sec for 16 cycles. The individual 10-μL reactions belonging to the same biological sample were then pooled, and residual primers removed using homemade AMPure beads at a PCR to bead ratio of 1:1.5 (v/v) eluting the PCR product in 60 μL of buffer TE. Subsequently, 500 ng of first round PCR product per biological sample was run on a 3% LAB agarose gel (300V, 55 min, cooling the gel-chamber in an ice-water bath during the run) and purified by blind excision of gel slices at 211 to 300 bp, avoiding the prominent DNA band corresponding to PCR products of the unexpanded array (i.e. no acquisition of novel spacers). Amplicons were then purified from the gel slices using the QIAquick Gel Extraction Kit (QIAGEN) and eluted into 22 μL of buffer EB. Illumina sequencing adaptors and indices were appended in a second round of PCR, using 6 μL of gel purified input DNA as a template in a 20 μL PCR reaction with universal second round deep sequencing primers attaching P5 and P7 handles for binding of PCR products to the flow cell in deep sequencing as well as barcoding the samples with (N)₈ barcodes corresponding to Illumina TruSeq HT indices. After this second round of PCR, products were purified using the QIAquick PCR Purification Kit (QIAGEN) and eluted in 22 μL buffer EB. Samples were then pooled and subjected to another round of gel purification using the same parameters as described above, this time excising products in the range of 280 to 350 bp.

### Selective Amplification of ExpaNdEd Crispr Arrays (SENECA)

FsCRISPRArray2 was amplified from pFS_160 using FS_871/FS_904, generating a minimal Fs CRISPR Array consisting of the leader sequence and a single DR followed by a FaqI restriction site (CTTCAG) on the bottom strand resulting in plasmid pFS_0235 as our standard recording plasmid. This plasmid was transformed into chemocompetent BL21(DE3) Gold bacteria or NovaBlue(DE3) (EMD Millipore) and subjected to spacer acquisition as described above. Following plasmid extraction and quantification using Quant-IT PicoGreen dsDNA Assay Kit (Thermo Scientific) read out with a Tecan M1000 Pro Microplate reader, plasmid DNA was subjected to SENECA-adapter ligation in a Golden Gate reaction. Oligonucleotides FS_0963/FS_0964 were annealed (2.5 μL each of 100 μM oligo, 5 μL NEBuffer 2 (NEB), 40 μL PCR grade H₂O), by heating to 95° C. for 5 min and cooling to 20° C. at 0.12° C./sec. Annealed oligos were diluted 1:100 in TE buffer. Next, 40 fmols of plasmid DNA (180.3 ng for pFS_0235), 0.25 μL T7 Ligase (Enzymatics), 1 μL FastDigest FaqI 0.5 μL of 20×SAM, 1 mM ATP, 1 mM DTT (all Thermo Scientific), 1 μL of annealed, diluted oligonucleotides FS_0963/FS_0964 in 10 μL total Volume were subjected to 99 cycles of 3 min 37° C., 3 min 20° C. followed by 15 min at 55° C. First round deep sequencing PCR was performed using NEBNext High-Fidelity 2×PCR Master Mix (NEB) (forward primers: FS_0968 to FS 0974, reverse primer: FS_0911). For each biosample one 30 μL reaction containing 10.38 μL of adapter ligated plasmid DNA were performed (98° C. for 30 s; 22 cycles at 98° C. for 10 s, 57° C. for 30 s and 72° C. for 20 s followed by 72° C. for 5 min), pooled and purified by magnetic beads (GE Healthcare) at a PCR to bead ratio of 1:1.6 (v/v) recovering the PCR product in 25 μL TE buffer (Primer Design Note 3 for details on primer design). Illumina sequencing adaptors and indices were appended in a second round of PCR (98° C. for 30 s, 8 cycles of 98° C. for 10 s, 65° C. for 30 s and 72° C. for 30 s, and 72° C. for 5 min) using 5 μL of first round PCR product as input in a 20 μL reaction. Samples were pooled, desalted using the QIAquick PCR Purification Kit (QIAGEN) and size selected on a E-Gel EX Agarose Gels, 2% (Thermo Scientific), loading 200-500 ng of DNA per lane, extracted using the QIAquick Gel Extraction Kit and subjected to deep sequencing on Illumina MiSeq or NextSeq500 platforms using the MiSeq Reagent Kit v3 (150-cycle) or NextSeq 500/550 Mid/High Output v2 kit (150 cycles) (both Illumina), respectively. Libraries were loaded at a concentration of 1.4 to 1.6 μM as determined by qPCR using the KAPA Library Quantification Kit for Illumina® Platforms (Roche). PhiX was included at 5-10%.

### SENECA Based Ortholog Screen

For the SENECA based CRISPR array directionality screen, putative CRISPR arrays were extracted from genomic sequences, assuming a standard leader length of 150 nt followed by a single DR. The FaqI restriction site required for SENECA was appended downstream of the DR and sequences were flanked by universal adapters for amplification and cloning. The final array sequences including these features are depicted under Sequences 2 and were ordered from Twist Biosciences as linear DNA fragments. These were PCR amplified using primers FS_1406/FS_1407 and cloned into CsiI/NotI-digested plasmids containing their respective RT-Cas1-Cas2 ortholog using HiFi DNA Assembly (NEB). Upon transformation into E. coli BL21(DE3), these constructs were subjected to the standard spacer acquisition assay in TB media. Plasmid DNA was extracted and subjected to SENECA adapter ligation. Following adapter ligation, a single 140 μL 1^{st} round PCR reaction was prepared for each ortholog using NEBNext High-Fidelity 2×PCR Master Mix and containing the entire 20 μL SENECA adapter ligation as a template. The 140 μL PCR reaction was split into 12 reactions of 11 μL along the row of a 96-well plate. This plate was subjected to a gradient PCR (53 to 68° C. in an Eppendorf Mastercycler Gradient). This procedure was chosen because SENECA leverages the fact that a DR matching primer will only bind to the full DR resulting from an acquisition event but not the truncated parental DR at a unique annealing temperature. By splitting the PCR reaction and subjecting it to a temperature gradient, it is ensured that without a prior knowledge, at least one of the 12 reactions is subjected to the annealing temperature at which selective amplification of expanded CRISPR arrays occurs. PCR was performed for 30 cycles upon which, the 12 reactions performed along the temperature gradient were pooled again and purified using 1.85×Ampure beads and eluted in 25 μL TE buffer. Five μL of this elution were used as a template for a standard 20 μL second round PCR at 65° C. annealing temperature for 12 cycles as described above. Subsequently, PCR products were purified using 2.2×Ampure beads, eluted into 22 μL TE buffer, size selected as described in the standard SENECA protocol (E-Gel Ex 2%, followed by gel extraction) and subjected to deep sequencing.

### Deep Sequencing

Small scale targeted deep sequencing of CRISPR Arrays for the ortholog screen was performed using the Illumina MiSeq v3 300 cycle kit on an Illumina MiSeq platform or Illumina HiSeq High Output High Output PE 200 cycle kit an Illumina HighSeq2500. Deep sequencing of spacer libraries prepared using SENECA were sequenced using the NextSeq 550/550 High Output Kit v2 150 cycle on Illumina NextSeq platform or the MiSeq Reagent Kit v3 150-cycle on a MiSeq.

### Data Analysis Pipeline

FASTQ files were quality filtered and trimmed using trimmomatic (trimmomatic SE LEADING:3 TRAILING:3 SLIDINGWINDOW:4:15 MINLEN:75) and subsequently converted to FASTA files using FASTX-Toolkit v0.0.14 (fastq-to-fasta) (http://hannonlab.cshl.edu/fastx_toolkit/). Using custom scripts written in python2.7, spacers were identified based on the identification of a 20-66 nucleotide sequence between two 10-nt DR segments, allowing for 2 and 3 mismatches in the first and second DR segment, respectively. Arrays with multiple spacers were identified based on the presence of a complete DR sequence, allowing for 3 mismatches. Only unique spacers (>1 mismatch) from a given sample were further processed. Spacers were aligned to a merged reference genome containing plasmid and E. coli sequences [E. coli B121(DE3) Gold (NC_012947.1) genome, E. coli K12 (NC_000913.3)] using bowtie2 (bowtie2—very-sensitive-local). In MS2 challenge experiments, the MS2 sequence [MS2 (NC_001417.2)] was also included in the merged reference genome. Identical alignments were collapsed using samtoolsv1.3, and alignments were visualized in Geneiousv10.2.3. Basic statistics about numbers of reads or alignment features were calculated using standard bash commands, and compiled and visualized using Prism7.0d. Gene body percentiles were calculated using RSeQC (geneBody_coverage.py v2.6.4). Nucleotide probabilities were determined and visualized using the weblogo webtool v2.8.2. Simulated spacer datasets were prepared using BEDtools v2.25 (bedtools random-n 500-l 38). Transcript quantification for RNA-seq and Record-seq was performed using featureCounts v1.5.0. Using custom scripts written in Matlab v9.1.0, RNA-seq and Record-seq transcript counts were normalized using transcripts per million (TPM) and used to compute cumulative spacer sums, a linear regression fit, coefficient of determination (R²), and Pearson linear correlation coefficient.

Record-seq datasets corresponding to oxidative or acid stress treatments were analyzed using custom scripts written in R v3.4.4. Briefly, transcripts with less than 5 counts across replicates were discarded. Heatmaps representing unsupervised hierarchical clustering of Pearson linear correlation with complete linkage (using raw transcript counts as inputs) were prepared using the ‘heatmap.2’, ‘hclust’, and ‘cor’ commands with default settings. Principal component analysis (PCA) was performed on log 2 transformed data (raw counts plus one pseudocount to tolerate zeros) for the 50 most variable (standard deviation) genes using the ‘prcomp’ command with default settings. Differential expression analyses (using raw counts plus one pseudocount as input) were performed using DEseq2v1.14.1, edgeRv3.16.5, and baySeqv2.8.0 encapsulations within R. Heatmaps representing unsupervised hierarchical clustering of signature differentially expressed genes were prepared using the ‘pheatmap’ command with default settings.

### Code Availability

The custom scripts used for the described data analysis are available on the Platt Lab website (platt.ethz.ch).

RNASeq of E. coli BL21(DE3)

RNA extraction from E. coli BL21(DE3) was performed after overnight growth under induction of FsRT-Cas1-Cas2 expression following the QIAGEN Supplementary Protocol: Purification of total RNA from bacteria using the RNeasy Mini Kit. To achieve the appropriate amount of input culture (corresponding to 5×10⁸ cells), serial dilutions of the overnight culture were prepared to achieve an OD₆₀₀ between 0.2 to 0.6 measured with a NanoDrop OneC (Thermo Scientific). Bacteria were lysed using acid-washed glass beads (G1277-10G, Sigma Aldrich). The additional on-column DNase digestion was performed using the RNase-Free DNase Set (QIAGEN). DNA free RNA was submitted to the Genomics Facility Basel for ribosomal RNA (rRNA) depletion using the Ribo-Zero rRNA Removal Kit (Illumina) and followed by library preparation and sequencing on an Illumina NextSeq platform using the NextSeq 500/550 High Output v2 kit (150 cycles).

### td Intron

The gBlock FS_gBlock_td_intron_acceptor (Sequences 3) was cloned into pFS_0235 using SphI/SgrAI yielding pFS_0238. This gBlock encoded the BBa_J23104 promoter, the ribosome binding site from bacteriophage T7 gene 10 as well as the td intron sequence including flanking regions facilitating efficient splicing. Furthermore, a BbsI-mediated Golden Gate cloning site was placed downstream and upstream of the td intron sequence, allowing for seamless assembly of upstream and downstream exon sequences in a single one-pot reaction as described above. As the inventors previously noticed, that the 5′ end of transcripts was preferentially acquired by the FsRT-Cas1-Cas2 complex, the inventors introduced the td intron within the first 23 to 31 nucleotides of the respective transcripts. The inventors created intron-interrupted sequences of three E. coli genes cspA, rpoS, argR (cold shock protein CspA, RNA polymerase sigma factor RpoS and Arginine repressor, respectively). These were selected based on the fact that they were well sampled by the FsRT-Cas1-Cas2 complex in preceding SENECA experiments. The flanking exon sequences were mutated in four to six positions to yield optimized sequences for td intron splicing, which also aided in unambiguously distinguishing the spliced and endogenous transcripts or DNA.

Accordingly, the inventors ordered complementary oligonucleotides for the fragment of the transcript to be cloned 5′ of the td intron and annealed them prior to Golden Gate Assembly, while the fragment to be cloned 3′ of the intron was amplified by PCR from genomic DNA. Oligonucleotides were FS_1054/1055 (5′ of the intron, annealed) and FS_1056/1057 (3′ of the intron, PCR) for CspA; FS_1038/1039 and FS_1040/1041 for RpoS; FS_1046/1047 and FS_1048/1049 for ArgR. The inventors ensured that mutating sequences of the respective genes to those of the td intron flanking sites did not generate a stop codon. The td intron containing FsRT-Cas1-Cas2 overexpression constructs were subjected to a standard acquisition assay followed by plasmid DNA extraction, SENECA and deep sequencing. Presence of td intron splice sites in DNA outside of the FsCRISPR array was tested by extracting gDNA from td-ArgR transformed cultures using the GenElute Bacterial Genomic DNA Kit (Sigma Aldrich). Libraries containing the td intron insertion site were amplified using a two-round PCR strategy method analogous to the ones described above using forward primers FS_1154 to FS_1157 and reverse primers FS_1158 to FS_1161. First-round PCR was performed at 57° C. annealing temperature and 20 sec elongation for 15 cycles. Second-round PCR was performed at 63° C. annealing temperature and 20 sec elongation for 8 cycles.

Infection with MS2 Phage

For infections with MS2 phage, the recording plasmid pFS_0235 was transformed into the F′, and thus MS2 susceptible NovaBlue(DE3) Competent Cells (EMD Millipore). Next morning, 15 mL of TB containing 100 μM of IPTG were inoculated with 10 colonies and grown at 37° C., 150 rpm in an orbital shaker until an OD₆₀₀ of 0.24. Then, MgSO₄ was added to 5 mM final concentration. Aliquots of 3 mL were split into bacterial culture tubes, infected with 200 μL of high-titre MS2 phage suspension and incubated for 1 h at room temperature without shaking to allow infection by MS2. Next, culture tubes were transferred to the orbital shaker and incubated overnight at 30° C., 80 rpm. Growth of E. coli in presence of MS2 phage at 30° C. rather than 37° C. prevents lysis of cells by productive MS2. Next morning, shaking was increased to 150 rpm. Another day later (~41 h post-infection), cultures were pelleted by centrifugation, plasmid DNA was extracted and subjected to SENECA followed by deep sequencing.

Synthetic Recording of sfGFP and Rluc Transcripts

The Pcat-tetR-term_PtetO encoding fragment was amplified with primers FS_1123/FS_1125 from pLP167 (kind gift from Luzi Pestalozzi), digested with BamHI/AgeI and cloned into AgeI/BbsI-digested pFS_0238 (see cloning of td intron constructs), yielding pFS_0270 which contains a BbsI-mediated Golden-Gate immediately downstream of the PtetA promoter. Subsequently, sfGFP was amplified from pLP167 with primers FS_1134/FS_1135 and R/uc was amplified using FS_1136/FS_1137 from BBa_J52008 (registry of standard biological parts). Both fragments were cloned into pFS_0270 using BbsI-mediated Golden Gate Assembly, yielding pFS_0271 (sfGFP) and pFS_0272 (Rluc), respectively. LuxR promoter parts were amplified with primers FS_1584/FS_1585 from plG0046 and FS_1586/FS_1587 from plG0059 (registry of standard biological parts) and cloned into AgeI-digested pFS_0270 using NEBuilder HiFi DNA Assembly Master Mix (NEB), resulting in pFS_0399. Oligos FS_1588/FS_1589 were annealed and cloned into pFS_0399 digested with SalI/BamHI—yielding pFS_0400. The Fluc coding sequence was amplified from Bbal712019 (registry of standard biological parts) using FS_1618/FS_1619, digested with BsaI and cloned into BbsI-digested pFS_0400, resulting in pFS_0412 that was used in RNA recording experiments. For each biological replicate, 50 mL of IPTG containing TB media were inoculated with 22 colonies of E. coli BL21(DE3) transformed with pFS_0271(sfGFP), pFS_0272 (Rluc) or pFS_0412(Fluc). When reaching an OD₆₀₀ of 0.25, cells were split into 3 mL aliquots in bacterial culture tubes and induced with aTc in case of P_{tetA} promoter or N-(3-Oxododecanoyl)-L-homoserine lactone (30C6-HSL) (Sigma) in case of P_{LuxR} promoter, and cultured in an orbital shaker for 12-14 hours at 300 rpm, followed by plasmid DNA extraction, SENECA and deep sequencing. Spacers aligning to sfGFP, Rluc and Fluc were quantified as described above (see “Data analysis pipeline”). Detected number of unique spacers per million sequencing reads was normalized defining the sum number of spacers per biological replicate as 100% and plotted using GraphPad Prism v7.0d. For RNA-recording with pFS_0271 and pFS_0272 RNA extraction from the same cultures was performed using the RNAsnap method followed by treatment with the TURBO DNA-free Kit (Thermo Scientific) using 1.5 μL of TURBO DNase to minimize DNA-background. Reverse transcription was performed using qScript cDNA SuperMix (Quanta Bio) with 500 ng of RNA sample as a template. cDNA was diluted 1:4 and quantification was performed in 2 technical replicates by real-time PCR (qRT-PCR) using TaqMan Fast Advanced Master Mix (Life Technologies) in a Roche LightCycler 96 System. Absolute copy number was calculated using standard curve method and 16 s rRNA was used as a housekeeper. To determine mRNA copy number corresponding to number of cells in a single SENECA reaction (6×10⁹) was calculated based on the average amount of 18700 16 s rRNA transcripts per single E. coli cell (BNID 102992).

### Orthogonal Synthetic Recording

The Rluc coding sequence was amplified using FS_1620/FS_1137 from pFS_0272 and cloned into pFS_0399 using BbsI-mediated Golden Gate Assembly, yielding pFS_0413. The Fluc coding sequence was amplified from Bba_1712019 (registry of standard biological parts) using FS_1621/FS_1619, digested with BbsI and cloned into BsaI-digested pFS_0413, resulting in pFS_0414 which was subsequently used in orthogonal synthetic recording experiments.

For each biological replicate, 50 mL of TB media containing 100 μM IPTG were inoculated with 33 colonies of E. coli BL21(DE3) transformed with pFS_0414, containing (3-Oxododecanoyl)-L-homoserine lactone (30C6-HSL)-inducible Fluc and aTc-inducible Rluc coding sequences. When reaching an OD₆₀₀ of 0.25, cells were split into 3 mL aliquots in bacterial culture tubes and induced with 75 ng/mL of anhydrotetracyclinehydrochloride (aTc) (Cayman Chemical) or 10 μM of 30C6-HSL (Sigma) or a combination of both and cultured in an orbital shaker for 12 hours at 300 rpm, followed by plasmid DNA extraction, SENECA, deep sequencing as well as parallelized RNA extraction from the same culture followed by reverse transcription and qPCR measurements. Data was analyzed as described above for recording of single synthetic transcripts.

### Transcriptional Response to Oxidative Stress

Per biological replicate 36 mL IPTG containing TB media containing 100 μM IPTG were inoculated with 24 colonies of E. coli BL21(DE3) transformed with pFS_0235 the evening before (resulting in 1 colony/1.5 mL) and shaken in a 250 mL baffled shaker flask until reaching an OD₆₀₀ of 0.24 to 0.25. Then cultures were split into 3 mL aliquots into bacterial culture tubes (Grainer) and treated with H₂O₂(30% w/w solution, Sigma Aldrich) to a final concentration of 1 mM or an equal volume of ddH₂O. Growth was continued for 12 hours at 300 rpm followed by harvesting of 2 mL of culture for plasmid DNA extraction, SENECA and deep sequencing. Data were analyzed as described above (see “Data analysis pipeline”).

### Transcriptional Response to Acid Stress

For pH-controlled growth, potassium-modified lysogenic broth (LB) (10 g/L tryptone, 5 g/L yeast extract, 7.45 g/L KCl) was buffered with 100 mM HOMOPIPES (Homopiperazine-1,4-bis(2-ethanesulfonic acid)). Subsequently, the pH of the medium was adjusted to either 5.0 (acid stress) or 7.0 (neutral) using KOH solution as described previously. For each biological replicate 50 mL of pH adjusted, IPTG containing LB media were inoculated with 33 colonies of E. coli BL21(DE3) transformed with pFS_0235 (resulting in 1 colony/1.5 mL). Samples were harvested between OD₆₀₀ of 0.3 to 0.6 for plasmid DNA extraction, SENECA and deep sequencing. Data were analyzed as described above (see “Data analysis pipeline”).

Cloning of aTc-Inducible FsRT-Cas1-Cas2 Expression Construct

For recording the transcriptional response to paraquat an aTc-inducible FsRT-Cas1-Cas2 expression construct was generated. Therefore, a fragment containing the tet repressor driven by a constitutive promoter as well as the P_{tetA} promoter was amplified from pFS_0271 using FS_1574/1575 and digested with BgII/SphI, furthermore the N-terminus of FsRT-Cas1-Cas2 was amplified with FS_1576/1577 and digested with SphI/BgIII. These two fragments were cloned into BgII/BgIII-digested pFS_0235 yielding pFS_0393. The codon optimized FsRT-Cas1-Cas2 sequence was obtained from Genscript, amplified using FS_1641/1642 and cloned into pFS_0393 using XhoI/SphI replacing the initial FsRT-Cas1-Cas2 coding sequence and yielding pFS_0453 (SEQ ID NO: 334).

### Transcriptional Response to 1 mM or 10 mM Paraquat

Paraquat dichloride hydrate (PESTANAL, Sigma Aldrich) was dissolved at 1 M in ddH₂0. For each biological replicate, 75 mL of TB media containing 30 ng/mL aTc were inoculated with 50 colonies of E. coli BL21(DE3) transformed with pFS_0393 and shaken in baffled shaker flasks until reaching an OD₆₀₀ of 0.24 to 0.25. Then cultures were split into 3 mL aliquots into bacterial culture tubes and treated with either 1 mM or 10 mM paraquat and cultured for an additional 11-12 hours before harvesting of 2 mL of culture for plasmid DNA extraction, SENECA and deep sequencing. Data were analyzed as described above (see “Data analysis pipeline”).

### Transcriptional Response to Transient Paraquat Exposure

For each biological replicate two colonies of E. coli BL21(DE3) transformed with pFS_0453 were inoculated into 3 mL of TB media containing 30 ng/mL aTC in standard bacterial culture tubes. For the first 12 h all cultures were cultivated in the absence of paraquat (300 rpm, 37° C.). Then 2 mL of culture were aspirated, while the remaining 1 mL was spun down (2300×g, 10 min) the supernatant was aspirated and the bacterial pellet resuspended in 3 mL of fresh TB media containing 30 ng/mL of aTc. For both the transient as well as the permanent stimulus conditions, paraquat was added to 10 mM final concentration and the cultures were grown for an additional 12 h as above. Then 2 mL of culture were removed, the remaining 1 mL was pelleted as above and resuspended in 3 mL of fresh TB media containing 30 ng/mL of aTc. Paraquat was added to 10 mM the permanent stimulus condition and cultures were grown for an additional 12 h as above. Then 2 mL of culture were harvested for plasmid DNA extraction, SENECA and deep sequencing. Additionally, 100 μL of culture were harvested for RNA-extraction by the RNASnap protocol as described above followed by treatment with the TURBO DNA-free Kit (Thermo Scientific) using 1.5 μL of TURBO DNase. Ribosomal RNA was depleted using Ribo-Zero rRNA Removal Kit (Illumina) followed by library prep using TruSeq Stranded mRNA (Illumina) and deep sequencing on an NextSeq 500/550 High Output v2 kit (75 cycles) sequencing each library at a depth of 4 million reads or greater.

Quantitative NERO (qNERO)

To quantify the efficiency of spacer acquisition efficiency the following qPCR based approach was deployed allowing a much higher throughput and faster turnaround than traditional NGS-based readouts. To quantify adapted CRISPR spacers a standard curve, consisting of a known percentage of DNA containing acquired spacers diluted into a background of non-expanded DNA, was generated maintaining a constant number of DNA molecules across all samples. The gradient of plasmids containing spacers in percent consist out of the following: 100, 10, 1, 0.1, 0.01, 0.001, 0.0001, 0.

qPCR reactions were performed in a total volume of 10 μL using the KAPA SYBR® FAST qPCR Master Mix (2×) Universal (Kapa Biosystems). Each reaction was supplemented with ROX Low Reference Dye (50×) to account for non-PCR related fluorescence variations. The reaction composition is detailed in the table below:

| | Volume | Final |
|---|---|---|
| Component | (μL) | Concentration |
| KAPA SYBR FAST qPCR Master Mix (2X) | 5.0 | 1X |
| Forward Primer (10 mM) | 0.2 | 200 nM |
| Reverse Primer (10 mM) | 0.2 | 200 nM |
| 50X ROX Low | 0.2 | 1X |
| Template DNA | 1 | 0.1 fmol |
| PCR-grade water | Up to 10.0 | |

A set of primer pairs were employed to amplify the target regions between two DRs: AK465 & AK466, which was optimized for an annealing temperature of 51° C. Thermocycling was performed on an “Applied Biosystems QuantStudio 5” 384-well PCR machine using a two-step protocol with an annealing temperature of 51° C. as depicted in detail in the table below:

| Stage | Time [s] | Temperature [° C.] | |
|---|---|---|---|
| Hold | 20 | 95 | |
| PCR | 1 | 95 | 40x |
| | 20 | 51 | |
| Melt Curve | 1 | 95 | |
| | 20 | 60 | |
| | 1 | 95 | |

### Deep Mutational Scan (DMS)

### DMS: Spacer Acquisition-Mediated Antibiotic Selection System

To enable the screening of FsRT-Cas1-Cas2 variants, we developed a modified version of the recording plasmid. This selection plasmid expresses the acquisition machinery FsRT-Cas1-Cas2 when induced by anhydrotetracycline and features the Fs CRISPR array, transcribed by the constitutive P_{cat} promoter, fused to a nonfunctional gentamicin resistance cassette lacking the start codon. When the system acquires new spacers, some of which contain an ATG start codon in the correct frame and an RBS sequence, the acquired spacer restores expression of the gentamicin resistance cassette, thereby conferring gentamicin resistance. Using the selection system, we developed a modified Record-seq assay to enrich for bacterial cells that have successfully recorded spacers. Bacterial cultures were grown in 50 ml of MOPS defined minimal medium and 10 ng/ml anhydrotetracycline for 24 h while expressing the recording machinery and transferred with a 1:5 dilution to nonselective medium flasks containing 50 ml LB with 50 μg/ml ampicillin, the antibiotic selection marker on the plasmid backbone, as well as with a 1:5 dilution in selective medium flasks containing 50 ml LB with 50 μg/ml ampicillin and 10 μg/ml gentamicin to enrich for bacterial cells that have acquired spacers. The selective flasks were shaken overnight for 16 h before collection to allow the cells to recover. When validating this approach with the wild-type machinery, we postulated that in a pooled screening setup, more efficient variants would acquire more spacers and would thus be overrepresented in the selective medium condition, while nonfunctional variants would be depleted.

### DMS: Variant Library Generation and Library Cloning

We generated a single-variant mutagenic library for the entire protein sequence of FsRT-Cas1-Cas2 with a total length of 2160 base pairs, of which 632 and 86 amino acid positions can be mutated for FsRT-Cas1 and FsCas2 respectively, excluding the final stop codon. For every amino acid position, if possible, we mutate the wild type codon to two missense codons for every possible amino acid, and as internal controls, two synonymous codons and two STOP codons. In total, we generated mutagenic sequences for 28333 variants, of which 25844 variants comprised missense mutations, with 1056 synonymous mutations and 1433 STOP-codon mutations serving as internal controls. The library design did not include higher order combinatorial variants.

For cloning, we chose to go with tiled non-overlapping synthetically generated oligo pools, which had to be ordered in 9 tiles across the protein sequence due to synthesis constraints limiting our ability to synthesize oligo pools above 300 base pairs. A computational pipeline was written to generate mutant libraries of FsRT-Cas1-Cas2 as described above, including BsaI Golden Gate-compatible overhangs that were used for Golden Gate cloning into 9 BsaI Golden Gate-compatible Record-seq selection plasmids with a sucrose counter-selection stuffer, of which 8 tiles were used for FsRT-Cas1 and 1 tile was used for FsCas2. Before the Golden Gate reaction, the mutagenic oligo pools were PCR-amplified using the following reaction mix: 4 μl of a total of 20 ng of mutagenic oligos, 3 μl of 10 μM primer mix, 25 μl 2×KAPA HiFi master mix, 18 μl H₂O in a total of 50 μl were cycled with the following parameters: 3 min at 95° C. for initial denaturation, 15 cycles of 98° C. for 20 s, 54° C. for 15 s, 72° C. for 15 s, and a final extension for 60 s at 72° C. The reaction was PCR purified and used as an input for a BsaI Golden Gate-reaction using 115 ng of amplified mutagenic oligo pools and 1050 ng of selection plasmid backbone in a 1:3 molar ratio, 1 μl of T4 DNA ligase, 1 μl BsaI, 2 μl 10×T4 ligase buffer in a total of 9 separate 20 μl reaction mixes. The Golden Gate reaction was performed by incubating the libraries at 37° C. for 1 h, and the entire reaction mix was dialyzed and electroporated into 100 μl DH5a ElectroMAX electrocompetent cells using their standard electroporation parameters, recovered in 50 ml of LB and then cultured overnight after adding 50 μl/ml ampicillin for plasmid library maintenance and 5% sucrose for counter-selection. The next day, the full cultures were spun down for plasmid isolation and dilution plates were counted to ensure having at least ≥300× coverage. For the 8 FsRT-Cas1 libraries, adjacent tiles were DNA quantified and 2000 ng pooled together equimolarly to create a total of 4 paired-pooled libraries. The 4 FsRT-Cas1 paired-pooled libraries and one single FsCas2 library were electroporated for the screen by adding 3000 ng of each library into 200 μl of laboratory-prepared electrocompetent E. coli MG1655, recovering in 50 ml of LB for 1 h before proceeding with the DMS experiment.

### DMS: Deep Mutational Scan of FsRT-Cas1-Cas2

The deep mutational scanning experiment was conducted as previously described using 4 FsRT-Cas1 paired-pooled libraries and one single FsCas2 library with 4 biological replicates each. The previously transformed libraries in E. coli MG1655 were inoculated in 50 ml MOPS defined minimal medium and induced in 50 μl/ml ampicillin and 10 ng/ml anhydrotetracycline to express the recording machinery. The cultures were grown for 24 h while expressing the recording machinery and transferred with a 1:5 dilution to nonselective medium flasks containing 50 ml LB with 50 μg/ml ampicillin, the antibiotic selection marker on the plasmid backbone, as well as with a 1:5 dilution in selective medium flasks containing 50 ml LB with 50 μg/ml ampicillin and 10 μg/ml gentamicin to enrich for variants that have acquired spacers. The selective flasks were shaken overnight for 16 h before collection to allow the cells to recover. The entire flask volumes were then spun down for collection and plasmid isolation. Dilution plates at each time point were plated with serial dilutions to ensure at least ≥500× coverage at each step.

### DMS: Barcoded Subamplicon Sequencing

The deep sequencing strategy was inspired by the barcoded-subamplicon sequencing approach described in Doud and Bloom (2016) using a similar setup, except that we designed our own approach for library generation, cloning, custom adaptor sequences, custom UMI sequence and length, NGS primers, NGS protocol, sequencing readout and enrichment analysis. This unique Illumina library preparation strategy increases sequencing accuracy by attaching unique random barcodes to the PCR product during library preparation, and then grouping sequencing reads by barcode to correct for sequencing errors. 9 primer pairs were ordered for the 9 libraries, which append random UMIs on both sides with a length of 8 base pairs and additional staggers from 1-4 base pairs to ensure good sequencing quality. The collected plasmids from the DMS experiment were amplified for the first round as follows: 50 ng of library, 12.5 μl of 2×KAPA HiFi reaction mix, 1.5 μl of 10 μM first round primer mix in a total volume of 25 μl were cycled with the following parameters: 3 min at 95° C. for initial denaturation, 1 cycle of 98° C. for 20 s, 56° C. for 20 s, 72° C. for 15 s, and a final extension for 60 s at 72° C. PCR products were AMPure beads purified in a 1:1 50 μl total volume ratio of PCR to beads to get rid of any <300 bp bands and primer dimers. The purified PCR products were amplified for the second round as follows: 5 μl of first round product, 15 μl of 2×KAPA HiFi reaction mix, 3 μl of 4 μM second round primer mix and 7 μl H₂O in a total volume of 30 μl were cycled with the following parameters: 3 min at 95° C. for initial denaturation, 20 cycles of 98° C. for 20 s, 62° C. for 20 s, 72° C. for 15 s, and a final extension for 60 s at 72° C. Illumina-compatible libraries were sequenced in paired-end mode on an AVITI Cloudbreak instrument and ensured to have ≥100× coverage per library.

### DMS: Analysis Pipeline and Enrichment Scoring

After deep sequencing the libraries, the raw reads were paired and merged and processed by an in-house developed computational pipeline to quantify the variants that are found in each subamplicon in a counts table. The method processes raw amplicon sequencing reads into quantified variant count tables by first filtering reads on per-base and per-read Phred quality scores and removing reads containing ambiguous bases. Filtered reads are aligned to a template sequence encoding fixed scaffold and N-degenerate variable regions using pairwise alignment, and reads with insertions, multiple deletions, sub-threshold scores, or deletions at region boundaries are discarded. Aligned sequences are partitioned into sub-regions (unique molecular identifiers, primers, target sequence) using structure-defined coordinates, and constant dinucleotide anchors flanking each UMI and primer are validated to discard non-conforming reads. To correct for PCR amplification bias, reads sharing a UMI are grouped and only the dominant target sequence per UMI is retained, subject to minimum read fraction and count thresholds, with final counts expressed as distinct supporting UMIs. Per-sample tables are merged by outer join on target sequence. Sequences are then translated and filtered against the expected codon set of the library design, removing non-design codons, with counts aggregated at the amino acid level. Finally, a reference variant library is provided such that observed sequences are matched first by exact identity, then by sequence matching at edit distance one using indexed dictionary search, excluding ambiguous multi-mappings and collapsing counts per reference variant. The processed data was then further analyzed using EdgeR. In short, the samples are grouped into selective vs. nonselective datasets, low-count features are filtered, library sizes are TMM normalized, and a model is fit per variant to estimate differential abundance between conditions. Final enrichment is reported as log 2 fold-change (log₂FC) and normalized by synonymous controls.

### Bacterial Population Inputs for Record-Seq Experiments and Achieved Recording Efficiencies

Record-seq experiments were performed in standard 12 mL culture tubes filled with 3 mL of terrific broth (TB) media, of which 2 mL were used for subsequent plasmid DNA extraction. In early experiments the inventors determined that using 40 fmols (180 ng of plasmid DNA) as an input to SENECA gave consistent results and left enough plasmid for archiving samples and performing several additional SENECA reactions on the same sample if necessary.

Accordingly, 40 fmols can be considered for contextualizing the number of cells used in a typical experiment. The construct (pFS_0235) has a size of 7293 bp, and 40 fmol of plasmid DNA was used as an input for a SENECA reaction. Using the formula [mass of dsDNA (g)=moles of dsDNA (mol)×((length of dsDNA (bp)×617.96 g/mol)+36.04 g/mol)], this equals a mass of 180.3 ng of plasmid DNA. These 40 fmol of plasmid DNA equals a total number of 2.4×10¹⁰ plasmids (using Avogadro's number of 1 mole being equal to 6.022×1023 particles and multiplying this by 40×10-15 to account for the 40 fmol used). Assuming a copy number of ~20 for the pET origin, this results in 1.2×10⁹ cells used as a standard input per SENECA reaction

A single SENECA reaction of pFS_0235 eventually yields ~6,126 spacers upon using the entire adapter ligated plasmid DNA for PCR amplification (two 30 μL PCR reaction, each containing 10 μL of adapter ligated plasmid DNA). Using the optimized FsRT-Cas1-Cas2 expression construct encoding an E. coli codon-optimized FsRT-Cas1-Cas2 coding sequence under transcriptional control of the aTc inducible PtetA promoter (pFS_0453), the efficiency increased ~10-fold to 61,462 spacer/SENECA reaction. Accordingly, 40 fmol of plasmid DNA acquired, 61,462 spacers. This is equal to one in 390,485 plasmids acquiring a new spacer. Assuming the copy number of pET30b to be 20, this results in every one in 19,524 cells acquiring a new spacer.

Based on the number of cells required to detect a specific stimulus, this calculation can be used to derive the number of cells used as a minimal input for the respective recording. For example, the inventors defined the minimum number of spacers to be required for assessing an arbitrary sequence (sfGFP) to be as low as 500 spacers, which corresponds to 8.8×10⁶ E. coli cells.

Likewise, the inventors estimated the number of spacers required to detect complex cellular behaviors to be 313 (7% of the original data). This equals 6.1×10⁶ E. coli cells used as an input. The total number of spacers required to record a complex stimulus happens to be lower than that required to record a defined stimulus (sfGFP), because in the complex case, spacers mapping to many different genes contribute to a‘usable output’ while in the case of a defined stimulus, only a subset of the required total of 500 spacers is mapping to the single gene of interest (sfGFP).

### Type III Versus Type I CRISPR-Cas Systems

Type III CRISPR-Cas systems like F. saccharivorans are generally several thousand-fold less efficient in spacer acquisition than the prototypical Type I systems (like the E. coli Type I-E). This necessitates multiple rounds of elaborate size selection procedures followed by deep sequencing to identify new spacers. Likewise, PCR products from extended CRISPR arrays cannot be detected on DNA gels (agarose or PAGE) due to their vanishingly low abundance. Taken together, while the classic spacer readout is applicable for highly efficient spacer acquisition systems, it precludes deep characterizations of most CRISPR-Cas systems, which motivated the development of SENECA.

### Assessing the Correlation Between RNA-Seq and Record-Seq

The inventors set out to assess the direct correlation between RNA-seq and Record-seq. However, given the distinct nature of the two techniques, namely RNA-seq being a snapshot in time and Record-seq being a cumulative record, the inventors expected the current transcript abundances (RNA-seq) to always precede its integration within a CRISPR array (Record-seq), thus leading to a weak correlation at any specific point in time. To investigate this potential asynchrony, the inventors performed RNA-seq and Record-seq from the same population of E. coli in stationary growth phase, and assessed the correlation between the two in the context of all genes, logarithmic-phase genes, stationary-phase genes, and plasmid-borne genes. While a weak correlation was observed between the two datasets when considering all genes (Pearson Correlation=0.61, R²=0.37), a much stronger correlation was observed when considering only logarithmic-phase genes (Pearson Correlation=0.72, R²=0.52). In contrast, the correlation was weakest when considering only stationary-phase genes (Pearson Correlation=0.49, R²=0.24), in which case the inventors expect that the spacers corresponding to stationary-phase growth have not yet been integrated. Performing this correlation analysis using stationary-phase or logarithmic-phase genes on Record-seq datasets obtained after 12, 24 and 36 hours of growth indeed revealed that the spacer repertoire shifted towards stationary-phase genes, while the correlation to logarithmic-phase genes decreased during extended growth indicating that spacer acquisition is still active at stationary phase. Furthermore, the plasmid-borne genes expressed under strong synthetic promoters, which are expected to be less affected by the growth phase, show the highest correlation (Pearson Correlation=0.84, R²=0.70). Taken together, the differences between RNA-seq and Record-seq highlight the respective features of transcript measurement by both methods, namely that RNA-seq represents a snapshot of the cellular transcriptome at the time of cell harvest, and Record-seq reveals the cumulative transcriptome sampled by FsRT-Cas1-Cas2 in a population of cells over time.

Analysis of Complex Cellular Behaviors with Record-Seq

The inventors set out to answer the following questions: (i) are the transcriptional-scale records broadly different between the treated and untreated conditions; (ii) do the most variable genes in the dataset distinguish the two populations; (iii) do standard RNA sequencing analysis tools identify genes that were cumulatively differentially expressed; (iv) are the cumulatively differentially expressed genes informative in the context of the initial stimulus; and (v) can the inventors unbiasedly classify the cellular populations into treated and untreated conditions based on broad, variable, or signature responses.

Questions (i-iv) are addressed in the main text, but here the inventors will elaborate on question (v). Among the signature genes the inventors identified several that were expected to dominate the cellular responses for each stimulus. For example, the inventors identified dps (DNA protection during starvation protein), which codes for a hallmark DNA damage repair protein, among the oxidative stress signature genes. Additionally, dps has previously been shown to be the top differentially expressed gene in response to oxidative stress. Furthermore, the inventors identified three members of the SUF system (i.e., sufABCDSE operon), which primarily operates under oxidative stress conditions to aid in the formation of iron-sulfur (Fe—S) clusters. Likewise, the inventors identified hallmark members of the acid stress response, including asr (acid-shock protein precursor) as well as several chaperones (e.g., dnaKand ibpB) and heat-shock proteins (e.g., grpE and ibpA) among the acid stress signature genes.

CRISPR Spacer Acquisition from RNA Versus DNA

The inventors present multiple lines of evidence showing CRISPR spacer acquisition from RNA, including spacer acquisition from an RNA only td intron splice junction (FIG. 1, panel B), spacer acquisition from an RNA virus, and RNA abundance-dependent spacer acquisition. While these observations strongly suggest that FsRT-Cas1-Cas2 is capable of acquiring spacers directly from RNA, they do not exclude the possibility that spacers are also being acquired from DNA. While the distinction between spacer acquisition from RNA versus DNA is fundamental to understanding the molecular mechanism of FsRT-Cas1-Cas2-mediated spacer acquisition, it does not confound Record-seq interpretation, whereby acquired spacers are preferentially derived from highly transcribed genes, correlate with gene expression at the genome-wide level, and highly correlate with RNA abundance.

### Benefits of Record-seq

The benefits of Record-seq include (i) the ability to heterologously express orthologous RT-Cas1-containing CRISPR acquisition systems in order to capture and store RNA species within DNA in an abundance-dependent process; (ii) the capacity to efficiently and scalably read out molecular histories permanently stored in DNA and reconstruct transcriptome-scale events; (iii) the application of this technology for recording specific inputs, such as virus infection or any single or orthogonal set of inducible expression system and (iv) the potential applications of this system for creating ‘sentinel’ cells for medical or biotechnology applications. Even if specific external stimuli cannot be recorded directly, the transcriptome-scale molecular signatures recorded within a bacterial population may be sufficient to report meaningful physiological states.

### Mice Experiments

For oral gavage, E. coli (BL21 (DE3) or MG1655) cells were transformed with pFS_0453 (SEQ ID NO: 334) and streaked on LB-agar plates containing 50 μg/mL kanamycin and grown overnight (12 h) at 37° C. The plasmid pFS_0453 encodes FsRT-Cas1-Cas2 under transcriptional control of an anhydrotetracycline inducible promoter (pTetA) as well as the FsCRISPR array 2 followed by a FaqI restriction site for the SENECA readout.

The following evening, a single colony was picked into 3 mL LB medium containing 50 μg/mL kanamycin under sterile conditions and grown overnight at 37° C. in a bacterial shaker (200-300 rpm). This culture was used to prepare a glycerol stock by mixing 500 μL of bacterial culture with 500 μL of sterile 50% (w/v) glycerol for long term storage at −80° C. For in vivo recording experiments, an overnight liquid culture was inoculated either directly from this glycerol stock or by streaking bacterial on an LB-agar plate containing 50 μg/mL kanamycin to obtain single bacterial colonies.

Gnotobiotic C57BL/6 mice were orally gavaged with 1×10⁹ colony forming units (CFU) of E. coli BL21(DE3) or MG1655 cells transformed with pFS_0453 in 500 μL PBS. Persistence of the plasmids was ensured by adding 100 μg/mL kanamycin sulfate (Sigma Aldrich) to the drinking water. Expression of FsRT-Cas1-Cas2 was induced by the addition of 10-30 μg/mL anhydrotetracycline (Cayman Chemical) to the drinking water.

For the DSS experiment, kanamycin (100 μg/mL) and anhydrotetracycline (30 μg/mL) were added to the drinking water of the germ-free C57BL/6 mice 24 hours prior to gavage. Animals were maintained under germ-free conditions. A colony of E. coli BL21(DE3) transformed with pFS_0453 was grown overnight in LB medium containing 50 μg/mL kanamycin. The resulting culture was pelleted and resuspended in 1×PBS. This bacterial resuspension was used to orally gavage each animal with 1×10⁹ colony forming units (CFU) of E. coli. Animals were maintained on water containing both kanamycin and anhydrotetracycline throughout the entire experiment. Fecal pellets were collected for 18 days starting 24 hours after the gavage. From day 5 to day 9 of the experiment, dextran sulfate sodium (DSS) (MPBio) was added to 1%, 2% or 3% (w/v) to the animals drinking water while maintaining kanamycin and anhydrotetracycline as described above. Animals were treated in groups of 3 and negative control animals received no DSS via the water.

The experiment was terminated on day 19 when colonal and cecal contents were also harvested for plasmid DNA extraction.

Plasmid DNA was extracted using the QIAprep Spin Miniprep Kit according to the manufacturer's instructions, volumes of buffers were increased to 500, 500 and 700 μL for buffers P1, P2 and N3, respectively to adjust for the increased biomass. Plasmid DNA was eluted in 150 μL of buffer EB and subsequently concentrated by precipitation. Therefore, 15 μL of 3M sodium acetate solution pH 5.2 (Sigma-Aldrich) and 105 μL isopropanol were added to each sample. Samples were incubated at −20° C. for at least 20 mins. Following centrifugation to precipitate nucleic acids (20,000×g, 30 mins, 4° C.), the supernatant was removed and the DNA pellet was washed with 150 μL of 70% (v/v) ethanol by centrifugation (20,000×g, 15 mins, 4° C.). Ethanol was aspirated and DNA pellets were briefly dried at 55° C. upon which the DNA pellet was resuspended in 15 μL of buffer EB. From this eluate, 7.5 μL were used for SENECA adapter ligation with all subsequent step of the SENECA protocol performed as described previously.

For the diet experiment comparing chow and starch diets, all animals were maintained on a chow-based diet (3307, Kliba Nafag) prior to the experiment. On Day 1 of the experiment, 5 animals were continuously maintained on the chow-based diet, while a second group of 5 animals was switched to a starch based diet (D12450Ji, Research Diets Inc.). On Day 2 of the experiment, anhydrotetracycline and kanamycin sulfate were added to the drinking water (30 μg/mL and 100 μg/mL, respectively). On Day 3 of the experiment, all animals were orally gavaged with 1×10⁹ colony forming units (CFU) of E. coli BL21(DE3) transformed with pFS_0453 as described above. Fecal pellets were collected from day 4 to day 9 of the experiment for the extraction of plasmid DNA as described above. Furthermore, on day 10 the animals were dissected to obtain cecal and colonic contents for plasmid DNA extraction as described above.

For the diet experiment comparing chow, starch and fat diets, all animals were maintained on a chow-based diet (3307, Kliba Nafag) prior to the experiment. On day 1 of the experiment, were put on either a chow-based diet (3307, Kliba Nafag), a starch-based diet (D12450Ji, Research Diets Inc.) or a fat-based diet (Fat-enriched diet D12492i, Research Diets Inc.). On Day 2 of the experiment, anhydrotetracycline and kanamycin sulfate were added to the drinking water (30 μg/mL and 100 μg/mL, respectively). On Day 3 of the experiment, all animals were orally gavaged with 1×10⁹ colony forming units (CFU) of E. coli MG1655 transformed with pFS_0453 as described above. Fecal pellets were collected from day 4 to day 10 of the experiment for the extraction of plasmid DNA as described above. Furthermore, on day 10 the animals were dissected to obtain cecal and colonic contents for plasmid DNA extraction as described above.

### Primers:

Primers are listed in the sequence protocol under SEQ ID NO: 105-140 and 191-333. SENECA adapter oligos are listed under SEQ ID NO: 141-190.

TABLE 1
RT-Cas1 orthologs
Host strains and protein accession number of RT-Cas1
orthologs idenfitied by HMMER-based
protein sequence homology search
Host and protein accession number
Bacteroides salyersiae 494745665 ref WP_007481073.1
Leptolyngbya sp. PCC 7375 493562087 ref WP_006515493.1
Photobacterium aphoticum 837770314 ref WP_047875592.1
Millisia brevis 1055178592 ref WP_066909103.1
Calothrix parietina 505008919 ref WP_015196021.1
Bacteroides fragilis str. 3397 T10 595923015 gb EXY33263.1
Pelodictyon phaeoclathratiforme 501500885 ref WP_012509117.1
Arthrospira platensis 493670156 ref WP_006620498.1
Calothrix sp. PCC 7507504941836 ref WP_015128938.1
Leptolyngbya sp. PCC 6406 495588276 ref WP_008312855.1
Lachnoanaerobaculum saburreum 987863574 ref WP_060932241.1
Candidatus Brocadia fulgida 816979878 gb KKO19838.1
Leptolyngbya sp. O-77984539873 dbj BAU44853.1
Tistrella mobilis KA081020-065 388530577 gb AFK55773.1
Smithella sp. SC K08D17745626258 gb KIE18281.1
Lachnospiraceae bacterium oral taxon 082 497051594
ref WP_009447486.1
Psychrobacter lutiphocae 518502663 ref WP_019672870.1
Propionicicella superfundia 916602138 ref WP_051209229.1
Loktanella vestfoldensis 518800937 ref WP_019956891.1
Desulfovibrio hydrothermalis 505147525 ref WP_015334627.1
Oceanospirillum beijerinckii 654849652 ref WP_028302067.1
Fischerella muscicola 737152142 ref WP_035139015.1
Desulfobacca acetoxidans 503473041 ref WP_013707702.1
Hippea sp. KM1 643957755 ref WP_025270209.1
Chlorobium limicola 501442438 ref WP_012465887.1
Desulfarculus baarsii 503023536 ref WP_013258512.1
Thiocapsa sp. KS1 971091367 emb CRI67871.1
Candidatus Accumulibacter sp. SK-02 668684200 gb KFB76584.1
Candidatus Magnetoglobus multicellularis str.
Araruama 571788307 gb ETR69258.1
Vibrio sinaloensis 740352375 ref WP_038188758.1
Campylobacter concisus 544653868 ref WP_021087740.1
Cellulomonas bogoriensis 917498396 ref WP_052104813.1
Teredinibacter turnerae 518435809 ref WP_019606016.1
Campylobacter fetus subsp. fetus 998762051 emb CZE46369.1
Gemmatimonadetes bacterium SCN 70-22 1063993205 gb ODT03821.1
Microcoleus sp. PCC 7113504999115 ref WP_015186217.1
Micromonospora rosaria 1000329745 gb KXK58998.1
Candidatus Entotheonella sp. TSY2575418691 gb ETX03376.1
Lachnoanaerobaculum sp. MSX33 570843978 gb ETO97675.1
Corynebacterium durum 492955761 ref WP_006063846.1
Anabaena cylindrica PCC 7122 428682296 gb AFZ61061.1
Pseudanabaena biceps 497311431 ref WP_009625648.1
Vibrio sp. MEBiC08052972247703 gb KUI97421.1
Actinomyces johnsonii 545331217 ref WP_021604855.1
Microlunatus phosphovorus 503627960 ref WP_013862036.1
Kamptonema 494597365 ref WP_007355619.1
Skermania piniformis 1054700955 ref WP_066466672.1
Fischerella sp. NIES-3754965689238 dbj BAU08380.1
Chlorobium phaeobacteroides 500067943 ref WP_011745868.1
Vibrio vulnificus 499466110 ref WP_011152750.1
Bacteroides fragilis 547947118 ref WP_022348096.1
Porphyromonas sp. COT-052 OH4946746384965 ref WP_039428138.1
Kutzneria sp. 744 918333650 ref WP_052396493.1
Porphyromonas crevioricanis 565855908 ref WP_023938229.1
Rubrivivax benzoatilyticus 497541412 ref WP_009855610.1
Streptomyces sp. F-3 1026350507 dbj GAT81929.1
Campylobacter gracilis 492518353 ref WP_005873073.1
Fusicatenibacter saccharivorans 941895202 ref WP_055226073.1
uncultured Thiohalocapsa sp. PB-PSB1 557040601 gb ESQ17084.1
Porphyromonas gingivalis 492529527 ref WP_005874916.1
uncultured Thiohalocapsa sp. PB-PSB1 557029821 gb ESQ08042.1
Azospirillum lipoferum 503954719 ref WP_014188713.1
Teredinibacter sp. 991H.S.0a.06797071444 ref WP_045826479.1
Tolypothrix campylonemoides 751570959 ref WP_041039832.1
Pseudoalteromonas rubra 800981085 ref WP_046007427.1
Rhodovulum sulfidophilum 985596740 ref WP_060836241.1
Teredinibacter turnerae 516642225 ref WP_018013804.1
Arcobacter thereius 1054172508 ref WP_066177132.1
Nocardiopsis baichengensis 516128787 ref WP_017559367.1
Arthrospira maxima 493720432 ref WP_006669920.1
Eubacteriaceae bacterium CHKCI0041016807618 emb CVI70780.1
Frankia sp. BMG5.1919937513 ref WP_052914180.1
Roseburia inulinivorans 937570588 emb CRL43259.1
Porphyromonas gingivalis 503581191 ref WP_013815267.1
Campylobacter fetus subsp. fetus 998759376 emb CZE50714.1
Microcystis aeruginosa 640538680 ref WP_024971209.1
Marinomonas mediterranea 503425197 ref WP_013659858.1
Candidatus Magnetomorum sp. HK-1927673953 gb KPA10619.1
Campylobacter fetus subsp. fetus 998758141 emb CZE46264.1
Synechococcus sp. NKBG042902780027826 ref WP_045442561.1
Chlorobaculum limnaeum 1071376969 ref WP_069809202.1
Nostoc sp. PCC 7107764929206 ref WP_044499977.1
Arthrospira platensis 504041557 ref WP_014275551.1
Woodsholea maritima 518804695 ref WP_019960649.1
Actinomyces cardiffensis F0333 478776992 gb ENO18597.1
Mastigocladus laminosus 764662524 ref WP_044448019.1
Clostridium 916986069 ref WP_051592781.1
Rhodococcus sp. YH3-3 1033138899 ref WP_064444911.1
Rhodobacter capsulatus 940623611 gb KQB14189.1
Lachnoanaerobaculum saburreum 496026892 ref WP_008751399.1
Vibrio metoecus 941008961 ref WP_055043549.1
Porphyromonas gingivicanis 739003123 ref WP_036885018.1
Smithella sp. D17683425608 gb KFZ44108.1
Candidatus Accumulibacter sp. BA-91 668677118 gb KFB71594.1
Nodosilinea nodulosa 515871661 ref WP_017302244.1
Phormidesmis priestleyi Ana 938299454 gb KPQ33062.1
Vibrio mexicanus 823288127 ref WP_047044098.1
Photobacterium marinum 494733933 ref WP_007469744.1
Candidatus Brocadia fulgida 816977369 gb KKO17867.1
Desulfovibrio bastinii 652926624 ref WP_027180402.1
Candidatus Magnetoovum chiemensis 778249022 gb KJR40057.1
Azospirillum lipoferum 502738680 ref WP_012973664.1
Cyanothece sp. PCC 7822503100147 ref WP_013334941.1
Clostridiales bacterium VE202-01 639695530 ref WP_024721321.1
Actinomycetaceae bacterium BA112 1032601389 ref WP_064231067.1
Bacteroides 495935708 ref WP_008660287.1
Candidatus Jettenia caeni 494421634 ref WP_007220853.1
Rhodobacter capsulatus SB 1003 294475643 gb ADE85031.1
Oscillatoriales cyanobacterium USR001 1049312742 gb OCQ91006.1
Nostoc sp. PCC 7120 499304863 ref WP_010995638.1
Vibrio metoecus 941038135 ref WP_055051199.1
Scytonema hofmanni UTEX B657929289 ref WP_029630506.1
Arthrospira sp. PCC 8005 495324841 ref WP_008049584.1
Phormidium willei 1057444347 ref WP_068790073.1
Vibrio rotiferianus 742405863 ref WP_038884984.1
Thermodesulfovibrio sp. N1 1057568519 ref WP_068860870.1
Bacteroides fragilis 492341859 ref WP_005815836.1
Rhodovulum sp. PH10750340320 ref WP_040622239.1
Porphyromonas gulae 807048030 ref WP_046200570.1
Arthrospira sp. TJSD091 809071417 ref WP_046320545.1
Streptomyces sp. AVP053U2 1057451804 gb ODA69832.1

TABLE 2
Reports deep mutational scanning-derived, position-specific interchangeability
metrics for FsRT-Cas1-Cas2. For each amino acid position, the wild-type residue,
mean enrichment effect, and “Percent Functionally Tolerated Substitutions” are provided.

| | | | | | |
|---|---|---|---|---|---|
| | | | | Percent | |
| | | Wildtype | | Functionally | |
| | | amino | Mean | Tolerated | |
| Protein | Position | acid | LFC | Substitutions | Category |

| | | | | | |
|---|---|---|---|---|---|
| RT- | 1 | M | −4.63 | 0.00 | Not interchangeable |
| Cas1 | | | | | |
| RT- | 2 | F | −4.32 | 2.86 | Not interchangeable |
| Cas1 | | | | | |
| RT- | 3 | T | −2.37 | 11.11 | Limited |
| Cas1 | | | | | interchangeability |
| RT- | 4 | — | −4.17 | 5.56 | Not interchangeable |
| Cas1 | | | | | |
| RT- | 5 | D | −1.22 | 16.67 | Limited |
| Cas1 | | | | | interchangeability |
| RT- | 6 | E | −3.03 | 8.33 | Not interchangeable |
| Cas1 | | | | | |
| RT- | 7 | M | −3.72 | 13.51 | Limited |
| Cas1 | | | | | interchangeability |
| RT- | 8 | L | −3.40 | 19.44 | Limited |
| Cas1 | | | | | interchangeability |
| RT- | 9 | S | −2.52 | 36.11 | Interchangeable |
| Cas1 | | | | | |
| RT- | 10 | K | 0.46 | 100.00 | Highly |
| Cas1 | | | | | interchangeable |
| RT- | 11 | N | 0.03 | 86.11 | Highly |
| Cas1 | | | | | interchangeable |
| RT | 12 | N | −3.89 | 0.00 | Not interchangeable |
| Cas1 | | | | | |
| RT- | 13 | Q | −2.27 | 30.56 | Interchangeable |
| Cas1 | | | | | |
| RT- | 14 | R | −0.68 | 66.67 | Highly |
| Cas1 | | | | | interchangeable |
| RT- | 15 | L | −0.83 | 38.89 | Interchangeable |
| Cas1 | | | | | |
| RT- | 16 | A | −3.97 | 2.78 | Not interchangeable |
| Cas1 | | | | | |
| RT- | 17 | F | −3.00 | 5.56 | Not interchangeable |
| Cas1 | | | | | |
| RT- | 18 | E | −1.27 | 36.11 | Interchangeable |
| Cas1 | | | | | |
| RT- | 19 | H | −2.68 | 0.00 | Not interchangeable |
| Cas1 | | | | | |
| RT- | 20 | F | −3.85 | 0.00 | Not interchangeable |
| Cas1 | | | | | |
| RT- | 21 | A | −1.27 | 14.29 | Limited |
| Cas1 | | | | | interchangeability |
| RT- | 22 | T | −0.72 | 36.11 | Interchangeable |
| Cas1 | | | | | |
| RT- | 23 | K | −2.94 | 0.00 | Not interchangeable |
| Cas1 | | | | | |
| RT- | 24 | N | −0.11 | 88.89 | Highly |
| Cas1 | | | | | interchangeable |
| RT- | 25 | D | −1.95 | 2.78 | Not interchangeable |
| Cas1 | | | | | |
| RT- | 26 | G | −2.32 | 11.11 | Limited |
| Cas1 | | | | | interchangeability |
| RT- | 27 | C | −0.92 | 44.44 | Interchangeable |
| Cas1 | | | | | |
| RT- | 28 | G | −4.73 | 0.00 | Not interchangeable |
| Cas1 | | | | | |
| RT- | 29 | P | −1.12 | 30.56 | Interchangeable |
| Cas1 | | | | | |
| RT- | 30 | D | −4.58 | 0.00 | Not interchangeable |
| Cas1 | | | | | |
| RT- | 31 | G | −2.88 | 0.00 | Not interchangeable |
| Cas1 | | | | | |
| RT- | 32 | M | −4.31 | 0.00 | Not interchangeable |
| Cas1 | | | | | |
| RT- | 33 | H | −2.20 | 25.00 | Limited |
| Cas1 | | | | | interchangeability |
| RT- | 34 | V | −2.36 | 13.89 | Limited |
| Cas1 | | | | | interchangeability |
| RT- | 35 | S | −1.28 | 5.56 | Not interchangeable |
| Cas1 | | | | | |
| RT- | 36 | E | −2.73 | 2.78 | Not interchangeable |
| Cas1 | | | | | |
| RT- | 37 | L | −4.02 | 2.78 | Not interchangeable |
| Cas1 | | | | | |
| RT- | 38 | E | −1.10 | 25.00 | Limited |
| Cas1 | | | | | interchangeability |
| RT- | 39 | K | 0.64 | 100.00 | Highly |
| Cas1 | | | | | interchangeable |
| RT- | 40 | Y | −4.43 | 0.00 | Not interchangeable |
| Cas1 | | | | | |
| RT- | 41 | W | −4.44 | 0.00 | Not interchangeable |
| Cas1 | | | | | |
| RT- | 42 | R | −0.35 | 69.44 | Highly |
| Cas1 | | | | | interchangeable |
| RT- | 43 | M | −2.53 | 16.22 | Limited |
| Cas1 | | | | | interchangeability |
| RT- | 44 | N | −3.57 | 0.00 | Not interchangeable |
| Cas1 | | | | | |
| RT- | 45 | H | −0.79 | 52.78 | Interchangeable |
| Cas1 | | | | | |
| RT- | 46 | D | 0.07 | 100.00 | Highly |
| Cas1 | | | | | interchangeable |
| RT- | 47 | Q | −0.98 | 25.00 | Limited |
| Cas1 | | | | | interchangeability |
| RT- | 48 | — | −3.83 | 2.78 | Not interchangeable |
| Cas1 | | | | | |
| RT- | 49 | — | −1.01 | 47.22 | Interchangeable |
| Cas1 | | | | | |
| RT- | 50 | S | −0.17 | 88.89 | Highly |
| Cas1 | | | | | interchangeable |
| RT- | 51 | D | −2.69 | 0.00 | Not interchangeable |
| Cas1 | | | | | |
| RT- | 52 | L | −4.05 | 0.00 | Not interchangeable |
| Cas1 | | | | | |
| RT- | 53 | K | −1.53 | 27.78 | Limited |
| Cas1 | | | | | interchangeability |
| RT- | 54 | N | −0.81 | 61.11 | Highly |
| Cas1 | | | | | interchangeable |
| RT- | 55 | Q | −0.93 | 50.00 | Interchangeable |
| Cas1 | | | | | |
| RT- | 56 | E | −1.73 | 5.56 | Not interchangeable |
| Cas1 | | | | | |
| RT- | 57 | Y | −4.27 | 2.78 | Not interchangeable |
| Cas1 | | | | | |
| RT- | 58 | Q | −1.35 | 5.56 | Not interchangeable |
| Cas1 | | | | | |
| RT- | 59 | P | −3.51 | 2.78 | Not interchangeable |
| Cas1 | | | | | |
| RT- | 60 | G | −3.84 | 0.00 | Not interchangeable |
| Cas1 | | | | | |
| RT- | 61 | I | −2.36 | 11.11 | Limited |
| Cas1 | | | | | interchangeability |
| RT- | 62 | — | −2.27 | 19.44 | Limited |
| Cas1 | | | | | interchangeability |
| RT- | 63 | L | −0.38 | 66.67 | Highly |
| Cas1 | | | | | interchangeable |
| RT- | 64 | I | −2.53 | 11.11 | Limited |
| Cas1 | | | | | interchangeability |
| RT- | 65 | R | −1.98 | 19.44 | Limited |
| Cas1 | | | | | interchangeability |
| RT- | 66 | E | −3.49 | 0.00 | Not interchangeable |
| Cas1 | | | | | |
| RT- | 67 | H | −0.56 | 61.11 | Highly |
| Cas1 | | | | | interchangeable |
| RT- | 68 | M | −2.71 | 16.22 | Limited |
| Cas1 | | | | | interchangeability |
| RT- | 69 | N | 0.14 | 83.33 | Highly |
| Cas1 | | | | | interchangeable |
| RT- | 70 | K | −1.51 | 8.57 | Not interchangeable |
| Cas1 | | | | | |
| RT- | 71 | T | −0.67 | 44.44 | Interchangeable |
| Cas1 | | | | | |
| RT- | 72 | G | −2.59 | 0.00 | Not interchangeable |
| Cas1 | | | | | |
| RT- | 73 | K | −3.09 | 0.00 | Not interchangeable |
| Cas1 | | | | | |
| RT- | 74 | R | −2.36 | 0.00 | Not interchangeable |
| Cas1 | | | | | |
| RT- | 75 | R | −4.75 | 0.00 | Not interchangeable |
| Cas1 | | | | | |
| RT- | 76 | N | −1.91 | 0.00 | Not interchangeable |
| Cas1 | | | | | |
| RT- | 77 | I | −3.89 | 8.33 | Not interchangeable |
| Cas1 | | | | | |
| RT- | 78 | A | −3.96 | 5.56 | Not interchangeable |
| Cas1 | | | | | |
| RT- | 79 | S | −1.22 | 28.57 | Limited |
| Cas1 | | | | | interchangeability |
| RT- | 80 | L | −3.78 | 0.00 | Not interchangeable |
| Cas1 | | | | | |
| RT- | 81 | N | −3.10 | 5.56 | Not interchangeable |
| Cas1 | | | | | |
| RT- | 82 | V | −3.66 | 0.00 | Not interchangeable |
| Cas1 | | | | | |
| RT- | 83 | I | −3.46 | 2.78 | Not interchangeable |
| Cas1 | | | | | |
| RT- | 84 | D | −4.63 | 0.00 | Not interchangeable |
| Cas1 | | | | | |
| RT- | 85 | R | −4.38 | 0.00 | Not interchangeable |
| Cas1 | | | | | |
| RT- | 86 | F | −4.18 | 5.56 | Not interchangeable |
| Cas1 | | | | | |
| RT- | 87 | — | −4.26 | 2.86 | Not interchangeable |
| Cas1 | | | | | |
| RT- | 88 | T | −3.17 | 5.56 | Not interchangeable |
| Cas1 | | | | | |
| RT- | 89 | R | −4.35 | 0.00 | Not interchangeable |
| Cas1 | | | | | |
| RT- | 90 | L | −4.11 | 8.33 | Not interchangeable |
| Cas1 | | | | | |
| RT- | 91 | L | −3.65 | 11.11 | Limited |
| Cas1 | | | | | interchangeability |
| RT- | 92 | S | −0.74 | 50.00 | Interchangeable |
| Cas1 | | | | | |
| RT- | 93 | Q | −2.57 | 0.00 | Not interchangeable |
| Cas1 | | | | | |
| RT- | 94 | K | −1.19 | 50.00 | Interchangeable |
| Cas1 | | | | | |
| RT- | 95 | L | −4.22 | 2.78 | Not interchangeable |
| Cas1 | | | | | |
| RT- | 96 | N | −1.13 | 30.56 | Interchangeable |
| Cas1 | | | | | |
| RT- | 97 | R | −0.11 | 83.33 | Highly |
| Cas1 | | | | | interchangeable |
| RT- | 98 | Y | −0.36 | 63.89 | Highly |
| Cas1 | | | | | interchangeable |
| RT- | 99 | L | −3.32 | 8.33 | Not interchangeable |
| Cas1 | | | | | |
| RT- | 100 | A | −0.98 | 27.78 | Limited |
| Cas1 | | | | | interchangeability |
| RT- | 101 | P | −0.03 | 86.11 | Highly |
| Cas1 | | | | | interchangeable |
| RT- | 102 | — | −0.43 | 66.67 | Highly |
| Cas1 | | | | | interchangeable |
| RT | 103 | F | −3.48 | 2.78 | Not interchangeable |
| Cas1 | | | | | |
| RT- | 104 | C | −0.86 | 55.56 | Interchangeable |
| Cas1 | | | | | |
| RT- | 105 | E | −0.23 | 75.00 | Highly |
| Cas1 | | | | | interchangeable |
| RT- | 106 | N | −1.01 | 52.78 | Interchangeable |
| Cas1 | | | | | |
| RT- | 107 | S | −4.34 | 0.00 | Not interchangeable |
| Cas1 | | | | | |
| RT- | 108 | Y | −3.82 | 8.33 | Not interchangeable |
| Cas1 | | | | | |
| RT- | 109 | A | −4.66 | 0.00 | Not interchangeable |
| Cas1 | | | | | |
| RT- | 110 | Y | −4.47 | 0.00 | Not interchangeable |
| Cas1 | | | | | |
| RT- | 111 | Q | −3.26 | 0.00 | Not interchangeable |
| Cas1 | | | | | |
| RT- | 112 | D | 0.84 | 94.44 | Highly |
| Cas1 | | | | | interchangeable |
| RT- | 113 | S | −0.59 | 66.67 | Highly |
| Cas1 | | | | | interchangeable |
| RT- | 114 | K | −4.51 | 0.00 | Not interchangeable |
| Cas1 | | | | | |
| RT- | 115 | G | −4.71 | 0.00 | Not interchangeable |
| Cas1 | | | | | |
| RT- | 116 | V | −1.91 | 11.11 | Limited |
| Cas1 | | | | | interchangeability |
| RT- | 117 | M | −0.13 | 70.27 | Highly |
| Cas1 | | | | | interchangeable |
| RT- | 118 | P | 0.15 | 88.89 | Highly |
| Cas1 | | | | | interchangeable |
| RT- | 119 | A | −3.78 | 5.56 | Not interchangeable |
| Cas1 | | | | | |
| RT- | 120 | V | −3.64 | 13.89 | Limited |
| Cas1 | | | | | interchangeability |
| RT- | 121 | L | −0.25 | 72.22 | Highly |
| Cas1 | | | | | interchangeable |
| RT- | 122 | K | −2.98 | 11.11 | Limited |
| Cas1 | | | | | interchangeability |
| RT- | 123 | A | −3.07 | 16.67 | Limited |
| Cas1 | | | | | interchangeability |
| RT- | 124 | K | −2.37 | 2.78 | Not interchangeable |
| Cas1 | | | | | |
| RT- | 125 | E | −0.59 | 69.44 | Highly |
| Cas1 | | | | | interchangeable |
| RT- | 126 | Y | −3.72 | 2.78 | Not interchangeable |
| Cas1 | | | | | |
| RT- | 127 | V | −2.32 | 30.56 | Interchangeable |
| Cas1 | | | | | |
| RT. | 128 | E | −0.16 | 88.89 | Highly |
| Cas1 | | | | | interchangeable |
| RT | 129 | L | −0.28 | 94.44 | Highly |
| Cas1 | | | | | interchangeable |
| RT- | 130 | G | −3.04 | 2.78 | Not interchangeable |
| Cas1 | | | | | |
| RT- | 131 | M | −0.56 | 64.86 | Highly |
| Cas1 | | | | | interchangeable |
| RT- | 132 | R | −0.73 | 19.44 | Limited |
| Cas1 | | | | | interchangeability |
| RT- | 133 | H | −0.55 | 75.00 | Highly |
| Cas1 | | | | | interchangeable |
| RT- | 134 | V | −2.47 | 25.00 | Limited |
| Cas1 | | | | | interchangeability |
| RT- | 135 | I | −3.01 | 30.56 | Interchangeable |
| Cas1 | | | | | |
| RT- | 136 | E | 0.46 | 83.33 | Highly |
| Cas1 | | | | | interchangeable |
| RT- | 137 | — | −3.51 | 16.67 | Limited |
| Cas1 | | | | | interchangeability |
| RT- | 138 | D | −4.69 | 0.00 | Not interchangeable |
| Cas1 | | | | | |
| RT | 139 | L | −3.87 | 8.33 | Not interchangeable |
| Cas1 | | | | | |
| RT- | 140 | K | −0.94 | 44.44 | Interchangeable |
| Cas1 | | | | | |
| RT- | 141 | N | −1.99 | 13.89 | Limited |
| Cas1 | | | | | interchangeability |
| RT- | 142 | Y | −4.36 | 5.56 | Not interchangeable |
| Cas1 | | | | | |
| RT- | 143 | F | −4.55 | 0.00 | Not interchangeable |
| Cas1 | | | | | |
| RT- | 144 | D | −3.95 | 0.00 | Not interchangeable |
| Cas1 | | | | | |
| RT- | 145 | T | −1.17 | 38.89 | Interchangeable |
| Cas1 | | | | | |
| RT- | 146 | — | −4.33 | 0.00 | Not interchangeable |
| Cas1 | | | | | |
| RT- | 147 | P | −0.50 | 61.11 | Highly |
| Cas1 | | | | | interchangeable |
| RT- | 148 | L | −3.24 | 2.78 | Not interchangeable |
| Cas1 | | | | | |
| RT- | 149 | E | −0.58 | 52.78 | Interchangeable |
| Cas1 | | | | | |
| RT- | 150 | N | −0.03 | 91.67 | Highly |
| Cas1 | | | | | interchangeable |
| RT- | 151 | L | −4.29 | 2.78 | Not interchangeable |
| Cas1 | | | | | |
| RT- | 152 | I | −2.27 | 5.56 | Not interchangeable |
| Cas1 | | | | | |
| RT- | 153 | P | −1.11 | 0.00 | Not interchangeable |
| Cas1 | | | | | |
| RT- | 154 | E | −0.76 | 36.11 | Interchangeable |
| Cas1 | | | | | |
| RT- | 155 | — | −3.72 | 8.33 | Not interchangeable |
| Cas1 | | | | | |
| RT- | 156 | E | −1.44 | 11.11 | Limited |
| Cas1 | | | | | interchangeability |
| RT- | 157 | R | −0.80 | 41.67 | Interchangeable |
| Cas1 | | | | | |
| RT- | 158 | Y | −1.96 | 14.29 | Limited |
| Cas1 | | | | | interchangeability |
| RT- | 159 | — | −3.26 | 10.00 | Limited |
| Cas1 | | | | | interchangeability |
| RT- | 160 | T | −0.35 | 69.44 | Highly |
| Cas1 | | | | | interchangeable |
| RT- | 161 | D | −3.35 | 13.89 | Limited |
| Cas1 | | | | | interchangeability |
| RT- | 162 | E | −0.31 | 80.56 | Highly |
| Cas1 | | | | | interchangeable |
| RT- | 163 | A | −1.06 | 55.56 | Interchangeable |
| Cas1 | | | | | |
| RT- | 164 | V | −3.33 | 5.56 | Not interchangeable |
| Cas1 | | | | | |
| RT- | 165 | L | −1.41 | 22.22 | Limited |
| Cas1 | | | | | interchangeability |
| RT- | 166 | H | −1.28 | 27.78 | Limited |
| Cas1 | | | | | interchangeability |
| RT- | 167 | L | −3.37 | 2.78 | Not interchangeable |
| Cas1 | | | | | |
| RT- | 168 | — | −3.41 | 2.78 | Not interchangeable |
| Cas1 | | | | | |
| RT- | 16 | K | −1.45 | 8.33 | Not interchangeable |
| Cas1 | | | | | |
| RT- | 170 | Q | −1.24 | 50.00 | Interchangeable |
| Cas1 | | | | | |
| RT- | 171 | Y | −3.81 | 2.78 | Not interchangeable |
| Cas1 | | | | | |
| RT- | 172 | L | −3.62 | 2.78 | Not interchangeable |
| Cas1 | | | | | |
| RT- | 173 | F | −1.51 | 16.67 | Limited |
| Cas1 | | | | | interchangeability |
| RT | 174 | C | −3.01 | 8.33 | Not interchangeable |
| Cas1 | | | | | |
| RT- | 175 | D | −1.45 | 5.56 | Not interchangeable |
| Cas1 | | | | | |
| RT- | 176 | I | −2.20 | 30.56 | Interchangeable |
| Cas1 | | | | | |
| RT- | 177 | S | −0.77 | 47.22 | Interchangeable |
| Cas1 | | | | | |
| RT | 178 | F | −1.37 | 27.78 | Limited |
| Cas1 | | | | | interchangeability |
| RT- | 179 | E | −2.11 | 11.11 | Limited |
| Cas1 | | | | | interchangeability |
| RT- | 180 | G | −2.98 | 0.00 | Not interchangeable |
| Cas1 | | | | | |
| RT- | 181 | K | −0.85 | 36.11 | Interchangeable |
| Cas1 | | | | | |
| RT- | 182 | — | −2.89 | 2.78 | Not interchangeable |
| Cas1 | | | | | |
| RT- | 183 | S | −0.62 | 63.89 | Highly |
| Cas1 | | | | | interchangeable |
| RT- | 184 | R | −1.78 | 8.33 | Not interchangeable |
| Cas1 | | | | | |
| RT- | 185 | K | −1.94 | 14.29 | Limited |
| Cas1 | | | | | interchangeability |
| RT- | 186 | T | −0.53 | 58.33 | Interchangeable |
| Cas1 | | | | | |
| RT- | 187 | Q | −0.50 | 72.22 | Highly |
| Cas1 | | | | | interchangeable |
| RT- | 188 | G | −4.30 | 0.00 | Not interchangeable |
| Cas1 | | | | | |
| RT- | 189 | — | −3.61 | 0.00 | Not interchangeable |
| Cas1 | | | | | |
| RT- | 190 | V | −3.02 | 13.89 | Limited |
| Cas1 | | | | | interchangeability |
| RT- | 191 | Q | −1.90 | 33.33 | Interchangeable |
| Cas1 | | | | | |
| RT- | 192 | G | −4.34 | 0.00 | Not interchangeable |
| Cas1 | | | | | |
| RT- | 193 | N | −2.75 | 8.33 | Not interchangeable |
| Cas1 | | | | | |
| RT- | 194 | A | −3.24 | 8.33 | Not interchangeable |
| Cas1 | | | | | |
| RT- | 195 | — | −3.21 | 5.56 | Not interchangeable |
| Cas1 | | | | | |
| RT- | 196 | S | −3.64 | 0.00 | Not interchangeable |
| Cas1 | | | | | |
| RT- | 197 | P | −3.52 | 5.56 | Not interchangeable |
| Cas1 | | | | | |
| RT- | 198 | — | −1.48 | 47.22 | Interchangeable |
| Cas1 | | | | | |
| RT- | 199 | L | −3.49 | 8.33 | Not interchangeable |
| Cas1 | | | | | |
| RT- | 200 | S | −3.81 | 2.78 | Not interchangeable |
| Cas1 | | | | | |
| RT- | 201 | N | −4.13 | 0.00 | Not interchangeable |
| Cas1 | | | | | |
| RT- | 202 | L | −3.49 | 8.33 | Not interchangeable |
| Cas1 | | | | | |
| RT- | 203 | Y | −3.47 | 2.78 | Not interchangeable |
| Cas1 | | | | | |
| RT- | 204 | L | −4.05 | 0.00 | Not interchangeable |
| Cas1 | | | | | |
| RT- | 205 | N | −2.48 | 11.11 | Limited |
| Cas1 | | | | | interchangeability |
| RT- | 206 | D | −1.34 | 25.00 | Limited |
| Cas1 | | | | | interchangeability |
| RT- | 207 | F | −3.07 | 0.00 | Not interchangeable |
| Cas1 | | | | | |
| RT- | 208 | D | −4.24 | 0.00 | Not interchangeable |
| Cas1 | | | | | |
| RT- | 209 | K | −0.62 | 72.22 | Highly |
| Cas1 | | | | | interchangeable |
| RT- | 210 | E | −0.61 | 61.11 | Highly |
| Cas1 | | | | | interchangeable |
| RT- | 211 | L | −2.92 | 13.89 | Limited |
| Cas1 | | | | | interchangeability |
| RT- | 212 | D | −0.77 | 44.44 | Interchangeable |
| Cas1 | | | | | |
| RT- | 213 | E | −0.30 | 91.67 | Highly |
| Cas1 | | | | | interchangeable |
| RT- | 214 | S | −0.31 | 80.56 | Highly |
| Cas1 | | | | | interchangeable |
| RT- | 215 | K | −0.48 | 72.22 | Highly |
| Cas1 | | | | | interchangeable |
| RT- | 216 | L | −0.91 | 52.78 | Interchangeable |
| Cas1 | | | | | |
| RT- | 217 | C | −0.50 | 69.44 | Highly |
| Cas1 | | | | | interchangeable |
| RT- | 218 | W | −3.63 | 0.00 | Not interchangeable |
| Cas1 | | | | | |
| RT- | 219 | — | −3.14 | 13.89 | Limited |
| Cas1 | | | | | interchangeability |
| RT- | 220 | R | −4.15 | 0.00 | Not interchangeable |
| Cas1 | | | | | |
| RT- | 221 | Y | −4.11 | 0.00 | Not interchangeable |
| Cas1 | | | | | |
| RT- | 222 | A | −3.42 | 16.67 | Limited |
| Cas1 | | | | | interchangeability |
| RT- | 223 | D | −4.15 | 0.00 | Not interchangeable |
| Cas1 | | | | | |
| RT- | 224 | N | −3.90 | 0.00 | Not interchangeable |
| Cas1 | | | | | |
| RT- | 225 | — | −2.92 | 16.67 | Limited |
| Cas1 | | | | | interchangeability |
| RT- | 226 | Y | −1.87 | 25.00 | Limited |
| Cas1 | | | | | interchangeability |
| RT- | 227 | — | −3.04 | 25.00 | Limited |
| Cas1 | | | | | interchangeability |
| RT- | 228 | Y | −3.13 | 2.78 | Not interchangeable |
| Cas1 | | | | | |
| RT- | 229 | M | −0.73 | 70.27 | Highly |
| Cas1 | | | | | interchangeable |
| RT- | 230 | D | −0.26 | 80.56 | Highly |
| Cas1 | | | | | interchangeable |
| RT | 23- | S | −0.29 | 72.22 | Highly |
| Cas1 | | | | | interchangeable |
| RT- | 232 | Y | −1.16 | 27.78 | Limited |
| Cas1 | | | | | interchangeability |
| RT | 233 | E | −1.44 | 16.67 | Limited |
| Cas1 | | | | | interchangeability |
| RT- | 234 | K | −0.41 | 66.67 | Highly |
| Cas1 | | | | | interchangeable |
| RT | 235 | A | −3.27 | 13.89 | Limited |
| Cas1 | | | | | interchangeability |
| RT. | 236 | L | −1.80 | 5.56 | Not interchangeable |
| Cas1 | | | | | |
| RT | 237 | L | −0.20 | 85.00 | Highly |
| Cas1 | | | | | interchangeable |
| RT | 238 | V | −0.41 | 82.35 | Highly |
| Cas1 | | | | | interchangeable |
| RT- | 239 | Y | −2.00 | 11.11 | Limited |
| Cas1 | | | | | interchangeability |
| RT- | 240 | S | −0.27 | 63.89 | Highly |
| Cas1 | | | | | interchangeable |
| RT- | 241 | E | −0.35 | 77.78 | Highly |
| Cas1 | | | | | interchangeable |
| RT- | 242 | L | −0.92 | 54.29 | Interchangeable |
| Cas1 | | | | | |
| RT- | 243 | T | −0.22 | 77.78 | Highly |
| Cas1 | | | | | interchangeable |
| RT- | 244 | E | −0.19 | 83.33 | Highly |
| Cas1 | | | | | interchangeable |
| RT- | 245 | R | −0.47 | 72.22 | Highly |
| Cas1 | | | | | interchangeable |
| RT- | 246 | L | −3.45 | 11.11 | Limited |
| Cas1 | | | | | interchangeability |
| RT- | 247 | E | −0.35 | 83.33 | Highly |
| Cas1 | | | | | interchangeable |
| RT- | 248 | R | −0.11 | 91.67 | Highly |
| Cas1 | | | | | interchangeable |
| RT- | 249 | R | −0.31 | 86.11 | Highly |
| Cas1 | | | | | interchangeable |
| RT- | 250 | K | −0.84 | 38.89 | Interchangeable |
| Cas1 | | | | | |
| RT- | 251 | L | −3.66 | 2.86 | Not interchangeable |
| Cas1 | | | | | |
| RT- | 252 | T | −0.36 | 69.44 | Highly |
| Cas1 | | | | | interchangeable |
| RT- | 253 | V | −2.45 | 19.44 | Limited |
| Cas1 | | | | | interchangeability |
| RT- | 254 | N | −2.45 | 5.56 | Not interchangeable |
| Cas1 | | | | | |
| RT- | 255 | K | 0.72 | 100.00 | Highly |
| Cas1 | | | | | interchangeable |
| RT- | 256 | E | −1.03 | 27.78 | Limited |
| Cas1 | | | | | interchangeability |
| RT- | 257 | K | −3.42 | 0.00 | Not interchangeable |
| Cas1 | | | | | |
| RT- | 258 | S | −3.69 | 0.00 | Not interchangeable |
| Cas1 | | | | | |
| RT- | 259 | G | −3.90 | 0.00 | Not interchangeable |
| Cas1 | | | | | |
| RT- | 260 | V | −3.20 | 13.89 | Limited |
| Cas1 | | | | | interchangeability |
| RT- | 261 | F | −3.04 | 5.56 | Not interchangeable |
| Cas1 | | | | | |
| RT- | 262 | D | −0.44 | 63.89 | Highly |
| Cas1 | | | | | interchangeable |
| RT- | 263 | V | −1.78 | 22.22 | Limited |
| Cas1 | | | | | interchangeability |
| RT- | 264 | S | −0.69 | 48.57 | Interchangeable |
| Cas1 | | | | | |
| RT- | 265 | T | −0.86 | 44.44 | Interchangeable |
| Cas1 | | | | | |
| RT- | 266 | R | −1.95 | 8.33 | Not interchangeable |
| Cas1 | | | | | |
| RT- | 267 | S | −0.76 | 47.22 | Interchangeable |
| Cas1 | | | | | |
| RT- | 268 | — | −2.64 | 22.22 | Limited |
| Cas1 | | | | | interchangeability |
| RT- | 269 | L | −3.81 | 0.00 | Not interchangeable |
| Cas1 | | | | | |
| RT- | 270 | G | −3.01 | 5.56 | Not interchangeable |
| Cas1 | | | | | |
| RT- | 271 | Y | −3.61 | 2.78 | Not interchangeable |
| Cas1 | | | | | |
| RT- | 272 | D | −2.97 | 2.78 | Not interchangeable |
| Cas1 | | | | | |
| RT- | 273 | — | −2.95 | 8.33 | Not interchangeable |
| Cas1 | | | | | |
| RT- | 274 | L | −1.13 | 22.22 | Limited |
| Cas1 | | | | | interchangeability |
| RT- | 275 | — | −0.86 | 33.33 | Interchangeable |
| Cas1 | | | | | |
| RT- | 276 | R | −1.06 | 36.11 | Interchangeable |
| Cas1 | | | | | |
| RT- | 277 | N | −0.65 | 44.44 | Interchangeable |
| Cas1 | | | | | |
| RT- | 278 | K | −1.26 | 19.44 | Limited |
| Cas1 | | | | | interchangeability |
| RT- | 279 | K | −1.73 | 16.67 | Limited |
| Cas1 | | | | | interchangeability |
| RT- | 280 | V | −2.41 | 16.67 | Limited |
| Cas1 | | | | | interchangeability |
| RT- | 281 | D | −2.76 | 5.56 | Not interchangeable |
| Cas1 | | | | | |
| RT- | 282 | V | −2.94 | 22.22 | Limited |
| Cas1 | | | | | interchangeability |
| RT- | 283 | R | −2.86 | 0.00 | Not interchangeable |
| Cas1 | | | | | |
| RT- | 284 | K | −2.18 | 16.67 | Limited |
| Cas1 | | | | | interchangeability |
| RT | 285 | H | −2.68 | 5.56 | Not interchangeable |
| Cas1 | | | | | |
| RT- | 286 | I | −1.16 | 27.78 | Limited |
| Cas1 | | | | | interchangeability |
| RT- | 287 | Y | −1.29 | 5.56 | Not interchangeable |
| Cas1 | | | | | |
| RT- | 288 | K | −0.55 | 66.67 | Highly |
| Cas1 | | | | | interchangeable |
| RT- | 289 | S | −0.59 | 36.11 | Interchangeable |
| Cas1 | | | | | |
| RT- | 290 | V | −1.07 | 41.67 | Interchangeable |
| Cas1 | | | | | |
| RT- | 291 | N | −0.54 | 61.11 | Highly |
| Cas1 | | | | | interchangeable |
| RT- | 292 | Q | −0.68 | 50.00 | Interchangeable |
| Cas1 | | | | | |
| RT- | 293 | Y | −2.55 | 8.33 | Not interchangeable |
| Cas1 | | | | | |
| RT- | 294 | S | −0.84 | 38.89 | Interchangeable |
| Cas1 | | | | | |
| RT- | 295 | N | −0.49 | 61.11 | Highly |
| Cas1 | | | | | interchangeable |
| RT- | 296 | W | −3.94 | 0.00 | Not interchangeable |
| Cas1 | | | | | |
| RT- | 297 | H | −1.43 | 11.11 | Limited |
| Cas1 | | | | | interchangeability |
| RT- | 298 | D | −1.29 | 11.11 | Limited |
| Cas1 | | | | | interchangeability |
| RT- | 299 | S | −2.37 | 13.89 | Limited |
| Cas1 | | | | | interchangeability |
| RT- | 300 | R | −1.27 | 30.56 | Interchangeable |
| Cas1 | | | | | |
| RT- | 301 | L | −3.75 | 0.00 | Not interchangeable |
| Cas1 | | | | | |
| RT- | 302 | E | −1.04 | 30.56 | Interchangeable |
| Cas1 | | | | | |
| RT- | 303 | F | −0.89 | 47.22 | Interchangeable |
| Cas1 | | | | | |
| RT- | 304 | — | −2.10 | 0.00 | Not interchangeable |
| Cas1 | | | | | |
| RT- | 305 | N | −2.55 | 0.00 | Not interchangeable |
| Cas1 | | | | | |
| RT- | 306 | G | −3.20 | 0.00 | Not interchangeable |
| Cas1 | | | | | |
| RT- | 307 | R | −2.00 | 8.33 | Not interchangeable |
| Cas1 | | | | | |
| RT- | 308 | Y | −3.31 | 5.56 | Not interchangeable |
| Cas1 | | | | | |
| RT- | 309 | H | −3.95 | 0.00 | Not interchangeable |
| Cas1 | | | | | |
| RT- | 310 | — | −3.99 | 0.00 | Not interchangeable |
| Cas1 | | | | | |
| RT- | 311 | T | −2.81 | 13.89 | Limited |
| Cas1 | | | | | interchangeability |
| RT- | 312 | S | −2.88 | 2.78 | Not interchangeable |
| Cas1 | | | | | |
| RT- | 313 | D | −3.68 | 0.00 | Not interchangeable |
| Cas1 | | | | | |
| RT- | 314 | G | −3.93 | 2.78 | Not interchangeable |
| Cas1 | | | | | |
| RT- | 315 | — | −3.80 | 8.33 | Not interchangeable |
| Cas1 | | | | | |
| RT- | 316 | L | −3.51 | 12.12 | Limited |
| Cas1 | | | | | interchangeability |
| RT- | 317 | N | −1.88 | 27.78 | Limited |
| Cas1 | | | | | interchangeability |
| RT- | 318 | R | −2.00 | 0.00 | Not interchangeable |
| Cas1 | | | | | |
| RT- | 319 | Q | −0.62 | 52.78 | Interchangeable |
| Cas1 | | | | | |
| RT- | 320 | D | −3.61 | 0.00 | Not interchangeable |
| Cas1 | | | | | |
| RT- | 321 | F | −3.32 | 5.56 | Not interchangeable |
| Cas1 | | | | | |
| RT- | 322 | G | −2.35 | 19.44 | Limited |
| Cas1 | | | | | interchangeability |
| RT- | 323 | L | −3.73 | 5.56 | Not interchangeable |
| Cas1 | | | | | |
| RT- | 324 | L | −4.16 | 2.78 | Not interchangeable |
| Cas1 | | | | | |
| RT- | 325 | F | −5.17 | 0.00 | Not interchangeable |
| Cas1 | | | | | |
| RT- | 326 | E | −2.75 | 0.00 | Not interchangeable |
| Cas1 | | | | | |
| RT- | 327 | N | −4.78 | 0.00 | Not interchangeable |
| Cas1 | | | | | |
| RT- | 328 | E | −1.40 | 16.67 | Limited |
| Cas1 | | | | | interchangeability |
| RT- | 329 | Q | −0.69 | 55.56 | Interchangeable |
| Cas1 | | | | | |
| RT- | 330 | K | −1.38 | 16.67 | Limited |
| Cas1 | | | | | interchangeability |
| RT- | 331 | K | −2.15 | 11.11 | Limited |
| Cas1 | | | | | interchangeability |
| RT- | 332 | H | −1.25 | 38.89 | Interchangeable |
| Cas1 | | | | | |
| RT- | 333 | Y | −1.48 | 22.22 | Limited |
| Cas1 | | | | | interchangeability |
| RT- | 334 | — | −4.01 | 5.56 | Not interchangeable |
| Cas1 | | | | | |
| RT- | 335 | P | −4.60 | 0.00 | Not interchangeable |
| Cas1 | | | | | |
| RT- | 336 | V | −1.21 | 33.33 | Interchangeable |
| Cas1 | | | | | |
| RT- | 337 | E | −3.61 | 0.00 | Not interchangeable |
| Cas1 | | | | | |
| RT- | 338 | V | −3.85 | 0.00 | Not interchangeable |
| Cas1 | | | | | |
| RT- | 339 | S | −2.44 | 16.67 | Limited |
| Cas1 | | | | | interchangeability |
| RT- | 340 | D | −3.44 | 5.56 | Not interchangeable |
| Cas1 | | | | | |
| RT- | 341 | Q | −2.25 | 27.78 | Limited |
| Cas1 | | | | | interchangeability |
| RT- | 342 | L | −3.86 | 11.11 | Limited |
| Cas1 | | | | | interchangeability |
| RT- | 343 | N | −4.41 | 0.00 | Not interchangeable |
| Cas1 | | | | | |
| RT- | 344 | — | −3.84 | 13.89 | Limited |
| Cas1 | | | | | interchangeability |
| RT- | 345 | Y | −3.84 | 11.11 | Limited |
| Cas1 | | | | | interchangeability |
| RT- | 346 | G | −3.94 | 11.11 | Limited |
| Cas1 | | | | | interchangeability |
| RT- | 347 | N | −3.29 | 0.00 | Not interchangeable |
| Cas1 | | | | | |
| RT- | 348 | V | −4.36 | 0.00 | Not interchangeable |
| Cas1 | | | | | |
| RT- | 349 | T | −2.07 | 16.67 | Limited |
| Cas1 | | | | | interchangeability |
| RT- | 350 | L | −4.23 | 0.00 | Not interchangeable |
| Cas1 | | | | | |
| RT | 351 | A | −2.39 | 16.67 | Limited |
| Cas1 | | | | | interchangeability |
| RT- | 352 | S | −1.61 | 16.67 | Limited |
| Cas1 | | | | | interchangeability |
| RT- | 353 | N | −2.14 | 13.89 | Limited |
| Cas1 | | | | | interchangeability |
| RT- | 354 | V | −3.87 | 2.78 | Not interchangeable |
| Cas1 | | | | | |
| RT- | 355 | L | −4.39 | 0.00 | Not interchangeable |
| Cas1 | | | | | |
| RT- | 356 | Q | −1.37 | 13.89 | Limited |
| Cas1 | | | | | interchangeability |
| RT- | 357 | S | −2.08 | 22.22 | Limited |
| Cas1 | | | | | interchangeability |
| RT- | 358 | F | −3.81 | 11.11 | Limited |
| Cas1 | | | | | interchangeability |
| RT- | 359 | S | −2.40 | 11.11 | Limited |
| Cas1 | | | | | interchangeability |
| RT- | 360 | N | −0.54 | 66.67 | Highly |
| Cas1 | | | | | interchangeable |
| RT- | 361 | R | −0.23 | 66.67 | Highly |
| Cas1 | | | | | interchangeable |
| RT- | 362 | E | −1.37 | 50.00 | Interchangeable |
| Cas1 | | | | | |
| RT- | 363 | — | −4.20 | 5.56 | Not interchangeable |
| Cas1 | | | | | |
| RT- | 364 | K | −1.48 | 41.67 | Interchangeable |
| Cas1 | | | | | |
| RT- | 365 | V | −4.17 | 0.00 | Not interchangeable |
| Cas1 | | | | | |
| RT- | 366 | S | −2.73 | 16.67 | Limited |
| Cas1 | | | | | interchangeability |
| RT- | 367 | F | −4.32 | 0.00 | Not interchangeable |
| Cas1 | | | | | |
| RT- | 368 | F | −4.63 | 0.00 | Not interchangeable |
| Cas1 | | | | | |
| RT- | 369 | D | −4.78 | 0.00 | Not interchangeable |
| Cas1 | | | | | |
| RT- | 370 | K | −2.09 | 5.56 | Not interchangeable |
| Cas1 | | | | | |
| RT- | 371 | Y | −2.88 | 2.78 | Not interchangeable |
| Cas1 | | | | | |
| RT- | 372 | G | −4.48 | 0.00 | Not interchangeable |
| Cas1 | | | | | |
| RT- | 373 | R | −1.31 | 11.11 | Limited |
| Cas1 | | | | | interchangeability |
| RT- | 374 | L | −3.12 | 8.33 | Not interchangeable |
| Cas1 | | | | | |
| RT- | 375 | — | −4.25 | 8.33 | Not interchangeable |
| Cas1 | | | | | |
| RT- | 376 | G | −4.66 | 0.00 | Not interchangeable |
| Cas1 | | | | | |
| RT. | 377 | S | −1.34 | 55.56 | Interchangeable |
| Cas1 | | | | | |
| RT- | 378 | F | −4.41 | 0.00 | Not interchangeable |
| Cas1 | | | | | |
| RT- | 379 | L | −3.00 | 8.33 | Not interchangeable |
| Cas1 | | | | | |
| RT- | 380 | P | −4.68 | 0.00 | Not interchangeable |
| Cas1 | | | | | |
| RT- | 381 | E | −2.58 | 5.56 | Not interchangeable |
| Cas1 | | | | | |
| RT- | 382 | K | −1.15 | 40.00 | Interchangeable |
| Cas1 | | | | | |
| RT- | 383 | T | −1.93 | 25.00 | Limited |
| Cas1 | | | | | interchangeability |
| RT- | 384 | K | −1.17 | 27.78 | Limited |
| Cas1 | | | | | interchangeability |
| RT- | 385 | K | −1.74 | 27.78 | Limited |
| Cas1 | | | | | interchangeability |
| RT- | 386 | S | −2.00 | 25.00 | Limited |
| Cas1 | | | | | interchangeability |
| RT- | 387 | A | −2.00 | 8.33 | Not interchangeable |
| Cas1 | | | | | |
| RT- | 388 | E | −0.91 | 41.67 | Interchangeable |
| Cas1 | | | | | |
| RT- | 389 | — | −3.28 | 8.33 | Not interchangeable |
| Cas1 | | | | | |
| RT- | 390 | I | −3.44 | 11.11 | Limited |
| Cas1 | | | | | interchangeability |
| RT- | 391 | L | −3.52 | 0.00 | Not interchangeable |
| Cas1 | | | | | |
| RT- | 392 | V | −0.37 | 88.89 | Highly |
| Cas1 | | | | | interchangeable |
| RT- | 393 | Q | −4.81 | 0.00 | Not interchangeable |
| Cas1 | | | | | |
| RT- | 394 | S | −2.37 | 36.11 | Interchangeable |
| Cas1 | | | | | |
| RT- | 395 | K | −1.48 | 33.33 | Interchangeable |
| Cas1 | | | | | |
| RT- | 396 | N | −1.49 | 36.11 | Interchangeable |
| Cas1 | | | | | |
| RT- | 397 | Y | −3.40 | 0.00 | Not interchangeable |
| Cas1 | | | | | |
| RT- | 398 | L | −1.79 | 16.67 | Limited |
| Cas1 | | | | | interchangeability |
| RT- | 399 | N | −1.86 | 11.11 | Limited |
| Cas1 | | | | | interchangeability |
| RT- | 400 | E | −0.74 | 36.11 | Interchangeable |
| Cas1 | | | | | |
| RT- | 401 | D | −1.09 | 11.11 | Limited |
| Cas1 | | | | | interchangeability |
| RT- | 402 | V | −1.20 | 30.56 | Interchangeable |
| Cas1 | | | | | |
| RT- | 403 | R | −4.44 | 5.56 | Not interchangeable |
| Cas1 | | | | | |
| RT- | 404 | M | −1.44 | 29.73 | Limited |
| Cas1 | | | | | interchangeability |
| RT- | 405 | D | −1.07 | 22.86 | Limited |
| Cas1 | | | | | interchangeability |
| RT- | 406 | T | −3.00 | 25.00 | Limited |
| Cas1 | | | | | interchangeability |
| RT- | 407 | A | −4.16 | 2.78 | Not interchangeable |
| Cas1 | | | | | |
| RT- | 408 | R | −3.90 | 5.56 | Not interchangeable |
| Cas1 | | | | | |
| RT- | 409 | R | −1.65 | 25.00 | Limited |
| Cas1 | | | | | interchangeability |
| RT- | 410 | M | −4.43 | 0.00 | Not interchangeable |
| Cas1 | | | | | |
| RT- | 411 | E | −2.14 | 19.44 | Limited |
| Cas1 | | | | | interchangeability |
| RT- | 412 | — | −0.89 | 58.33 | Interchangeable |
| Cas1 | | | | | |
| RT | 413 | A | −4.62 | 0.00 | Not interchangeable |
| Cas1 | | | | | |
| RT- | 414 | G | −2.19 | 22.22 | Limited |
| Cas1 | | | | | interchangeability |
| RT- | 415 | L | −3.73 | 2.78 | Not interchangeable |
| Cas1 | | | | | |
| RT- | 416 | H | −4.09 | 0.00 | Not interchangeable |
| Cas1 | | | | | |
| RT | 417 | N | −4.53 | 0.00 | Not interchangeable |
| Cas1 | | | | | |
| RT- | 418 | I | −3.86 | 5.56 | Not interchangeable |
| Cas1 | | | | | |
| RT- | 419 | R | −4.53 | 0.00 | Not interchangeable |
| Cas1 | | | | | |
| RT- | 420 | A | −3.96 | 0.00 | Not interchangeable |
| Cas1 | | | | | |
| RT- | 421 | N | −3.64 | 5.56 | Not interchangeable |
| Cas1 | | | | | |
| RT- | 422 | L | −4.50 | 0.00 | Not interchangeable |
| Cas1 | | | | | |
| RT- | 423 | R | −4.22 | 2.78 | Not interchangeable |
| Cas1 | | | | | |
| RT- | 424 | Y | −4.18 | 8.33 | Not interchangeable |
| Cas1 | | | | | |
| RT- | 425 | Y | −4.75 | 0.00 | Not interchangeable |
| Cas1 | | | | | |
| RT- | 426 | D | −1.53 | 11.11 | Limited |
| Cas1 | | | | | interchangeability |
| RT- | 427 | K | −4.30 | 0.00 | Not interchangeable |
| Cas1 | | | | | |
| RT- | 428 | K | −2.97 | 8.33 | Not interchangeable |
| Cas1 | | | | | |
| RT- | 429 | H | −1.72 | 11.11 | Limited |
| Cas1 | | | | | interchangeability |
| RT- | 430 | K | −1.51 | 27.78 | Limited |
| Cas1 | | | | | interchangeability |
| RT- | 431 | G | −1.26 | 16.67 | Limited |
| Cas1 | | | | | interchangeability |
| RT- | 432 | D | −0.23 | 77.78 | Highly |
| Cas1 | | | | | interchangeable |
| RT- | 433 | F | −3.97 | 8.33 | Not interchangeable |
| Cas1 | | | | | |
| RT- | 434 | K | −1.28 | 25.00 | Limited |
| Cas1 | | | | | interchangeability |
| RT- | 435 | E | −0.93 | 25.00 | Limited |
| Cas1 | | | | | interchangeability |
| RT- | 436 | K | −0.49 | 61.11 | Highly |
| Cas1 | | | | | interchangeable |
| RT- | 437 | V | −3.47 | 5.56 | Not interchangeable |
| Cas1 | | | | | |
| RT- | 438 | D | −2.27 | 2.78 | Not interchangeable |
| Cas1 | | | | | |
| RT- | 439 | A | −1.04 | 41.67 | Interchangeable |
| Cas1 | | | | | |
| RT- | 440 | — | −4.26 | 2.78 | Not interchangeable |
| Cas1 | | | | | |
| RT- | 441 | S | −2.70 | 11.11 | Limited |
| Cas1 | | | | | interchangeability |
| RT- | 442 | G | −0.42 | 72.22 | Highly |
| Cas1 | | | | | interchangeable |
| RT- | 443 | Y | −2.74 | 13.89 | Limited |
| Cas1 | | | | | interchangeability |
| RT- | 444 | — | −4.17 | 2.78 | Not interchangeable |
| Cas1 | | | | | |
| RT- | 445 | D | −0.73 | 52.78 | Interchangeable |
| Cas1 | | | | | |
| RT- | 446 | A | −1.33 | 25.00 | Limited |
| Cas1 | | | | | interchangeability |
| RT- | 447 | L | −3.83 | 2.78 | Not interchangeable |
| Cas1 | | | | | |
| RT- | 448 | N | 0.70 | 94.44 | Highly |
| Cas1 | | | | | interchangeable |
| RT- | 449 | R | 0.88 | 94.44 | Highly |
| Cas1 | | | | | interchangeable |
| RT- | 450 | A | −3.02 | 19.44 | Limited |
| Cas1 | | | | | interchangeability |
| RT- | 451 | P | −0.20 | 72.22 | Highly |
| Cas1 | | | | | interchangeable |
| RT- | 452 | S | −2.43 | 8.33 | Not interchangeable |
| Cas1 | | | | | |
| RT- | 453 | V | −2.89 | 5.56 | Not interchangeable |
| Cas1 | | | | | |
| RT- | 454 | N | 0.09 | 94.44 | Highly |
| Cas1 | | | | | interchangeable |
| RT- | 455 | D | −1.48 | 19.44 | Limited |
| Cas1 | | | | | interchangeability |
| RT- | 456 | M | −3.95 | 2.70 | Not interchangeable |
| Cas1 | | | | | |
| RT- | 457 | M | −1.84 | 13.51 | Limited |
| Cas1 | | | | | interchangeability |
| RT- | 458 | L | −2.79 | 8.33 | Not interchangeable |
| Cas1 | | | | | |
| RT- | 459 | L | −2.56 | 16.67 | Limited |
| Cas1 | | | | | interchangeability |
| RT- | 460 | E | −5.04 | 0.00 | Not interchangeable |
| Cas1 | | | | | |
| RT- | 461 | A | −4.11 | 5.56 | Not interchangeable |
| Cas1 | | | | | |
| RT- | 462 | K | −2.24 | 13.89 | Limited |
| Cas1 | | | | | interchangeability |
| RT- | 463 | A | −4.21 | 0.00 | Not interchangeable |
| Cas1 | | | | | |
| RT- | 464 | R | −4.45 | 2.78 | Not interchangeable |
| Cas1 | | | | | |
| RT- | 465 | Q | −1.10 | 27.78 | Limited |
| Cas1 | | | | | interchangeability |
| RT- | 466 | L | −1.79 | 16.67 | Limited |
| Cas1 | | | | | interchangeability |
| RT- | 467 | Y | −4.85 | 0.00 | Not interchangeable |
| Cas1 | | | | | |
| RT | 468 | Y | −4.42 | 0.00 | Not interchangeable |
| Cas1 | | | | | |
| RT- | 469 | T | −0.21 | 77.78 | Highly |
| Cas1 | | | | | interchangeable |
| RT- | 470 | C | −3.19 | 11.11 | Limited |
| Cas1 | | | | | interchangeability |
| RT- | 471 | F | −4.64 | 2.78 | Not interchangeable |
| Cas1 | | | | | |
| RT- | 472 | N | −3.16 | 2.78 | Not interchangeable |
| Cas1 | | | | | |
| RT- | 473 | Q | −1.31 | 13.89 | Limited |
| Cas1 | | | | | interchangeability |
| RT- | 474 | I | −4.99 | 0.00 | Not interchangeable |
| Cas1 | | | | | |
| RT | 475 | L | −2.96 | 5.56 | Not interchangeable |
| Cas1 | | | | | |
| RT | 476 | E | 0.95 | 94.44 | Highly |
| Cas1 | | | | | interchangeable |
| RT- | 477 | T | −0.05 | 69.44 | Highly |
| Cas1 | | | | | interchangeable |
| RT- | 478 | S | −0.70 | 52.78 | Interchangeable |
| Cas1 | | | | | |
| RT- | 479 | D | −2.25 | 13.89 | Limited |
| Cas1 | | | | | interchangeability |
| RT- | 480 | F | −3.84 | 0.00 | Not interchangeable |
| Cas1 | | | | | |
| RT- | 481 | Q | −0.27 | 80.56 | Highly |
| Cas1 | | | | | interchangeable |
| RT- | 482 | F | −3.40 | 5.56 | Not interchangeable |
| Cas1 | | | | | |
| RT- | 483 | E | −0.47 | 61.11 | Highly |
| Cas1 | | | | | interchangeable |
| RT- | 484 | K | −0.80 | 55.56 | Interchangeable |
| Cas1 | | | | | |
| RT- | 485 | R | −4.03 | 0.00 | Not interchangeable |
| Cas1 | | | | | |
| RT- | 486 | T | −2.81 | 5.56 | Not interchangeable |
| Cas1 | | | | | |
| RT- | 487 | K | −1.31 | 13.89 | Limited |
| Cas1 | | | | | interchangeability |
| RT- | 488 | R | −1.81 | 22.22 | Limited |
| Cas1 | | | | | interchangeability |
| RT- | 489 | P | −3.61 | 2.78 | Not interchangeable |
| Cas1 | | | | | |
| RT- | 490 | P | −3.88 | 0.00 | Not interchangeable |
| Cas1 | | | | | |
| RT- | 491 | K | −1.79 | 13.89 | Limited |
| Cas1 | | | | | interchangeability |
| RT- | 492 | D | −3.80 | 0.00 | Not interchangeable |
| Cas1 | | | | | |
| RT- | 493 | A | −2.82 | 8.33 | Not interchangeable |
| Cas1 | | | | | |
| RT- | 494 | — | −3.44 | 8.33 | Not interchangeable |
| Cas1 | | | | | |
| RT- | 495 | N | −3.94 | 0.00 | Not interchangeable |
| Cas1 | | | | | |
| RT- | 496 | A | −3.56 | 0.00 | Not interchangeable |
| Cas1 | | | | | |
| RT- | 497 | C | −3.11 | 11.11 | Limited |
| Cas1 | | | | | interchangeability |
| RT- | 498 | — | −3.63 | 2.78 | Not interchangeable |
| Cas1 | | | | | |
| RT- | 499 | S | −3.65 | 0.00 | Not interchangeable |
| Cas1 | | | | | |
| RT- | 500 | F | −3.88 | 0.00 | Not interchangeable |
| Cas1 | | | | | |
| RT- | 501 | G | −3.88 | 0.00 | Not interchangeable |
| Cas1 | | | | | |
| RT- | 502 | N | −3.71 | 0.00 | Not interchangeable |
| Cas1 | | | | | |
| RT- | 503 | T | −2.52 | 5.56 | Not interchangeable |
| Cas1 | | | | | |
| RT- | 504 | L | −3.54 | 0.00 | Not interchangeable |
| Cas1 | | | | | |
| RT- | 505 | L | −3.91 | 0.00 | Not interchangeable |
| Cas1 | | | | | |
| RT- | 506 | Y | −3.03 | 0.00 | Not interchangeable |
| Cas1 | | | | | |
| RT- | 507 | N | −2.60 | 5.56 | Not interchangeable |
| Cas1 | | | | | |
| RT- | 508 | L | −1.27 | 38.89 | Interchangeable |
| Cas1 | | | | | |
| RT- | 509 | F | −3.81 | 0.00 | Not interchangeable |
| Cas1 | | | | | |
| RT- | 510 | V | −0.83 | 47.22 | Interchangeable |
| Cas1 | | | | | |
| RT- | 511 | N | −1.73 | 22.22 | Limited |
| Cas1 | | | | | interchangeability |
| RT- | 512 | — | −3.53 | 0.00 | Not interchangeable |
| Cas1 | | | | | |
| RT- | 513 | — | −3.49 | 5.56 | Not interchangeable |
| Cas1 | | | | | |
| RT- | 514 | W | −2.78 | 8.11 | Not interchangeable |
| Cas1 | | | | | |
| RT- | 515 | K | −1.95 | 13.89 | Limited |
| Cas1 | | | | | interchangeability |
| RT- | 516 | K | −3.03 | 0.00 | Not interchangeable |
| Cas1 | | | | | |
| RT- | 517 | G | −4.08 | 0.00 | Not interchangeable |
| Cas1 | | | | | |
| RT- | 518 | L | −3.79 | 2.78 | Not interchangeable |
| Cas1 | | | | | |
| RT- | 519 | D | −3.91 | 0.00 | Not interchangeable |
| Cas1 | | | | | |
| RT | 520 | P | −3.85 | 0.00 | Not interchangeable |
| Cas1 | | | | | |
| RT- | 521 | R | −3.46 | 0.00 | Not interchangeable |
| Cas1 | | | | | |
| RT- | 522 | F | −3.78 | 0.00 | Not interchangeable |
| Cas1 | | | | | |
| RT | 523 | G | −3.92 | 0.00 | Not interchangeable |
| Cas1 | | | | | |
| RT- | 524 | V | −3.47 | 2.78 | Not interchangeable |
| Cas1 | | | | | |
| RT- | 525 | V | −2.93 | 13.89 | Limited |
| Cas1 | | | | | interchangeability |
| RT. | 526 | H | −4.08 | 0.00 | Not interchangeable |
| Cas1 | | | | | |
| RT- | 527 | A | −1.58 | 13.89 | Limited |
| Cas1 | | | | | interchangeability |
| RT | 528 | S | −2.02 | 13.89 | Limited |
| Cas1 | | | | | interchangeability |
| RT- | 529 | N | −1.37 | 11.11 | Limited |
| Cas1 | | | | | interchangeability |
| RT- | 530 | K | −0.19 | 75.00 | Highly |
| Cas1 | | | | | interchangeable |
| RT- | 531 | R | −3.10 | 0.00 | Not interchangeable |
| Cas1 | | | | | |
| RT | 532 | N | −0.62 | 52.78 | Interchangeable |
| Cas1 | | | | | |
| RT- | 533 | Q | −0.94 | 50.00 | Interchangeable |
| Cas1 | | | | | |
| RT- | 534 | S | −3.13 | 2.78 | Not interchangeable |
| Cas1 | | | | | |
| RT- | 535 | L | −3.92 | 0.00 | Not interchangeable |
| Cas1 | | | | | |
| RT- | 536 | N | −3.54 | 0.00 | Not interchangeable |
| Cas1 | | | | | |
| RT- | 537 | L | −3.44 | 0.00 | Not interchangeable |
| Cas1 | | | | | |
| RT- | 538 | D | −3.83 | 0.00 | Not interchangeable |
| Cas1 | | | | | |
| RT- | 539 | F | −3.93 | 2.78 | Not interchangeable |
| Cas1 | | | | | |
| RT- | 540 | A | −3.59 | 0.00 | Not interchangeable |
| Cas1 | | | | | |
| RT- | 541 | D | −3.97 | 0.00 | Not interchangeable |
| Cas1 | | | | | |
| RT- | 542 | — | −3.63 | 0.00 | Not interchangeable |
| Cas1 | | | | | |
| RT- | 543 | F | −3.60 | 0.00 | Not interchangeable |
| Cas1 | | | | | |
| RT- | 544 | K | −3.91 | 0.00 | Not interchangeable |
| Cas1 | | | | | |
| RT- | 545 | P | −3.80 | 0.00 | Not interchangeable |
| Cas1 | | | | | |
| RT- | 546 | — | −3.52 | 2.78 | Not interchangeable |
| Cas1 | | | | | |
| RT- | 547 | V | −3.47 | 5.56 | Not interchangeable |
| Cas1 | | | | | |
| RT- | 548 | — | −3.10 | 11.11 | Limited |
| Cas1 | | | | | interchangeability |
| RT- | 549 | D | −3.95 | 2.78 | Not interchangeable |
| Cas1 | | | | | |
| RT- | 550 | R | −3.81 | 0.00 | Not interchangeable |
| Cas1 | | | | | |
| RT- | 551 | — | −3.20 | 11.11 | Limited |
| Cas1 | | | | | interchangeability |
| RT- | 552 | — | −4.05 | 0.00 | Not interchangeable |
| Cas1 | | | | | |
| RT- | 553 | F | −4.43 | 0.00 | Not interchangeable |
| Cas1 | | | | | |
| RT- | 554 | T | −3.30 | 0.00 | Not interchangeable |
| Cas1 | | | | | |
| RT- | 555 | M | −3.09 | 8.1 | Not interchangeable |
| Cas1 | | | | | |
| RT- | 556 | — | −3.19 | 5.56 | Not interchangeable |
| Cas1 | | | | | |
| RT- | 557 | N | −3.73 | 0.00 | Not interchangeable |
| Cas1 | | | | | |
| RT- | 558 | K | −3.86 | 0.00 | Not interchangeable |
| Cas1 | | | | | |
| RT- | 559 | K | −2.73 | 13.89 | Limited |
| Cas1 | | | | | interchangeability |
| RT- | 560 | M | −3.17 | 10.81 | Limited |
| Cas1 | | | | | interchangeability |
| RT- | 561 | L | −3.36 | 0.00 | Not interchangeable |
| Cas1 | | | | | |
| RT- | 562 | T | −1.07 | 38.89 | Interchangeable |
| Cas1 | | | | | |
| RT- | 563 | L | −0.83 | 36.11 | Interchangeable |
| Cas1 | | | | | |
| RT- | 564 | L | 1.29 | 100.00 | Highly |
| Cas1 | | | | | interchangeable |
| RT- | 565 | T | −0.05 | 61.11 | Highly |
| Cas1 | | | | | interchangeable |
| RT- | 566 | D | −2.15 | 27.78 | Limited |
| Cas1 | | | | | interchangeability |
| RT- | 567 | F | −3.99 | 0.00 | Not interchangeable |
| Cas1 | | | | | |
| RT- | 568 | E | −1.22 | 16.67 | Limited |
| Cas1 | | | | | interchangeability |
| RT- | 569 | T | −0.48 | 63.89 | Highly |
| Cas1 | | | | | interchangeable |
| RT- | 570 | S | −0.39 | 80.56 | Highly |
| Cas1 | | | | | interchangeable |
| RT- | 571 | N | −0.52 | 58.33 | Interchangeable |
| Cas1 | | | | | |
| RT- | 572 | Q | −0.48 | 50.00 | Interchangeable |
| Cas1 | | | | | |
| RT- | 573 | G | −2.75 | 0.00 | Not interchangeable |
| Cas1 | | | | | |
| RT- | 574 | V | −2.79 | 8.33 | Not interchangeable |
| Cas1 | | | | | |
| RT- | 575 | Y | −1.81 | 8.33 | Not interchangeable |
| Cas1 | | | | | |
| RT- | 576 | L | −3.74 | 0.00 | Not interchangeable |
| Cas1 | | | | | |
| RT- | 577 | S | −2.78 | 8.33 | Not interchangeable |
| Cas1 | | | | | |
| RT- | 578 | R | −1.09 | 27.78 | Limited |
| Cas1 | | | | | interchangeability |
| RT- | 579 | E | −0.28 | 58.33 | Interchangeable |
| Cas1 | | | | | |
| RT- | 580 | G | −3.66 | 0.00 | Not interchangeable |
| Cas1 | | | | | |
| RT- | 581 | K | −3.55 | 0.00 | Not interchangeable |
| Cas1 | | | | | |
| RT- | 582 | N | −0.98 | 36.11 | Interchangeable |
| Cas1 | | | | | |
| RT- | 583 | — | −1.09 | 36.11 | Interchangeable |
| Cas1 | | | | | |
| RT- | 584 | F | −3.64 | 0.00 | Not interchangeable |
| Cas1 | | | | | |
| RT- | 585 | L | −3.36 | 2.78 | Not interchangeable |
| Cas1 | | | | | |
| RT- | 586 | Q | −0.87 | 44.44 | Interchangeable |
| Cas1 | | | | | |
| RT- | 587 | M | −1.14 | 48.65 | Interchangeable |
| Cas1 | | | | | |
| RT- | 588 | Y | −2.76 | 11.11 | Limited |
| Cas1 | | | | | interchangeability |
| RT- | 589 | E | −3.48 | 0.00 | Not interchangeable |
| Cas1 | | | | | |
| RT- | 590 | E | 1.06 | 91.67 | Highly |
| Cas1 | | | | | interchangeable |
| RT- | 591 | K | −4.01 | 0.00 | Not interchangeable |
| Cas1 | | | | | |
| RT- | 592 | L | −3.92 | 0.00 | Not interchangeable |
| Cas1 | | | | | |
| RT- | 593 | K | −1.71 | 8.33 | Not interchangeable |
| Cas1 | | | | | |
| RT- | 594 | S | −2.17 | 11.11 | Limited |
| Cas1 | | | | | interchangeability |
| RT- | 595 | R | −1.83 | 13.89 | Limited |
| Cas1 | | | | | interchangeability |
| RT | 596 | — | −2.66 | 11.43 | Limited |
| Cas1 | | | | | interchangeability |
| RT- | 597 | T | −2.38 | 2.78 | Not interchangeable |
| Cas1 | | | | | |
| RT- | 598 | — | −1.49 | 22.22 | Limited |
| Cas1 | | | | | interchangeability |
| RT- | 599 | K | −1.90 | 2.78 | Not interchangeable |
| Cas1 | | | | | |
| RT- | 600 | G | −2.48 | 0.00 | Not interchangeable |
| Cas1 | | | | | |
| RT- | 601 | K | −0.90 | 41.67 | Interchangeable |
| Cas1 | | | | | |
| RT- | 602 | E | −0.69 | 47.22 | Interchangeable |
| Cas1 | | | | | |
| RT- | 603 | M | −2.04 | 2.70 | Not interchangeable |
| Cas1 | | | | | |
| RT- | 604 | S | −2.37 | 8.33 | Not interchangeable |
| Cas1 | | | | | |
| RT- | 605 | Y | −3.62 | 0.00 | Not interchangeable |
| Cas1 | | | | | |
| RT- | 606 | Y | −1.84 | 16.67 | Limited |
| Cas1 | | | | | interchangeability |
| RT- | 607 | Q | −1.58 | 19.44 | Limited |
| Cas1 | | | | | interchangeability |
| RT- | 608 | L | −3.44 | 0.00 | Not interchangeable |
| Cas1 | | | | | |
| RT- | 609 | L | −3.19 | 8.33 | Not interchangeable |
| Cas1 | | | | | |
| RT | 610 | E | −2.75 | 0.00 | Not interchangeable |
| Cas1 | | | | | |
| RT- | 611 | S | −0.44 | 58.33 | Interchangeable |
| Cas1 | | | | | |
| RT- | 612 | E | −3.87 | 0.00 | Not interchangeable |
| Cas1 | | | | | |
| RT- | 613 | V | −4.07 | 0.00 | Not interchangeable |
| Cas1 | | | | | |
| RT- | 614 | Q | −2.12 | 0.00 | Not interchangeable |
| Cas1 | | | | | |
| RT- | 615 | N | −2.02 | 19.44 | Limited |
| Cas1 | | | | | interchangeability |
| RT- | 616 | Y | −3.87 | 0.00 | Not interchangeable |
| Cas1 | | | | | |
| RT- | 617 | K | −3.83 | 0.00 | Not interchangeable |
| Cas1 | | | | | |
| RT- | 618 | N | −1.96 | 8.33 | Not interchangeable |
| Cas1 | | | | | |
| RT- | 619 | F | −2.46 | 11.11 | Limited |
| Cas1 | | | | | interchangeability |
| RT- | 620 | — | −3.31 | 5.56 | Not interchangeable |
| Cas1 | | | | | |
| RT- | 621 | L | −2.99 | 2.86 | Not interchangeable |
| Cas1 | | | | | |
| RT- | 622 | T | −0.49 | 72.22 | Highly |
| Cas1 | | | | | interchangeable |
| RT- | 623 | G | −3.20 | 0.00 | Not interchangeable |
| Cas1 | | | | | |
| RT- | 624 | E | −1.26 | 25.00 | Limited |
| Cas1 | | | | | interchangeability |
| RT- | 625 | T | −0.48 | 61.11 | Highly |
| Cas1 | | | | | interchangeable |
| RT- | 626 | Y | −3.93 | 0.00 | Not interchangeable |
| Cas1 | | | | | |
| RT- | 627 | K | −1.79 | 5.56 | Not interchangeable |
| Cas1 | | | | | |
| RT- | 628 | P | −3.91 | 0.00 | Not interchangeable |
| Cas1 | | | | | |
| RT- | 629 | Y | −3.76 | 0.00 | Not interchangeable |
| Cas1 | | | | | |
| RT- | 630 | K | −3.82 | 0.00 | Not interchangeable |
| Cas1 | | | | | |
| RT- | 631 | Y | −3.73 | 0.00 | Not interchangeable |
| Cas1 | | | | | |
| RT- | 632 | Y | −3.58 | 0.00 | Not interchangeable |
| Cas1 | | | | | |
| Cas2 | 1 | M | −5.29 | 0.00 | Not interchangeable |
| Cas2 | 2 | Y | −2.99 | 5.56 | Not interchangeable |
| Cas2 | 3 | V | −4.03 | 11.11 | Limited |
| | | | | | interchangeability |
| Cas2 | 4 | — | −3.28 | 5.56 | Not interchangeable |
| Cas2 | 5 | L | −4.30 | 2.78 | Not interchangeable |
| Cas2 | 6 | V | −2.44 | 11.11 | Limited |
| | | | | | interchangeability |
| Cas2 | 7 | Y | −4.01 | 0.00 | Not interchangeable |
| Cas2 | 8 | D | −3.77 | 0.00 | Not interchangeable |
| Cas2 | 9 | — | −2.88 | 16.67 | Limited |
| | | | | | interchangeability |
| Cas2 | 10 | H | −1.91 | 36.11 | Interchangeable |
| Cas2 | 11 | Q | −0.51 | 55.56 | Interchangeable |
| Cas2 | 12 | K | −2.38 | 2.78 | Not interchangeable |
| Cas2 | 13 | R | −2.17 | 5.56 | Not interchangeable |
| Cas2 | 14 | V | −1.56 | 27.78 | Limited |
| | | | | | interchangeability |
| Cas2 | 15 | G | −0.76 | 47.22 | Interchangeable |
| Cas2 | 16 | K | −1.21 | 13.89 | Limited |
| | | | | | interchangeability |
| Cas2 | 17 | A | −2.74 | 22.22 | Limited |
| | | | | | interchangeability |
| Cas2 | 18 | L | −1.54 | 19.44 | Limited |
| | | | | | interchangeability |
| Cas2 | 19 | K | −1.18 | 36.11 | Interchangeable |
| Cas2 | 20 | I | −2.24 | 16.67 | Limited |
| | | | | | interchangeability |
| Cas2 | 21 | C | −3.03 | 22.22 | Limited |
| | | | | | interchangeability |
| Cas2 | 22 | R | −2.83 | 0.00 | Not interchangeable |
| Cas2 | 23 | K | −2.24 | 27.78 | Limited |
| | | | | | interchangeability |
| Cas2 | 24 | Y | −4.54 | 5.56 | Not interchangeable |
| Cas2 | 25 | L | −4.46 | 2.78 | Not interchangeable |
| Cas2 | 26 | I | −1.11 | 36.11 | Interchangeable |
| Cas2 | 27 | H | −1.39 | 41.67 | Interchangeable |
| Cas2 | 28 | — | −1.79 | 13.89 | Limited |
| | | | | | interchangeability |
| Cas2 | 29 | Q | −3.54 | 0.00 | Not interchangeable |
| Cas2 | 30 | K | −1.99 | 5.56 | Not interchangeable |
| Cas2 | 31 | S | −3.98 | 0.00 | Not interchangeable |
| Cas2 | 32 | V | −2.35 | 19.44 | Limited |
| | | | | | interchangeability |
| Cas2 | 33 | F | −3.72 | 8.33 | Not interchangeable |
| Cas2 | 34 | E | −1.95 | 38.89 | Interchangeable |
| Cas2 | 35 | G | −5.07 | 0.00 | Not interchangeable |
| Cas2 | 36 | N | −1.17 | 33.33 | Interchangeable |
| Cas2 | 37 | — | −3.87 | 11.43 | Limited |
| | | | | | interchangeability |
| Cas2 | 38 | T | −1.53 | 19.44 | Limited |
| | | | | | interchangeability |
| Cas2 | 39 | E | −1.21 | 2.78 | Not interchangeable |
| Cas2 | 40 | S | −0.80 | 41.67 | Interchangeable |
| Cas2 | 41 | K | −2.69 | 0.00 | Not interchangeable |
| Cas2 | 42 | L | −2.07 | 33.33 | Interchangeable |
| Cas2 | 43 | K | −0.51 | 75.00 | Highly |
| | | | | | interchangeable |
| Cas2 | 44 | A | −0.52 | 61.11 | Highly |
| | | | | | interchangeable |
| Cas2 | 45 | L | −4.24 | 8.33 | Not interchangeable |
| Cas2 | 46 | K | −2.12 | 19.44 | Limited |
| | | | | | interchangeability |
| Cas2 | 47 | E | −0.81 | 44.44 | Interchangeable |
| Cas2 | 48 | E | −2.52 | 0.00 | Not interchangeable |
| Cas2 | 49 | L | −4.45 | 8.33 | Not interchangeable |
| Cas2 | 50 | G | −0.38 | 86.11 | Highly |
| | | | | | interchangeable |
| Cas2 | 51 | H | 0.05 | 97.22 | Highly |
| | | | | | interchangeable |
| Cas2 | 52 | L | −0.57 | 66.67 | Highly |
| | | | | | interchangeable |
| Cas2 | 53 | — | −2.16 | 27.78 | Limited |
| | | | | | interchangeability |
| Cas2 | 54 | D | −2.25 | 0.00 | Not interchangeable |
| Cas2 | 55 | T | −0.53 | 44.44 | Interchangeable |
| Cas2 | 56 | Q | −1.43 | 16.67 | Limited |
| | | | | | interchangeability |
| Cas2 | 57 | M | −1.17 | 27.03 | Limited |
| | | | | | interchangeability |
| Cas2 | 58 | D | −4.92 | 0.00 | Not interchangeable |
| Cas2 | 59 | S | −1.36 | 38.89 | Interchangeable |
| Cas2 | 60 | V | −3.69 | 19.44 | Limited |
| | | | | | interchangeability |
| Cas2 | 61 | I | −1.61 | 19.44 | Limited |
| | | | | | interchangeability |
| Cas2 | 62 | — | −3.65 | 13.89 | Limited |
| | | | | | interchangeability |
| Cas2 | 63 | Y | −3.59 | 0.00 | Not interchangeable |
| Cas2 | 64 | H | −0.42 | 66.67 | Highly |
| | | | | | interchangeable |
| Cas2 | 65 | L | −2.06 | 5.56 | Not interchangeable |
| Cas2 | 66 | D | −0.65 | 44.44 | Interchangeable |
| Cas2 | 67 | S | −2.00 | 0.00 | Not interchangeable |
| Cas2 | 68 | V | −2.13 | 11.11 | Limited |
| | | | | | interchangeability |
| Cas2 | 69 | K | −2.26 | 5.56 | Not interchangeable |
| Cas2 | 70 | Y | −3.82 | 8.57 | Not interchangeable |
| Cas2 | 71 | T | −1.77 | 19.44 | Limited |
| | | | | | interchangeability |
| Cas2 | 72 | K | −1.16 | 27.78 | Limited |
| | | | | | interchangeability |
| Cas2 | 73 | K | −3.14 | 0.00 | Not interchangeable |
| Cas2 | 74 | E | −0.21 | 83.33 | Highly |
| | | | | | interchangeable |
| Cas2 | 75 | Q | −0.57 | 58.33 | Interchangeable |
| Cas2 | 76 | I | −1.55 | 30.56 | Interchangeable |
| Cas2 | 77 | G | −5.63 | 0.00 | Not interchangeable |
| Cas2 | 78 | — | −2.18 | 0.00 | Not interchangeable |
| Cas2 | 79 | V | −1.94 | 30.56 | Interchangeable |
| Cas2 | 80 | Q | −0.95 | 38.89 | Interchangeable |
| Cas2 | 81 | S | −1.79 | 5.71 | Not interchangeable |
| Cas2 | 82 | T | −1.45 | 19.44 | Limited |
| | | | | | interchangeability |
| Cas2 | 83 | S | −1.30 | 13.89 | Limited |
| | | | | | interchangeability |
| Cas2 | 84 | N | −1.15 | 36.11 | Interchangeable |
| Cas2 | 85 | V | −1.52 | 27.78 | Limited |
| | | | | | interchangeability |
| Cas2 | 86 | — | −3.36 | 8.33 | Not interchangeable |

Percent Functionally Tolerated Substitutions is defined as the percentage of missense substitutions at that position with log fold-change (LFC) greater than or equal to wildtype. percentages indicate greater functional permissiveness to amino acid substitution at that position, supporting broader residue interchangeability.
Positions are classified into four categories of amino acid interchangeability based on the fraction of tolerated substitutions (LFC ≥ −0.5), enabling straightforward identification of residues suitable for functional variants: Not interchangeable (<10%), Limited interchangeability (10-<30%), Interchangeable (30-<60%), and Highly interchangeable (≥60%).

TABLE 3

| Validation results for engineered FsRT-Cas1- | | |
|---|---|---|
| Cas2 variants designed from | | |
| deep mutational scanning (DMS) data. The table lists | | |
| each tested variant, its amino-acid substitution(s) relative to | | |
| wild-type, and the corresponding log₂ fold-change in | | |
| spacer acquisition efficiency relative to wild-type. | | |
| Variants correspond to those | | |
| summarized in Fig. 4 and 5. | | |
| | | Log2 fold- |
| Variant | Domain | change |
| E381V | Cas1 | -1.53 |
| T477H | Cas1 | -0.34 |
| E476K | Cas1 | 0.17 |
| L564R | Cas1 | 0.44 |
| R361Y | Cas1 | 1.17 |
| T565R | Cas1 | 1.18 |
| E590K | Cas1 | 1.34 |
| E476T | Cas1 | 1.63 |
| R449D | Cas1 | 1.74 |
| E590R | Cas1 | 1.84 |
| 1583K | Cas1 | 1.93 |
| E590T | Cas1 | 2.43 |
| N448K | Cas1 | 3.24 |
| L564R, T565R, 1583K | Cas1 | -0.62 |
| E381V, T477H | Cas1 | -0.13 |
| R361Y, E381V | Cas1 | 0.19 |
| E381V, R449D | Cas1 | 0.23 |
| E381V, E476T | Cas1 | 0.36 |
| L564R, 1583K | Cas1 | 2.63 |
| T565R, 1583K, E590K | Cas1 | 2.82 |
| T565R, 1583K | Cas1 | 2.94 |
| L564R, 1583K, E590K | Cas1 | 3.28 |
| E476T, T477H | Cas1 | 3.84 |
| N448K, R449D | Cas1 | 4.24 |
| R449D, T477H | Cas1 | 4.36 |
| N448K, R449D, E476T | Cas1 | 4.51 |
| R449D, E476T | Cas1 | 4.63 |
| N448K, E476T | Cas1 | 4.89 |
| N448K, T477H | Cas1 | 4.90 |
| N448K, R449D, T477H | Cas1 | 5.05 |
| 1583K, E590K | Cas1 | 5.53 |
| N448K, E476T, T477H | Cas1 | 5.61 |
| L564R, T565R | Cas1 | 5.64 |
| T565R, E590K | Cas1 | 6.19 |
| L564R, E590K | Cas1 | 6.20 |
| N150H | RT | 0.58 |
| L63W | RT | 0.84 |
| E136F | RT | 0.99 |
| S92H | RT | 1.18 |
| D112P | RT | 1.28 |
| M117V | RT | 1.30 |
| D1121 | RT | 1.78 |
| N69E | RT | 1.94 |
| E136L | RT | 3.15 |
| L63W, N69E | RT | 2.32 |
| D1121, N150H | RT | 2.61 |
| L63W, N69E, S92H | RT | 3.32 |
| L63W, S92H | RT | 3.61 |
| N69E, S92H | RT | 3.69 |
| M117V, N150H | RT | 3.75 |
| D1121, M117V, N150H | RT | 4.46 |
| D1121, M117V | RT | 4.51 |
| D1121, E136L | RT | 4.84 |
| M117V, E136L | RT | 4.86 |
| D1121, E136L, N150H | RT | 5.25 |
| D1121, M117V, E136L | RT | 5.33 |
| D1121, M117V, E136L, N150H | RT | 5.58 |
| M117V, E136L, N150H | RT | 6.04 |
| V14N | Cas2 | -0.30 |
| H27G | Cas2 | 0.75 |
| 128L | Cas2 | 0.86 |
| Q75G | Cas2 | 1.52 |
| Q75A | Cas2 | 2.15 |
| V14N, H27G | Cas2 | 0.54 |
| V14N, 128L | Cas2 | 0.84 |
| H27G, 128L | Cas2 | 0.90 |
| 128L, Q75G | Cas2 | 0.98 |
| V14N, Q75G | Cas2 | 1.42 |
| V14N, H27G, Q75G | Cas2 | 2.12 |
| D1121, M117V, N150H, | RT-Cas1 | 3.07 |
| L564R, E590K | | |
| D1121, M117V, E136L, N150H, | RT-Cas1 | 3.06 |
| T565R, E590K | | |
| D1121, M117V, E136L, N150H, | RT-Cas1 | 2.81 |
| L564R, E590K | | |
| D1121, M117V, N150H, | RT-Cas1 | 2.76 |
| T565R, E590K | | |
| D1121, M117V, E136L, N150H, | RT-Cas1 | 2.55 |
| T565R, 1583K, E590K | | |
| D1121, M117V, N150H, T565R, | RT-Cas1 | 2.54 |
| 1583K, E590K | | |
| R361Y, N448K, E476T, T477H, | RT-Cas1 | 2.42 |
| T565R, 1583K, E590K | | |
| R361Y, N448K, E476T, T477H, | RT-Cas1 | 2.15 |
| L564R, E590K | | |
| N69E, S92H, D1121, M117V, | RT-Cas1 | 2.01 |
| N150H, R361Y, N448K, | | |
| E476T, T477H, T565R, E590K | | |
| N448K, E476T, T477H, | RT-Cas1 | 1.94 |
| L564R, E590K | | |
| R361Y, N448K, E476T, T477H, | RT-Cas1 | 1.89 |
| T565R, E590K | | |
| D1121, M117V, N150H, N448K, | RT-Cas1 | 1.86 |
| E476T, T477H | | |
| N69E, S92H, D1121, M117V, | RT-Cas1 | 1.68 |
| N150H, R361Y, N448K, E476T, | | |
| T477H, L564R, E590K | | |
| D1121, M117V, E136L, N150H, | RT-Cas1 | 1.60 |
| N448K, E476T, T477H | | |
| N69E, S92H, D1121, M117V, | RT-Cas1 | 1.52 |
| N150H | | |
| N448K, E476T, T477H, T565R, | RT-Cas1 | 1.42 |
| E590K | | |
| N69E, S92H, D1121, M117V, | RT-Cas1 | 1.38 |
| E136L, N150H, R361Y | | |
| N69E, S92H, D1121, M117V, | RT-Cas1 | 1.35 |
| N150H, R361Y, N448K, E476T, | | |
| T477H, T565R, 1583K, E590K | | |
| N448K, E476T, T477H, T565R, | RT-Cas1 | 1.23 |
| 1583K, E590K | | |
| N69E, S92H, D1121, M117V, | RT-Cas1 | 0.95 |
| E136L, N150H, R361Y, N448K, | | |
| E476T, T477H, T565R, E590K | | |
| N69E, S92H, D1121, M117V, | RT-Cas1 | 0.63 |
| E136L, N150H, R361Y, N448K, | | |
| E476T, T477H, L564R, E590K | | |

Sequeces

Fusicatenibacter saccharivorans, RT-Cas1, Amino Acid:
MFTIDEMLSKNNQRLAFEHFATKNDGCGPDGMHVSELEKYWRMNHDQIISDLKNQE
YQPGIILIREHMNKTGKRRNIASLNVIDRFITRLLSQKLNRYLAPIFCENSYAYQDSKGVMPAVL
KAKEYVELGMRHVIEIDLKNYFDTIPLENLIPEIERYITDEAVLHLIKQYLFCDISFEGKISRKTQG
IVQGNAISPILSNLYLNDFDKELDESKLCWIRYADNIYIYMDSYEKALLVYSELTERLERRKLTV
NKEKSGVFDVSTRSILGYDILIRNKKVDVRKHIYKSVNQYSNWHDSRLEFINGRYHITSDGILN
RQDFGLLFENEQKKHYIPVEVSDQLNIYGNVTLASNVLQSFSNREIKVSFFDKYGRLIGSFLPE
KTKKSAEIILVQSKNYLNEDVRMDTARRMEIAGLHNIRANLRYYDKKHKGDFKEKVDAISGYID
ALNRAPSVNDMMLLEAKARQLYYTCFNQILETSDFQFEKRTKRPPKDAINACISFGNTLLYNL
FVNIIWKKGLDPRFGVVHASNKRNQSLNLDFADIFKPIVIDRIIFTMINKKMLTLLTDFETSNQGV
YLSREGKNIFLQMYEEKLKSRITIKGKEMSYYQLLESEVQNYKNFILTGETYKPYKYY (SEQ ID
NO: 001)
Fusicatenibacter saccharivorans, Cas2, Amino Acid:
MYVILVYDIHQKRVGKALKICR
KYLIHIQKSVFEGNITESKLKALKEELGHLIDTQMDSVIIYHLDSVKYTKKEQIGIVQSTSNVI
(SEQ ID NO: 021)
Fusicatenibacter saccharivorans, Array1, DNA, genomic:
AGAATTAAATTGGAAAAAGTCGGTCGATCTCATGCCTGAAATCATGAATTCCGCA
AAATGGCGGAAATTTAAGGAAAATCAGGAATCTCAGAAAAACGATCGACCGACTTTTGTG
ATAAAATGGTTGCAAAAAAGAGAAAAATTTGATTTAATAGAATGTGAAAATAGCGGAAATG
CTGATGTTGTACCTTACCTATGAGGAATTGAAACGCTTTTTCGATTGCTTCTTCTACAATC
TGCTGAGCTTTGTTGTACCTTACCTATGAGGAATTGAAACTTGCATTCGTTACAATATTTG
CTGTCAGTCTTGGTGTGTTCGTTGTACCTTACCTATGAGGAATTGAAAC (SEQ ID NO:
040)
Fusicatenibacter saccharivorans, Array1, DNA, genomic:
GCTGAAAGGAGGAACTATATCCGGATACCAGGTATGCCTGAAATCATGAATTCCG
CAAAATGGCGGAAATTTAAGGAAAATCAGGAATCTCAGAAAAACGATCGACCGACTTTTG
TGATAAAATGGTTGCAAAAAAGAGAAAAATTTGATTTAATAGAATGTGAAAATAGCGGAAA
TGCTGATGTTGTACCTTACCTATGAGGAATTGAAACGTCCCGCGGCCGCTATTCTTTTGA
TTTATAAGGGATTTTGCGATCCTCTGGAGTGAATACCACGACGATTTCCGGCAGTTTCTC
CACA
(SEQ ID NO: 041)
Fusicatenibacter saccharivorans, Array1 RC, DNA, genomic:
GCTGAAAGGAGGAACTATATCCGGATACCAGGTATGCCTGAAATCATGAATTCCG
CAAAATGGCGGAAATTTAAGGAAAATCAGGAATCTCAGAAAAACGATCGACCGACTTTTG
TGATAAAATGGTTGCAAAAAAGAGAAAAATTTGATTTAATAGAATGTGAAAATAGCGGAAA
TGCTGATGTTGTACCTTACCTATGAGGAATTGAAACGTCCCGCGGCCGCTATTCTTTTGA
TTTATAAGGGATTTTGCGATCCTCTGGAGTGAATACCACGACGATTTCCGGCAGTTTCTC
CACA
(SEQ ID NO: 042)
Fusicatenibacter saccharivorans, Array2, DNA, genomic:
CGGGGAAGCTTATGTTCCATAGCAAAAAGTCGGTCAGTCTCGTGGCTGAAATCAT
GAGTTCCACAAAATGGCTGAAATTCAAGGAAAATCAGGAATCTCAGAAAAACGATCGACC
GACTTTTTCGATAAAATGGTTGCAAAAATGAGAAAAATCTGATTTAATAGAATCTGAAAAC
AGCGGAAATGCTGTTGTCGTACTTTACCTAAAAGGAATTGAAACTATTATATGAGACTTCA
ATTGCTGCATATTCCCTAGCGTCGTACTTTACCTAAAAGGAATTGAAACACTTACCAGAAT
ATACGTATTACGTAAATCCTTTGCTTTGTCGTACTTTACCTAAAAGGAATTGAAAC (SEQ ID
NO: 043)
Fusicatenibacter saccharivorans, Array2, DNA, genomic:
GCTGAAAGGAGGAACTATATCCGGATACCAGGTGTGGCTGAAATCATGAGTTCC
ACAAAATGGCTGAAATTCAAGGAAAATCAGGAATCTCAGAAAAACGATCGACCGACTTTT
TCGATAAAATGGTTGCAAAAATGAGAAAAATCTGATTTAATAGAATCTGAAAACAGCGGAA
ATGCTGTTGTCGTACTTTACCTAAAAGGAATTGAAACGTCCCGCGGCCGCTATTCTTTTG
ATTTATAAGGGATTTTGCGATCCTCTGGAGTGAATACCACGACGATTTCCGGCAGTTTCT
CCACA
(SEQ ID NO: 044)
Fusicatenibacter saccharivorans, Array2 RC, DNA, genomic:
GCTGAAAGGAGGAACTATATCCGGATACCAGGTTTTGTTCGGTGTTATTTTTGTA
CTCATTTTTAGAGAAAATAAAGAAGAATGGTTATGGCAAAGAAAAAGGCAGCAATATCAT
CACCGCTTCCAAGCTCTGACCTGGTTGATCTTTATATTAGAGTTTCTGTGCCACATTGGG
GTCTGACCCCATATAGCACCTTTAGGTAAAGTACGACGTCCCGCGGCCGCTATTCTTTTG
ATTTATAAGGGATTTTGCGATCCTCTGGAGTGAATACCACGACGATTTCCGGCAGTTTCT
CCACA
(SEQ ID NO: 045)
Fusicatenibacter saccharivorans, RT-Cas1, DNA, genomic:
ATGTTCACTATAGACGAGATGCTATCAAAGAACAACCAAAGATTGGCTTTCGAGC
ACTTCGCTACTAAGAACGACGGATGTGGCCCTGATGGTATGCACGTATCTGAGCTGGAG
AAATATTGGCGTATGAATCACGATCAGATCATCTCCGACTTGAAGAACCAAGAGTACCAA
CCCGGCATTATTTTGATTAGAGAACACATGAACAAAACAGGTAAGAGAAGGAACATTGCA
TCTCTCAACGTGATCGACAGGTTCATAACTCGACTGCTGTCGCAAAAGTTGAATAGATAC
TTAGCCCCCATTTTCTGCGAGAACTCATACGCTTACCAAGACTCAAAAGGGGTGATGCCC
GCCGTACTAAAAGCCAAGGAGTACGTCGAGTTGGGTATGAGACACGTTATCGAGATAGA
CCTCAAGAATTACTTCGACACGATCCCGTTAGAGAACTTAATCCCAGAGATCGAGCGTTA
CATAACAGATGAGGCAGTGCTGCACTTGATTAAACAGTACTTATTCTGTGACATCTCGTTC
GAGGGTAAGATATCACGTAAAACTCAAGGGATCGTTCAGGGTAACGCAATTTCCCCAATA
CTATCCAACCTCTACCTGAACGACTTCGACAAGGAGCTGGACGAGTCGAAGCTGTGCTG
GATCAGATACGCCGACAACATATACATCTATATGGACAGCTACGAGAAGGCCCTACTGGT
TTATTCCGAGCTTACCGAGAGACTTGAGAGAAGGAAATTAACCGTTAACAAGGAGAAATC
AGGGGTGTTTGATGTTAGTACCCGTTCTATACTAGGTTACGACATCCTGATCAGAAACAA
GAAGGTTGACGTGCGTAAACACATCTACAAGTCTGTTAACCAGTACTCCAACTGGCACGA
CTCTCGATTGGAGTTCATCAACGGTCGTTACCACATCACTTCCGACGGTATCCTCAATAG
ACAAGACTTCGGTCTACTGTTCGAGAATGAGCAGAAGAAGCACTACATACCTGTTGAGGT
TTCTGACCAGTTGAATATTTACGGAAACGTTACGCTCGCCAGCAACGTTCTTCAATCATTC
TCCAACCGAGAGATCAAAGTGTCCTTCTTCGACAAGTACGGGAGACTTATTGGATCTTTC
CTGCCCGAGAAAACTAAGAAAAGCGCCGAGATAATACTGGTTCAGTCTAAGAACTACCTT
AACGAGGACGTTAGGATGGACACGGCCCGACGTATGGAGATCGCCGGTTTGCATAACAT
CAGAGCCAACCTCAGATACTACGACAAGAAGCACAAAGGTGACTTTAAGGAAAAGGTAG
ACGCCATTTCTGGCTATATTGACGCCCTCAATAGAGCACCATCCGTGAACGACATGATGC
TCTTAGAGGCTAAAGCTCGTCAGCTGTACTACACATGCTTCAACCAGATCTTAGAGACAT
CCGACTTTCAGTTCGAGAAGCGTACCAAGCGACCACCAAAAGACGCCATCAACGCCTGT
ATTTCATTCGGCAACACATTACTGTACAACTTGTTTGTGAATATCATTTGGAAGAAGGGAC
TTGATCCAAGGTTCGGGGTTGTTCACGCCAGCAACAAGCGTAATCAGTCCCTTAACCTGG
ATTTTGCTGACATCTTCAAGCCTATAGTCATAGATAGGATCATCTTCACAATGATCAACAA
GAAGATGCTGACCTTGTTGACGGACTTCGAGACTTCTAACCAGGGAGTTTACCTTTCCCG
AGAGGGGAAGAACATCTTCTTGCAGATGTACGAAGAAAAGCTCAAATCCAGAATCACTAT
CAAGGGAAAGGAGATGAGTTACTATCAGTTGCTAGAATCAGAGGTGCAGAACTACAAGAA
CTTCATCCTGACCGGCGAGACATACAAGCCGTACAAGTACTACTAG (SEQ ID NO:
113)
Fusicatenibacter saccharivorans, Cas2, DNA, genomic:
ATGTATGTCATTCTAGTTTACGACATTCACCAGAAGCGAGTAGGTAAGGCTCTAA
AGATTTGTAGGAAGTACCTGATCCACATCCAGAAGTCAGTTTTCGAAGGCAACATAACGG
AGAGTAAGCTAAAGGCCTTAAAAGAGGAGCTGGGGCACCTGATCGACACTCAGATGGAC
TCGGTAATTATCTACCACCTGGACTCCGTGAAGTACACTAAGAAGGAGCAGATCGGCATC
GTCCAATCGACATCCAATGTCATTTGA (SEQ ID NO: 133)
Candidatus Accumulibacter sp. BA-91, RT-Cas1, Amino Acid:
MSTPDYCWQRIGLTLRCTFAHPGRKAHPLSVLEAIVKGIGEAAGLTAPTMRSVFRWS
GMRSSQMTIESGRILSLEILLFGTDAGAACNWHERAIHYFDPGAPGRNFQVTASEAPVERRW
AELLAGRQAPAESNDECCLDFLTPLPFTPAQGRGRTWLDGEGLRRAMQDRLRRLFGAEAEL
PPIPEVLPAYWYYCQIVHAASSQPGHNKYLNGCLGPLLLRGEHLGEWWPWLVLGEEIGLGG
QVSFGQGLFRLHAKSVPILDARLTDPNQIAAIIDQLLLRHDDLAVRLSNTPQAPDLHELAVELA
QNLREGAAPLPFQAIRVPRSDGRLRQFETPAARDLVILNHLTRLLSEPFDRLFSVHSIGYRKG
HSREDAVERVRAAIAEGCTHVLESDISDFFPSVDLKRLLARLDDVLPRRDVRLRQTLAAYLGA
GWRYGEGSVQARNRGLPLGSPLSPLLANLYLDSFDSQLGATVPGVRLIRYADDFIILTESEAA
ARALLDTARDAAAALGLALNLEKTAIRPLSDGFDFLGIRFSADAAAEQAGDESADSLRKVLYIT
EPYAFVGSNHGTIEVHAGSKSLGSFPLARTAGVVTLVPCTLSSALIARLADQCIPLAIAGTQGR
QIATVAGDTARRFATAATQANRHASLGEAGRCRAAGAFATAKLANYIALIRQRGPAGTAALVA
RLENGIAAIASATDIDAIRGVEGDCARECFPFIAGWINSPDFPWQGRRRHGEFPDRLNSLLNF
GYHLLFTRINALLRVSGLNPYLGFLHAANGRYEALACDVQEAFRPHIDRLVVRLLNLKVIEAAD
FEESEEGWWLIRPARTRFLQQFAREIERRPMRRRYSLGEAIEGQVRALHAWLIEDRELVLYR
WSDSDV (SEQ ID NO: 002)
Candidatus Accumulibacter sp. BA-91, Cas2, Amino Acid:
MSDMALYIVAYDITDDRERRQAEHILQGFGFRRQKSVFECRLTRGYLARLQHELATIG
METGFVMIYHLAPNTRPFTIGEVPHFPDDDWAWVT (SEQ ID NO: 022)
Candidatus Accumulibacter sp. BA-91, Array1, DNA, genomic:
CTCTTGCCAAACCCTGTGGATAAGCTGCTAGACTTGCCGGAGTCATCGAGGTTG
GGCAAGCGACGGTACGCAAGTCTCCGGCAAGTCAGCCTTTTTTGACACCCAGTGCTGAA
TAGGGAATCCTGTTCGAACAGGCTTGAAACGAAAAGAGTCACTATATAATCTTCAAGTGA
GCTGAATAGGGAATCCTGTTCGAACAGGCTTGAAACCCTCTGGAAGTTCCACCTCATCCC
CAAAGTGCTGAATAGGGAATCCTGTTCGAACAGGCTTGAAAC (SEQ ID NO: 046)
Candidatus Accumulibacter sp. BA-91, Array1, DNA, genomic:
GCTGAAAGGAGGAACTATATCCGGATACCAGGTGGTACCCCATTTTCCTGATGAC
GATTGGGCATGGGTGACCTGACTCTTGCCAAACCCTGTGGATAAGCTGCTAGACTTGCC
GGAGTCATCGAGGTTGGGCAAGCGACGGTACGCAAGTCTCCGGCAAGTCAGCCTTTTTT
GACACCCAGTGCTGAATAGGGAATCCTGTTCGAACAGGCTTGAAACGTCCCGCGGCCGC
TATTCTTTTGATTTATAAGGGATTTTGCGATCCTCTGGAGTGAATACCACGACGATTTCCG
GCAGTTTCTCCACA (SEQ ID NO: 047)
Candidatus Accumulibacter sp. BA-91, RT-Cas1, DNA:
ATGTCTACTCCAGACTACTGTTGGCAGCGTATAGGTCTTACACTAAGATGTACATT
TGCTCACCCAGGGAGAAAAGCCCATCCACTTTCTGTCCTAGAGGCAATAGTTAAAGGTAT
TGGAGAAGCAGCTGGGTTGACTGCTCCTACTATGAGATCAGTATTTAGATGGTCCGGAAT
GAGATCGTCGCAAATGACTATTGAGAGTGGTAGGATCTTGTCTCTCGAAATTTTGTTATTT
GGTACTGACGCTGGAGCTGCTTGTAATTGGCACGAAAGAGCCATACACTACTTTGATCCT
GGTGCACCGGGTAGAAACTTTCAGGTTACTGCTTCCGAGGCCCCTGTCGAAAGACGATG
GGCAGAGCTATTGGCTGGACGACAGGCTCCAGCAGAATCCAATGATGAGTGCTGTTTGG
ACTTTCTTACCCCATTACCCTTTACCCCAGCTCAAGGACGTGGAAGAACATGGCTTGATG
GTGAAGGTCTACGTAGAGCCATGCAAGACAGATTGAGAAGATTATTTGGTGCTGAGGCT
GAACTTCCCCCTATTCCTGAGGTCCTGCCTGCTTATTGGTATTATTGTCAGATTGTTCACG
CTGCTTCCTCTCAGCCAGGTCATAATAAATACCTTAATGGATGTTTGGGTCCTTTGTTGCT
TAGAGGAGAACACCTTGGGGAATGGTGGCCTTGGCTGGTTCTAGGTGAGGAGATTGGAC
TTGGTGGACAAGTTTCTTTTGGACAAGGTTTATTCAGACTCCATGCTAAGTCAGTTCCAAT
TCTTGACGCCAGGTTGACGGACCCAAATCAAATTGCTGCTATTATTGATCAGCTTCTATTA
AGACATGATGACTTAGCAGTAAGACTCTCTAATACCCCTCAAGCCCCAGATCTTCATGAG
TTGGCTGTGGAGTTGGCTCAAAATCTCAGAGAGGGAGCAGCTCCTCTCCCATTCCAAGC
TATCAGAGTTCCAAGATCGGACGGTAGACTCCGTCAATTTGAGACACCCGCAGCACGAG
ACTTAGTTATTTTGAATCATTTGACGAGACTTTTATCAGAGCCGTTTGATCGATTGTTTTCT
GTGCACAGTATCGGATACAGAAAAGGTCACTCCAGAGAGGATGCAGTCGAAAGAGTTAG
AGCAGCTATTGCAGAGGGTTGTACTCACGTTTTGGAGTCTGATATTTCTGACTTCTTTCCG
TCTGTTGACTTAAAACGACTCTTGGCACGTCTTGATGATGTTTTGCCGCGTCGTGATGTA
AGATTGAGACAAACACTCGCTGCATATCTTGGTGCAGGATGGCGTTACGGTGAAGGGTC
GGTCCAAGCTAGAAATCGTGGACTTCCATTAGGATCACCATTGAGCCCACTCCTAGCAAA
CCTTTACTTGGATTCATTTGATTCCCAACTGGGAGCTACTGTTCCAGGAGTCAGGTTAATT
AGGTACGCTGATGATTTTATTATACTTACTGAATCAGAAGCAGCCGCAAGAGCTTTATTAG
ACACTGCTAGAGATGCCGCCGCCGCTTTAGGTCTAGCTTTGAATTTAGAAAAGACTGCTA
TTAGACCACTCTCGGATGGTTTTGACTTCTTGGGAATAAGATTTAGTGCTGACGCTGCTG
CTGAACAAGCTGGAGATGAATCAGCTGATAGTTTAAGAAAAGTTCTTTATATTACTGAGCC
ATACGCTTTTGTTGGATCAAATCATGGTACTATTGAAGTGCACGCCGGTTCCAAATCCTTG
GGATCATTCCCACTGGCTAGAACAGCTGGTGTCGTTACTTTGGTGCCATGCACTCTCTCA
TCTGCTCTAATTGCTCGTCTTGCTGATCAATGTATTCCCTTGGCTATTGCAGGCACACAAG
GTAGACAAATTGCTACTGTAGCTGGGGATACGGCAAGGAGATTCGCAACTGCTGCAACT
CAAGCAAATAGACACGCTTCATTAGGTGAGGCCGGTAGATGTAGGGCTGCTGGAGCCTT
CGCTACTGCTAAACTTGCTAATTACATAGCACTTATAAGACAAAGAGGACCAGCAGGAAC
AGCAGCTCTAGTGGCACGTCTTGAGAATGGTATAGCTGCTATTGCTTCAGCAACTGATAT
AGATGCAATTAGAGGTGTAGAAGGTGACTGTGCTCGTGAGTGTTTCCCATTTATTGCTGG
ATGGATTAATTCTCCAGACTTCCCTTGGCAGGGCAGAAGAAGGCATGGTGAGTTTCCTGA
CAGATTAAATTCTTTGCTAAATTTTGGATATCATCTGTTATTTACGAGAATTAATGCCCTAT
TGCGTGTTTCCGGCCTGAACCCTTATTTGGGTTTTCTTCACGCAGCAAATGGAAGGTATG
AAGCCCTGGCTTGTGATGTACAAGAGGCATTTAGACCTCATATTGATAGGTTGGTCGTAA
GACTACTAAATCTCAAGGTTATTGAGGCAGCTGACTTTGAAGAATCTGAGGAAGGATGGT
GGCTAATTCGTCCAGCACGAACAAGATTTCTTCAGCAATTTGCTAGAGAGATAGAGCGAC
GACCCATGAGGCGACGTTATTCCCTTGGTGAGGCTATTGAGGGCCAAGTCAGAGCCCTG
CACGCTTGGTTAATTGAGGATAGAGAGTTGGTCTTGTATAGATGGTCTGATTCAGACGTG
TAA (SEQ ID NO: 114)
Candidatus Accumulibacter sp. BA-91, Cas2, DNA:
ATGAGTGATATGGCCCTGTATATCGTCGCCTATGACATAACTGACGATAGAGAAA
GGCGTCAAGCTGAACACATCCTTCAAGGATTCGGGTTCAGAAGGCAAAAGTCTGTTTTCG
AGTGCAGATTGACTAGGGGATATTTAGCAAGGTTGCAGCATGAGTTAGCTACAATCGGAA
TGGAGACAGGGTTCGTTATGATTTATCACTTAGCCCCAAATACCCGACCTTTTACAATAG
GAGAAGTGCCTCACTTCCCAGACGACGACTGGGCCTGGGTAACTTAA (SEQ ID NO: 134)
Candidatus Accumulibacter sp. SK-02, RT-Cas1, Amino Acid:
MSTLPTPSSTDQDSPPPFWTLARLAEALEHVSARQGGAGADEQTLAEFAADAEAQL
GLLALQLTQGSYRPAPARLIPVAKPGGGVRELLLPAVRDRIVQSALARYLADLLEPDFGEASH
AYRPGHSVATALHRLQALRDGGLVFVAVCDIHHFFDSVDHRRLFSLLDDLPLERRLREQMKT
CVRIEVADVQGQGAWSLARGLAQGSPLSPVLANLFLMAFDAACARAGLALVRYADDCVLAC
ASETEAQSALAFAADALENIGLALNTRKSRLASFAEGFEFLGAFCGAEGMLGGRPGEAACLP
PTTGPVHEAAAADDERPPSHGHRPRLRTLYLLENGAVLNKEGERFIVARHGEVLLQVPMMRI
DQIMVFGNVQITTPALHECLERGIPVMLLSGRGRFFGVIDPLDARSVPLQRAQFALESDEPAR
LALARPLIAGKILNCRTFLGRLARARQTNMDAPLAALKSAAQAAGQAADLEILRGIEGAAARTY
FAAWQTVLPAKWQFTGRNRQPPTDPVNALLSYGYTIVFYNVLALVRARGLNTHVGVLHDVR
PGHPALASDLMEEFRAPVVDAVVMHLVFDGKLQPGDFSWPETPGQPCLMADGSRKHFIHLL
EQKLNTTVSHAGQRLDYRRWMDMQVLQYAAALRTPGLPYVPFAIR (SEQ ID NO: 003)
Candidatus Accumulibacter sp. SK-02, Cas2, Amino Acid:
MEQTWLVTYDVSDDGCRRRVERILLGHGEREQYSVFRCRLSSREVRDLRARLAGHL
KGSDSIRYYPLCAACLPRQPQRTLVDSAEAGIAWYFAV (SEQ ID NO: 023)
Candidatus Accumulibacter sp. SK-02, Array, DNA, genomic:
GAACTTCGGGGCATATGGCGCTGGTGCACATTTTGCCGGACATGGCTTTTTGGA
CAATGGCTTGCGAAAATACTTCGGCATACGGGCGCCGCGTGTTGATTTTCAAGGGAAAA
GAGGATTTTCGTGCGCCTGGACGGGAGCGCGGGAGCGTTGTTTCGCCCGCGCCCCGAA
AAGAGCCGTTAAATTCCTTGACGATCATCGGGTTGTGGAAGTAGAGTGAAAACTAAGCCC
TGCCGATAAAGGGATTGAGACCTGTCCAACAAATGCTTCTTTGGTACCGTTTTGAGGTGA
AAACTAAGCCCTGCCGATAAAGGGATTGAGACTCGACCACAGGGTCGACTAGGTCGCTA
TTGTACAGTGAAAACTAAGCCCTGCCGATAAAGGGATTGAGAC (SEQ ID NO: 048)
Candidatus Accumulibacter sp. SK-02, Array1, DNA, genomic:
GCTGAAAGGAGGAACTATATCCGGATACCAGGTAAAATACTTCGGCATACGGGC
GCCGCGTGTTGATTTTCAAGGGAAAAGAGGATTTTCGTGCGCCTGGACGGGAGCGCGG
GAGCGTTGTTTCGCCCGCGCCCCGAAAAGAGCCGTTAAATTCCTTGACGATCATCGGGT
TGTGGAAGTAGAGTGAAAACTAAGCCCTGCCGATAAAGGGATTGAGACGTCCCGCGGCC
GCTATTCTTTTGATTTATAAGGGATTTTGCGATCCTCTGGAGTGAATACCACGACGATTTC
CGGCAGTTTCTCCACA (SEQ ID NO: 049)
Candidatus Accumulibacter sp. SK-02, Array1 RC, DNA, genomic:
GCTGAAAGGAGGAACTATATCCGGATACCAGGTCAATGAAGAGGTTTGTGCCGG
GCATTTACTCCAGAGCATATTTATTGGCCAGGCTTGGGAACCGCTGATTAACACATCCTT
CGGAGAAACCTGGCAACGCGAAGCCGTCCTCGGTCGTGCCCAAGTGAATGGTGATGAC
TGGAGGATGAGGTCTCAATCCCTTTATCGGCAGGGCTTAGTTTTCACGTCCCGCGGCCG
CTATTCTTTTGATTTATAAGGGATTTTGCGATCCTCTGGAGTGAATACCACGACGATTTCC
GGCAGTTTCTCCACA (SEQ ID NO: 050)
Candidatus Accumulibacter sp. SK-02, RT-Cas1, DNA:
ATGTCTACTTTACCCACCCCTTCCAGCACTGACCAAGACTCACCACCACCTTTCT
GGACATTAGCTAGGTTGGCAGAAGCTCTTGAGCACGTGTCAGCTCGACAAGGCGGAGCT
GGAGCTGATGAGCAAACTTTGGCTGAGTTCGCAGCAGACGCAGAGGCCCAGTTAGGGC
TTTTGGCCTTGCAGCTAACACAAGGAAGTTACAGACCAGCCCCTGCTAGACTTATACCAG
TCGCTAAACCAGGAGGAGGTGTTCGTGAGCTTTTACTGCCAGCAGTCCGAGATAGAATT
GTCCAAAGTGCTTTGGCTCGTTACCTTGCTGACCTTTTGGAACCAGATTTTGGTGAGGCT
AGTCACGCTTACAGACCGGGACACTCCGTAGCTACTGCTTTACATAGGCTTCAAGCCTTA
CGAGATGGCGGATTGGTTTTCGTGGCAGTCTGTGATATACATCATTTCTTTGATTCGGTA
GATCACAGAAGGCTCTTTTCTCTTTTAGATGACCTTCCATTGGAAAGACGTTTGCGAGAG
CAAATGAAAACTTGTGTTCGTATAGAGGTAGCTGATGTACAAGGCCAGGGAGCTTGGTCC
TTAGCTAGAGGATTGGCCCAAGGTTCTCCTCTGTCTCCAGTCTTAGCTAACTTGTTCTTGA
TGGCTTTTGATGCTGCATGTGCTCGTGCTGGGTTGGCTCTTGTCAGATATGCTGATGACT
GTGTCCTTGCTTGCGCTTCCGAGACTGAAGCCCAGTCCGCCCTAGCATTTGCTGCTGAT
GCTCTTGAGAATATTGGGTTGGCCCTTAATACCAGAAAGTCGCGTCTTGCCTCTTTCGCA
GAAGGGTTTGAGTTTCTAGGTGCATTTTGTGGAGCTGAGGGCATGTTGGGTGGCCGACC
AGGAGAAGCAGCTTGTTTACCACCAACTACCGGACCAGTACATGAGGCTGCTGCTGCTG
ACGATGAGAGGCCACCTTCACATGGTCATCGTCCAAGGTTACGTACATTGTACTTGTTGG
AGAATGGCGCAGTTCTCAATAAAGAAGGGGAGAGATTTATAGTAGCTCGACATGGTGAG
GTATTACTTCAGGTTCCTATGATGCGTATTGATCAAATTATGGTCTTTGGTAATGTCCAAA
TTACTACTCCAGCTTTGCATGAGTGTTTAGAAAGAGGAATCCCTGTTATGCTTCTAAGTGG
TCGAGGACGTTTCTTTGGTGTTATTGATCCCCTTGATGCAAGATCCGTCCCTTTACAAAG
GGCTCAATTCGCACTAGAATCTGATGAACCAGCTAGACTAGCCCTCGCTAGGCCTTTAAT
TGCTGGAAAGATTTTGAATTGTAGAACCTTCTTGGGAAGATTGGCTAGAGCCAGACAAAC
AAATATGGACGCTCCATTAGCTGCTCTCAAGAGCGCAGCCCAAGCAGCCGGACAAGCTG
CCGATTTGGAAATTTTGAGAGGGATTGAGGGAGCAGCAGCTAGAACATACTTTGCAGCTT
GGCAGACTGTCTTACCAGCAAAATGGCAGTTTACTGGTAGAAATAGGCAACCGCCAACT
GATCCTGTAAATGCCCTGCTGAGTTACGGTTATACAATCGTCTTTTATAACGTGCTGGCAT
TGGTTAGAGCAAGAGGACTAAATACTCATGTTGGGGTATTGCATGACGTTAGACCTGGAC
ACCCCGCCTTAGCTTCGGACTTAATGGAAGAATTTCGTGCACCAGTTGTTGACGCAGTTG
TTATGCACTTGGTTTTCGATGGAAAATTGCAGCCAGGTGATTTCAGCTGGCCAGAAACCC
CCGGACAACCCTGTCTCATGGCAGACGGCTCACGAAAACACTTTATTCACTTATTAGAAC
AGAAATTGAATACAACAGTTTCTCATGCTGGTCAGCGACTTGACTATAGGAGATGGATGG
ATATGCAAGTTCTTCAATATGCAGCAGCTCTTAGAACTCCAGGGTTGCCGTATGTTCCCTT
TGCTATTAGATAA (SEQ ID NO: 115)
Candidatus Accumulibacter sp. SK-02, Cas2, DNA:
ATGGAGCAAACGTGGCTGGTTACGTATGATGTAAGTGATGATGGTTGTAGAAGG
AGAGTAGAAAGGATATTGTTGGGTCATGGAGAGCGTGAGCAATATAGCGTGTTTAGGTGT
AGACTATCTTCTAGGGAAGTTAGGGATTTACGAGCTAGATTGGCAGGACACTTGAAGGGT
TCTGATAGTATTCGATACTACCCCCTTTGTGCTGCATGTTTACCAAGGCAACCCCAACGT
ACCCTAGTCGATTCAGCTGAGGCTGGTATCGCTTGGTATTTCGCAGTTTAA (SEQ ID NO:
135)
Eubacterium saburreum DSM 3986, RT-Cas1, Amino Acid:
MKKLTMEDIFISEQIEEALDHLSTKKDTCGIDGLYLSELRDDWNINGERYLSLLRKGKY
KPGIVQIYEIVNYTGKRRSISSFNSIDRLVLRCLATSLEKYYDSIFSSSSFAFRPGLGVDKAVAT
FANNLNTGLTRVAIIDIKHYFDSIPIDRLEMILKRIIDDNVLLSLFHNLLYCRISEENVIKTKSKGIL
QGSPISPFLGNLYLSLLDTQLESMHVSFCRYCDDIAMFFASFEEAKETYTKVYDILKNDLEMDI
NPQKSGIYEGIKQNYLGYSFTKNKKEHQILAIKKKKAPPQIYQHWSTTAIQRVDRNYHIINNGIL
NRKDFTLLFENDRGKKYLPVEATESLNVYTSVIFSSDFFKYVGNKKICVNIFDKYGELAGTFSP
PESLHGGLTMLKQAAIYLDDEKRKLIARKLEIASLHNMRSNLKYYERHHSSENLKDGITSFSEY
ITAMNEATNIVMLLTIEARARQLYYSLFHEIICDPAFEFTKRTRRPPKDPLNALISFGNVFLYNRI
ATEIQKTSLDIRIGFVHATNRRNQSLNLDIADLFKPLIVDRAIFTIINRHMIHASEHFEKTEDGGIY
LNKEGKQIFINELENKVYQKQTEENKPRTYDTRIREEIHKIFRLVCYDEKYKPFKYN (SEQ ID
NO: 004)
Eubacterium saburreum DSM 3986, Cas2, Amino Acid:
MFVIITYDVKAKRDPKVMKTIRKYLTHEQRSVFEGLITPGRLKHLKEELKRIVNVSEDCI
NIYSLETLRYSKKESIGKQVYHGNII (SEQ ID NO: 024)
Eubacterium saburreum DSM 3986, Array1, DNA, genomic:
CAGGGGCATTGATTTTCTCCTATTTTTTATGTACGAATCATTATAGAATAAAAAGTC
TGTCGCCCTCTATTTTTGCTTTTAAATATGTAATTACATATTCATACTATAGTATGTGTTTGT
AATTACATTATTTGATTAATTTTTATCATATCTTGATATATAAGTTCTTGTTAAGTGTTTTTAA
TATAAGTTTACTGTTTTAAACGGATTTTATTCGAAATAGTGCTATATTTTTGCTTTAATATAC
TTATTTTTAAAACAATACTTACAATTACTCTTTTCATGCAATAAAGAAAAAAAGTCTGTCACT
GATTGTATACAATCACGACACCCTGAAAAGTCCCATTTTAAGCCATTCTTAAAAATAGATT
GACAGACTTTTTAGCCTGTAATACAATAGTTTTAGGTCAAAACAAGAGTTTTTACCTATCA
CAACGGCTTAAATGCAGTTGTAAGTACCTTACCTATAAGGAATGGAAACGAATAAGCTGC
ATAGCAGTGATTCCAATCTGATTTAGTAAGTACCTTACCTATAAGGAATGGAAACAATTCA
AGTAGACAAAGCCTGCTGGATAGTACTTGGTAAGTACCTTACCTATAAGGAATGGAAAC
(SEQ ID NO: 051)
Eubacterium saburreum DSM 3986, Array2, DNA, genomic:
AATGTATGAACTTCATATACTCATGCCATTTATATATATTTAGTGAAAAAGTCTGTC
AATCTCTACTTTTACTTTACGGATATGTATTACAAACACATATTATGGTATGGGTTATGCAT
GTATATTGCATGTTCTTCTTTATCTTATTTTTTATAATAATATGATACAAAACCTCTGTAGAG
CTATTTCGGATGTAGATTTAATATTTTAAGCATATTTTACTTCAAAAAAGTATATTATATAAT
TTTAAATACTGCATTTATTATACAATATTTACAAAGAGCACTTTTGCTCATTAAGATCAAAA
AGTCTGTCACTGATTGTATACAATCAGTGACAGACTTAAAAGCACCATTTTAAGCCATTCT
TAAAAATAGATTGACAGACTTTTTCACCTGTACTACAATAAATTTAGCACAAAGCGAGGGC
TTTCCCATATTACAACGGCTTAAATGCAGTTGTAAGTACCTTACCTATAAGGAATGGAAAC
TGGTCGACCGTCACATTCTGATGAGACTGATCCGTAAGTACCTTACCTATAAGGAATGGA
AACGAAACCCATCCACCCATACCTTCATCAAAATATTGGTCAGTTTCTCCGTAAGTACCTT
ACCTATAAGGAATGGAAAC (SEQ ID NO: 052)
Eubacterium saburreum DSM 3986, Array1, DNA, genomic:
GCTGAAAGGAGGAACTATATCCGGATACCAGGTTCTGTCACTGATTGTATACAAT
CACGACACCCTGAAAAGTCCCATTTTAAGCCATTCTTAAAAATAGATTGACAGACTTTTTA
GCCTGTAATACAATAGTTTTAGGTCAAAACAAGAGTTTTTACCTATCACAACGGCTTAAAT
GCAGTTGTAAGTACCTTACCTATAAGGAATGGAAACGTCCCGCGGCCGCTATTCTTTTGA
TTTATAAGGGATTTTGCGATCCTCTGGAGTGAATACCACGACGATTTCCGGCAGTTTCTC
CACA (SEQ ID NO: 053)
Eubacterium saburreum DSM 3986, Array1 RC, DNA, genomic:
GCTGAAAGGAGGAACTATATCCGGATACCAGGTTATAATCGTTTCAATTCCTTATA
GGTAAGGTACTTACGCAGAGGCTAGAAGAAAAGATATAGAAGAGCAATTGTTTCAATTCC
TTATAGGTAAGGTACTTACGAGAACTCGATAAGCATAAGAATGATAGATTTGTATCACCAG
CTAATAGTTTCCATTCCTTATAGGTAAGGTACTTACGTCCCGCGGCCGCTATTCTTTTGAT
TTATAAGGGATTTTGCGATCCTCTGGAGTGAATACCACGACGATTTCCGGCAGTTTCTCC
ACA (SEQ ID NO: 054)
Eubacterium saburreum DSM 3986, Array2, DNA, genomic:
GCTGAAAGGAGGAACTATATCCGGATACCAGGTCTGTCACTGATTGTATACAATC
AGTGACAGACTTAAAAGCACCATTTTAAGCCATTCTTAAAAATAGATTGACAGACTTTTTC
ACCTGTACTACAATAAATTTAGCACAAAGCGAGGGCTTTCCCATATTACAACGGCTTAAAT
GCAGTTGTAAGTACCTTACCTATAAGGAATGGAAACGTCCCGCGGCCGCTATTCTTTTGA
TTTATAAGGGATTTTGCGATCCTCTGGAGTGAATACCACGACGATTTCCGGCAGTTTCTC
CACA (SEQ ID NO: 055)
Eubacterium saburreum DSM 3986, Array2 RC, DNA, genomic:
GCTGAAAGGAGGAACTATATCCGGATACCAGGTGAACACTGAACGTTGCTCATG
TGTAAGGTATTTTCGGATGGTTTTCATCACCTTGGGATCTCGCTTAGCCTTAACATCATAT
GTGATAATGACGAACATCTCTTCCTCCTTGTATTTTGATTTTAGTATTCCAACCTGGTGAAT
ATAGGTGTTTCCATTCCTTATAGGTAAGGTACTTACGTCCCGCGGCCGCTATTCTTTTGAT
TTATAAGGGATTTTGCGATCCTCTGGAGTGAATACCACGACGATTTCCGGCAGTTTCTCC
ACA (SEQ ID NO: 056)
Eubacterium saburreum DSM 3986, RT-Cas1, DNA:
ATGAAGAAACTGACTATGGAAGATATCTTCATATCCGAGCAAATCGAGGAAGCCC
TCGATCACCTGTCTACTAAGAAGGACACATGCGGTATTGACGGTTTGTACTTGTCCGAGC
TTAGGGACGACTGGAACATCAACGGTGAGAGGTACTTGTCCCTCTTGCGTAAGGGGAAA
TATAAGCCGGGTATCGTGCAGATTTACGAGATTGTTAACTACACCGGGAAGCGTAGATCC
ATTTCCTCTTTTAATTCCATAGATAGGCTAGTCCTGAGATGCCTTGCTACAAGTCTGGAGA
AGTACTACGACTCGATCTTCTCTAGCTCCTCGTTCGCTTTCCGACCAGGGCTCGGGGTG
GACAAAGCAGTCGCCACGTTCGCCAACAACCTGAACACCGGACTGACCAGGGTAGCAAT
CATCGACATCAAACACTACTTCGATTCTATTCCAATCGATCGTTTGGAGATGATACTGAAG
CGAATCATAGACGACAACGTCTTATTGAGTCTATTCCACAACTTACTTTATTGCCGTATCT
CTGAGGAGAACGTAATCAAGACTAAGTCTAAGGGCATACTCCAGGGATCACCAATATCTC
CATTCCTTGGTAACTTGTACTTATCTTTACTAGATACACAGCTGGAGTCTATGCACGTATC
ATTTTGCAGATACTGCGACGACATCGCTATGTTCTTCGCTTCTTTCGAGGAAGCTAAGGA
AACTTACACTAAGGTCTACGATATCTTGAAGAACGACCTGGAGATGGACATTAACCCACA
GAAGTCAGGGATCTACGAAGGCATCAAGCAGAACTACTTGGGTTATAGTTTCACTAAGAA
CAAGAAGGAGCACCAGATCTTAGCTATCAAGAAGAAGAAGGCTCCACCTCAGATTTACCA
GCACTGGTCCACCACCGCAATCCAGAGGGTCGATAGGAATTATCATATCATTAACAACGG
TATTCTTAACCGTAAGGACTTCACCCTCTTGTTCGAGAACGACAGAGGCAAGAAATATCT
CCCTGTTGAAGCCACCGAGTCCCTAAACGTGTACACAAGTGTAATTTTCTCCTCAGACTT
CTTCAAGTACGTAGGCAACAAGAAAATCTGCGTCAACATCTTTGATAAGTATGGGGAGTT
GGCCGGAACTTTCAGTCCACCGGAGTCCCTTCACGGTGGCCTCACCATGTTAAAACAAG
CTGCCATTTACTTGGACGACGAGAAAAGGAAGCTGATCGCCAGGAAATTGGAAATTGCAT
CCCTGCATAACATGAGGTCTAATCTGAAGTACTATGAGCGTCACCACTCCTCTGAAAACC
TGAAGGACGGTATTACCTCGTTCTCTGAATACATTACCGCTATGAACGAGGCTACAAACA
TCGTTATGCTATTAACCATTGAGGCACGAGCACGTCAGTTGTATTATTCTCTGTTTCACGA
GATCATTTGCGACCCAGCTTTCGAGTTCACGAAGCGTACCCGTAGGCCACCAAAAGACC
CCTTGAATGCCTTAATCTCATTCGGCAACGTTTTCTTGTATAACAGAATCGCCACGGAAAT
CCAGAAAACATCGTTGGACATAAGGATCGGGTTCGTTCACGCTACAAACAGGCGTAACC
AAAGCCTGAACCTAGATATAGCCGACCTATTCAAGCCCCTGATCGTGGATAGAGCTATTT
TCACCATCATTAACCGACACATGATCCACGCTTCAGAGCACTTCGAGAAAACAGAGGACG
GTGGCATTTACCTCAACAAAGAGGGTAAGCAGATCTTCATTAACGAGTTGGAGAACAAGG
TTTATCAGAAACAAACAGAGGAGAACAAACCTCGTACCTACGATACTCGTATCCGAGAGG
AGATCCACAAGATTTTCCGATTAGTTTGTTACGACGAGAAGTATAAGCCATTCAAGTATAA
CTAA (SEQ ID NO: 116)
Eubacterium saburreum DSM 3986, Cas2, DNA:
ATGTTTGTAATCATAACCTACGACGTAAAGGCCAAGAGGGACCCGAAGGTCATGA
AAACTATAAGGAAGTACTTAACCCACGAGCAGAGGTCTGTTTTCGAGGGGCTCATCACGC
CCGGTAGATTGAAGCACTTGAAGGAAGAACTTAAAAGGATTGTGAACGTGTCAGAGGACT
GTATCAACATTTACTCCCTGGAAACCCTGCGTTACAGTAAGAAGGAGTCCATCGGGAAGC
AAGTTTACCACGGCAACATCATTTAA (SEQ ID NO: 136)
Bacteroides fragilis S14, RT-Cas1, Amino Acid:
MPDYYHSITTLHALQNAWRAVRAKNAAGGIDGFTLSHFEKRLNDNLIELQHELISQTW
NPEPYLRIEITKNETEKRKLGLLCIKDKIVQQAIKTAIEPQLEKTFLNLSYGYRPNKGPERAIKRV
VHDLKKLKSGYVAKLDIDNYFDTINHERLFTRLANWLKDDETLRLIRLCIQTGIVTPQLQWQEIN
KGVPQGAILSPLLANFYLHPFDQFAANKVPMYIRYADDFLIATSTEKQIKEAVELVKEELESQF
YLQLNTPIIHNFHDGIEFLGITISDTGLSITEKKKKTLQERINSIKFIKSSLSSQSKETLQGIKNYYA
KLLPESTLKELDCFLMNRLNALIIRNQNSINNKKELVSNLQKIEFYSENSNKNKSQLIQQLCSTY
IVHSTKSKTRLTSTHIDNTKLITQKKKEYQKRENEGAELVISIPGSYIGATYKGITVKLQGKIINKP
SPALKHITVVGKGISLSSNAITYCMNHKIPIDFFDGRGKQYGTVLNPVFLDGTLWNKQVELPLE
QKIKLATQIIIGKLKNQLNLIKYYHKYHKDILGGKLSEKYVEVVLKIDKLIEKAKNYSQRNEKYTA
ELMAIESQAAIAYWSYIRVLTADDGIDFIRREHQGATDLLNSLLNYGYAILYARVWKNILAAKLN
PSIGVLHAKQDGKPTLVFDVVELFRAQMVDRVVISLIQKKVSLKMHDGLLNESSKRVLIRYILE
RLNRYEKYRGEEITFSQIILRQAQEIALFISGDNLIFKPYVAKW (SEQ ID NO: 005)
Bacteroides fragilis S14, Cas2, Amino Acid:
MVKAKKIFCVVAYDIQDDRSRIQISKILEKYGTRINYSVFECMFTDRQFQKIQINLERWI
NRRYDTVVYYPMCINCYTRIIYQPIRKKIIKTVEIV (SEQ ID NO: 025)
Bacteroides fragilis S14, Array, DNA, genomic:
ACCTCTTGCGAAAATCAGAATTTGTTTAATTCATTGATTTTCTGCTTATTCTTTTAA
ATGAAAAATAATATATAATGTATAAAAGCTACGAAAGAACTAGCTTTTTTGTATATTAGTAA
ATCATTTGCGAAAAAAAACAAATAAGCAATTCGTTCTTTGACATAATGCACTTATTATCTGA
TAATTATAAATATAAGATTAATCTTATTTTCGAAATAGGCTATAATATAACAAATGAAGCAG
GCATTTGCGAATAATGAATCGATCATATCAAAGTGATAATCAATTAAATATCTCTATATTCT
ACAAATTATTTATTTAAATTACAGAAAAAAAGAGTACATTTGCGAAAATCAGTTCAACACTT
CATCTATCTAACTGAATAATAAGTTATTATATATTTATACAGATAGAATACATATTTTATAAT
AATCTGATTATCAATATATGTAGATGTATTCCAGTATAATAAGGATTAAGACAATAGGCGT
AGCCGTTCATAACATAACTAGCCTCTAATGTAGATGTATTCCAGTATAATAAGGATTAAGA
CTTGAATTCCAAGACCTAAAGCCGTACCGATTGATGTAGATGTATTCCAGTATAATAAGGA
TTAAGAC (SEQ ID NO: 057)
Bacteroides fragilis S14, Array1, DNA, genomic:
GCTGAAAGGAGGAACTATATCCGGATACCAGGTTATATTCTACAAATTATTTATTT
AAATTACAGAAAAAAAGAGTACATTTGCGAAAATCAGTTCAACACTTCATCTATCTAACTG
AATAATAAGTTATTATATATTTATACAGATAGAATACATATTTTATAATAATCTGATTATCAA
TATATGTAGATGTATTCCAGTATAATAAGGATTAAGACGTCCCGCGGCCGCTATTCTTTTG
ATTTATAAGGGATTTTGCGATCCTCTGGAGTGAATACCACGACGATTTCCGGCAGTTTCT
CCACA (SEQ ID NO: 058)
Bacteroides fragilis S14, Array1 RC, DNA, genomic:
GCTGAAAGGAGGAACTATATCCGGATACCAGGTAATGTTAAAAGCAATACCGAAG
TCCATGACGATCTTATCCAAGCCGTTATGTCTGAACTTCTCCCCTTATTCTATTTAATGTA
GTTAAATTTTTATATTGGAATACATCTACATGCTAATAAGGAAAGTTTGCAGTATTTCTGGG
CATTGGTCTTAATCCTTATTATACTGGAATACATCTACATGTCCCGCGGCCGCTATTCTTT
TGATTTATAAGGGATTTTGCGATCCTCTGGAGTGAATACCACGACGATTTCCGGCAGTTT
CTCCACA (SEQ ID NO: 059)
Bacteroides fragilis S14, RT-Cas1, DNA:
ATGCCAGACTACTACCACTCCATCACTACTCTCCATGCACTTCAGAACGCTTGGA
GAGCAGTTAGAGCTAAGAACGCTGCCGGTGGTATAGACGGTTTTACGCTTTCCCACTTC
GAGAAAAGACTTAATGACAACTTAATCGAGTTGCAGCACGAGTTGATCTCACAGACTTGG
AACCCTGAGCCCTATCTGAGAATCGAGATAACGAAGAACGAAACGGAGAAGCGTAAGCT
AGGTTTGCTCTGTATCAAAGATAAGATTGTCCAGCAGGCTATCAAGACCGCTATCGAGCC
ACAACTGGAAAAGACTTTCCTTAACCTGTCCTATGGATATAGACCTAATAAGGGCCCAGA
GAGAGCCATTAAGAGAGTTGTGCATGACTTGAAGAAGCTGAAATCTGGATACGTCGCTAA
GTTGGACATCGATAATTACTTCGACACAATAAACCACGAGAGACTATTTACCAGACTAGC
AAACTGGCTAAAGGACGACGAGACTTTGAGATTAATTAGGTTGTGCATTCAGACCGGCAT
TGTCACGCCTCAGCTACAGTGGCAGGAGATTAACAAGGGCGTCCCCCAAGGGGCCATTC
TCTCACCGTTGCTGGCCAATTTCTACTTACACCCGTTCGACCAATTCGCAGCTAACAAGG
TTCCAATGTACATCAGATATGCCGATGACTTCCTGATAGCCACTTCGACTGAGAAGCAGA
TCAAGGAAGCCGTGGAGCTAGTCAAAGAGGAGCTTGAGTCACAGTTCTACTTGCAGTTAA
ACACTCCCATCATTCACAACTTTCACGACGGTATTGAGTTCCTGGGTATTACCATTAGCGA
CACTGGACTGTCCATTACCGAGAAGAAGAAGAAAACCTTGCAGGAAAGGATTAACTCCAT
TAAGTTCATCAAGTCATCCCTGAGTTCGCAGTCTAAGGAAACATTACAGGGCATTAAGAA
CTATTACGCTAAATTATTGCCTGAGTCTACCCTGAAAGAGCTAGACTGTTTTCTGATGAAT
CGTCTGAACGCTCTCATCATAAGAAATCAGAATAGTATCAACAATAAGAAGGAGCTAGTC
TCTAACTTGCAGAAGATCGAGTTTTACTCTGAGAACTCAAACAAGAACAAGAGCCAGCTC
ATCCAGCAGTTGTGCTCAACTTACATTGTCCACAGTACTAAGTCCAAAACGAGATTGACTT
CCACTCACATCGACAACACCAAATTAATTACTCAGAAGAAGAAGGAGTACCAAAAGAGAG
AGAACGAGGGAGCAGAGCTTGTTATTAGCATTCCCGGAAGTTACATCGGCGCTACATAC
AAGGGTATAACTGTTAAGCTACAAGGCAAAATCATAAACAAGCCCTCGCCGGCCTTGAAG
CATATCACTGTCGTTGGAAAAGGTATCTCTCTTTCTTCAAACGCTATAACCTACTGTATGA
ATCATAAGATCCCTATCGATTTCTTTGACGGAAGGGGTAAGCAGTACGGAACAGTCTTAA
ACCCCGTTTTCCTTGACGGCACGCTGTGGAACAAGCAAGTCGAGCTTCCCTTAGAGCAG
AAGATTAAGCTGGCCACCCAGATAATCATCGGAAAGCTGAAGAACCAGCTTAACTTGATC
AAATACTATCACAAGTATCACAAGGACATTCTAGGCGGCAAACTTTCCGAGAAGTACGTA
GAGGTGGTGTTGAAGATTGATAAATTGATCGAAAAGGCAAAGAACTACTCGCAACGAAAC
GAGAAGTACACCGCTGAGTTGATGGCTATCGAATCGCAAGCCGCCATTGCTTACTGGTC
CTATATTAGAGTCCTAACCGCCGACGATGGCATCGACTTCATTAGGCGAGAACATCAGG
GAGCTACTGACCTGTTGAATAGTTTGCTGAATTACGGTTACGCCATCTTGTACGCCAGGG
TGTGGAAGAACATCTTGGCAGCAAAGCTCAACCCCTCAATCGGTGTTCTGCACGCCAAA
CAGGACGGAAAGCCAACCTTGGTGTTCGACGTGGTGGAACTGTTCAGGGCACAGATGGT
CGATCGTGTCGTTATCTCTTTAATCCAGAAGAAGGTGAGCTTGAAGATGCACGATGGATT
ATTGAATGAGTCCAGTAAGAGAGTGCTTATAAGGTACATCTTGGAAAGATTAAACAGGTA
CGAGAAGTACAGGGGCGAGGAGATTACTTTTAGTCAGATCATCCTCAGGCAGGCACAGG
AGATTGCTTTGTTCATATCGGGCGATAACCTTATCTTCAAGCCATACGTAGCTAAGTGGT
GA (SEQ ID NO: 117)
Bacteroides fragilis S14, Cas2, DNA:
ATGGTTAAGGCCAAGAAGATCTTCTGCGTCGTCGCTTATGACATCCAGGACGACA
GGTCTAGGATTCAGATCAGCAAGATCCTTGAGAAGTACGGGACCAGAATAAACTACTCCG
TCTTTGAGTGCATGTTCACCGACCGTCAGTTCCAGAAAATACAGATAAACCTGGAGCGAT
GGATAAACAGACGTTACGACACGGTTGTCTACTACCCTATGTGTATTAACTGTTACACCC
GTATCATCTACCAGCCAATTAGGAAGAAGATTATCAAGACTGTGGAGATCGTGTGA (SEQ
ID NO: 137)
Campylobacter fetus subsp. Fetus, RT-Cas1, Amino Acid:
MFEKTLEQILSDKNIQIALNSLKKTALGIDNLSELNEHFIHKLKQSCLNQTYAPEPVLQK
LIPKSDGENYRKLAISSLKDKLIQKVLANELTWYFDKHFSDKSYAYRPGKSYKNAIFRLRDFLR
VKPYFVIKSDIKDCFESINHSKLVALLAKYIKDKRVLNLVEIWIKNGIFNRQTYIKHSKFGIHQGD
VLSPLLANIYLNQMDKFLETNNEIFIRYADDFVILVDDEKFVQAKINSLKTFLSTIDLSLKDTKTAI
YSPTQSFEFLGVSFYGSNLSINEAKFDKIQEKIYALSKSNDFKTDFNSYIAHLQTISLNLIKTDQT
QLKRFIYALKKCVSLYIKNKTTLKTRLEIFTFLDTLNFALNFKSKGEKDEFYNQIYALTREKQALK
AKPQPNPQHVLNKKKKHYLEQFAQNSLLHISTPHCFLGVSNVNFVIRYKGKVILKVRIDQIHQIII
ACDISLSTNAIKAATKRNISIDFLGFNNQIYASLFSHTSTITPAYKAQIDFLNSPNSLNLAKEFIKA
KATNQINYLKYLDKHYKILASNIDKMHKNLKKALISATTTSELMGYEGAISSLYFDAIASTLEDKE
FKRVGKGATDLVNSLLNYGYAILYSTVQSALIKAGLYLNISYFHVSAKFSLSFDFIEEFRVVAVD
RVVFTLLHQKTKLSLKDGLLDVPTKKKLTQKVALALLSTHKYKNEELNLEQIIQAQAYLLRKQIF
NEAKYKGFLVRFQG (SEQ ID NO: 006)
Campylobacter fetus subsp. Fetus, Cas2, Amino Acid:
MHYIICYDIAQTRRRTRLATLLEAIGTRANKSVFEAKLTAKELELFIAKAKAIIEPKTDSV
LIYPLCFDCMIKSLSLGQKGVFEVKDSFV (SEQ ID NO: 026)
Campylobacter fetus subsp. Fetus, Array, DNA, genomic:
GAGCTATTTGGTCGCTTTTGATTTGTAAATTTTACGAGTTACATCGTGCTATATAA
ATTTGATTATCTATAAATTTTTAAACACGGTTTTTATTTTTGGTTCCGCCATAATTTCAATAT
ATTTTTAACAAATTTAGCACTTTAGAGTTTCTAACTTTTTATATTTTAAAACTCCTAAATTTA
GAGCTTTTTGGTTTTTTGCTCTTTTAAAAAAGTTAGAAACTCAATAAATTTAGCATTTTTAAA
AACACACTTTCTAACTTTTATAATTATTTAAATTTTAAAAGCCCATTTTTGCGGTATTTTAAA
ACTTAGATTTTTCTTAAATTTGGAAACTATACGCAGTTTAAAAATTTCCCCATTCGACTTTC
TAAATTTTTAAATCTCAAAAACCACCATTTTACCGATACTTTAAGGACAAATTTAGCAAAAC
TATCGATTTATACATTTTAAAAATCAATAGAGTTTCGAATAAATTCGCTCGAATTCAGCTCT
CACAGGCTTTTGCTTAAAGAATTTATAATCATTAAAAAAGTATAATCATCTTGCAAATAACT
AAATTTGCAGTGTTTAGACACTTTCTCCGATTATTAGGGGATTGAAACAGAATTTGGCAAT
TTTTTTAAATTTTAATTTTACTAGTTTAGACACTTTCTCCGATTATTAGGGGATTGAAACTTT
AAATCTGTAGAGCTAAAACTTTGTAATCTAAAGTCTATAGTTTAGACACTTTCTCCGATTAT
TAGGGGATTGAAAC (SEQ ID NO: 060)
Campylobacter fetus subsp. Fetus, Array1, DNA, genomic:
GCTGAAAGGAGGAACTATATCCGGATACCAGGTCAAATTTAGCAAAACTATCGAT
TTATACATTTTAAAAATCAATAGAGTTTCGAATAAATTCGCTCGAATTCAGCTCTCACAGG
CTTTTGCTTAAAGAATTTATAATCATTAAAAAAGTATAATCATCTTGCAAATAACTAAATTTG
CAGTGTTTAGACACTTTCTCCGATTATTAGGGGATTGAAACGTCCCGCGGCCGCTATTCT
TTTGATTTATAAGGGATTTTGCGATCCTCTGGAGTGAATACCACGACGATTTCCGGCAGT
TTCTCCACA (SEQ ID NO: 061)
Campylobacter fetus subsp. Fetus, Array1 RC, DNA, genomic:
GCTGAAAGGAGGAACTATATCCGGATACCAGGTATAATAGTAACATAGTCCATAT
AAGTTATAGCAGACATAAAAAATTCGCCCATATCTTCTCCAAGTTTTATAATTTAAATTTAA
TTATACCTCTTTTTTATTTGGCTAAAGCTAAATTTTTATAAAACCTGCTACCTATATACAAAT
TCTGTTTCAATCCCCTAATAATCGGAGAAAGTGTCTAAACGTCCCGCGGCCGCTATTCTT
TTGATTTATAAGGGATTTTGCGATCCTCTGGAGTGAATACCACGACGATTTCCGGCAGTT
TCTCCACA (SEQ ID NO: 062)
Campylobacter fetus subsp. Fetus, RT-Cas1, DNA:
ATGTTCGAGAAAACCCTAGAGCAGATCCTAAGTGACAAGAACATTCAGATTGCTC
TGAACTCTCTGAAGAAAACCGCCCTGGGTATCGACAACTTATCCGAACTGAACGAACACT
TCATCCATAAACTCAAGCAGTCCTGCCTGAATCAGACCTACGCCCCTGAACCAGTCTTAC
AGAAGTTGATTCCTAAGTCCGACGGAGAGAATTACAGGAAACTCGCTATTTCCTCATTGA
AGGATAAGCTAATCCAGAAGGTATTGGCCAATGAACTGACTTGGTACTTCGACAAACACT
TCTCTGACAAGAGCTACGCATACAGGCCTGGTAAGTCATACAAGAACGCCATCTTCAGGC
TGAGAGACTTCTTGCGAGTGAAGCCCTACTTCGTGATCAAGTCCGACATTAAGGACTGCT
TCGAATCTATCAACCACAGTAAATTAGTGGCTTTACTGGCCAAGTACATCAAGGACAAGC
GTGTCTTAAATCTGGTTGAGATATGGATTAAGAACGGCATATTCAACAGACAGACATATAT
CAAGCATAGCAAGTTCGGCATTCACCAAGGGGACGTGCTGTCACCACTGCTCGCAAATA
TCTACCTCAATCAGATGGACAAGTTTCTCGAAACAAACAACGAGATCTTTATTCGATACGC
AGACGACTTCGTCATCCTTGTGGACGACGAGAAATTCGTACAGGCAAAGATCAACAGCTT
AAAGACCTTCCTCTCAACTATTGACTTGTCCTTGAAGGACACTAAGACCGCTATTTACTCT
CCGACCCAGTCTTTCGAATTTCTAGGAGTCTCCTTCTACGGATCTAACCTGAGTATTAAC
GAGGCAAAGTTCGATAAGATCCAGGAGAAGATCTATGCCCTGAGTAAGTCTAACGACTTC
AAGACCGACTTCAACTCCTATATTGCTCATCTCCAGACAATCAGCCTGAACCTGATCAAG
ACCGATCAGACTCAACTAAAGAGGTTCATTTACGCCTTAAAGAAGTGTGTGTCCTTGTAC
ATCAAGAATAAGACAACTCTGAAAACCCGATTAGAGATATTCACATTCCTCGACACTCTGA
ACTTCGCACTTAACTTCAAGTCCAAAGGTGAGAAGGACGAGTTCTACAATCAGATCTACG
CTCTCACCAGAGAGAAGCAAGCACTGAAGGCCAAGCCACAACCTAACCCGCAGCATGTC
TTGAATAAGAAGAAGAAGCATTACCTTGAGCAGTTCGCTCAAAATAGCTTGCTCCATATTA
GTACCCCTCACTGCTTCTTGGGCGTCAGCAACGTGAATTTCGTTATCAGATATAAGGGAA
AGGTGATTCTGAAGGTCCGAATAGACCAGATCCACCAGATTATCATAGCCTGTGACATCT
CATTGTCCACTAATGCTATTAAGGCCGCAACCAAGCGAAACATATCTATCGACTTTCTTGG
ATTCAATAATCAGATATACGCCAGCCTGTTCAGTCACACCAGTACAATAACACCGGCTTAT
AAGGCCCAGATCGACTTCCTAAATTCCCCAAACTCATTGAATCTGGCTAAGGAATTTATAA
AGGCAAAGGCAACCAATCAGATCAACTATTTGAAGTACTTGGACAAGCACTACAAGATCT
TGGCATCCAATATCGACAAGATGCACAAGAACCTGAAGAAGGCCCTTATTTCAGCCACCA
CCACCTCGGAGCTGATGGGATACGAGGGAGCCATCAGTAGCCTGTACTTCGATGCCATT
GCCTCAACTCTCGAAGATAAAGAGTTTAAGCGTGTTGGTAAGGGTGCTACCGACCTTGTT
AACTCGCTTCTGAACTACGGTTACGCCATCTTGTACTCCACCGTCCAGTCTGCACTGATT
AAGGCAGGCTTATACCTGAACATTTCCTACTTCCACGTTTCAGCCAAGTTCAGTTTGTCTT
TCGACTTCATCGAAGAATTTAGGGTGGTGGCCGTGGACCGTGTGGTATTCACCTTACTCC
ACCAGAAAACTAAGCTGTCCTTGAAGGACGGGCTGCTAGATGTCCCGACCAAGAAGAAG
TTAACACAGAAAGTCGCCCTGGCACTACTCAGCACTCACAAGTACAAGAACGAGGAGCT
GAACCTAGAGCAGATCATTCAGGCCCAGGCTTACCTCTTGAGAAAACAGATCTTCAATGA
GGCCAAGTATAAAGGGTTCCTGGTTCGATTCCAGGGTTAG (SEQ ID NO: 118
Campylobacter fetus subsp. Fetus, Cas2, DNA:
ATGCACTACATTATTTGCTACGATATTGCTCAGACTCGACGAAGAACAAGGCTGG
CTACCCTACTAGAGGCCATTGGTACACGAGCTAATAAGTCTGTTTTCGAGGCCAAATTGA
CTGCCAAGGAATTGGAATTGTTCATCGCTAAGGCTAAGGCAATAATAGAACCAAAGACGG
ACTCTGTCCTCATTTACCCGCTTTGCTTCGATTGCATGATCAAGTCTCTGTCGCTAGGTCA
GAAGGGAGTGTTCGAGGTGAAGGACTCTTTCGTATGA (SEQ ID NO: 138)
Teredinibacter turnerae T8412, RT-Cas1, Amino Acid:
MVTRVEEFDPLPLENLEQAALLEQLLPLRSLAVHLQFIDESHPRFFHQAAVSALLRHL
LPSADDYSHYLILDAPETARINYQAGASYCFSIVCLAGGESLLATLMLALRQLPYSAPTGNPLA
SFGANLRWRGFSCNFCDHPVDQVEDLGCYGIEHLAQEAALWAEHHTLFHWQWLTPVRLLK
EKSARSTAKGEARYCADAGDLSAALLLARLYDTVNALLTERGDRPLPPRQAAPHMNVEAQH
CFWLDAGYQNSSGKEQVMGGLLGEFTYSTPVPLPPPWAHFLVLGQYIGLGQRRSFGWGRY
QLVTTENQVSCRRQLAAQPLLERALEPANLRLALQHKTQKTKAKREPLYKWQRDAQECDLS
QYECNEETDAEGDQAELPPTLLKRANALAQGRYDVPPLRGVIIPKTDGEWRALAVAPFFDAV
LQRAVAQILAPSLDRVMDNRSYGYRRGRSRLDAKEQIQLAYRNGARWVLEADIEDFFDSVAF
SLVAQRLRALFHQDPINEAILAWLSAPVDYDGLRLQRKAGLPQGSPLSPVLANLLLDDFDSDM
RKAGFNCLRFADDFVVVCQSREEAERAWQRAASSLNEHGLFLAENKTRVISFERGFRFLGYL
FVNELALDVGSKALKQHDKLSSPGHAKPQCASGWLADFLAQRPQALLPPNAPHSEQRVVEK
TTAMVHSQAVALGNRENDGTFLCVSGAPALISTDHGRVLVQRDDETVMSVPWQGLRSVLLL
GRHHLTHPAMIAALSQGVAIHFASRGGQYQGLLDGNQPRLGPRLWLLQEERVADAAACVAV
ARELTNARIRHQREVLRQRALSGWHTLGDSLSQVDACTDLSALQGIEGAAARTYFAALAQAV
HPQWGFHGRNRRPPRAPFNALLSLGFTLLYAHTETLLIIDGLNPRAGFYHKPHGSHSTLASD
MMEPFRHLVERCALSFLSSGKVKPADFSVKDNGACELSNAARRLYLERLSERFETSMKGRD
GSEGKLIQLLRWQNRSLIELIRAKGPFTAWLQR (SEQ ID NO: 007)
Teredinibacter turnerae T8412, Cas2, Amino Acid:
MKTYLVCFDITDDKSRNKVGNLLLAYGERVQLSVFEIALNNNRELAALKTQLQTLMEE
GDDLRFYYLSKETRRQSEDVYGNAIADFPSVIIV (SEQ ID NO: 027)
Teredinibacter turnerae T8412, Array, DNA, genomic:
CGCCAGTGGCGAAGCAGAGTTATCGGTTTCGGGCGAAGCGAAGGACAAGCCAC
GCCAGTTCAGCACAAAACAACATAAAAATATGAAAATTCAAAAACATATAAGAATAACTTC
ACGTATTACATTAGCTTTGTGTCGCGCAGGCAAAAAAATCAAACACTTAGCGTTGCCGCA
CAGGCAAATTAGCGCGATAATGCGCCCTAAATCAGCCCAAACTAACCGTTTGCAAAGAGC
ATCGCCCAAACGGGGTTCTAGCATGCTGATTTATAAAGGGAAAAATGGGGAGCTGTCGC
AATCGACATTAGCCGCTAAGGCTGTGAAACAAAAGTTCAAGCATAGTAGTCTCCTTAATAT
GCTTTTGTCGCAATCGACATTAGCCGCTAAGGCTGTGAAACAATAAGTATGAAGATTTGTT
TTAAGATTTTATAGGTCGCAATCGACATTAGCCGCTAAGGCTGTGAAAC (SEQ ID NO:
063)
Teredinibacter turnerae T8412, Array1, DNA, genomic:
GCTGAAAGGAGGAACTATATCCGGATACCAGGTCGCAGGCAAAAAAATCAAACA
CTTAGCGTTGCCGCACAGGCAAATTAGCGCGATAATGCGCCCTAAATCAGCCCAAACTAA
CCGTTTGCAAAGAGCATCGCCCAAACGGGGTTCTAGCATGCTGATTTATAAAGGGAAAAA
TGGGGAGCTGTCGCAATCGACATTAGCCGCTAAGGCTGTGAAACGTCCCGCGGCCGCT
ATTCTTTTGATTTATAAGGGATTTTGCGATCCTCTGGAGTGAATACCACGACGATTTCCGG
CAGTTTCTCCACA (SEQ ID NO: 064)
Teredinibacter turnerae T8412, Array1 RC, DNA, genomic:
GCTGAAAGGAGGAACTATATCCGGATACCAGGTCTGGTTGTCGGCTCGCACATT
ACATTGCGCCGCAGTGTTCCAGTGCACTCGCCTCACTCTACCGGTTTTTACCGGCTTCTA
CCGCCACCTTCCCTCTCAGCCGATTACATCAAACTAGCGGCGGATTGAGTCCATTTTAAG
CACATTGTGGTTTCACAGCCTTAGCGGCTAATGTCGATTGCGACGTCCCGCGGCCGCTA
TTCTTTTGATTTATAAGGGATTTTGCGATCCTCTGGAGTGAATACCACGACGATTTCCGGC
AGTTTCTCCACA (SEQ ID NO: 065)
Teredinibacter turnerae T8412, RT-Cas1, DNA:
ATGGTAACTAGAGTCGAGGAGTTTGATCCTCTCCCCCTTGAGAATTTAGAACAAG
CAGCACTATTGGAGCAGCTGTTACCTTTGCGTTCTCTTGCTGTTCATCTTCAGTTCATTGA
CGAGTCCCATCCAAGGTTCTTTCATCAAGCTGCTGTTTCAGCTCTCCTAAGACACTTGCT
ACCATCCGCTGATGATTACTCACACTATCTTATTTTGGACGCTCCCGAGACTGCTAGAAT
CAATTATCAAGCTGGAGCTTCTTATTGCTTCTCTATAGTCTGCTTGGCTGGGGGGAGTC
GCTTTTAGCCACTCTAATGCTTGCTTTGAGACAGTTACCATATTCCGCACCTACAGGGAA
CCCTTTAGCTTCATTTGGTGCTAATTTGCGTTGGAGAGGGTTTTCGTGTAACTTTTGCGAC
CATCCCGTGGATCAAGTCGAGGACTTGGGTTGTTATGGGATAGAGCACCTAGCACAAGA
GGCTGCTTTATGGGCCGAACACCATACTTTGTTCCACTGGCAGTGGCTTACTCCAGTCAG
GCTTTTGAAGGAGAAGTCTGCTAGATCTACAGCTAAAGGAGAGGCAAGATACTGCGCTG
ACGCTGGAGATTTGTCAGCTGCACTCCTCTTGGCCAGACTATATGATACTGTGAACGCAT
TGCTGACTGAGAGAGGGGACCGACCACTACCCCCACGACAAGCAGCTCCACACATGAAT
GTAGAGGCTCAACATTGTTTCTGGCTTGATGCTGGTTACCAAAATAGTTCTGGAAAGGAG
CAAGTGATGGGTGGACTTTTAGGGGAGTTTACATATTCTACTCCTGTTCCACTTCCACCC
CCTTGGGCTCACTTCCTAGTTCTAGGTCAGTACATTGGGTTGGGACAAAGAAGGTCATTC
GGTTGGGGAAGATATCAATTAGTCACGACCGAGAATCAAGTGTCGTGCAGGCGTCAGTT
AGCCGCTCAACCACTGTTAGAGAGAGCTTTGGAACCAGCTAATCTAAGGCTTGCTTTGCA
GCACAAGACTCAGAAAACTAAGGCCAAGCGAGAGCCACTTTATAAATGGCAGCGTGACG
CTCAAGAGTGTGATTTATCCCAGTATGAATGCAATGAGGAAACTGATGCCGAGGGTGACC
AAGCCGAACTCCCACCAACTTTGCTTAAACGAGCTAACGCTTTAGCTCAGGGACGTTATG
ATGTCCCACCCTTGAGAGGCGTTATTATTCCTAAGACAGATGGAGAGTGGAGAGCTTTAG
CTGTCGCTCCCTTCTTCGACGCAGTATTGCAGAGAGCAGTCGCCCAAATTCTCGCTCCCT
CACTAGATCGAGTTATGGATAATAGGTCATACGGTTACAGGCGTGGCAGAAGTAGGTTAG
ATGCCAAGGAGCAGATTCAACTGGCCTATCGTAATGGAGCTAGATGGGTTCTCGAAGCT
GACATCGAAGATTTCTTCGATTCTGTTGCCTTCTCTTTAGTTGCTCAAAGGCTAAGAGCAT
TGTTTCATCAAGATCCCATTAACGAAGCTATCCTGGCTTGGCTGTCTGCTCCCGTAGACT
ATGATGGTCTTAGATTACAGAGAAAGGCAGGATTGCCTCAAGGTTCTCCTCTTAGCCCAG
TTCTAGCTAATTTGTTGCTAGATGATTTTGACTCCGACATGAGGAAGGCTGGATTTAATTG
TTTACGATTTGCAGATGATTTCGTCGTTGTTTGTCAGTCACGAGAAGAAGCAGAAAGAGC
ATGGCAGAGGGCAGCTTCTTCTTTGAATGAGCATGGTCTTTTCCTAGCTGAGAATAAGAC
TAGAGTCATAAGTTTCGAGCGAGGCTTTAGGTTCCTCGGTTACCTATTTGTTAATGAGTTA
GCTCTTGACGTAGGATCGAAGGCTTTAAAACAACATGACAAACTTTCATCGCCTGGCCAC
GCAAAGCCACAGTGTGCTTCCGGATGGCTGGCCGACTTTCTGGCACAAAGGCCACAGG
CATTGCTGCCACCTAATGCACCTCACTCAGAACAAAGAGTAGTAGAGAAAACTACAGCTA
TGGTGCACTCTCAAGCTGTAGCTTTGGGTAATCGTGAGAACGATGGTACTTTCCTATGCG
TGTCGGGAGCCCCAGCATTGATTTCCACTGATCATGGGCGAGTGCTCGTCCAAAGAGAT
GATGAGACTGTGATGTCTGTTCCCTGGCAGGGATTGAGATCAGTTCTTCTTTTGGGACGA
CACCATTTGACTCATCCCGCCATGATCGCTGCATTGTCGCAAGGAGTAGCAATCCACTTC
GCAAGTAGAGGTGGACAATACCAAGGTCTTTTGGACGGAAACCAGCCGAGACTAGGTCC
GAGATTATGGCTGTTACAAGAGGAGAGAGTAGCTGATGCTGCTGCATGTGTAGCTGTAG
CTAGAGAGTTGACTAATGCCAGGATAAGACACCAGAGAGAGGTTCTAAGACAAAGGGCA
TTATCTGGATGGCACACTCTAGGAGATTCATTGTCCCAGGTAGACGCATGTACAGACCTT
TCAGCTCTACAGGGTATTGAGGGTGCTGCTGCTCGAACTTATTTCGCTGCTCTGGCTCAA
GCTGTTCATCCTCAGTGGGGCTTCCACGGGAGAAACAGACGACCGCCACGAGCCCCATT
CAATGCTCTGCTATCTCTGGGATTTACATTGTTATATGCACATACCGAGACTCTTTTAATC
ATTGACGGCCTTAATCCCCGAGCTGGATTCTATCACAAGCCTCATGGGTCACACTCAACT
TTGGCTAGTGATATGATGGAACCATTTAGGCATCTGGTTGAGAGGTGCGCCCTATCTTTC
TTATCAAGTGGTAAGGTTAAACCCGCTGATTTCAGTGTAAAGGATAACGGTGCCTGTGAG
TTATCTAATGCTGCTCGTAGATTGTATTTGGAGAGATTATCCGAGAGATTCGAGACATCTA
TGAAGGGTCGTGACGGAAGTGAGGGGAAGCTAATCCAGTTATTGAGGTGGCAGAATAGA
AGTCTTATTGAGCTGATCCGAGCAAAGGGACCCTTTACTGCTTGGCTTCAACGATAG
(SEQ ID NO: 119)
Teredinibacter turnerae T8412, Cas2, DNA:
ATGAAAACTTACTTGGTATGCTTTGATATCACCGACGATAAGAGTAGAAACAAAGT
CGGTAACCTTCTTCTGGCATACGGAGAGCGTGTTCAGTTGTCCGTGTTCGAGATAGCACT
CAACAACAATCGAGAACTAGCAGCCTTAAAGACCCAGCTCCAGACCCTAATGGAAGAAG
GGGACGACCTTAGGTTCTACTACTTATCTAAGGAAACCCGTCGTCAGTCAGAGGATGTGT
ATGGAAATGCTATAGCCGACTTCCCAAGCGTGATCATAGTATGA (SEQ ID NO: 139)
Woodsholea maritima, RT-Cas1, Amino Acid:
MMRLEACLTVKSLMTAWKKVYANQGGPGGDGQTLAQFQRTVLLHLHRLGDDVRAG
LYMPGPHRVVSIPKRAGGWRSLSIPCVRDRVLQTAVAQRLQPILEPEFEPESYGYRPGRSVA
QAIARVATLRRQGFRWTVDADIERFFDCVPHGPLLERLRPFLGDPGLVGLVEMWLAGAGPH
GRGLPQGSPISPLLANLYLDDVDEGLKSTHTRLVRFADDFVILTRNEDEALQALERARGLLDK
LGLSLNLEKTRIVPFEGGLDFLGRKFVRALVVDDVYEDEGEALAAGEPARTHARAMPGEDAL
DLASLEAGADTSRAPRLRVLYLLEKGHVLGTHNSSFTVRNAQGDPVSTLPTTRVDRIEVGSQ
ARIADEAIRLALDEDVEMRWINGRGQTEGYLSRPERGHGALHLAQLRLYDNAEARLAAARILV
EGRLRNQRALLRRLNRRRKRPFIAERAKQIGGVLKYLPEAQTIEALMGREGQAGALYWPALG
ACLEHGWTFTQRVRRPPPDPVNLVLSYLASLLCRDIASLAARQGLHVGIGALHAVQDEPRDT
LAFDLAEEFRAPLVEGLCIWMLNTRTLGHQMFHTREDGQVYAHGEGIKTILRSWENWLDRPV
RSPRSGQDVLWRGLIEEQILCWRDHVSGKSVYQPYRMEY (SEQ ID NO: 008)
Woodsholea maritima, Cas2, Amino Acid:
MADPLFVFAYDISQDRVRRRVAGLLEAEAVRVQGSVFEARLSKSRAKALGQRIAIELE
RGDTLRVYCLSQRDLKSSFQWGGAPMAEAQDFYIL (SEQ ID NO: 028)
Woodsholea maritima, Array, DNA, genomic:
CTGAGAAAGGCCTGTAGAATTAGGTCAAAACATTGATTATATGAATTTAAAATCAT
TCCAGATCGGGATTTATGTCCACCTCAGCCCTTTGGCGCTTATTATGAGCCTAAGCCTGT
AGATCATGGAGCTGAATGTCACATCTTGTCAATGATGTAGAAGGGGGTAGGCTCTGCTC
GAATCTTTCAGCAGAGCTGATCAAAACTTCTCCGCTGCTTGAGATTCCAGCATTGTGATG
GCGCTCTGCTCGAATCTTTCAGCAGAGCTGATCAAAACGCTGTTGGGCGATGTTATCGGT
AAATTGCATCAGCTCTGCTCGAATCTTTCAGCAGAGCTGATCAAAAC (SEQ ID NO: 066)
Woodsholea maritima, Array1, DNA, genomic:
GCTGAAAGGAGGAACTATATCCGGATACCAGGTGAATTAGGTCAAAACATTGATT
ATATGAATTTAAAATCATTCCAGATCGGGATTTATGTCCACCTCAGCCCTTTGGCGCTTAT
TATGAGCCTAAGCCTGTAGATCATGGAGCTGAATGTCACATCTTGTCAATGATGTAGAAG
GGGGTAGGCTCTGCTCGAATCTTTCAGCAGAGCTGATCAAAACGTCCCGCGGCCGCTAT
TCTTTTGATTTATAAGGGATTTTGCGATCCTCTGGAGTGAATACCACGACGATTTCCGGCA
GTTTCTCCACA (SEQ ID NO: 067)
Woodsholea maritima, Array1 RC, DNA, genomic:
GCTGAAAGGAGGAACTATATCCGGATACCAGGTGCACGCAGCGCAGCGCCTTTC
GGGTTGGCCCCGCTTATGAACATCGGTGTGATGATCTTCAATGTCAGGTTCAGTTGCTGC
ATCTTTTCCCCATCCCGGCGTGCACGTTAAATGTGCATTTTGCGGCTTTCAAGGGAAAAT
TAACGCGAAGTTTTGATCAGCTCTGCTGAAAGATTCGAGCAGAGCGTCCCGCGGCCGCT
ATTCTTTTGATTTATAAGGGATTTTGCGATCCTCTGGAGTGAATACCACGACGATTTCCGG
CAGTTTCTCCACA (SEQ ID NO: 068)
Woodsholea maritima, RT-Cas1, DNA:
ATGATGAGATTAGAGGCATGTTTAACTGTTAAGTCCCTTATGACAGCTTGGAAGA
AAGTATACGCAAACCAAGGTGGTCCAGGTGGAGATGGACAGACTCTAGCACAGTTTCAG
AGAACTGTTCTCTTACACCTTCACAGATTGGGTGACGATGTTCGAGCAGGACTATACATG
CCTGGACCTCACAGGGTTGTTTCGATCCCAAAACGAGCCGGTGGATGGAGATCACTTTC
TATTCCGTGTGTTCGAGATCGAGTTTTGCAGACTGCTGTTGCTCAAAGGTTACAGCCGAT
ACTCGAACCTGAATTTGAGCCAGAAAGCTACGGATACCGTCCAGGTAGATCTGTTGCTCA
AGCTATTGCTAGGGTGGCTACTCTTAGAAGGCAAGGCTTCAGATGGACTGTTGACGCTG
ACATAGAACGTTTCTTCGATTGCGTCCCGCACGGACCACTATTGGAAAGACTTAGGCCCT
TTCTTGGCGACCCAGGTTTAGTAGGTCTAGTCGAAATGTGGTTGGCTGGAGCTGGGCCC
CACGGCAGAGGATTGCCTCAGGGTAGTCCAATTAGTCCACTCCTTGCTAACTTGTACTTG
GACGACGTAGACGAAGGTCTAAAGTCAACACACACTAGATTGGTTAGATTTGCTGACGAC
TTTGTTATCTTGACTCGTAACGAGGACGAAGCTCTACAAGCCCTAGAACGAGCTAGGGGT
TTGCTAGACAAACTGGGTCTTTCATTGAATTTGGAGAAAACTAGGATTGTACCATTCGAG
GGTGGTCTTGATTTCTTGGGTAGAAAGTTCGTAAGGGCTCTTGTTGTGGACGACGTTTAC
GAGGACGAGGGTGAAGCCCTAGCTGCTGGCGAGCCAGCTCGTACCCATGCCAGAGCTA
TGCCTGGTGAAGATGCTTTAGACTTGGCTTCCCTTGAGGCAGGCGCTGATACCAGTAGA
GCACCTAGACTAAGAGTTTTGTATCTGTTAGAGAAGGGACACGTCTTAGGTACTCACAAT
AGTTCATTTACTGTCAGAAACGCTCAAGGTGACCCAGTGAGTACCCTTCCGACAACGAGA
GTAGATAGAATCGAAGTAGGTTCTCAAGCTAGAATTGCTGACGAAGCTATCAGGTTGGCC
CTTGACGAGGATGTCGAGATGAGGTGGATTAACGGTCGAGGTCAGACTGAGGGATACTT
GTCACGTCCCGAGAGAGGACACGGTGCTTTACATTTGGCTCAACTTAGACTGTACGATAA
TGCAGAAGCTAGGCTTGCAGCTGCTAGAATCTTAGTCGAAGGTAGATTGCGTAACCAAAG
AGCTTTACTGAGAAGGCTCAATAGAAGAAGAAAACGACCCTTTATCGCTGAAAGGGCTAA
ACAAATCGGTGGTGTTTTAAAGTATCTCCCTGAGGCCCAGACTATCGAAGCTCTGATGGG
TAGGGAAGGCCAAGCTGGTGCCTTGTATTGGCCAGCTTTGGGTGCTTGTTTGGAACACG
GATGGACTTTTACCCAAAGGGTAAGGCGACCGCCCCCAGACCCTGTAAACTTGGTACTG
TCGTACTTGGCTTCTTTGCTGTGCAGGGACATAGCTTCCTTGGCCGCCAGACAAGGGCT
CCACGTAGGTATTGGCGCTCTACATGCAGTTCAGGATGAACCGAGGGATACTTTAGCATT
CGACCTCGCTGAGGAGTTCAGAGCACCGCTCGTTGAAGGACTTTGCATTTGGATGTTAAA
CACACGTACATTGGGTCACCAGATGTTCCATACTAGAGAGGACGGACAAGTTTACGCACA
CGGAGAAGGAATTAAAACAATACTGAGATCCTGGGAAAACTGGTTGGATAGACCAGTAAG
ATCTCCTAGATCTGGCCAGGATGTCTTGTGGAGAGGGCTTATTGAAGAACAGATCTTGTG
TTGGAGAGATCACGTGAGTGGAAAATCCGTGTACCAACCGTACAGAATGGAGTACTAA
(SEQ ID NO: 120)
Woodsholea maritima, Cas2, DNA:
ATGGCAGATCCATTGTTTGTTTTCGCATACGACATCTCACAGGACCGTGTGAGAA
GGAGAGTCGCAGGACTACTTGAAGCAGAAGCCGTAAGAGTACAAGGTAGCGTCTTTGAA
GCTAGATTGTCCAAAAGTAGAGCTAAGGCTCTGGGACAAAGAATTGCTATAGAATTAGAA
AGGGGAGACACCTTGAGAGTCTACTGCTTGTCTCAGAGGGACCTCAAGAGTTCTTTCCA
GTGGGGTGGCGCTCCAATGGCTGAGGCACAAGACTTTTATATTCTGTAA (SEQ ID NO:
151)
Desulfarculus baarsii DSM 2075, RT-Cas1, Amino Acid:
MVDMRLYAKIIAKSSLMLAWEKVLANKGAPGGDRQTLDDFAESLERNLEGLHAALRS
ASYRPGPIRNVSIPKRDGSPRRLSIPSVADRVVQTALCQGLTPILEPEMEDASFAYRPGRSVQ
MAVERVGRYFRQGYHWVVDGDIDDYFDSIPHHGLMAVLRRYVDDQDVLGLIAQWLAHAHAG
GVGVSQGSPLSPLLANIYLDDMDERIGRTGARLVRFADDFLLLCKSEERARESLAAMSALLAE
YGLGLNPDKTRIVNFEQGFEFLGRLFVRSMALEREQESDAPQETPPGPTPDDPSPPVEPLHQ
ASEGPGFQDLSPRLRVMYLSRKGCRLDVRGRAFVVRSGPEPDAPELMVVLPSQLDRVELW
PGCDISQKAQRFALECRTPVAYVDGWGRTLGVLEPMVADKAALHLAQAAVALDETKRLALA
RLICAGRVRGQRALLMRLNRRRKNSDIESNLAAFKQLPRRIATATTISELLGLEGEAAKRYWA
SLALLLDKSWGFSSRQRRPPRDGVNMVISYVASMLYRDLRCLAARHGLHPGFASLHGSLDG
KPGCISDLVEEFRAPLCEGLAVYLANNHILKKEMFYKTDKWPCHVTPEGRETIIRAYEAWLDR
PVKSPRSGEKVKWRGLLEEQVLAYRDHVMGRSVYAPYDMKY (SEQ ID NO: 009)
Desulfarculus baarsii DSM 2075, Cas2, Amino Acid:
MSGAEMLVVFAYDVEDDSRRRRLARVLGNHAVRVQKSVFEAWLDEGAAKIIASRAA
AELGPRDSLRVYALDASGVGKTLVFGVTAPPQSHDYYFV (SEQ ID NO: 029)
Desulfarculus baarsii DSM 2075, Array, DNA:
GCCAAGCCGTCGGCGCGGGGGCTATTTGGCTTGTTTGCAATATATTTCGCCATTT
TATGTGTTTTTTAACCGTGTGGAATTGATATGGCGCCGTATTCATTATGTTTTGCGCGCAA
ACGCCCAAAAAATGCGCGGAACGGCCCTGTTGGCGCGCCGAATTTGGCAAACCATGTG
GAAACAGCCCCAAAAATAAATATTGCAATCGGGGGGTTGGCTGGGGTACCCTATTGAGG
GATGCCGCTAGAGGAAGCGGATTGAAACCTAGACTCCACGGCCCCGGCGTTGCCATTTT
TTCTATTGAGGGATGCCGCTAGAGGAAGCGGATTGAAACCAAGCCGATGCTTTCCGACT
TCCTCTCTTTCTCCTCGCTATTGAGGGATGCCGCTAGAGGAAGCGGATTGAAAC (SEQ ID
NO: 069)
Desulfarculus baarsii DSM 2075, Array1 RC, DNA:
GCTGAAAGGAGGAACTATATCCGGATACCAGGTTGGAATTGATATGGCGCCGTA
TTCATTATGTTTTGCGCGCAAACGCCCAAAAAATGCGCGGAACGGCCCTGTTGGCGCGC
CGAATTTGGCAAACCATGTGGAAACAGCCCCAAAAATAAATATTGCAATCGGGCGGTTGG
CTGGGGTACCCTATTGAGGGATGCCGCTAGAGGAAGCGGATTGAAACGTCCCGCGGCC
GCTATTCTTTTGATTTATAAGGGATTTTGCGATCCTCTGGAGTGAATACCACGACGATTTC
CGGCAGTTTCTCCACA (SEQ ID NO: 070)
Desulfarculus baarsii DSM 2075, Array2, DNA:
GCTGAAAGGAGGAACTATATCCGGATACCAGGTTGCTTCCGGGTTGGCTGCTAT
ATTTGGCGATAAATAAATGGCGTGGCCGGCGTAAAAAACGCCGAAAATAGGCCCGCCTT
TTCGCCAGCAACAATCAGGCGATAATGATAAAAGCCGTGGCAAAAACGCCACGGCTTATT
TTTTTGCCCCGTTTCAATCCGCTTCCTCTAGCGGCATCCCTCAATAGGTCCCGCGGCCGC
TATTCTTTTGATTTATAAGGGATTTTGCGATCCTCTGGAGTGAATACCACGACGATTTCCG
GCAGTTTCTCCACA (SEQ ID NO: 071)
Desulfarculus baarsii DSM 2075, Array2 RC, DNA:
GCTGAAAGGAGGAACTATATCCGGATACCAGGTAAACGATGTTTGTCAAATTTAA
TCAAATGATTATATTGATCATGGCCCGAAATGGCAAGGTTTTTGCCAGGCTGACGACCGA
TAATGGCATGTCCGTGTGGAAACAGCCCCAAAAATAAATATTGCAATCGGGGGGTTGGC
CAGGGTACCCTATTGAGGGATGCCGCTAGAGGAAGCGGATTGAAACGTCCCGCGGCCG
CTATTCTTTTGATTTATAAGGGATTTTGCGATCCTCTGGAGTGAATACCACGACGATTTCC
GGCAGTTTCTCCACA (SEQ ID NO: 072)
Desulfarculus baarsii DSM 2075, Array2 RC, DNA:
GCTGAAAGGAGGAACTATATCCGGATACCAGGTCTTGAGCGGGGCCGAGGCCC
CGACCAAGAGCCGGCCGCTGGCGGTCCGGGCGATGATCGGGCGTGGCTGGCCGCGGG
CGCGTCGCCCGGCCAAAGGCCGGACAAGACTAGACGTTAGCATGCGCTGCAAGGCCAG
GCTGGCGCAAGCAAAGTTTCAATCCGCTTCCTCTAGCGGCATCCCTCAATAGGTCCCGC
GGCCGCTATTCTTTTGATTTATAAGGGATTTTGCGATCCTCTGGAGTGAATACCACGACG
ATTTCCGGCAGTTTCTCCACA (SEQ ID NO: 073)
Desulfarculus baarsii DSM 2075, RT-Cas1, DNA:
ATGGTGGATATGAGGTTGTATGCCAAAATTATTGCTAAGTCTAGCCTTATGCTTGC
TTGGGAGAAAGTTTTGGCTAATAAAGGAGCACCGGGCGGCGATCGTCAGACCCTAGACG
ACTTCGCAGAGTCTTTAGAAAGAAATTTAGAGGGATTACATGCTGCATTGAGGAGCGCTA
GTTACCGTCCAGGACCAATTAGAAATGTTTCCATCCCAAAGAGAGATGGAAGCCCTCGTC
GTCTGAGCATTCCATCCGTCGCTGACAGAGTTGTCCAGACAGCTCTTTGTCAGGGTTTAA
CCCCAATTTTGGAACCTGAAATGGAAGATGCTTCTTTTGCATACAGGCCAGGTAGATCAG
TACAGATGGCTGTAGAAAGGGTAGGAAGGTACTTTAGACAAGGTTACCATTGGGTCGTA
GATGGTGATATTGATGACTACTTCGATTCTATTCCGCATCATGGCCTGATGGCTGTCCTG
AGGAGATATGTTGATGACCAAGATGTCCTGGGATTGATCGCACAGTGGTTAGCTCATGCA
CACGCTGGTGGAGTCGGAGTGTCTCAAGGAAGTCCATTATCACCTCTCTTAGCTAATATA
TACCTGGATGATATGGATGAGAGAATTGGGAGAACTGGTGCTAGACTCGTTAGATTCGCT
GACGACTTCCTATTGCTCTGTAAGTCAGAAGAAAGAGCTAGAGAATCGTTAGCAGCTATG
TCTGCCCTGTTAGCTGAATACGGATTGGGTTTGAATCCGGATAAGACTAGAATAGTTAATT
TCGAACAGGGGTTCGAGTTCCTGGGGAGACTGTTCGTCCGTAGCATGGCACTTGAGAGA
GAACAAGAATCTGATGCACCCCAGGAGACTCCACCAGGACCTACTCCTGATGACCCTAG
TCCCCCAGTAGAACCATTACACCAAGCATCCGAAGGTCCTGGGTTTCAGGATTTGTCTCC
GAGATTGAGAGTAATGTACCTATCAAGAAAAGGATGTCGTTTGGACGTAAGAGGTCGTGC
TTTCGTTGTTAGAAGCGGTCCCGAGCCCGATGCACCTGAACTTATGGTTGTACTCCCGTC
CCAACTGGATAGGGTTGAGCTATGGCCTGGGTGTGATATCTCTCAGAAGGCTCAACGAT
TTGCTCTTGAATGCAGGACTCCTGTTGCTTATGTAGACGGATGGGGTAGAACTCTCGGAG
TCCTTGAGCCTATGGTAGCAGATAAAGCTGCCCTTCATTTGGCTCAAGCTGCCGTCGCTT
TGGATGAAACAAAGAGGTTAGCTCTTGCTCGTTTAATTTGTGCTGGTAGAGTAAGGGGTC
AGAGAGCACTTCTAATGAGATTAAATAGAAGAAGAAAGAACTCAGACATAGAGAGTAACT
TGGCTGCTTTTAAACAGCTTCCTCGTAGGATTGCTACCGCTACTACTATTTCAGAACTTCT
TGGACTTGAGGGCGAAGCCGCTAAAAGGTACTGGGCCTCTCTTGCTTTGCTACTTGATAA
GTCGTGGGGCTTCAGTAGTCGTCAGAGAAGGCCACCACGTGACGGCGTCAATATGGTTA
TTAGCTACGTTGCTTCAATGTTATATAGGGACTTGCGATGTCTCGCTGCAAGACATGGAC
TACACCCTGGGTTTGCATCCCTTCACGGATCTCTGGATGGAAAACCTGGATGTATTTCAG
ACTTAGTTGAGGAGTTCCGAGCCCCTTTGTGTGAAGGCCTTGCTGTCTACCTGGCTAACA
ACCATATTCTGAAGAAGGAAATGTTCTATAAGACTGACAAGTGGCCCTGTCACGTTACAC
CAGAGGGTCGAGAGACAATAATCCGTGCCTATGAAGCATGGCTTGATAGACCTGTAAAG
TCTCCAAGATCTGGTGAGAAGGTAAAGTGGCGAGGGTTGTTGGAAGAACAGGTTTTAGC
ATATAGGGACCACGTTATGGGAAGATCTGTCTACGCCCCTTATGATATGAAGTACTAA
(SEQ ID NO: 121)
Desulfarculus baarsii DSM 2075, Cas2, DNA:
ATGAGTGGTGCCGAGATGCTAGTTGTATTTGCTTATGACGTTGAGGACGACTCTC
GTAGGCGACGACTGGCACGTGTACTAGGCAATCATGCTGTTAGAGTTCAGAAGTCTGTTT
TCGAGGCTTGGTTAGACGAAGGTGCTGCTAAGATAATTGCTAGTCGTGCCGCCGCAGAA
TTGGGACCGAGGGACTCTCTGAGAGTATACGCATTAGATGCTTCTGGAGTCGGCAAGAC
CTTGGTGTTCGGTGTTACTGCACCACCACAATCCCACGACTACTATTTCGTCTAA (SEQ
ID NO: 140)
Azospirillum lipoferum 4B, RT-Cas1, Amino Acid:
MPTAPPDNADLFEEVTRLDTLERAWGRVLRNAGAAGGDGLTVGRFAEAAPSRLLAL
HRTLRMGDYRPGPLRRLSIPKPDGALRPLAIPPVTDRVAQTAAALVLTPLLDGEFEDASFGYR
PGRSVPQAVARVARWRDQGYDWVVDADIERYFERVPHDRLLIRLERSIGAGPLTELIAVWLE
SGAENGVGLPQGSPLSPLLSNLYLDDLDEALDGRGLRLVRFADDFVLLCRSRERAERALDHA
AAVLEEHGLRLNRDKTRIVPFDQGFRFLGHLFVRSLVLPSPRPDDGEETEGDALLRALAIRDA
AAEAEEAAAEERDRQSRAAGLDPALRTLHVQTPRRRLALRNEAFTVVERDDLGGERELIAIH
HHHLDRIDLGPEADADAEALRWALATDTELAFVDGHGATHGRLTRPEARRAALHLDQARHAL
DEGLRLDLARRIVDGRLRNQRALLRRLNRSRKLGVVEDAVLAINALLRRLDTAADVAALLGFE
GQGAARYWPALAAQIEGEWEFEGRRRRPPPDPVNAVLSYLSGLLERDVAALIARHGLHPGF
GVLHSPQDRHDAGIYDLMEEFRAPLMEGLAVTLFNRRTLRPDHFSRRETGEGEIKGCRIDPD
AVGAIIRAYEQWVRRPVASPRDGKRTTWRGLIGFQAQALAAHVQGREPYRAYVMDY (SEQ
ID NO: 010)
Azospirillum lipoferum 4B, Cas2, Amino Acid:
MSETPMVMVFCYDVANDRRRRRVSAVLEEWGVRVQKSVFEARLTEPQARALLARA
GKELGPRDSLRMYALSAHGLAHSAALGGAPVPEQQDFWLL (SEQ ID NO: 030)
Azospirillum lipoferum 4B, Array, DNA:
GGCGAGGCCGCGCGCAAGGACGTTGCGCGGAGGATCGCGGTTGTGGATGTTTG
TGTCAAGTTTGGTGTGGAAGGATGTGTAGAACGTTGATTGCTATCGGGATTTGGTGTGTT
TGATGGTGGATCTTAACCAAAAAATAGCGTTTCCGGCTTTTTTATCCACAGGATGCGTGG
AAAGGCGGCTGAAAAAGCAACTTGAATCAAAGGACTGCGAGGGGCACTGTTCCGCCGGT
GTCCGCGTTTGGGAGCGGATTGAAACATACGCAGCAGCTTCGGTTCGTTGAGTTGGAGT
TCGACGTTCCGCCGGTGTCCGCGTTTGGGAGCGGATTGAAACCCTTGGGCGGTGTTGG
ACGAGCCGTTGACGCCGGTGTCCTGCGTTCCGCCGGTGTCCGCGTTTGGGAGCGGATT
GAAAC (SEQ ID NO: 074)
Azospirillum lipoferum 4B, Array2, DNA:
GCGGTGGCTGTGTCTTGCGCCGGTTGTACCCCTTTCCGGGCTATCCGGCGAGG
AGGAAGGGCAGGCGGCGGGGTAACGGAATGATGCGGGACGGGGGTGGGATTTTGGCG
GTTGTTTCAAATCTGGTGTGGAAGAGCTGGTAGACCATTGAAGATATTGGGATATTGGGC
TGTTCATTGGGTGGTGTTAACCACTTTTCCGGGATTTTGGAGATTTTATCCACACCATGCG
TGGAAACCGTTGGGAATTTTCCCGTTGAATCAAGOGGATGCGAGGGATACCGTCGCGAC
GGCATCCGCGTTCGGGAGCGGATTGAAACCATACAGGCTTGGTCATGTCGGGCTCCGG
GAAAAAGAGTCGCGACGGCATCCGCGTTCGGGAGCGGATTGAAACGTTCAGCCGCCGT
GGGGGGAGATCAGCGGGGCTTTCGTCGCGACGGCATCCGCGTTCGGGAGCGGATTGAA
AC (SEQ ID NO: 075)
Azospirillum lipoferum 4B, Array1, DNA:
GCTGAAAGGAGGAACTATATCCGGATACCAGGTAGAGCTGGTAGACCATTGAAG
ATATTGGGATATTGGGCTGTTCATTGGGTGGTGTTAACCACTTTTCCGGGATTTTGGAGA
TTTTATCCACACCATGCGTGGAAACCGTTGGGAATTTTCCCGTTGAATCAAGOGGATGCG
AGGGATACCGTCGCGACGGCATCCGCGTTCGGGAGCGGATTGAAACGTCCCGCGGCCG
CTATTCTTTTGATTTATAAGGGATTTTGCGATCCTCTGGAGTGAATACCACGACGATTTCC
GGCAGTTTCTCCACA (SEQ ID NO: 076)
Azospirillum lipoferum 4B, Array1 RC, DNA:
GCTGAAAGGAGGAACTATATCCGGATACCAGGTTCGGCGCACGGGTTGGCGCA
CAGCGCCGCGCTGGGGGGCGCCCCGGTGCCGGAACAGCAGGATTTCTGGTTGCTGTAG
GGGGGAGGAAGCCGGCCGCGACAATTGGCTCTCGGCCGGGGGGATCATCTTCCTCGAC
GAAATCACGCGAGCGTTTCAATCCGCTCCCGAACGOGGATGCCGTCGCGACGTCCCGC
GGCCGCTATTCTTTTGATTTATAAGGGATTTTGCGATCCTCTGGAGTGAATACCACGACG
ATTTCCGGCAGTTTCTCCACA (SEQ ID NO: 077)
Azospirillum lipoferum 4B, Array2A, DNA:
GCTGAAAGGAGGAACTATATCCGGATACCAGGTAGGATGTGTAGAACGTTGATT
GCTATCGGGATTTGGTGTGTTTGATGGTGGATCTTAACCAAAAAATAGCGTTTCCGGCTT
TTTTATCCACAGGATGCGTGGAAAGGCGGCTGAAAAAGCAACTTGAATCAAAGGACTGC
GAGGGGCACTGTTCCGCCGGTGTCCGCGTTTGGGAGCGGATTGAAACGTCCCGCGGCC
GCTATTCTTTTGATTTATAAGGGATTTTGCGATCCTCTGGAGTGAATACCACGACGATTTC
CGGCAGTTTCTCCACA (SEQ ID NO: 078)
Azospirillum lipoferum 4B, Array2 RC, DNA:
GCTGAAAGGAGGAACTATATCCGGATACCAGGTCTTCCGCTTCGCAATCTGGAA
CGCCTCCGGCCTCCTCATGACCTGTTCCCCAGGGGCTAGTCGGAAGGGCCATCACGTC
CAAAGGCGCGATTGACCTGAGTTTGTTGCCCGGCCGGATCGCGCCGCCTGCGCGGGAC
GGTCGATCCAAGAGTTTCAATCCGCTCCCAAACGCGGACACCGGCGGAACGTCCCGCG
GCCGCTATTCTTTTGATTTATAAGGGATTTTGCGATCCTCTGGAGTGAATACCACGACGAT
TTCCGGCAGTTTCTCCACA (SEQ ID NO: 079)
Azospirillum lipoferum 4B, RT-Cas1, DNA:
ATGCCTACGGCACCCCCTGATAATGCAGACCTATTTGAAGAAGTGACCAGATTGG
ACACACTTGAGAGAGCTTGGGGTAGAGTCTTGCGAAATGCTGGTGCTGCTGGTGGTGAC
GGTTTGACTGTGGGTCGTTTTGCAGAAGCTGCTCCATCTAGGCTTTTGGCATTACATCGT
ACTCTTAGAATGGGTGACTATAGGCCTGGTCCCCTTAGAAGATTATCTATTCCAAAACCA
GATGGTGCTTTAAGACCACTGGCTATCCCACCAGTCACTGATCGTGTCGCTCAAACTGCT
GCTGCTCTTGTATTGACGCCACTTTTGGACGGAGAATTTGAGGACGCTTCATTTGGGTAT
CGACCTGGCAGATCAGTTCCTCAAGCAGTAGCCAGGGTAGCTAGATGGAGAGATCAAGG
ATACGATTGGGTCGTGGATGCAGATATTGAGAGATACTTTGAGCGTGTTCCACATGATAG
ACTTCTAATACGATTGGAAAGGAGTATTGGTGCTGGTCCACTTACTGAATTGATTGCAGT
GTGGTTAGAGTCGGGAGCCGAAAATGGTGTTGGTTTACCTCAAGGTTCTCCATTAAGTCC
ATTACTATCTAATTTGTATCTTGATGACTTAGATGAAGCTCTTGATGGCAGAGGTCTACGT
CTTGTTAGGTTTGCAGATGACTTTGTTCTATTGTGTAGAAGCAGAGAACGTGCTGAGAGA
GCTTTAGATCACGCAGCTGCTGTATTGGAAGAACACGGTTTGAGACTGAATAGAGATAAA
ACCAGAATTGTTCCCTTTGATCAAGGATTTAGGTTCTTGGGTCACCTATTTGTAAGATCCT
TGGTTTTGCCTTCACCAAGACCAGACGATGGTGAAGAAACCGAAGGTGACGCATTATTGA
GAGCACTCGCAATTAGAGATGCCGCCGCTGAAGCAGAAGAAGCTGCCGCAGAAGAAAG
AGATAGGCAAAGTCGTGCTGCTGGGCTTGATCCTGCACTTAGAACGTTACACGTACAAAC
GCCTAGAAGGAGACTAGCTTTAAGGAATGAGGCTTTTACTGTTGTTGAGAGGGATGACCT
TGGTGGTGAAAGAGAACTCATTGCTATTCATCATCATCATTTGGATCGTATTGATCTAGGC
CCAGAAGCAGATGCAGACGCTGAAGCATTGAGATGGGCCCTTGCTACAGATACTGAGTT
GGCATTTGTTGATGGCCACGGTGCTACTCACGGTAGACTAACGAGGCCTGAAGCTAGGA
GAGCTGCTTTGCATTTAGACCAAGCCCGTCATGCTCTTGATGAAGGTTTGCGTTTAGATC
TAGCAAGAAGAATTGTAGATGGAAGATTGCGAAATCAAAGAGCACTTCTTAGAAGATTGA
ATCGTTCCAGAAAACTTGGAGTTGTCGAGGACGCTGTCCTCGCTATAAATGCACTACTTA
GAAGATTAGATACAGCTGCTGATGTTGCTGCATTGTTAGGATTTGAAGGTCAGGGAGCTG
CTCGATACTGGCCAGCTTTAGCTGCCCAAATTGAAGGAGAGTGGGAATTTGAAGGAAGA
AGGCGTAGACCACCCCCTGACCCTGTAAATGCTGTACTCTCTTACCTCTCGGGTTTATTG
GAGAGAGATGTAGCTGCTCTTATTGCTAGGCATGGGCTACATCCGGGATTTGGAGTACTA
CATTCACCACAAGATCGACATGATGCAGGAATTTATGATTTGATGGAAGAATTTAGAGCC
CCCTTAATGGAAGGCCTGGCTGTTACTTTGTTTAATAGGAGAACGCTCAGGCCAGATCAC
TTTAGTAGACGTGAAACGGGTGAGGGAGAAATTAAAGGATGTCGTATCGACCCCGATGC
TGTTGGAGCTATTATTAGGGCTTACGAGCAATGGGTCCGTCGTCCTGTTGCATCTCCAAG
AGATGGTAAAAGAACTACTTGGAGAGGTCTTATAGGATTCCAAGCACAAGCATTGGCTGC
ACACGTCCAAGGAAGGGAACCCTATAGAGCTTACGTGATGGACTATTAG (SEQ ID NO:
122)
Azospirillum lipoferum 4B,, Cas2, DNA:
ATGTCTGAAACACCAATGGTTATGGTGTTCTGCTACGATGTCGCCAATGATAGAA
GAAGAAGGAGAGTCTCAGCAGTACTTGAGGAGTGGGGAGTTAGGGTACAAAAGTCCGTA
TTTGAAGCAAGACTAACTGAGCCTCAAGCTAGAGCTTTGTTAGCAAGAGCTGGAAAAGAA
CTGGGTCCAAGAGATTCTCTTAGAATGTATGCACTTAGCGCACATGGCCTCGCTCATTCT
GCTGCCCTCGGTGGGGCTCCAGTTCCCGAGCAACAAGACTTTTGGCTCTTGTAA (SEQ
ID NO: 141)
Cellulomonas bogoriensis 69B4, RT-Cas1, Amino Acid:
MARVADEGALTRAWSEVLEAAERDGRVPVSVRRFERGVAASLVRLSGELSSGRYQ
PSRVSEVSLRTGSGSERVLRIGAVVDRVVERSLLNALTPVIDPLLSPFAFGFRRGLGVKDAVA
ALARARDEGSTHVLRSDIAAAFDSVPRARAVQALSRLVPDRRVCDVVASLLARLDDYGLEGV
GIAQGSAVSPLLLNLYLLPFDEALMANGFTPLRYADDIAVPAMSESQAQSAAQDVAHQLECL
GLACSAPKTSIRSFDEGVHFLGVTLRARPSSTAPPPARSRPQRISMVVSEGGAVVRTRRGRV
RVDRDGETVASMSLSRVARIVVQGRVGLTTPLLHEAAQRGIDVVMLSRSGGYVGRLSRRRP
GDPSLRRAQARAYDSGADLERLTTAFVSGKITNMRVAVLRHQRGAGTSEEGARVAAQLAEA
RARASVMRSVPSLMGVEGAATRAYFGWLGSRVGEEWGFHGRARRPPPDPVNSMLSYGYV
LLCAEGVSACEQAGLDPDMGFLHSDRWGRPSLALDLMEEWRPVIVDSTVLRIISNKRLKPSD
FTFDAKQGARMTAHARQTFLREYEARMLTLAGSDAGAGRQPYRRLIATQAMRLAEALRTPG
GPYRPYVWR (SEQ ID NO: 011)
Cellulomonas bogoriensis 69B4, Cas2, Amino Acid:
MIWILACYDIADDDRRSRLSDLLAELGPRVQQSVFECRLPSKKALRRLLGELAALIDPV
EDQVRVYELGGQGPRPQIVGTRILEEWRDMWVV (SEQ ID NO: 031)
Cellulomonas bogoriensis 69B4, Array, DNA:
AGCGCGGGGACCGCGCGCGCGACACATCTGCGCTGGTCACAGCCATCGATCCG
CCCCAGTCCCCCGCTGGCGCGCACGCCGTCCCGCGCGCACGCCCTCGCGCGCGCGTT
CGCCCGCGTCAGCCCTGGTCAGCCCGGGGTCAAAACGGGTAGGGTTCCACCCATGGCC
CGAAACTGAGGGCATTGAAACTAAAACATCCCCTGAGGGGATGTAAAGGGTTTAAAGTTC
CACCCATGGCCCGAAACTGAGGGCATTGAAACGGGTACTCCAAACGCCAAGCCTTAATC
GTGTCAGTTCCACCCATGGCCCGAAACTGAGGGCATTGAAAC (SEQ ID NO: 080)
Cellulomonas bogoriensis 69B4, Array1, DNA:
GCTGAAAGGAGGAACTATATCCGGATACCAGGTGCGGGGACCGCGCGCGCGAC
ACATCTGCGCTGGTCACAGCCATCGATCCGCCCCAGTCCCCCGCTGGCGCGCACGCCG
TCCCGCGCGCACGCCCTCGCGCGCGCGTTCGCCCGCGTCAGCCCTGGTCAGCCCGGG
GTCAAAACGGGTAGGGTTCCACCCATGGCCCGAAACTGAGGGCATTGAAACGTCCCGC
GGCCGCTATTCTTTTGATTTATAAGGGATTTTGCGATCCTCTGGAGTGAATACCACGACG
ATTTCCGGCAGTTTCTCCACA (SEQ ID NO: 081)
Cellulomonas bogoriensis 69B4, Array1 RC, DNA:
GCTGAAAGGAGGAACTATATCCGGATACCAGGTCACACGGCACGCGACAGCGA
CTCGACCATCGTGAGAGCTCATCAGAACGCCCCGCCGTGCACAAGGGTGAAATTGATAC
CGCTCGCTGACCTGAGGCTGGAGCCGCCCGAACGCGCGGCCGCCCGCCTCTCGTTGG
CGTAGCCGCCCGCCGTTTCAATGCCCTCAGTTTCGGGCCATGGGTGGAACGTCCCGCG
GCCGCTATTCTTTTGATTTATAAGGGATTTTGCGATCCTCTGGAGTGAATACCACGACGAT
TTCCGGCAGTTTCTCCACA (SEQ ID NO: 082)
Cellulomonas bogoriensis 69B4, RT-Cas1, DNA:
ATGGCTAGAGTCGCAGATGAAGGAGCCTTGACGAGAGCTTGGTCCGAAGTTCTT
GAGGCTGCTGAAAGAGATGGCAGAGTACCAGTGAGCGTCCGAAGATTTGAGAGAGGTGT
TGCTGCTAGTCTAGTGCGACTTTCTGGAGAATTGTCATCTGGAAGATATCAGCCATCAAG
AGTTTCAGAAGTTAGCCTAAGAACTGGTAGTGGCTCAGAAAGAGTTTTGAGAATTGGCGC
AGTTGTAGATCGTGTTGTAGAAAGGAGTTTGCTTAACGCTTTGACTCCCGTTATTGATCCT
CTACTGTCCCCTTTTGCTTTTGGTTTTAGGAGAGGTCTAGGCGTTAAAGATGCCGTAGCA
GCTTTGGCTAGAGCTAGAGATGAAGGTTCTACACATGTGCTTAGAAGTGATATTGCCGCC
GCTTTTGATTCCGTGCCAAGGGCACGAGCTGTTCAGGCACTTTCACGTTTAGTACCAGAT
AGACGTGTTTGTGATGTCGTTGCCTCTTTACTGGCTAGACTAGATGATTATGGTTTAGAAG
GTGTGGGTATTGCTCAAGGTTCAGCTGTATCTCCACTCCTCTTAAATTTGTATTTACTTCC
CTTTGATGAGGCTTTGATGGCTAATGGTTTTACACCACTTAGATACGCTGATGATATTGCT
GTTCCAGCTATGTCGGAAAGTCAAGCCCAAAGTGCTGCCCAAGATGTCGCCCATCAATTA
GAATGTCTAGGTTTGGCTTGTAGTGCACCCAAAACTAGCATCAGGTCCTTTGATGAAGGT
GTTCATTTCTTAGGGGTAACTTTGAGAGCTAGACCTTCTTCGACAGCACCACCCCCAGCT
CGATCAAGGCCTCAAAGAATTTCTATGGTCGTTTCTGAGGGTGGTGCTGTAGTTAGAACA
AGACGTGGTCGAGTTAGAGTAGACCGAGATGGAGAAACTGTTGCCTCAATGTCATTGTC
CAGAGTAGCACGTATTGTTGTTCAGGGTCGTGTAGGTTTGACGACTCCACTTCTTCATGA
AGCTGCTCAGAGAGGTATTGATGTTGTAATGCTTAGTAGATCGGGTGGGTATGTTGGTAG
GCTATCAAGAAGGCGTCCAGGTGATCCTTCTTTGAGGAGAGCACAAGCCAGAGCATACG
ATTCTGGAGCTGATTTGGAGAGGCTTACAACTGCTTTTGTTTCCGGTAAGATTACTAATAT
GAGAGTAGCCGTATTGCGACACCAAAGAGGCGCCGGAACTTCCGAAGAAGGAGCCAGA
GTCGCTGCACAACTTGCTGAAGCCCGAGCAAGGGCATCTGTTATGAGGTCTGTACCTTC
TTTAATGGGAGTAGAAGGGGCCGCAACAAGAGCCTATTTTGGTTGGCTAGGCTCGCGAG
TTGGCGAAGAATGGGGATTTCATGGACGTGCTAGAAGGCCACCCCCTGACCCTGTTAAT
AGTATGTTATCCTATGGATATGTATTATTGTGCGCTGAAGGAGTTTCTGCTTGTGAACAAG
CTGGTCTTGATCCTGATATGGGCTTTCTACATTCTGATCGATGGGGTCGTCCGAGTTTGG
CACTAGATTTGATGGAAGAATGGCGTCCTGTCATTGTTGATTCGACTGTGTTGAGGATTA
TTTCGAATAAAAGACTAAAGCCATCAGACTTTACATTTGATGCCAAACAAGGAGCAAGGAT
GACCGCTCATGCTAGACAAACTTTTCTGAGAGAATATGAAGCAAGAATGTTGACGCTTGC
TGGATCAGATGCCGGGGCTGGTCGTCAACCATATAGAAGATTGATTGCTACTCAAGCTAT
GAGACTTGCAGAGGCTTTGAGAACACCCGGTGGTCCATATCGTCCTTATGTTTGGAGATA
G (SEQ ID NO: 123)
Cellulomonas bogoriensis 69B4, Cas2, DNA:
ATGACCAGGGTGTCTATCCCTAAACCCGATGGCGGAATACGTAGCTTGGCTATA
GGAGCTATTGAAGATAGAATTGTCGAAAGAGCTGTTCTGGATGTTCTTGATCCAGTTGTG
GACCCCACACTTTCCCCGTGGAGTTTTGCATACAGGAGAGGCTTAGGCGTGAGGGATGC
AGTTCGAGCTTTGGCAGAAGCTAGGGAATCTGGTCTAGCTTTTGTTGTTCGTTGTGATAT
TGATGATTGTTTTGATTCGATCCCGAGATGGCCTCTACTTAGGAGACTAAGAGAATTGGT
TTCTGACGCTGAATTGGTTGCCTTGGTAGAAAGATTGGTTGGCAGACCAGTCACTGGAGA
AAGGGCTTCCGGTGGTAGGGGATTGCATCAGGGOGGATCGCTCAGTCCTTTGTTGGCCA
ACCTATATTTGGATACCTTTGATCGAGCATTGATGCGTCATGGACACCGTGTAGTTAGATA
TGGAGATGATATAGCTATATCTGTTCCCGATAGGCCAACTGGACTTAGAGTATTGGATTT
GGCTGATGCAGAAGCAGAAGCCCTGTCTTTGAGACTAAATACTGATGATAGACAAGTCAT
AGCTTTTGATGAAGGAGTCCCTTTCTGTGGTCAGGTTGTTACTGCATCAAGCGGGCCAAC
AGCTGATTTACAAGCTAAACCTCTGCAAGGTACAGTCTTTGTTACAACCCAAGGAGCATT
GCTAAGAGTAAAAGGAGAACGACTTCGTGTCGAAGATGGAGACAGGCTACTGGCTAATG
TAAATTTGAAGCGTGTGAGGCAAATAGTCTGTTTTGGTAGAGTAGGAGTGACTTCTACCT
TGTTACAAAGAATTGTAGAACGAGGAATAGAACTTGCTTGGTTATATGAGGATGGTAGAC
ATGCAGCTAGAGTAAGTGGGTTGGATGGTACTGATCCAGAGGTCCGTTTAGCCCAATATA
GAGCTGCTGATGATGCACGTCAGGCACTGAGAATAGCTAGGCAGTTAGTCGCTGGTAAA
GTAACAAATATGAGAGTTGGTCTTTTGCGAGCTGCTAGGGCTCAACAAGCTCCAGAGCTT
GCTGATCGTCAGGCACGATTGGCCACAGCTCGACAGTCAGCTTTGATTGCAGATTCTACT
GCTGAATTGATGGGTTATGAGGGTTCAGCTACTAGAGATTATTTTGCTGGTCTTTCACAAA
TTTTGGGTCCTGAGTGGGGCTTTACTACTCGTCAAAGAAGGCCTCCTCCTGACCCCGTG
AATGCCATGCTTAGTTTTGGATATACCTTGCTGACTAATGAGGCACTCACGGCCTGTCAG
CTGGCTGGACTTGATCCATATCTCGGTATGTTACATTCTCCTAGAAGAAATAGACCTTCTT
TGGCATTGGATTTAATCGAAGAACTCCGACCCGTTGTTGTTGATGCTACCGTTATTAGGC
TAGTACGAACAGGACAAGTTACTCCTAAGAACTTTACTCTGACAGATGATAGAGGATGTA
GATTGGATGATCATGGTAGACGTGCCTTTCTGGATGCTTATGAGCGTAGAATGCTAACAC
TTGTCCATCATCCTGTAGAACAAAGAAGAATACCGTGGAGACATGTTTTGTTAGCACAAG
CTAGAACACTTGCTGCTGTATTGTCATCGCGTCGTCCCGAATATAGACCCGTAGTATGGC
GTTAA (SEQ ID NO: 142)
Micromonospora rosaria, RT-Cas1, Amino Acid:
MTRVSIPKPDGGIRSLAIGAIEDRIVERAVLDVLDPVVDPTLSPWSFAYRRGLGVRDA
VRALAEARESGLAFVVRCDIDDCFDSIPRWPLLRRLRELVSDAELVALVERLVGRPVTGERAS
GGRGLHQGGSLSPLLANLYLDTFDRALMRHGHRVVRYGDDIAISVPDRPTGLRVLDLADAEA
EALSLRLNTDDRQVIAFDEGVPFCGQVVTASSGPTADLQAKPLQGTVFVTTQGALLRVKGER
LRVEDGDRLLANVNLKRVRQIVCFGRVGVTSTLLQRIVERGIELAWLYEDGRHAARVSGLDG
TDPEVRLAQYRAADDARQALRIARQLVAGKVTNMRVGLLRAARAQQAPELADRQARLATAR
QSALIADSTAELMGYEGSATRDYFAGLSQILGPEWGFTTRQRRPPPDPVNAMLSFGYTLLTN
EALTACQLAGLDPYLGMLHSPRRNRPSLALDLIEELRPVVVDATVIRLVRTGQVTPKNFTLTD
DRGCRLDDHGRRAFLDAYERRMLTLVHHPVEQRRIPWRHVLLAQARTLAAVLSSRRPEYRP
VVWR (SEQ ID NO: 012)
Micromonospora rosaria, Cas2, Amino Acid:
MSRVCYVVAYDIADDDRRADLAMFLSGYGPRVQLSVFEVELPDADTAVAFRERLRSL
IDPNDDQVRLYRLTPQALGQRIIYGRRTIEERVDFWIV (SEQ ID NO: 032)
Micromonospora rosaria, Array1, DNA:
CCGCCGCACTCACCACTTCCTTATGTAGTTGGACGAAACACCTGAACCCGGACT
CACCCAGCGATCCCCGCAGGAGCACAGATGCGCGCAAACCTCTTCTCGCAAGTCAAGCC
AGCATCTCCCAACGCCTGCGACCTGGACCACACCCGCCACAGCACCTCTTCGCCACGGT
CCGCGCGACGATCTCCATCCCAGCACGTCAGTCCCACCGATTTTCGGGTAGCATCGCCA
TCCACAGCCCAACCCAGCGGGCATAGAAACTTCACGAACGCACGGAAGGCGTCCGGCG
TGAAACCGAGGACCGGAATCGCCATCCACAGCCCAACCCAGCGGGCATAGAAACGCCA
TGCCGGGCTGCCCTGCATGATCTTCTGCCGGGTGGTGTATCGCCATCCACAGCCCAACC
CAGCGGGCATAGAAAC (SEQ ID NO: 083)
Micromonospora rosaria, Array2, DNA:
CCGATACAGGCATCGAAGCCCTCGACCATCGAACCTAAACCCACCGAAACCGGA
CTACCCAGAAGTGACCGCCAGAAGCCGAGGTGCGCGCCGCCCTGTTTACGCACGTCAA
AGCCCACATTCACCACCATCATGGCCTAGGTCACACCGCCCTCGAAGGAGAACTTCACG
AGGTGCGCGCTACGATCTCTGTTCCAGCAGCTCAAGACCCACCGTTTTCGGGTAGGATT
GCCATCCACAGCCCAACCCAGCGGGCATAGAAACATGGCTACGCCATAGTTGGCGTCGT
TGTTGAACGCAAAATTGCCATCCACAGCCCAACCCAGCGGGCATAGAAACAGGTCAGGA
CTGGACCGGCCTGGTTCTTGCTGTCATTGCCATCCACAGCCCAACCCAGCGGGCATAGA
AAC (SEQ ID NO: 084)
Micromonospora rosaria, Array1A, DNA:
GCTGAAAGGAGGAACTATATCCGGATACCAGGTAGCACAGATGCGCGCAAACCT
CTTCTCGCAAGTCAAGCCAGCATCTCCCAACGCCTGCGACCTGGACCACACCCGCCACA
GCACCTCTTCGCCACGGTCCGCGCGACGATCTCCATCCCAGCACGTCAGTCCCACCGAT
TTTCGGGTAGCATCGCCATCCACAGCCCAACCCAGCGGGCATAGAAACGTCCCGCGGC
CGCTATTCTTTTGATTTATAAGGGATTTTGCGATCCTCTGGAGTGAATACCACGACGATTT
CCGGCAGTTTCTCCACA (SEQ ID NO: 085)
Micromonospora rosaria, Array1 RC, DNA:
GCTGAAAGGAGGAACTATATCCGGATACCAGGTCAGAATTTGCGATCCCTCCAG
GGTTGTACCTCTTTCTGGACCTGGAGCCGGAGGATCGTTGCGATCCTTTCAGGGGTGAT
GAGCGACAAGCAGGTTGGGCGGTGGGTCGCGATGTTATCGAGGGCGAACTCCCTCCCA
CGCTTAGCAACAGTTTCTATGCCCGCTGGGTTGGGCTGTGGATGGCGATGTCCCGCGGC
CGCTATTCTTTTGATTTATAAGGGATTTTGCGATCCTCTGGAGTGAATACCACGACGATTT
CCGGCAGTTTCTCCACA (SEQ ID NO: 086)
Micromonospora rosaria, Array2 A, DNA:
GCTGAAAGGAGGAACTATATCCGGATACCAGGTGCCGAGGTGCGCGCCGCCCT
GTTTACGCACGTCAAAGCCCACATTCACCACCATCATGGCCTAGGTCACACCGCCCTCG
AAGGAGAACTTCACGAGGTGCGCGCTACGATCTCTGTTCCAGCAGCTCAAGACCCACCG
TTTTCGGGTAGGATTGCCATCCACAGCCCAACCCAGCGGGCATAGAAACGTCCCGCGGC
CGCTATTCTTTTGATTTATAAGGGATTTTGCGATCCTCTGGAGTGAATACCACGACGATTT
CCGGCAGTTTCTCCACA (SEQ ID NO: 087)
Micromonospora rosaria, Array2 RC, DNA:
GCTGAAAGGAGGAACTATATCCGGATACCAGGTTCGAACGCTACGACTGCGCTG
CTTGGCTGCCCGTCGTCGTAAGCGTTCTCCCGTACCTCTTCCCATGCCCGCAGCAGTGC
CTCATCAGTCACCGCCCGCGCCAACAACGCGCCCAATAGCCACCCCTTCCCAAAATCGA
TCCCAACGGGTGTTTCTATGCCCGCTGGGTTGGGCTGTGGATGGCAATGTCCCGCGGC
CGCTATTCTTTTGATTTATAAGGGATTTTGCGATCCTCTGGAGTGAATACCACGACGATTT
CCGGCAGTTTCTCCACA (SEQ ID NO: 088)
Micromonospora rosaria, Array 3A , DNA:
GCTGAAAGGAGGAACTATATCCGGATACCAGGTGCCACCCCCGGTGCTTGTTGG
CCAGCTTGTCCCCGGGGCGATGAGTGACTGGTCGGCGAGGGGGGCAGCCCAGTGTC
GTACGAGTTTCGATCCCAGGGATTGTGAGCGACCCGAGCGCGGCACGTGGGCCGCATA
CGTGACGGCGACTCGTTGCGATCCCTCCAGGGGTGATGAGCGACGTCCCGCGGCCGCT
ATTCTTTTGATTTATAAGGGATTTTGCGATCCTCTGGAGTGAATACCACGACGATTTCCGG
CAGTTTCTCCACA (SEQ ID NO: 089)
Micromonospora rosaria, RT-Cas1, DNA:
ATGACCAGGGTGTCTATCCCTAAACCCGATGGCGGAATACGTAGCTTGGCTATA
GGAGCTATTGAAGATAGAATTGTCGAAAGAGCTGTTCTGGATGTTCTTGATCCAGTTGTG
GACCCCACACTTTCCCCGTGGAGTTTTGCATACAGGAGAGGCTTAGGCGTGAGGGATGC
AGTTCGAGCTTTGGCAGAAGCTAGGGAATCTGGTCTAGCTTTTGTTGTTCGTTGTGATAT
TGATGATTGTTTTGATTCGATCCCGAGATGGCCTCTACTTAGGAGACTAAGAGAATTGGT
TTCTGACGCTGAATTGGTTGCCTTGGTAGAAAGATTGGTTGGCAGACCAGTCACTGGAGA
AAGGGCTTCCGGTGGTAGGGGATTGCATCAGGGCGGATCGCTCAGTCCTTTGTTGGCCA
ACCTATATTTGGATACCTTTGATCGAGCATTGATGCGTCATGGACACCGTGTAGTTAGATA
TGGAGATGATATAGCTATATCTGTTCCCGATAGGCCAACTGGACTTAGAGTATTGGATTT
GGCTGATGCAGAAGCAGAAGCCCTGTCTTTGAGACTAAATACTGATGATAGACAAGTCAT
AGCTTTTGATGAAGGAGTCCCTTTCTGTGGTCAGGTTGTTACTGCATCAAGGGGCCAAC
AGCTGATTTACAAGCTAAACCTCTGCAAGGTACAGTCTTTGTTACAACCCAAGGAGCATT
GCTAAGAGTAAAAGGAGAACGACTTCGTGTCGAAGATGGAGACAGGCTACTGGCTAATG
TAAATTTGAAGCGTGTGAGGCAAATAGTCTGTTTTGGTAGAGTAGGAGTGACTTCTACCT
TGTTACAAAGAATTGTAGAACGAGGAATAGAACTTGCTTGGTTATATGAGGATGGTAGAC
ATGCAGCTAGAGTAAGTGGGTTGGATGGTACTGATCCAGAGGTCCGTTTAGCCCAATATA
GAGCTGCTGATGATGCACGTCAGGCACTGAGAATAGCTAGGCAGTTAGTCGCTGGTAAA
GTAACAAATATGAGAGTTGGTCTTTTGCGAGCTGCTAGGGCTCAACAAGCTCCAGAGCTT
GCTGATCGTCAGGCACGATTGGCCACAGCTCGACAGTCAGCTTTGATTGCAGATTCTACT
GCTGAATTGATGGGTTATGAGGGTTCAGCTACTAGAGATTATTTTGCTGGTCTTTCACAAA
TTTTGGGTCCTGAGTGGGGCTTTACTACTCGTCAAAGAAGGCCTCCTCCTGACCCCGTG
AATGCCATGCTTAGTTTTGGATATACCTTGCTGACTAATGAGGCACTCACGGCCTGTCAG
CTGGCTGGACTTGATCCATATCTCGGTATGTTACATTCTCCTAGAAGAAATAGACCTTCTT
TGGCATTGGATTTAATCGAAGAACTCCGACCCGTTGTTGTTGATGCTACCGTTATTAGGC
TAGTACGAACAGGACAAGTTACTCCTAAGAACTTTACTCTGACAGATGATAGAGGATGTA
GATTGGATGATCATGGTAGACGTGCCTTTCTGGATGCTTATGAGCGTAGAATGCTAACAC
TTGTCCATCATCCTGTAGAACAAAGAAGAATACCGTGGAGACATGTTTTGTTAGCACAAG
CTAGAACACTTGCTGCTGTATTGTCATCGCGTCGTCCCGAATATAGACCCGTAGTATGGC
GTTAA (SEQ ID NO: 124)
Micromonospora rosaria, Cas2, DNA:
ATGTCCCGTGTTTGTTATGTAGTTGCCTATGATATAGCCGATGACGACCGAAGGG
CCGATCTCGCTATGTTTCTTTCAGGGTACGGTCCGCGTGTTCAGTTGTCTGTGTTTGAAG
TCGAATTGCCGGATGCTGATACAGCTGTTGCTTTTAGGGAGCGTTTGCGTAGTTTAATAG
ACCCTAACGACGATCAAGTTCGTCTGTATCGACTCACTCCACAGGCATTGGGACAGAGAA
TTATTTACGGAAGAAGGACCATTGAGGAGAGAGTTGACTTTTGGATCGTGTAA (SEQ ID
NO: 143)
Tolypothrix campylonemoides, RT-Cas1, Amino Acid:
MFTIEQITSAWLLVRAGSRGAGVDGMTVDLFAAGVNEQLRILLRQLQQESYRASPAK
GFFVAKKSGGKRLIGIPTVRDRIVQRLLLEELYFPLEDTFLDCSYAYRPGRNIQQAVQHLYSYY
QYQPKWIIKADIAEFFDNLCWALLFTALEDLQLEPILLQLLEQQLKSGIVIAGKPIYPGKGVLQG
GVLSGALANLYLTSFERKCLSYGINLVRYGDDFAIACSSWLEANRILDKITTWLGELYLNLQPE
KTQIFAPDDEFTFLGYRFAGGEVYAPPPPVMTREGEWVTNESGLPYFRPKSRPVKFVSRPP
KACSIASPIKFPTAPISHLWQEFMTTLYVTDQGAYLSVKNQQFQVFYQGELKIKVPATRVNNIV
MFGCCNVSHGAVSMALRRRIPIMYLSQKGRYFGHTAVQGDARVEYLMQQVKCCENTQFTR
QQAEAIVAAKLHNSRILLMRLNRRRPTEIATQAIDLIEILIDSLPQAESLDALRGYEGKAATVYFQ
ALGSLFTGFFAFDKRTKRPPTDPINSLMSLGYTLLSQQVFSFIQSVGLHTHFGNLHTPRDNHP
ALVSDLMEEFRAQIVDSFVAYLVNKKILTPEDFTPPDERGGVYLQASALKKYLKHWEEKLQTE
TTHPHTGYKVAYRRCIELQVREYIACLVGEVEVYRPMVWKL (SEQ ID NO: 013)
Tolypothrix campylonemoides, Cas2, Amino Acid:
MFYLVCYDIVSDNRRNKVSKLLEAYGLRVQKSVFECVLDEKQYEMLSKYLMRLVNRR
EDQVRFYPMSAHNRCKVAVLGTQPEFVVDDAAFIV (SEQ ID NO: 033)
Tolypothrix campylonemoides, Array, DNA:
CCTACTATACCATTTGTGGGATAGATACAATGCCTGAAACCCTGATGATTTCGTTC
TATCCCACAAATCAGCTTCCTCACAAAGGTTTGAGGTACTTTGGCTGGATAGTTGTTGAG
AGTAATTCGCATTTATTTGGACGAAAATTTGCATCCCACAAAAAAGCGCTGTAGAATTATT
TCAGGGTAAGGCTTCTAGGAGCTGGTCTTTAAACTTCTTCGGAAGTTGAATTAATGGAAA
CCCGAACCTCCATCCCATATCGCATCCTTACGAAAGTCTTTAAACTTCTTCGGAAGTTGAA
TTAATGGAAACTTTGTGAGCCATTCCTGAGTCGTCACCATCATCCACTTTAAACTTCTTCG
GAAGTTGAATTAATGGAAAC(SEQ ID NO: 090)
Tolypothrix campylonemoides, Array1, DNA:
GCTGAAAGGAGGAACTATATCCGGATACCAGGTTTCTATCCCACAAATCAGCTTC
CTCACAAAGGTTTGAGGTACTTTGGCTGGATAGTTGTTGAGAGTAATTCGCATTTATTTGG
ACGAAAATTTGCATCCCACAAAAAAGCGCTGTAGAATTATTTCAGGGTAAGGCTTCTAGG
AGCTGGTCTTTAAACTTCTTCGGAAGTTGAATTAATGGAAACGTCCCGCGGCCGCTATTC
TTTTGATTTATAAGGGATTTTGCGATCCTCTGGAGTGAATACCACGACGATTTCCGGCAG
TTTCTCCACA (SEQ ID NO: 091)
Tolypothrix campylonemoides, Array1 RC, DNA:
GCTGAAAGGAGGAACTATATCCGGATACCAGGTGACAGTCCATTTTAGGCGATAA
CAATTTTTACATTAACAGAATGAGTGAAAAGTAGAAAATAAAATTTCAGGATATATTTAGCT
GGTTTTGAAAAAGAGGTGATGACTTTTCCACTCAACTCACCTCTTTTTACTTGCCTGTTAA
CAGCCGTTTCCATTAATTCAACTTCCGAAGAAGTTTAAAGGTCCCGCGGCCGCTATTCTT
TTGATTTATAAGGGATTTTGCGATCCTCTGGAGTGAATACCACGACGATTTCCGGCAGTT
TCTCCACA (SEQ ID NO: 092)
Tolypothrix campylonemoides, Array2, DNA:
GCTGAAAGGAGGAACTATATCCGGATACCAGGTGTTGACTTCCGCGAACGTCCT
ACAGATAAGGGTTTGAGGCTTATTGATATCTTTTTATTGAGAAATGATTGCAGTTATTCTG
ACACAATTTGACGTTCCGCGCAAAATGGTTGTAGACTTGGCTCAAGGGAAAGCTTTGAGA
ACCCTGCTCTGTTAAACTTCTCCCGAAGTTGAATTAATGGAAACGTCCCGCGGCCGCTAT
TCTTTTGATTTATAAGGGATTTTGCGATCCTCTGGAGTGAATACCACGACGATTTCCGGCA
GTTTCTCCACA (SEQ ID NO: 093)
Tolypothrix campylonemoides, Array2 RC, DNA:
GCTGAAAGGAGGAACTATATCCGGATACCAGGTACCAAATTCTCAACAAGGTAAT
TTGAGTTCTGAAAAAGAGGGTTTAACACTAACCCTCTTTCTTTTTACTTTTTACTTCTGCTA
AATGACTCTTTTCGGAATCTGAGTTAACCCTTTTACTTTTTACTTTTTACTTGGCTCCTGTT
TATCGTTTCCATTAATTCAACTTCGGGAGAAGTTTAACAGGTCCCGCGGCCGCTATTCTTT
TGATTTATAAGGGATTTTGCGATCCTCTGGAGTGAATACCACGACGATTTCCGGCAGTTT
CTCCACA (SEQ ID NO: 094)
Tolypothrix campylonemoides, Array3, DNA:
GCTGAAAGGAGGAACTATATCCGGATACCAGGTTTCTATCCCACAAATTAGCTTC
CTCACAAAGGTTTGAGGTACTTTGGCTGGAGATTTGTTGAGAGTAATTCGCATTTGTTTG
GACGAAAATTTGCATCCCACAAAAAAGCGCTGTAGAATTATTTCAGGGTAAGGCTTCTAG
GAGCTGGTCTTTAAACTTCTTCGGAAGTTGAATTAATGGAAACGTCCCGCGGCCGCTATT
CTTTTGATTTATAAGGGATTTTGCGATCCTCTGGAGTGAATACCACGACGATTTCCGGCA
GTTTCTCCACA (SEQ ID NO: 095)
Tolypothrix campylonemoides, Array3 RC, DNA:
GCTGAAAGGAGGAACTATATCCGGATACCAGGTTTGGAATCATTAAGTTTTCCAA
ATAAAACACAAAATAAATTTGCTAATTAAATAAAAAATTAAAAGATACATTATCAATTTGGTA
GTTTCCATTTTTAAAGAGAGGGTTTAACACTAACCCTCTTACTTTTTACTTGGCTCCTGTTT
ATCGTTTCCATTAATTCAACTTCCGAAGAAGTTTAAAGGTCCCGCGGCCGCTATTCTTTTG
ATTTATAAGGGATTTTGCGATCCTCTGGAGTGAATACCACGACGATTTCCGGCAGTTTCT
CCACA (SEQ ID NO: 096)
Tolypothrix campylonemoides, RT-Cas1, DNA:
ATGTTTACTATAGAGCAGATTACTTCGGCTTGGTTACTTGTGAGAGCCGGCTCAA
GAGGAGCCGGGGTCGATGGTATGACTGTCGATCTATTCGCAGCAGGAGTTAACGAGCAA
CTTAGAATATTACTGAGGCAGTTGCAGCAGGAGAGTTATAGAGCCTCTCCTGCTAAGGGA
TTCTTCGTCGCAAAGAAGTCGGGTGGAAAGAGGCTGATCGGAATCCCAACTGTTCGAGA
TAGGATAGTTCAGAGATTACTTTTGGAAGAATTGTACTTCCCCCTAGAGGACACCTTCCTA
GACTGTAGTTATGCTTACAGACCGGGTAGAAACATCCAACAGGCCGTGCAGCACCTGTA
CAGCTATTATCAATACCAGCCCAAGTGGATCATTAAGGCTGATATCGCTGAGTTCTTCGA
TAATCTATGCTGGGCTCTTTTATTTACAGCCCTGGAAGATCTCCAACTCGAACCTATACTT
CTACAGCTGTTAGAGCAACAGTTAAAGTCCGGCATAGTTATTGCAGGCAAGCCAATCTAT
CCAGGGAAAGGTGTATTGCAGGGGGGGGTCTTGAGTGGAGCACTGGCTAACTTGTATCT
TACGTCATTCGAGCGAAAGTGCCTATCCTACGGTATCAACCTTGTTAGATATGGTGACGA
CTTCGCTATAGCTTGCTCGTCCTGGCTTGAGGCTAACAGGATATTGGATAAGATCACTAC
ATGGTTAGGCGAGCTGTACCTCAACCTACAACCTGAGAAAACCCAGATCTTCGCTCCGG
ACGACGAATTTACTTTTCTAGGTTATAGATTCGCTGGCGGTGAGGTGTATGCCCCGCCCC
CACCTGTAATGACGAGGGAAGGTGAGTGGGTCACCAATGAGTCCGGACTGCCATACTTC
AGGCCTAAATCTCGTCCTGTTAAGTTCGTGAGTAGACCACCCAAAGCCTGTTCTATTGCT
TCACCAATTAAGTTCCCAACGGCCCCAATTAGCCACTTATGGCAGGAGTTCATGACTACC
CTATATGTTACAGATCAGGGTGCATACCTTTCTGTTAAGAACCAACAATTCCAAGTTTTCT
ACCAGGGCGAGCTGAAAATCAAGGTGCCTGCTACTAGAGTCAATAACATTGTAATGTTCG
GATGCTGCAACGTCAGTCATGGCGCTGTCAGTATGGCCTTGAGGAGAAGAATACCTATTA
TGTACTTATCACAGAAGGGTAGATACTTCGGCCACACCGCTGTTCAGGGCGACGCACGA
GTGGAGTACCTCATGCAGCAAGTTAAATGTTGCGAAAATACACAATTCACCCGACAACAG
GCCGAGGCTATCGTGGCTGCTAAATTACACAACTCCAGAATCTTACTGATGAGACTCAAT
AGGAGAAGGCCGACTGAGATAGCAACACAGGCCATTGACTTGATAGAAATCCTTATTGAC
TCACTGCCACAGGCCGAGTCCCTCGACGCCCTAAGAGGTTACGAGGGAAAGGCCGCTA
CCGTCTACTTTCAGGCACTCGGCTCACTATTCACTGGCTTCTTCGCTTTCGACAAACGTA
CTAAAAGACCACCCACAGATCCCATAAATTCTCTGATGTCTCTGGGGTATACCCTTTTGTC
TCAACAAGTATTCAGTTTCATTCAATCAGTAGGCTTGCATACACATTTCGGTAACCTACAC
ACACCAAGGGACAACCACCCTGCCCTCGTTAGCGACCTGATGGAAGAATTTCGAGCCCA
GATCGTGGACTCTTTCGTAGCATATCTGGTTAACAAGAAGATCCTTACCCCTGAGGACTT
CACACCCCCAGATGAGAGAGGTGGAGTGTATCTCCAGGCTTCAGCTCTGAAGAAATACC
TGAAACACTGGGAAGAAAAGTTGCAGACAGAAACTACTCACCCACATACTGGTTACAAAG
TTGCCTACAGAAGATGTATTGAGCTACAAGTTAGGGAATACATCGCTTGCCTGGTCGGAG
AGGTCGAAGTCTATCGACCTATGGTCTGGAAGTTGTAG (SEQ ID NO: 125)
Tolypothrix campylonemoides, Cas2, DNA:
ATGTTCTACTTGGTATGCTATGACATTGTGTCAGATAATCGAAGAAACAAAGTATC
AAAGCTGTTGGAAGCATACGGACTTAGAGTCCAGAAGTCTGTGTTTGAATGCGTCCTAGA
CGAAAAGCAATATGAGATGTTGTCCAAGTATTTGATGCGTCTGGTTAACAGGCGAGAGGA
CCAAGTCAGGTTTTATCCAATGTCCGCCCACAACAGATGCAAGGTCGCTGTGCTGGGCA
CCCAGCCCGAGTTCGTTGTTGACGATGCTGCATTCATCGTGTGA (SEQ ID NO: 144)
Oxcillatoriales cyanobacterium, RT-Cas1, Amino Acid:
MEFTPEPLHAAWLQVRKGSRSAGIDNITVDLFAGVARYQLQVLLWQLQQENYFPRP
AKGFYLRKASGGQRLIGIPTVRDRIVQRFLLDELYWPLEDVFLDCSYAYRPGRGIQMAVKHLY
SYYQLGQAWVIKADIEKFFDNLCWPLLLTDLEKLQFEPILRQLIEQHLASGIVVKGQHFHPNQG
VLQGGILSGALANLYLNEFDRLCLSHGFNLVRFGDDFAIACADSIQANRCLEQISSWLGSFYL
KLQPEKTRIFAPDEEFTFLGYLFRNGEVFAPEKAQPTTQVIRASGTSFSRPATRKVPVFSGKP
LACSIDVKPIVLPRANSEHFWREPMSTLYVTNQGSYLSIYNQQFQVFYQRELEIKVPASRVSHI
ILFGCCNLSHGAVSCALHRRIPILYLSQRGRYFGRLQSDGRAKVEYLTRQIICSLNPEFVRLQA
EAIVRAKLHNSRILLMRLNRYRKSKNADEMSVLQAVEMLEILMDSLHKADSMDALRGYEGKA
ATVYFQALGSLFSGPFAFEIRTKRPPTDPINSLLSLGYTLLSQNVSSFVEAMGLHTHFGNLHV
PRDNHPALVSDLMEEFRAQVVDSLVAYLINSQIFIADDFTPPDERGGVFLQPHALKKFLKHWD
EKLLSEVTHPHTGYKVAYRRCLELQVREYVSALMGEVEVYRPMMWKI
(SEQ ID NO: 014)
Oxcillatoriales cyanobacterium, Cas2, Amino Acid:
MFYLICYDIVDDRRRVKVSKLLETCGCRVQKSVFECVLDEKRQEQLQKRLLKLLNKR
QDQIRFYPLSEHCRCKVTVLGVQPKFVIDDEAFIV (SEQ ID NO: 034)
Oxcillatoriales cyanobacterium, Array, DNA:
GGTATTGGTGGCTTTTGGCTTAATGATTGGTATTTGTTATCAATTGTCAGCCTGTT
GATAAGAGTTGGGGGGGGTAGGCGATCTCTGAAACCCGCATTCTGTCGTTCCCCCCCCC
AATTTGCTCTCTGGTTATGGGTTTGAGGGCTGTATTTGGCAGAATTTTTCCAGCTTATTGA
AAAAATTATGCCATTATTGACGAATAAACTTGACCCCCCCAAAAAAGTGCTGTAGAATGAG
CTTGGGGCAAGGCTCCTAGAGACTGGCCTTCTCGACTTCTCTGAAGTCAAATTAATGGAA
ACATGATCTTAATCCTCAAAAAGAAAGTGAACTGCTACTTCTCGACTTCTCTGAAGTCAAA
TTAATGGAAACTCTCCTGTGAAATTGAAAGTCGCCGTGGTACCGGTGCTTCTCGACTTCT
CTGAAGTCAAATTAATGGAAAC (SEQ ID NO: 097)
Oxcillatoriales cyanobacterium, Array1, DNA:
GCTGAAAGGAGGAACTATATCCGGATACCAGGTCAATTTGCTCTCTGGTTATGGG
TTTGAGGGCTGTATTTGGCAGAATTTTTCCAGCTTATTGAAAAAATTATGCCATTATTGAC
GAATAAACTTGACCCCCCCAAAAAAGTGCTGTAGAATGAGCTTGGGGCAAGGCTCCTAG
AGACTGGCCTTCTCGACTTCTCTGAAGTCAAATTAATGGAAACGTCCCGCGGCCGCTATT
CTTTTGATTTATAAGGGATTTTGCGATCCTCTGGAGTGAATACCACGACGATTTCCGGCA
GTTTCTCCACA (SEQ ID NO: 098)
Oxcillatoriales cyanobacterium, Array1 RC, DNA:
GCTGAAAGGAGGAACTATATCCGGATACCAGGTCTCTTGAATCGCAAATTCTGTG
GCTGTTACAGCGTGTAAGCGTTCTAAAATATCTCGCTGTAAATTCTGGACGATGATGATA
CTTTCATCAGGGAGCAAAGCCATGATTACTGTCGGTTTATTAGAGTGTTATTGCCGATAAT
ATCATTATTTACCATTAATTTGACTTCAGAGAAGTCGAGAAGGTCCCGCGGCCGCTATTCT
TTTGATTTATAAGGGATTTTGCGATCCTCTGGAGTGAATACCACGACGATTTCCGGCAGT
TTCTCCACA (SEQ ID NO: 099)
Oxcillatoriales cyanobacterium, Array2, DNA:
GCTGAAAGGAGGAACTATATCCGGATACCAGGTCGAAATCCTTTTTTGGTAAGGG
TTTCAAAGCCTTATTTTACCATAGATTTCTAACTTATTGAGAATATCCTGCTTTTATTGACA
AGAAAACTTGACCCTCACGAAAAAGTGCTGTAGAATGAGCTTGGGGCAAGGCTCCTAGA
GACTGGCCTTTCGACTTCTTAGGAAGTCAAATTAATGGAAACAGTCCCGCGGCCGCTATT
CTTTTGATTTATAAGGGATTTTGCGATCCTCTGGAGTGAATACCACGACGATTTCCGGCA
GTTTCTCCACA (SEQ ID NO: 100)
Oxcillatoriales cyanobacterium, Array2 RC, DNA:
GCTGAAAGGAGGAACTATATCCGGATACCAGGTGCGCTCTTAGAGTTGAGATTG
CATCGACGGGGCTAAACTTAAGTTTATTGAACAATTTTTCTCTTCAGGCTTTATCAACAAA
TAACGCGATCGCCTCATAAGATTTATTAACTAAAATCCCTAGCTTTTTTTAAATGGCTAGG
GATTTATTGTTTCCATTAATTTGACTTCCTAAGAAGTCGAAAGGTCCCGCGGCCGCTATTC
TTTTGATTTATAAGGGATTTTGCGATCCTCTGGAGTGAATACCACGACGATTTCCGGCAG
TTTCTCCACA (SEQ ID NO: 101)
Oxcillatoriales cyanobacterium, RT-Cas1, DNA:
ATGGAATTTACGCCAGAGCCTCTCCATGCCGCTTGGTTACAGGTTAGAAAGGGTA
GCCGTTCTGCCGGCATCGATAATATCACAGTGGACCTTTTCGCTGGAGTCGCAAGGTAT
CAACTGCAAGTACTTTTGTGGCAGCTTCAACAAGAGAACTACTTCCCAAGGCCGGCTAAG
GGGTTCTATTTGAGGAAGGCTAGTGGTGGACAACGACTTATCGGGATTCCTACTGTAAGA
GACAGAATCGTCCAACGTTTCTTACTAGATGAGTTATACTGGCCACTTGAAGATGTGTTC
CTTGACTGTTCCTACGCATATCGACCAGGGAGAGGCATTCAGATGGCCGTTAAGCACCT
CTACTCTTACTACCAGTTAGGACAGGCATGGGTGATTAAAGCAGACATCGAAAAGTTCTT
CGATAACTTGTGCTGGCCCTTGTTGCTCACCGACCTGGAGAAATTACAGTTCGAACCGAT
ACTGAGGCAACTGATAGAACAACACTTAGCTTCAGGCATCGTGGTAAAGGGACAACACTT
CCACCCAAACCAGGGTGTGCTTCAGGGTGGAATATTATCCGGGGCATTAGCAAACCTCT
ATCTTAATGAATTTGATCGACTGTGCCTGTCACATGGATTTAACCTTGTTAGGTTCGGTGA
CGATTTCGCTATTGCTTGCGCTGACTCAATACAGGCAAACAGATGCTTAGAACAGATCTC
ATCGTGGTTGGGCTCATTCTACTTGAAGTTACAACCAGAAAAGACTCGTATCTTCGCCCC
AGACGAGGAGTTCACGTTCTTGGGTTACTTGTTCAGGAACGGTGAGGTATTCGCCCCAG
AGAAGGCACAGCCAACAACCCAGGTAATTAGAGCCAGCGGCACTTCTTTCTCAAGGCCT
GCAACACGAAAGGTTCCAGTCTTTAGCGGCAAACCGTTAGCATGTAGTATCGACGTTAAA
CCTATCGTCTTACCAAGAGCAAACAGTGAGCACTTCTGGAGAGAGCCAATGTCTACACTG
TACGTGACAAACCAGGGATCATACCTTTCTATATATAACCAACAGTTTCAGGTATTCTACC
AAAGAGAGTTAGAAATAAAGGTCCCCGCTTCTAGGGTCAGTCATATCATCCTATTCGGTT
GTTGTAACTTGTCCCACGGTGCTGTGTCCTGCGCATTGCATCGTAGAATCCCTATCCTGT
ACCTCTCTCAAAGAGGAAGATACTTCGGTAGACTTCAGAGTGACGGGAGAGCCAAAGTC
GAGTACCTTACTAGACAAATAATATGCTCACTCAACCCAGAGTTCGTGCGTCTGCAAGCT
GAAGCCATCGTGCGTGCTAAATTGCATAACTCTAGGATACTCTTAATGCGTCTAAATCGTT
ATAGAAAATCTAAGAACGCTGACGAGATGTCTGTATTACAGGCTGTTGAGATGCTAGAGA
TCTTAATGGATTCGCTTCACAAAGCCGACTCAATGGACGCATTGCGTGGATACGAGGGTA
AAGCTGCTACCGTCTACTTCCAGGCCCTTGGCTCACTTTTCAGTGGTCCATTCGCATTCG
AGATCAGAACCAAAAGACCGCCGACGGACCCAATAAACTCACTCTTGTCTCTTGGTTACA
CGCTGCTCTCGCAAAACGTATCTTCCTTCGTTGAGGCTATGGGACTTCACACCCACTTCG
GTAACCTACACGTGCCTAGAGACAACCATCCTGCACTTGTTTCCGACCTTATGGAGGAGT
TCAGGGCACAAGTCGTAGACTCGCTAGTTGCTTACCTGATTAACTCACAAATCTTCATCG
CCGACGACTTCACCCCACCTGACGAAAGGGGTGGGGTGTTCTTGCAGCCCCACGCATTA
AAGAAATTCCTGAAACACTGGGACGAGAAATTGCTTTCTGAAGTCACACACCCGCACACA
GGATATAAAGTTGCTTACAGAAGATGCTTGGAGCTACAGGTCAGGGAATACGTATCTGCT
TTAATGGGTGAAGTAGAAGTCTACCGTCCAATGATGTGGAAAATTTAG (SEQ ID NO: 126)
Oxcillatoriales cyanobacterium, Cas2, DNA:
ATGTTCTACCTGATTTGTTACGATATAGTTGACGACCGTAGACGAGTCAAGGTAA
GTAAGCTCTTAGAGACATGCGGGTGTAGGGTCCAAAAGTCAGTCTTTGAATGCGTGTTAG
ACGAGAAGCGACAGGAGCAGCTGCAAAAGCGTCTTTTGAAGTTACTCAATAAGCGTCAG
GATCAGATTAGATTCTACCCCTTGAGCGAGCATTGCCGATGCAAAGTTACTGTGCTCGGT
GTCCAGCCTAAATTCGTCATCGACGACGAAGCCTTTATCGTGTAA (SEQ ID NO: 145)
Psychrobacter lutiphocae DSM 21542, RT-Cas1, Amino Acid:
MVTASHTLDWQSADCQLIWHSDTTHRKPENIYIRLWGLIQTINTTEDQYLEQLHRCR
DEHKKNIIIQHLEAKKQYFNHPNFPLIAFHPTQTSYHKVSLKKGEIIPISLVFGGKYSHLAQAWL
DWFAVRLTLENTGFSLANIPSLKPHQIQLPPLTANKLAKCQIANKQAKAPIDAQELETAPVQGS
PQEMTILIDTPANFSLNKTQQKQVSNLSTADILKYAFIHNSQKRLCQWFPQQNKVIESWFKQW
TPLLLKSPLSTYQLHERHEIKTVSKSSSARSQSKIQFHKGHAGWLTFKGAWLEVDELLTVLSSI
HLLGQRMHINGLGYFLTQQQLEHSAPTLWQRHLMDKHKIKHAIEEVLERHELEPVVDDKGRI
MQVDALTELIYTQLSTGVYAPKPTRAIFVPKPKGGKRCIEELEQVDMMVHRLVFNSIAKTIESY
QSPLSLGYRKGYSRQMARDKVQALIDSGFGWVVEADIESFFDNVPFERLWQRLATILPQREL
QTIALIKKLMQVGYTVSNASGTVVKEHLRFKGLMQGSPLSPVLANLYLAMLDEQINAEHFAFV
RYADDVLMFCRSEADANTTLAWLDQHLSELGLNLSLSKTAITAVNNGFEFLGYRFDKEGSDD
KSIVPVLRQRKPIMITGSRKYLGINGGALEVRQKQARKQPGRSLSTSPLSNQLVQVIPLRRISQ
LVVMGNHSLSSPLLTACAKHHISVHLVNQWGFQVGTFKPSHAEYYAVSAEQYQRHQQLKPS
ERMAIAADLVLAKINNYQTWIINSYRKGDAQVNKQLDQIANQASSATTIEALMGYEGQAAKICF
QRLQSVMIGDQQAAFSSKRRSRGGPDRLNSMLNFGYYWLFTRISGLLHSHGLNPYLSFLHE
PEQNYETLVYDIMELFRVQVDKTVLRLINRKQIQADSFHLDAKKGWKLNNDALHLLSNQLQST
FASKINQTFLEDIILIQVRTLLHWATQQQSLVWFYWYADKDNVQFSLPDESPQTLIIDNIESQ
(SEQ ID NO: 015)
Psychrobacter lutiphocae DSM 21542, Cas2, Amino Acid:
MSNVTKQQAPLYVFVYDISNDKERSKVDGVLRDYGFRVQKSVYECHLTRGDKKRLL
DKLESLTIETGHIRCYGVNSKVIKIGAPPEDLDEEYIYFIE (SEQ ID NO: 035)
Psychrobacter lutiphocae DSM 21542, Array (DR), DNA:
CGGCGTATCTTGTGACCTATTTTGTATAAACTATTTTTTAATCAACAATCAAAAAAG
TGAAAAATCCATTGGCGACCTATGATATACTTAACCTCGAACAGCCTTTTGAAATTCGACA
TCGCTAATTTTGTCCTCTGGACGCTATATGTGGTGTGGCTTTGAAAATGGCAGGGTTCTG
CACTATGCAGAGGTACGTACCGATTAAGAC (SEQ ID NO: 102)
Psychrobacter lutiphocae DSM 21542, RT-Cas1, DNA:
ATGGTAACAGCTTCACACACTCTAGATTGGCAATCGGCGGACTGTCAGTTGATCT
GGCATAGCGATACTACGCATCGTAAACCGGAGAACATTTATATTCGTTTATGGGGTCTGA
TCCAAACCATCAACACCACCGAAGATCAGTATCTGGAACAACTCCATCGTTGCCGTGATG
AGCACAAAAAAAACATCATTATTCAGCATCTGGAGGCTAAAAAGCAATACTTCAACCACCC
GAATTTCCCGCTGATCGCTTTCCATCCGACCCAAACGTCCTATCATAAGGTGAGCCTGAA
AAAGGGTGAGATCATCCCGATCTCACTGGTATTTGGTGGCAAATATTCCCACTTGGCTCA
GGCGTGGCTGGACTGGTTCGCAGTGCGGCTGACTCTGGAGAACACCGGTTTTAGCTTG
GCGAATATCCCGTCTCTGAAGCCGCATCAGATCCAGCTGCCTCCGCTGACTGCGAACAA
GCTCGCGAAGTGCCAGATCGCGAATAAACAAGCGAAGGCGCCAATTGACGCCCAAGAGT
TGGAAACCGCTCCAGTTCAAGGTAGCCCTCAAGAAATGACCATTCTGATCGACACCCCG
GCAAATTTCAGCCTCAACAAGACCCAACAAAAACAGGTGAGCAATCTGAGCACTGOGGAT
ATTCTTAAGTACGCCTTCATCCACAATAGTCAGAAAAGATTATGCCAGTGGTTCCCGCAA
CAAAACAAAGTGATTGAGAGCTGGTTCAAACAGTGGACCCCGTTGCTGTTGAAGAGCCC
GCTGTCTACGTACCAGCTGCACGAGCGCCACGAGATCAAAACCGTGAGCAAAAGCAGCT
CCGCGCGTAGCCAGAGCAAGATCCAGTTCCACAAAGGTCATGCGGGTTGGCTGACCTTC
AAGGGCGCATGGCTGGAGGTTGATGAGCTGCTGACTGTCTTGAGCAGCATTCATCTGTT
AGGCCAGCGTATGCATATCAACGGTCTGGGCTACTTCCTGACCCAGCAGCAACTCGAAC
ACTCCGCGCCGACCCTCTGGCAACGTCATCTGATGGATAAACACAAAATTAAACACGCCA
TTGAGGAAGTTCTGGAGCGCCACGAGTTGGAGCCGGTGGTTGATGATAAGGGTCGTATT
ATGCAGGTAGACGCCCTGACCGAGCTGATATACACCCAGCTGTCCACCGGTGTTTACGC
GCCGAAACCAACGCGTGCAATATTTGTGCCGAAACCGAAGGGCGGCAAACGTTGCATCG
AAGAACTGGAACAGGTCGACATGATGGTTCATCGTTTAGTCTTTAATAGTATCGCGAAAA
CCATCGAGTCCTATCAGAGCCCGCTTTCCCTCGGCTACCGCAAGGGCTACAGCCGCCAG
ATGGCGAGAGACAAGGTGCAAGCGCTGATCGACTCTGGTTTCGGTTGGGTTGTGGAAGC
TGATATTGAATCGTTCTTTGATAATGTGCCGTTTGAGCGCCTGTGGCAGCGCCTGGCTAC
CATACTCCCGCAACGTGAATTGCAAACGATCGCGTTGATCAAGAAACTTATGCAAGTGGG
CTACACCGTTTCTAATGCATCTGGCACCGTTGTCAAAGAACACCTGCGTTTTAAAGGTCT
GATGCAGGGCAGCCCGTTGTCGCCGGTGCTTGCTAATCTGTATCTGGCAATGCTGGACG
AGCAGATAAACGCAGAGCACTTTGCATTCGTGCGTTATGCCGACGACGTACTGATGTTTT
GTAGAAGCGAAGCAGATGCTAACACCACCTTGGCGTGGCTGGACCAACATCTGAGCGAA
CTGGGCCTGAATCTCTCTCTGTCAAAGACGGCCATTACAGCAGTCAACAACGGTTTCGAG
TTTCTGGGTTACCGCTTCGACAAAGAAGGCTCGGACGACAAGTCCATTGTGCCGGTTCT
GCGCCAGCGTAAGCCGATTATGATCACCGGTAGCCGTAAGTATCTTGGTATTAACGGCG
GCGCCTTGGAGGTTCGCCAGAAACAAGCGCGCAAGCAGCCGGGTCGTAGCCTGTCCAC
CAGCCCGTTGTCCAACCAATTGGTTCAGGTCATCCCGCTGCGTCGCATCAGCCAGCTGG
TGGTTATGGGGAACCACTCCCTGAGCAGCCCATTACTGACGGCCTGCGCGAAGCACCAT
ATTTCTGTGCACTTAGTTAACCAGTGGGGTTTTCAGGTGGGTACATTCAAACCGAGCCAT
GCGGAGTATTACGCGGTTAGCGCCGAGCAGTACCAGCGCCACCAGCAGTTGAAGCCGT
CTGAACGCATGGCCATCGCCGCTGACCTGGTGTTGGCGAAGATCAACAATTACCAGACC
TGGATTATTAACAGCTACCGTAAGGGCGACGCGCAGGTCAATAAACAATTAGACCAAATC
GCGAACCAAGCGAGTAGCGCCACCACCATCGAAGCACTAATGGGTTACGAGGGTCAGG
CGGCAAAGATTTGTTTTCAACGTCTCCAGTCTGTGATGATTGGTGATCAGCAAGCGGCGT
TTAGCAGCAAGCGTCGTAGTCGTGGTGGCCCTGATCGTCTGAACAGCATGCTGAACTTC
GGCTACTACTGGCTTTTCACCCGTATCTCTGGTTTGCTGCACTCACACGGCCTGAACCCG
TATCTGTCCTTTCTGCACGAACCGGAACAGAACTATGAAACCTTGGTGTATGATATTATGG
AATTATTTCGTGTTCAGGTTGATAAAACGGTGTTGCGTCTGATTAATCGTAAACAAATCCA
GGCTGACTCGTTCCACCTTGATGCAAAAAAGGGCTGGAAACTGAACAATGACGCGCTGC
ACTTGCTGAGCAACCAGTTGCAGAGCACCTTCGCTAGCAAAATTAACCAAACCTTTCTGG
AAGATATCATCCTGATCCAAGTGCGCACGCTGCTGCACTGGGCAACCCAGCAACAATCTT
TGGTTTGGTTCTATTGGTATGCTGATAAGGACAACGTGCAATTTAGCCTGCCGGACGAAA
GCCCACAGACTCTGATCATTGACAACATCGAGAGCCAGTAA (SEQ ID NO: 127)
Psychrobacter lutiphocae DSM 21542, Cas2, DNA:
ATGTCAAATGTAACAAAACAGCAAGCTCCCTTATATGTTTTTGTTTACGACATCAG
CAATGATAAAGAGCGCTCCAAAGTGGACGGCGTGCTGCGTGATTACGGCTTCCGTGTTC
AGAAATCTGTTTACGAGTGCCACTTGACGCGTGGTGATAAGAAGCGCCTGTTGGACAAG
CTGGAAAGCCTGACCATTGAAACCGGTCATATTCGTTGTTATGGTGTCAACAGCAAGGTG
ATTAAAATCGGCGCGCCGCCGGAAGATCTGGACGAGGAATATATCTACTTCATCGAGTAA
(SEQ ID NO: 146)
Ruminococcus sp. TF08-4, RT-Cas1, Amino Acid:
MFSMEEMLSKKNQREAFSFLKNKKDGIGADGMPLSELEDYWKINSERICSELKKGKY
TPGIIKCTEIINNRGKRRVISNLCTVDRFITRLLYQKLRRYISPEFLENSYAYQENKGILNAVQKV
QKYISEGSKYTMEIDLKDYFDTIPHEQMMSLIKERITDDRVVDLIYKYMHCSVSQDNEIKEKKK
GLVQGNAISTVLSNLYLHSLDQYLQEREYKWVRFADNINVYCRKENDAIRVYNEINIYIKEKLH
LTINEKKSGIYPVLDRIFLGYKFYKYNGKYEAKKYKYQKTDCFHNWHESALQRVNHEYHIVQN
GVLNKKDYSLIFENEEEKCDIPVGVVDQINIYSEVTIAATALQLISKKKIRLSIIDKYGNLIGNYIPA
GYGRSSIEFLKQAVFYESSHRFEIAQKIEVASIHNLRANVKYYSRKNAHALDAVVEQLSEYMN
EMRNAPNIEELMIIEAQARQQYYASFNIILKQESFFFEKRTRRPPKDAINALISFGNTLLYNFVL
QAIWKTSLDPRIGIIHATTNRNYSLNLDFADLFKPVIVDRVIFSLVNLLQLQSEVHFEEKESGAIY
LNKEGKRIFIKAFEEKLADKIKLGEKTYTYKQIIEEEIRKFQRYIVKEEKYKPYKYW (SEQ ID
NO: 016)
Ruminococcus sp. TF08-4, Cas2, Amino Acid:
MFVIVVYDINRKRVGRALKICRKYLVHVQKSVFEGNITEGQLKQLKNELFQLIKIEEDA
VCIYRFESPKYARKEELGTIERKSHII (SEQ ID NO: 036)
Ruminococcus sp. TF08-4, Array (DR), DNA:
TGTCTACCTCGAAAGCCTTGAAAATACTGGGGAAAATGGCGATTGAAGTGGCTGA
AAACAAAGGGAAAAGCTATAAAAATCTCAAAAAAATGAAAAAAAGTCTGTCTACCGAGAC
GAAAAAATAAACTTTTGAAAAAAGCTTGATTTTTCAAGGATATTTGCGATAATGAAAAGTGA
GGTCGACAGACTTTTTAATGTGATTAGAGGGAGAAAAAAGAATACAGAGTCGGAAAACGG
CGAAAATACTGTTGTAATACCGTACCTATAAGGAATTGAAAC (SEQ ID NO: 103)
Ruminococcus sp. TF08-4, RT-Cas1, DNA:
ATGTTTAGTATGGAAGAGATGCTATCAAAAAAGAACCAGCGTGAAGCGTTTAGCT
TCTTGAAAAACAAGAAGGACGGCATTGGTGCGGATGGCATGCCGCTGAGCGAGTTGGAA
GATTATTGGAAAATCAACTCCGAGCGTATCTGCTCTGAACTCAAAAAGGGCAAGTACACC
CCGGGTATTATCAAATGCACCGAAATTATTAACAACCGTGGTAAGCGCCGTGTCATTAGC
AACCTGTGTACCGTGGACCGCTTCATTACCCGTCTGCTGTACCAGAAACTGCGCCGTTAT
ATCTCTCCGGAGTTCCTGGAAAACTCGTATGCATATCAAGAGAATAAAGGCATCCTGAAC
GCGGTGCAGAAGGTGCAGAAATACATCTCTGAGGGCTCGAAATACACGATGGAAATTGA
TCTTAAGGACTATTTCGATACCATCCCGCATGAGCAGATGATGTCCTTAATCAAGGAGCG
CATCACCGATGATCGTGTAGTTGACCTGATCTATAAGTACATGCACTGCAGCGTTAGCCA
GGATAACGAGATCAAGGAGAAGAAAAAGGGCCTGGTTCAGGGTAACGCGATCTCGACCG
TTCTGTCTAATCTTTATCTGCACAGCCTGGATCAGTATCTGCAAGAGCGCGAATACAAGT
GGGTTCGTTTCGCTGACAACATCAACGTATACTGCCGTAAGGAGAATGACGCCATCCGT
GTGTATAACGAGATCAATATCTACATTAAAGAGAAATTGCACCTGACCATTAACGAAAAGA
AATCCGGTATCTACCCGGTCCTGGACCGGATTTTCTTAGGTTATAAATTCTATAAGTACAA
TGGCAAGTACGAAGCTAAAAAGTACAAATACCAGAAAACCGACTGTTTTCATAATTGGCA
CGAGAGCGCGCTGCAACGTGTGAATCATGAATATCATATTGTTCAAAACGGGGTGCTGAA
CAAAAAGGATTATAGCCTGATTTTCGAGAACGAAGAGGAGAAGTGCGATATTCCGGTTGG
TGTCGTTGACCAGATTAACATTTACTCTGAGGTCACCATCGCGGCTACCGCACTGCAACT
GATCAGCAAAAAAAAGATCCGTCTCTCCATCATCGATAAATACGGCAACCTCATTGGCAA
CTATATCCCGGCAGGTTATGGCCGCTCTAGCATTGAGTTTTTAAAGCAGGCTGTGTTTTA
CGAATCAAGCCATCGTTTCGAAATCGCCCAGAAAATTGAGGTTGCATCCATTCACAACCT
GCGGGCAAATGTTAAATACTATAGCCGTAAAAACGCGCACGCGTTGGACGCTGTGGTGG
AACAGCTGAGCGAGTATATGAATGAAATGCGTAATGCTCCGAATATTGAAGAACTGATGA
TTATCGAAGCACAAGCGCGTCAGCAGTACTACGCGTCTTTTAATATTATTCTGAAACAAGA
ATCGTTCTTCTTTGAGAAACGTACGCGTAGACCACCGAAAGACGCCATCAACGCGCTGAT
CAGCTTCGGTAATACCCTGCTGTACAATTTTGTTCTGCAAGCGATCTGGAAAACCAGCCT
GGACCCGCGTATCGGCATCATCCACGCGACTACGAACCGCAACTACAGCTTGAATTTGG
ACTTCGCCGATTTGTTTAAACCAGTGATCGTGGACCGCGTCATATTCAGTTTGGTGAACT
TGCTACAATTACAAAGCGAAGTTCACTTTGAAGAAAAGGAGTCCGGTGCGATTTACCTGA
ACAAAGAGGGTAAACGCATTTTTATTAAGGCTTTCGAAGAGAAGTTGGCGGACAAAATTA
AGCTGGGTGAAAAAACCTACACGTACAAGCAAATTATCGAGGAGGAAATCCGCAAGTTCC
AGCGTTATATCGTTAAGGAGGAGAAGTATAAGCCGTATAAGTACTGGTAA (SEQ ID NO:
128)
Ruminococcus sp. TF08-4, Cas2, DNA:
ATGTTTGTTATAGTAGTCTATGATATTAATAGAAAACGTGTTGGTCGTGCACTGAA
GATCTGTCGTAAGTACCTGGTGCACGTCCAAAAAAGCGTTTTTGAGGGCAATATTACCGA
AGGCCAGCTCAAGCAGTTGAAGAACGAATTGTTCCAACTGATTAAAATCGAGGAGGACG
CGGTGTGCATTTATCGCTTCGAGAGCCCGAAATACGCTCGCAAAGAAGAGCTGGGTACG
ATCGAACGTAAGAGCCATATTATCTAA (SEQ ID NO: 147)
Vibrio sinaloensis strain T08, RT-Cas1, Amino Acid:
MTNPILNSIEPIAQLLPLRAMVVTLRFTQDATFQFYHHVAAHAWVRHLCGSPDNFSQ
QVTVQTLENGKTDYKAGELYRFKLVFTPLGGNLIQPLIDALERLPSSAKALPRGACINNNVELV
ELKCGVGNEEIHHAIDLYPYDLQALAQDVAHWQQRDDIYMESLTPARLLKEKASQESSRGDA
RYCRDKVHFSATTITRRFVDTIIGLVESHTGQRYQRDIEASFEIQPELSFWVEHRYGRGEKWQ
KKPMSGALFSLKVRNVPRLEKWQIALLVLGQWLGIGQSRSMGLGLYWLRDVSETLGGHSNR
LIKEYFNQRRLVQLVQESLDSQFSEQERNQYKNKVIGLSHTILAGDYKAPVLTQVEIDKSDGG
VRTLSIPPLADRILQKAIARPLAVSLDGLWKTHSYGYRKDLSRHDAKFAINQAIQQGYEWVLES
DVDSFFDNVDWRNLQTRLKLLLPNDFLVDVIMAWVKAPVKTPSGQILERTQGLPQGSPLSPL
LANLVLDDFDADMLALDYKLIRYADDFVLLFKKQSEAQMALDHVIASLNEHGLNIKAKKTQIVH
ANKGFRYLGFWFVDGYAIETSRHYRQEEQEYQQAITHHQAQLAESKQREKQQIGEREQLGT
LLVIAGEVAMLTCENKRLKITQQDESRYYAWEELETILILGPHNISTPCIRQAMHHQVAIHFASR
YGQYQGVACSNMPSQGHQLWQLQIAYLQKVDVALTWSIELVCAKIDGHIHLIRNRERQSPLL
EKLRTIKRKARRSDDLQVLLGIEGEAAKLQWEFFKQHLDCEWQFSGRNRRPPKDPINAMLSL
GYTYLYHLTDSLIQSNGLCPWAGFYHQPHGAHRTLASDLMEPLRVVVDRTVLALVAKRQIKP
DDFLILEDGCEMSREARKVLLTQLLADLTTKRKKSDRVIDQMITQVKEVKIATKLALNPNFWRP
(SEQ ID NO: 017)
Vibrio sinaloensis strain T08, Cas2, Amino Acid:
MKVYLVCFDIENDKKRRRLSKLLLSYGERVQYSVFEITLKSDASLKTLTKQCKRHVEQ
GDSLRFYALPFNARQASFDIDGEPIARFPQAEIL (SEQ ID NO: 037)
Vibrio sinaloensis strain T08, Array, DNA:
TGTACTGCTCTTTTACAATTTGGACTTTAAGTACCGGAATAGTTTCGCTATTATTG
AGCACACTAATAAGCTGTAACAAGTGTCAGTTTGTGGATAAGTTTAGGCCAACCAAAAGT
GTATTTTATCTTGTTGAATCAAAAGGTTTAATCGCAGGTAGGCTTCATTAGTACTGGTCGT
TCAGACCGTTGAGACATTCCTGTTTATACAGGTGACTTTTGGGTTTCGGGCTTCATTAGTA
CTGGTCGTTCAGACCGTTGAGACAACATGGTTAGTTTTTTATGATCACCCAAGCATCGGC
TTCATTAGTACTGGTCGTTCAGACCGTTGAGAC (SEQ ID NO: 104)
Vibrio sinaloensis strain T08, RT-Cas1, DNA:
ATGACAAATCCTATCCTTAACTCAATCGAACCAATAGCTCAATTACTGCCACTCCG
AGCAATGGTTGTTACTCTAAGATTCACTCAGGATGCCACTTTCCAATTCTACCACCACGTG
GCTGCCCACGCATGGGTTAGACACTTGTGOGGATCACCCGATAACTTTTCTCAACAGGTA
ACCGTCCAGACACTAGAGAACGGAAAGACGGATTACAAGGCAGGAGAACTGTATAGATT
TAAGCTAGTTTTCACCCCTCTTGGTGGTAACCTAATACAACCATTGATCGATGCTCTGGAA
AGACTGCCGTCATCGGCAAAGGCCCTACCAAGGGGAGCTTGCATTAATAATAATGTAGA
GCTGGTAGAATTGAAGTGCGGTGTGGGGAACGAGGAAATCCACCACGCCATTGATCTGT
ATCCTTACGATCTGCAAGCATTGGCTCAGGACGTTGCTCATTGGCAGCAAAGAGATGATA
TCTATATGGAATCTCTGACTCCTGCTAGGTTGCTTAAAGAAAAGGCATCACAGGAATCCT
CAAGAGGTGACGCTAGATACTGCCGAGATAAGGTCCACTTCTCTGCAACAACTATAACCA
GAAGATTCGTGGACACAATAATTGGCCTTGTCGAGAGTCATACGGGACAGAGATATCAGA
GGGATATTGAGGCTTCCTTCGAAATTCAACCAGAACTATCCTTTTGGGTCGAACACCGAT
ACGGCCGAGGAGAGAAGTGGCAGAAGAAACCTATGTCAGGTGCCTTGTTCTCCTTGAAG
GTTCGTAATGTACCAAGATTGGAGAAGTGGCAGATTGCCTTGTTAGTTTTGGGTCAGTGG
TTGGGTATCGGTCAGAGCAGAAGTATGGGTTTGGGTCTTTACTGGCTTAGGGACGTAAG
TGAGACTCTAGGTGGGCACTCTAACAGACTCATTAAAGAGTACTTCAATCAGCGTAGATT
GGTCCAACTGGTGCAGGAATCATTAGATAGTCAGTTCTCAGAGCAGGAAAGAAACCAGTA
TAAGAACAAGGTGATCGGGCTTTCCCATACTATCCTGGCTGGTGATTACAAGGCACCCGT
GTTGACTCAGGTTGAGATCGATAAGAGCGACGGTGGTGTCAGGACTCTGAGCATCCCAC
CCCTGGCTGATCGTATTCTTCAGAAAGCCATCGCAAGACCTCTCGCTGTATCTCTGGACG
GTCTTTGGAAAACCCATTCCTACGGGTACAGGAAGGACCTATCAAGACATGACGCTAAGT
TTGCTATCAATCAGGCTATACAGCAAGGTTACGAGTGGGTTTTGGAGTCCGATGTCGATT
CATTCTTCGATAATGTTGACTGGAGAAATCTACAGACCAGGCTTAAACTTCTTTTGCCGAA
CGACTTCTTAGTTGACGTTATCATGGCCTGGGTGAAGGCCCCTGTGAAAACCCCTAGCG
GACAGATCCTGGAGCGTACTCAGGGCCTACCGCAGGGTTCACCTCTGTCCCCTTTACTC
GCTAACTTGGTTCTGGATGATTTCGATGCAGACATGCTGGCACTGGACTATAAGTTAATC
AGATACGCAGACGACTTCGTTCTATTATTCAAGAAGCAGAGTGAGGCTCAGATGGCTTTA
GATCACGTTATTGCCTCTTTAAATGAGCACGGATTAAACATTAAAGCTAAGAAAACTCAAA
TTGTTCACGCTAACAAAGGCTTCAGGTACTTGGGTTTCTGGTTCGTAGACGGATATGCTA
TAGAAACCTCTCGACATTACCGTCAAGAGGAACAGGAGTATCAGCAGGCCATCACACAC
CACCAAGCCCAACTCGCCGAAAGTAAACAGAGAGAGAAGCAACAAATTGGAGAAAGAGA
GCAACTCGGTACGCTTCTTGTCATTGCCGGCGAAGTGGCTATGTTAACCTGTGAGAATAA
GAGACTGAAAATAACACAACAGGACGAGTCTCGATATTATGCTTGGGAAGAATTGGAGAC
AATACTGATTCTGGGACCTCACAATATAAGCACACCCTGCATCCGACAGGCTATGCACCA
TCAGGTGGCCATTCACTTTGCTTCTCGTTACGGTCAGTACCAGGGTGTCGCATGTTOGAA
CATGCCAAGCCAGGGGCACCAGTTGTGGCAGCTTCAGATTGCTTACTTGCAGAAGGTAG
ACGTTGCTCTTACTTGGAGCATTGAGCTGGTTTGTGCCAAAATCGACGGTCATATTCATTT
GATCAGAAATAGAGAAAGGCAGAGTCCTCTTTTGGAGAAGTTGCGAACTATCAAGAGAAA
GGCAAGGCGATCCGACGATTTACAAGTACTTTTGGGAATTGAAGGCGAGGCCGCCAAAC
TCCAGTGGGAGTTCTTCAAGCAACATCTGGATTGTGAATGGCAGTTCTCCGGAAGGAAC
CGAAGGCCCCCAAAGGACCCAATCAACGCTATGTTGTCCTTAGGATATACATATCTATAC
CATCTGACAGATTCCTTGATTCAATCAAACGGGCTTTGTCCCTGGGCTGGGTTCTACCAT
CAGCCACACGGAGCCCACAGAACTCTGGCCAGCGACCTCATGGAACCACTCCGAGTCG
TCGTGGACCGAACTGTTCTGGCATTGGTAGCCAAGCGTCAGATTAAGCCTGACGATTTCC
TTATATTGGAAGATGGATGCGAAATGTCTAGGGAAGCCCGTAAGGTGCTACTGACTCAGT
TGTTAGCAGACCTTACAACGAAAAGGAAGAAAAGCGATAGGGTTATCGACCAAATGATTA
CCCAAGTAAAAGAGGTTAAGATTGCTACTAAGTTGGCTTTAAACCCAAATTTCTGGCGTC
CTTGA (SEQ ID NO: 129)
Vibrio sinaloensis strain T08, Cas2, DNA:
ATGAAGGTGTATCTGGTGTGTTTCGACATTGAGAACGACAAGAAAAGGCGTAGAC
TGAGTAAGTTGTTACTATCTTATGGAGAACGAGTGCAGTATTCTGTATTCGAGATAACTCT
GAAGTCTGATGCCTCGCTGAAAACCCTTACTAAGCAATGCAAGAGACACGTTGAGCAGG
GAGATAGTTTGAGATTCTACGCACTTCCATTTAACGCTCGTCAGGCCAGCTTCGACATCG
ACGGCGAACCTATAGCAAGGTTCCCCCAAGCCGAGATCTTGTAG (SEQ ID NO: 148)
Blautia sp. AM42-2, RT-Cas1, Amino Acids:
MFTIDEMLSKNNQRLAFEHFATKNDGCGPDGMHVSELEKYWRMNHDQIISDLKNQE
YQPGIILIREHMNKTGKRRNIASLNVIDRFITRLLSQKLNRYLAPIFCENSYAYQDSKGVMPAVL
KAKEYVELGMRHVIEIDLKNYFDTIPLENLIPEIERYITDEAVLHLIKQYLFCDISFEGKISRKTQG
IVQGNAISPILSNLYLNDFDKELDESKLCWIRYADNIYVYMDSYEKALLVYSELTERLERRKLTV
NKEKSGVFDVSTRSILGYDILIRNKKVDVRKHIYKSVNQYSNWHDSRLEFINGRYHITSDGILN
RQDFGLLFENEQKKHYIPVEVSDQLNIYGNVTLASNVLQSFSNREIKVSFFDKYGRLIGSFLPE
KTKKSAEIILVQSKNYLNEDVRMDTARRMEIAGLHNIRANLRYYDKKHKGDFKEKVDAISGYID
ALNRAPSVNDMMLLEAKARQLYYTCFNQILETSDFQFEKRTKRPPKDAINACISFGNTLLYNL
FVNIIWKKGLDPRFGVVHASNKRNQSLNLDFADIFKPIVIDRIIFTMINKKMLTLLTDFETSNQGV
YLSREGKNIFLQMYEEKLKSRITIKGKEMSYYQLLESEVQNYKNFILTGETYKPYKYY (SEQ ID
NO: 018)
Blautia sp. AM42-2, Cas2, Amino Acids:
MYVILVYDIHQKRVGKALKICRKYLIHIQKSVFEGNITESKLKALKEELGHLIDTQMDSVI
IYHLDSVKYTKKEQIGIVQSTSNVI (SEQ ID NO: 038)
Blautia sp. AM42-2, Array1 (DR1), DNA:
AAAAAGTCGGTCGATCTCATGCCTGAAATCATGAATTCTGCAAAATGGCGGAAAT
TTAAGGAAAATCAGGAATCTCAGAAAAACGATCGACCGACTTTTGTGATAAAATGGTTGC
AAAAAAGAGAAAAATTTGATTTAATAGAATGTGAAAATAGCGGAAATGCTGATGTTGTACC
TTACCTATGAGGAATTGAAAC (SEQ ID NO: 105)
Blautia sp. AM42-2, Array2 (DR2), DNA:
AAAAAGTCGGTCAGTCTCGTGGCTGAAATCATGAGTTCCACAAAATGGCTGAAAT
TCAAGGAAAATCAGGAATCTCAGAAAAACGATCGACCGACTTTTTCGATAAAATGGTTGC
AAAAATGAGAAAAATCTGATTTAATAGAATCTGAAAACAGCGGAAATGCTGTTGTCGTACT
TTACCTAAAAGGAATTGAAAC (SEQ ID NO: 106)
Blautia sp. AM42-2, RT-Cas1, DNA:
ATGTTTACAATAGATGAAATGCTATCAAAAAACAACCAACGTCTGGCGTTTGAGCA
CTTTGCGACCAAGAACGACGGTTGTGGTCCGGATGGTATGCATGTTTCCGAGCTGGAAA
AATACTGGCGTATGAATCATGATCAAATTATCAGCGATTTGAAGAACCAAGAATACCAACC
GGGTATTATCCTCATTCGTGAACACATGAACAAGACTGGTAAGCGTAGAAACATTGCGTC
TCTGAACGTGATTGACCGCTTCATCACCCGTCTGCTTAGTCAGAAGCTGAACCGTTACCT
GGCTCCGATCTTCTGCGAAAACAGCTACGCATATCAAGATAGCAAGGGCGTGATGCCGG
CGGTGTTGAAGGCTAAGGAGTACGTGGAGTTGGGCATGCGTCACGTCATCGAGATCGAC
CTGAAAAATTATTTTGATACCATCCCACTGGAGAATCTGATCCCGGAAATCGAACGCTAC
ATCACGGATGAAGCGGTTCTGCACCTGATCAAACAGTATTTGTTTTGTGATATTAGCTTCG
AAGGTAAAATTTCGAGGAAAACCCAGGGTATCGTGCAGGGTAACGCCATCTCCCCGATT
CTCTCTAACCTGTACTTGAATGACTTCGACAAAGAGTTAGACGAAAGCAAATTATGCTGG
ATTCGCTATGCGGATAACATTTACGTTTACATGGATTCTTACGAGAAGGCGCTGCTGGTG
TACAGCGAGCTGACGGAACGTCTTGAGCGCCGCAAGCTGACCGTTAATAAAGAGAAGAG
CGGTGTCTTTGACGTTTCCACCCGTTCAATTCTGGGCTATGATATCCTGATCCGCAACAA
AAAGGTGGACGTTCGTAAACACATCTACAAGTCCGTCAATCAATATAGCAATTGGCATGA
TAGCCGTCTGGAATTTATCAATGGTCGTTATCATATCACGAGCGACGGCATCTTGAACCG
CCAGGATTTCGGTCTGTTGTTCGAGAACGAACAGAAGAAGCACTATATCCCGGTTGAAGT
GAGCGACCAGCTCAACATCTATGGCAATGTCACCCTGGCAAGCAACGTGCTGCAGAGCT
TCTCCAACCGTGAAATTAAAGTGTCGTTTTTCGACAAGTACGGCCGTTTGATAGGCAGCT
TTCTTCCGGAAAAGACCAAAAAATCCGCGGAAATCATCCTGGTCCAGTCCAAGAACTATC
TAAACGAGGATGTTCGTATGGACACCGCGCGTCGTATGGAAATCGCCGGTCTGCACAAC
ATTCGCGCAAATTTGCGTTATTACGACAAAAAGCACAAAGGTGATTTCAAAGAGAAGGTG
GACGCCATTAGCGGTTATATTGACGCTCTGAATCGCGCTCCGAGCGTTAACGACATGAT
GCTGTTGGAGGCGAAAGCGCGCCAGCTGTATTACACGTGCTTTAATCAAATTTTGGAAAC
CTCCGACTTTCAGTTTGAGAAACGTACCAAACGTCCGCCTAAAGATGCAATCAATGCGTG
CATTTCGTTCGGCAACACCCTGCTTTACAATCTGTTCGTGAATATCATCTGGAAAAAAGGC
CTGGACCCGCGTTTTGGCGTAGTTCATGCAAGCAATAAGCGGAACCAATCTTTGAACCTG
GACTTCGCCGACATCTTCAAACCGATTGTTATTGATCGTATTATCTTCACCATGATTAACA
AGAAGATGCTCACGCTGCTGACTGATTTCGAGACATCTAACCAAGGTGTTTATCTGAGCC
GTGAAGGCAAAAATATATTCCTGCAAATGTATGAGGAAAAGCTGAAAAGTCGCATTACCA
TTAAGGGCAAAGAGATGAGCTACTACCAGCTGTTGGAGAGCGAGGTTCAGAATTACAAAA
ACTTTATTCTGACTGGTGAAACCTATAAGCCGTATAAATACTACTAA (SEQ ID NO: 130
Blautia sp. AM42-2, Cas2, DNA:
ATGTATGTTATTCTAGTATACGATATACACCAAAAGCGCGTTGGTAAGGCGCTGA
AGATCTGCCGTAAATACCTGATCCACATTCAGAAGAGCGTGTTCGAGGGCAACATTACGG
AATCCAAACTGAAGGCGTTGAAGGAGGAACTGGGTCATTTAATCGACACCCAGATGGAC
TCGGTCATCATCTATCACCTGGATTCTGTGAAATACACCAAAAAGGAGCAGATTGGCATT
GTTCAAAGCACCAGCAATGTTATCTAA (SEQ ID NO: 149)
Rivularia sp. PCC 7116, Cas1, Amino Acid:
MGYENIRYLHKPYFAETITEKPPQTEEDDSPFQKNIWNAEMAAIYLIEQGTNIYKDYQ
RFIIHVSEKPKLEVPIRDVQQIIVFGNIQLSTPVIQACLKEQIPVVFLSQTGTYHGHLWSEKSIHL
DNQLVQAERRNDDLFRFSVSRAVVLGKLLNSKQLLMRFNRRRKIGKVEEAIYGINQDIDALNY
VDNLDTLRGYEGIAAARYFPAFGNLITNPKFSFSQRFRQPPTDEINSLLSFGYTLLFNNVLSFII
TEGLSPYIGHFHYGDKQKTYLAFDLMEEFRSPIVDSLVLKIINKSLFKPQDFDVVASTGGVYLS
QTSRRVFLKQFENRMNEEISHPDLISQVTYRHAIQLQVRRYKRCLLSDNIYESFLRAD (SEQ
ID NO: 019)
Rivularia sp. PCC 7116, RT, Amino Acid:
MISSQFIDINNFQRAWEKVADKRGCAGVDGETISSFASNQTVNVYQLMNSVADGSY
QPFPCKQVIIPKRNGSQRELKIPTIRDRIVQQALLNVISPLMEEKFSPVSFAYRPNLSYINAVEKI
ADWRDMGYVWVLDADIVKFFDNIDHHRLLQQVRLHIDHPGILCLIKAWISVGVETREGLILPQK
GIPQGAVISPILANIYLHEFDEIISASDLEIVRYADDFLVLSTSQERIAIAKSQVIDLLDSLGLEINT
DKTQITSFERGFRFLGHGFLSDAIFPVDTNKAKLKSGIETNREKTRTRKTSKKKLYHNPYRNK
KVV (SEQ ID NO: 020)
Rivularia sp. PCC 7116, Cas2, Amino Acid:
MLVVVVYDIPNDKRRTKLSNFLEGYGQRVQFSVFECFLNLDEMRQLFEKSKKIVKPS
EDNVRFYWISEDAVSRVLTIGSEHPNAPPNYYVI (SEQ ID NO: 039)
Rivularia sp. PCC 7116, Array1, DNA:
GGTTTTAGTTCTACTACGGAGATAAAGTGAACATGAAAATCGACGCATCTCGCCA
AATCGCTCAAATGCCTATTATTCCGTTGAGATGCGTCGATTGCTTGCTAGGCAAGGGTTT
GAGGTCGGTGTTTTGCTTGATTTTCGGGCTCATATATAATGTTTTTCAGAGATGCGTCGAT
TTGGCTCCTGTAACCCTTACTGGGTAAGGGCTGCTGAGCGAACTCCCCACCGATTGGGT
TAATTCGGATTAGTTGGAAACTTGGGGGAAGTTTGCATATTAACTCGTTACCTCCCCACC
GATTGGGTTAATTCGGATTAGTTGGAAACTTTTACCAATGAAGTTTCCAGTGATAGCAATT
ATGTTACCCCACCGATTGGGTTAATTCGGATTAGTTGGAAAC (SEQ ID NO: 107)
Rivularia sp. PCC 7116, Array2, DNA:
GTCTGCTGTTTCAGCTATAAAGAAACTGCGGTCAAATTAAAGTATAAGCTTGCCAT
TCCGGCAAACAATTTCTTTAAGTCACATATAGCTTTATCCTTCCAATTTGCGTCTTAGGAC
AAGTTATGCTTCCATTGTTAGAATTTGTTGACAATCGATTAACGCAACAATAAGGGTTTCA
GCCCTTAAATTACTTGGTTATGCTTCCAGGGGCTGACAGCTTTTGGGTATAGTGACAGAA
CCTGATTACGAAGGTTTCCAACTAATCCGAATTAACCCAATCGGTGGGGGATGAAGAACG
TTGGCATTTATCCTTACTGGGTAGGTTTCCAACTAATCCGAATTAACCCAATCGGTGGGG
GATGAATACATTCTTTTGGTTAGACATGATCAAATTCTGTTTCCAACTAATCCGAATTAACC
CAATCGGTGGGG (SEQ ID NO: 108)
Rivularia sp. PCC 7116, Array1, DNA:
GCTGAAAGGAGGAACTATATCCGGATACCAGGTTATTATTCCGTTGAGATGCGTC
GATTGCTTGCTAGGCAAGGGTTTGAGGTCGGTGTTTTGCTTGATTTTCGGGCTCATATAT
AATGTTTTTCAGAGATGCGTCGATTTGGCTCCTGTAACCCTTACTGGGTAAGGGCTGCTG
AGCGAACTCCCCACCGATTGGGTTAATTOGGATTAGTTGGAAACGTCCCGCGGCCGCTA
TTCTTTTGATTTATAAGGGATTTTGCGATCCTCTGGAGTGAATACCACGACGATTTCCGGC
AGTTTCTCCACA (SEQ ID NO: 109)
Rivularia sp. PCC 7116, Array1 RC, DNA:
GCTGAAAGGAGGAACTATATCCGGATACCAGGTATTTGCGTCTTAGGACAAGTTA
TGCTTCCATTGTTAGAATTTGTTGACAATCGATTAACGCAACAATAAGGGTTTCAGCCCTT
AAATTACTTGGTTATGCTTCCAGGGGCTGACAGCTTTTGGGTATAGTGACAGAACCTGAT
TACGAAGGTTTCCAACTAATCCGAATTAACCCAATCGGTGGGGGTCCCGCGGCCGCTAT
TCTTTTGATTTATAAGGGATTTTGCGATCCTCTGGAGTGAATACCACGACGATTTCCGGCA
GTTTCTCCACA (SEQ ID NO: 110)
Rivularia sp. PCC 7116, Array2, DNA:
GCTGAAAGGAGGAACTATATCCGGATACCAGGTAAAGCAGAAGTGGAAGCACTT
TGTGGCAGTACGCTTGTGGAAGTACTTCTTGGCAGTTACTGCCATCTTCTCGTTCAAGTT
ACAAGATGCTCGGTTGAAACCCCACCGATTGGGTTAATTCGGATTAGTTGGAAACGTCCC
GCGGCCGCTATTCTTTTGATTTATAAGGGATTTTGGGATATACCACCGTTGATATATCCCA
ATGGCATCGTAAAGAACATTTTGAGGCATTTCAGTCAGTTGCTCAATGTACCTATAACCAG
ACCGTTCAG (SEQ ID NO: 111)
Rivularia sp. PCC 7116, Array2 RC, DNA:
GCTGAAAGGAGGAACTATATCCGGATACCAGGTTAAGTTAATTACAGCTTGATTT
TTCATCCAATTTTTATATTAAAAGCCTACCGCTTAAATTGGTAGGCTTTTTTATTGCAAGTC
ATTGTAACTCTTTACTGTTTCCAACTAATCCGAATTAACCCAATCGGTGGGGGTCCCGCG
GCCGCTATTCTTTTGATTTATAAGGGATTTTGGGATATACCACCGTTGATATATCCCAATG
GCATCGTAAAGAACATTTTGAGGCATTTCAGTCAGTTGCTCAATGTACCTATAACCAGACC
GTTCAG (SEQ ID NO: 112)
Rivularia sp. PCC 7116, RT, DNA:
ATGGGATACGAGAACATCAGATACCTTCACAAGCCGTACTTCGCAGAAACGATTA
CTGAGAAGCCACCACAGACTGAGGAAGATGACTCACCATTCCAGAAGAACATCTGGAAC
GCTGAGATGGCTGCAATCTATCTTATCGAGCAGGGTACAAACATCTACAAAGACTACCAG
CGTTTCATAATACACGTTAGTGAGAAGCCAAAACTTGAGGTCCCTATCAGGGACGTACAA
CAGATCATCGTCTTTGGAAATATCCAGCTAAGCACGCCTGTTATCCAGGCCTGCCTGAAG
GAGCAGATCCCAGTTGTTTTCCTGTCTCAGACCGGAACATACCATGGCCATCTGTGGTCG
GAGAAGTCAATCCACCTTGATAACCAGTTGGTTCAGGCTGAGCGAAGGAACGACGACTT
ATTCAGATTCTCAGTTAGCCGAGCAGTGGTTCTAGGGAAGCTTCTGAACTCTAAGCAACT
ACTGATGAGATTCAACAGAAGGAGAAAGATTGGTAAGGTCGAAGAGGCAATCTACGGTAT
CAACCAGGACATAGACGCATTGAATTACGTGGACAACCTGGACACGCTGAGGGGATACG
AGGGTATCGCAGCCGCCCGTTACTTCCCCGCCTTCGGTAACTTGATAACAAACCCAAAGT
TCAGCTTCAGTCAAAGGTTCAGGCAGCCACCAACTGACGAAATTAACTCTCTACTGTCCT
TCGGTTACACATTGTTATTCAACAACGTTTTATCCTTCATCATCACTGAGGGCTTGTCGCC
GTACATAGGACACTTCCATTACGGTGACAAGCAGAAAACTTACCTCGCATTCGACCTTAT
GGAAGAGTTCAGATCGCCTATCGTAGACTCTCTTGTATTGAAGATAATTAACAAGAGCCT
ATTCAAGCCCCAAGACTTTGACGTTGTCGCCTCTACCGGCGGTGTTTATTTGTCTCAGAC
CAGCCGTAGGGTGTTCCTGAAGCAATTCGAGAACAGAATGAACGAGGAAATCTCCCACC
CGGACCTGATCTCCCAGGTTACTTACCGTCATGCTATCCAGTTGCAGGTCAGACGATACA
AAAGGTGCCTGCTGAGTGACAACATCTACGAGTCTTTCCTAAGGGCTGACTAA (SEQ ID
NO: 131)
Rivularia sp. PCC 7116, Cas1, DNA:
ATGATATCATCTCAGTTCATAGACATAAACAACTTCCAGAGAGCATGGGAGAAAG
TCGCAGACAAGAGAGGGTGTGCTGGAGTAGACGGAGAGACAATCTCCTCATTCGCCAGT
AACCAGACTGTGAACGTCTACCAACTCATGAATAGTGTGGCAGACGGATCTTACCAGCC
GTTCCCATGTAAACAAGTCATCATCCCAAAGAGGAATGGGTCCCAGAGGGAACTAAAGAT
ACCGACCATCAGGGATAGGATAGTGCAGCAGGCTCTCCTAAACGTCATCTCACCTCTTAT
GGAAGAAAAGTTCTCTCCAGTCTCTTTCGCATACAGGCCAAACTTGAGCTACATTAACGC
CGTGGAGAAGATCGCAGATTGGAGAGATATGGGGTACGTTTGGGTCCTGGACGCCGAC
ATTGTGAAATTCTTCGACAACATCGATCACCACAGACTTTTGCAGCAAGTGCGACTACAC
ATCGATCACCCTGGCATCCTGTGCCTGATAAAGGCATGGATCTCTGTCGGCGTCGAGAC
ACGAGAGGGTTTGATTCTTCCACAGAAAGGAATCCCACAGGGCGCTGTCATCTCACCTAT
CTTAGCTAACATCTACCTGCACGAGTTCGACGAGATAATCTCTGCATCTGACCTAGAGAT
AGTAAGATACGCCGACGACTTCTTAGTACTATCCACCAGCCAGGAGAGAATAGCCATAGC
CAAGTCCCAGGTTATAGACTTGCTCGACTCATTGGGTCTAGAGATTAACACAGACAAGAC
ACAGATCACGTCTTTCGAGAGAGGATTCAGATTCCTTGGACACGGGTTTCTGTCCGACGC
AATATTTCCAGTGGACACCAACAAGGCTAAACTGAAAAGTGGAATCGAGACAAACAGAGA
AAAGACTAGGACCCGAAAGACGAGCAAGAAGAAGCTCTACCATAATCCTTACAGAAATAA
GAAGGTCGTATGA (SEQ ID NO: 132)
Rivularia sp. PCC 7116, Cas2, DNA:
ATGCTTGTGGTGGTGGTTTATGACATTCCCAATGACAAGAGGCGAACAAAACTGT
CTAACTTCTTGGAAGGGTACGGACAGCGTGTGCAGTTCTCAGTTTTCGAGTGCTTCTTAA
ACCTGGACGAAATGAGACAGCTATTCGAGAAAAGCAAGAAGATTGTAAAGCCGTCTGAG
GATAACGTGAGATTCTACTGGATATCAGAGGACGCAGTGTCTCGTGTGCTTACCATAGGC
TCTGAGCACCCCAATGCCCCACCTAATTATTACGTCATCTAA (SEQ ID NO: 150)
Human codon-optimized FsRT-Cas1-T7RBS-Cas2, DNA:
GCGGAGGAGCATGCATGTTTACCATCGACGAGATGCTGAGCAAGAACAACCAAC
GTCTGGCGTTCGAACACTTTGCGACCAAAAACGACGGTTGCGGCCCGGACGGTATGCAC
GTTAGCGAACTGGAGAAGTACTGGCGTATGAATCACGACCAGATCATTAGCGATCTGAA
GAACCAGGAATACCAGCCGGGTATTATCCTGATTCGTGAGCACATGAATAAAACCGGCAA
ACGCCGTAATATCGCGAGCCTGAACGTTATTGATCGTTTCATTACCCGTCTGCTGAGCCA
GAAACTGAACCGTTATCTGGCGCCGATTTTCTGCGAGAACAGCTATGCGTACCAGGACA
GCAAAGGCGTTATGCCGGCGGTTCTGAAAGCGAAGGAGTACGTTGAGCTGGGTATGCGT
CACGTGATTGAAATCGATCTGAAAAACTACTTTGACACCATTCCGCTGGAAAACCTGATC
CCGGAGATCGAACGCTACATCACCGATGAGGCGGTTCTGCATCTGATCAAGCAATACCT
GTTTTGCGATATTAGCTTCGAGGGCAAAATCAGCCGTAAAACCCAAGGTATCGTTCAAGG
TAACGCGATCAGCCCGATCCTGAGCAACCTGTACCTGAACGACTTCGACAAGGAACTGG
ACGAGAGCAAGCTGTGCTGGATTCGTTACGCGGACAATATCTACATCTATATGGATAGCT
ATGAGAAGGCGCTGCTGGTGTATAGCGAGCTGACCGAGCGTCTGGAGCGTCGCAAGCT
GACCGTTAACAAGGAGAAAAGCGGCGTTTTTGATGTGAGCACCCGTAGCATTCTGGGCT
ACGACATCCTGATTCGTAACAAGAAAGTGGACGTTCGTAAGCATATTTACAAAAGCGTTA
ATCAGTACAGCAACTGGCACGATAGCCGCCTGGAGTTTATCAACGGTCGTTACCACATCA
CCAGCGATGGCATCCTGAATCGCCAAGACTTCGGCCTGCTGTTCGAGAATGAGCAGAAA
AAACACTACATCCCGGTTGAGGTTAGCGATCAGCTGAACATCTACGGTAATGTTACCCTG
GCGAGCAATGTGCTGCAAAGTTTCAGCAACCGCGAAATCAAAGTGAGCTTCTTCGACAA
GTATGGTCGTCTGATTGGTAGCTTCCTGCCGGAAAAAACCAAGAAAAGCGCGGAGATCA
TTCTGGTGCAGAGCAAGAACTATCTGAACGAAGATGTGCGTATGGATACCGCGCGCCGT
ATGGAAATTGCGGGTCTGCACAACATCCGTGCGAATCTGCGCTACTACGATAAGAAACAC
AAGGGCGACTTTAAAGAGAAGGTTGACGCGATTAGCGGCTACATTGACGCGCTGAACCG
TGCGCCGAGCGTTAATGATATGATGCTGCTGGAAGCGAAAGCGCGCCAACTGTATTATA
CCTGTTTTAACCAAATCCTGGAAACCAGCGACTTCCAGTTCGAAAAGCGTACCAAGCGTC
CGCCGAAAGATGCGATCAATGCGTGCATTAGCTTCGGCAATACCCTGCTGTATAATCTAT
TTGTGAATATTATCTGGAAGAAGGGTCTGGACCCGCGCTTTGGCGTTGTTCACGCGAGC
AACAAACGCAATCAAAGCCTGAACCTGGACTTCGCGGACATCTTTAAACCGATCGTGATT
GACCGCATTATCTTCACCATGATCAATAAGAAAATGCTGACCCTGCTGACCGATTTTGAAA
CCAGCAACCAGGGTGTGTATCTGAGCCGTGAGGGCAAGAACATCTTCCTGCAAATGTAC
GAGGAGAAGCTGAAAAGCCGCATTACCATCAAGGGTAAAGAGATGAGCTATTACCAGCT
GCTGGAGAGCGAAGTGCAGAATTACAAGAATTTCATTCTGACCGGCGAAACCTATAAACC
GTATAAGTATTATTAACGTACGTTAACTTTAAGAAGGAGAACTTAAGATGTATGTTATCCT
GGTGTACGACATCCACCAGAAGCGTGTTGGTAAAGCGCTGAAAATCTGCCGTAAGTACC
TGATCCACATCCAAAAGAGCGTGTTCGAAGGCAACATCACCGAGAGCAAACTGAAGGCG
CTGAAAGAGGAACTGGGTCACCTGATTGACACCCAGATGGATAGCGTGATTATCTACCAC
CTGGACAGCGTTAAGTACACCAAAAAGGAGCAGATCGGTATTGTGCAGAGCACCAGCAA
TGTGATCTAACTCGAGATCCGGCTG (SEQ ID NO: 152)

1. An expression vector comprising the following sequence elements:
a first transgene nucleic acid sequence encoding a first amino acid sequence of a fusion protein of a reverse transcriptase and a Cas1 polypeptide, and a second transgene nucleic acid sequence encoding a second amino acid sequence of a Cas2 polypeptide, wherein said first transgene nucleic acid sequence and said second transgene nucleic acid sequence are under transcriptional control of an inducible promoter sequence, and
a CRISPR array sequence comprising a CRISPR direct repeat (DR) sequence;
wherein said first amino acid sequence consists of a sequence being ≥90% identical to a sequence selected from the group consisting of SEQ ID NO: 1-18;
and wherein said second amino acid sequence consists of a sequence being ≥90% identical to a sequence selected from the group consisting of SEQ ID NO: 21-38;
and wherein said CRISPR array sequence consists of a sequence being ≥98% identical to a sequence selected from the group consisting of SEQ ID NO: 40-106.

2. The expression vector according to claim 1, wherein said CRISPR array sequence further comprises a CRISPR leader sequence, wherein said CRISPR leader sequence and said CRISPR direct repeat sequence are separated by 10 to 0 bp.

3. The expression vector according to claim 1, wherein said CRISPR array sequence does not comprise any further CRISPR repeat sequence specifically recognizable by said RT-Cas1-Cas2 complex.

4. The expression vector according to claim 1, further comprising an endonuclease recognition site sequence downstream or within of said CRISPR direct repeat, wherein said endonuclease recognition site sequence is specifically recognizable by a site-specific endonuclease, and said CRISPR direct repeat and said restriction site sequence are separated by 10 bps to 0 bps.

5. The expression vector according to claim 1, wherein said site-specific endonuclease is a Type IIS or Type IIG restriction endonuclease.

6. The expression vector according to claim 1, wherein said inducible promoter sequence is operable in E. coli and is selected from T7 promoter, lac promoter, tac promoter, Pₜₑₜ promoter, P_{C} promoter and P_{BAD} promoter.

7. The expression vector according to claim 1, wherein said first transgene nucleic acid sequence and said second transgene nucleic acid sequence are codon-optimized for E. coli.

8. The expression vector according to claim 1, wherein said first amino acid sequence being ≥90% identical to a sequence selected from the group consisting of SEQ ID NO: 1-18 comprise only substitutions in relation to SEQ ID NO: 1-18, and no insertions or deletions.

9. The expression vector according to claim 1, wherein said second amino acid sequence being ≥90% identical to a sequence selected from the group consisting of SEQ ID NO: 21-38 comprise only substitutions in relation to SEQ ID NO: 21-38, and no insertions or deletions.

10. The expression vector according to claim 8,
wherein said first amino acid sequence comprises 0 to 20 substitutions with respect to SEQ ID NO: 1, wherein following fixed amino acid positions with respect to SEQ ID NO: 1 are not substituted:
M1, F2, I4, E6, N12, A16, F17, H19, F20, K23, D25, G28, D30, G31, M32, S35, E36, L37, Y40, W41, N44, 148, D51, L52, E56, Y57, Q58, P59, G60, E66, K70, G72, K73, R74, R75, N76, 177, A78, L80, N81, V82, 183, D84, R85, F86, 187, T88, R89, L90, Q93, L95, L99, F103, S107, Y108, A109, Y110, Q111, K114, G115, A119, K124, Y126, G130, D138, L139, Y142, F143, D144, I146, L148, L151, I152, P153, I155, V164, L167, I168, K169, Y171, L172, C174, D175, G180, I182, R184, G188, I189, G192, N193, A194, I195, S196, P197, L199, S200, N201, L202, Y₂O₃, L204, F207, D208, W218, R220, Y221, D223, N224, Y228, L236, L251, N254, K257, S258, G259, F261, R266, L269, G270, Y271, D272, I273, D281, R283, H285, Y287, Y293, W296, L301, I304, N305, G306, R307, Y308, H309, I310, S312, D313, G314, I315, R318, D320, F321, L323, L324, F325, E326, N327, I334, P335, E337, V338, D340, N343, N347, V348, L350, V354, L355, I363, V365, F367, F368, D369, K370, Y371, G372, L374, I375, G376, F378, L379, P380, E381, A387, I389, L391, Q393, Y397, R403, A407, R408, M410, A413, L415, H416, N417, I418, R419, A420, N421, L422, R423, Y424, Y425, K427, K428, F433, V437, D438, I440, I444, L447, S452, V453, M456, L458, E460, A461, A463, R464, Y467, Y468, F471, N472, I474, L475, F480, F482, R485, T486, P489, P490, D492, A493, I494, N495, A496, I498, S499, F500, G501, N502, T503, L504, L505, Y506, N507, F509, I512, I513, W514, K516, G517, L518, D519, P520, R521, F522, G523, V524, H526, R531, S534, L535, N536, L537, D538, F539, A540, D541, I542, F543, K544, P545, I546, V547, D549, R550, I552, F553, T554, M555, I556, N557, K558, L561, F567, G573, V574, Y575, L576, S577, G580, K581, F584, L585, E589, K591, L592, K593, T597, K599, G600, M603, S604, Y605, L608, L609, E610, E612, V613, Q614, Y616, K617, N618, I620, L621, G623, Y626, K627, P628, Y629, K630, Y631, Y632
and wherein following conserved amino acid positions with respect to SEQ ID NO: 1 can only be substituted according to substitution rules given below:
T3, D5, M7, L8, A21, G26, H33, V34, E38, M43, Q47, K53, I61, I62, I64, R65, M68, S79, L91, A100, V116, V120, K122, A123, R132, V134, I137, N141, E156, Y158, I159, D161, L165, H166, F173, F178, E179, K185, V190, N205, D206, L211, I219, A222, I225, Y226, I227, Y232, E233, A235, Y239, L246, V253, E256, V260, V263, I268, L274, K278, K279, V280, V282, K284, I286, H297, D298, S299, T311, L316, N317, G322, E328, K330, K331, Y333, S339, Q341, L342, 1344, Y345, G346, T349, A351, S352, N353, Q356, S357, F358, S359, S366, R373, T383, K384, K385, S386, 1390, L398, N399, D401, M404, D405, T406, R409, E411, G414, D426, H429, K430, G431, K434, E435, S441, Y443, A446, A450, D455, M457, L459, K462, Q465, L466, C470, Q473, D479, K487, R488, K491, C497, N511, K515, V525, A527, S528, N529, I548, I551, K559, M560, D566, E568, R578, Y588, S594, R595, I596, I598, Y606, Q607, N615, F619, E624
Substitution rules:
glycine (G) and alanine (A) are interchangeable; valine (V), leucine (L), and isoleucine (1) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) and histidine (H) are interchangeable;
W, F, Y, A, I, L, and V are interchangeable;
or wherein 0-20 amino acid positions of each sequence of SEQ ID NO: 2-17 are substituted, wherein amino acids homologous to the fixed amino acids of SEQ ID NO: 1 cannot be substituted, and wherein amino acids homologous to conserved amino acids of SEQ ID NO: 1 can only be substituted according to the substitution rules.

11. The expression vector according to claim 9, wherein said second amino acid sequence comprises 1 to 8 substitutions with respect to SEQ ID NO: 21, wherein following fixed amino acid positions with respect to SEQ ID NO: 21 are not substituted:
M1, Y2, I4, L5, Y7, D8, K12, R13, R22, Y24, L25, Q29, K30, S31, F33, G35, E39, K41, L45, E48, L49, D54, D58, Y63, L65, S67, K69, Y70, K73, G77, I78, S81, I86
and wherein following conserved amino acid positions with respect to SEQ ID NO: 21 can only be substituted according to substitution rules given below:
V3, V6, I9, V14, K16, A17, L18, I20, C21, K23, I28, V32, I37, T38, K46, I53, Q56, M57, V60, I61, I62, V68, T71, K72, T82, S83, V85
Substitution rules:
glycine (G) and alanine (A) are interchangeable; valine (V), leucine (L), and isoleucine (I) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) and histidine (H) are interchangeable;
W, F, Y, A, I, L, and V are interchangeable;
or wherein 0-8 amino acid positions of each sequence of SEQ ID NO: 22-37 are substituted, wherein amino acids homologous to the fixed amino acids of SEQ ID NO: 21 cannot be substituted, and wherein amino acids homologous to conserved amino acids of SEQ ID NO: 21 can only be substituted according to the substitution rules.

12. A bacterial cell comprising the first transgene nucleic acid sequence, the second transgene nucleic acid sequence and the CRISPR array sequence of claim 1,
wherein said first transgene nucleic acid sequence, said second transgene nucleic acid sequence and said CRISPR array sequence are
comprised in an expression vector according to claim 1 or
integrated into the genome of said cell.

13. The cell according to claim 12, additionally comprising
a fourth transgene nucleic acid sequence encoding a fourth transgene product, wherein said fourth transgene product is capable of modulating the expression of a record gene inside the cell, and wherein such modulating the expression of said record gene is dependent on the presence or absence of an analyte molecule.

14. The cell according to claim 12, wherein said fourth transgene product is a sensor which will be activated when contacted with a molecule of interest yielding an activated sensor, wherein said activated sensor will induce the expression of a record gene inside the cell.

15. The cell according to claim 12, wherein said cell is an E. coli cell.

16. The cell according to claim 12, wherein said first amino acid sequence being ≥90% identical to a sequence selected from the group consisting of SEQ ID NO: 1-18 comprise only substitutions in relation to SEQ ID NO: 1-18, and no insertions or deletions.

17. The cell according to claim 12, wherein said second amino acid sequence being ≥90% identical to a sequence selected from the group consisting of SEQ ID NO: 21-37 comprise only substitutions in relation to SEQ ID NO: 21-37, and no insertions or deletions.

18. The cell according to claim 16,
wherein said first amino acid sequence comprises 0 to 20 substitutions with respect to SEQ ID NO: 1, wherein following fixed amino acid positions with respect to SEQ ID NO: 1 are not substituted:
M1, F2, I4, E6, N12, A16, F17, H19, F20, K23, D25, G28, D30, G31, M32, S35, E36, L37, Y40, W41, N44, 148, D51, L52, E56, Y57, Q58, P59, G60, E66, K70, G72, K73, R74, R75, N76, 177, A78, L80, N81, V82, 183, D84, R85, F86, 187, T88, R89, L90, Q93, L95, L99, F103, S107, Y108, A109, Y110, Q111, K114, G115, A119, K124, Y126, G130, D138, L139, Y142, F143, D144, I146, L148, L151, 1152, P153, I155, V164, L167, I168, K169, Y171, L172, C174, D175, G180, I182, R184, G188, I189, G192, N193, A194, I195, S196, P197, L199, S200, N201, L202, Y₂O₃, L204, F207, D208, W218, R220, Y221, D223, N224, Y228, L236, L251, N254, K257, S258, G259, F261, R266, L269, G270, Y271, D272, I273, D281, R283, H285, Y287, Y293, W296, L301, I304, N305, G306, R307, Y308, H309, I310, S312, D313, G314, I315, R318, D320, F321, L323, L324, F325, E326, N327, I334, P335, E337, V338, D340, N343, N347, V348, L350, V354, L355, 1363, V365, F367, F368, D369, K370, Y371, G372, L374, 1375, G376, F378, L379, P380, E381, A387, I389, L391, Q393, Y397, R403, A407, R408, M410, A413, L415, H416, N417, I418, R419, A420, N421, L422, R423, Y424, Y425, K427, K428, F433, V437, D438, I440, I444, L447, S452, V453, M456, L458, E460, A461, A463, R464, Y467, Y468, F471, N472, I474, L475, F480, F482, R485, T486, P489, P490, D492, A493, I494, N495, A496, I498, S499, F500, G501, N502, T503, L504, L505, Y506, N507, F509, I512, I513, W514, K516, G517, L518, D519, P520, R521, F522, G523, V524, H526, R531, S534, L535, N536, L537, D538, F539, A540, D541, I542, F543, K544, P545, I546, V547, D549, R550, I552, F553, T554, M555, I556, N557, K558, L561, F567, G573, V574, Y575, L576, S577, G580, K581, F584, L585, E589, K591, L592, K593, T597, K599, G600, M603, S604, Y605, L608, L609, E610, E612, V613, Q614, Y616, K617, N618, I620, L621, G623, Y626, K627, P628, Y629, K630, Y631, Y632
and wherein following conserved amino acid positions with respect to SEQ ID NO: 1 can only be substituted according to substitution rules given below:
T3, D5, M7, L8, A21, G26, H33, V34, E38, M43, Q47, K53, I61, I62, I64, R65, M68, S79, L91, A100, V116, V120, K122, A123, R132, V134, I137, N141, E156, Y158, I159, D161, L165, H166, F173, F178, E179, K185, V190, N205, D206, L211, I219, A222, I225, Y226, I227, Y232, E233, A235, Y239, L246, V253, E256, V260, V263, I268, L274, K278, K279, V280, V282, K284, I286, H297, D298, S299, T311, L316, N317, G322, E328, K330, K331, Y333, S339, Q341, L342, I344, Y345, G346, T349, A351, S352, N353, Q356, S357, F358, S359, S366, R373, T383, K384, K385, S386, 1390, L398, N399, D401, M404, D405, T406, R409, E411, G414, D426, H429, K430, G431, K434, E435, S441, Y443, A446, A450, D455, M457, L459, K462, Q465, L466, C470, Q473, D479, K487, R488, K491, C497, N511, K515, V525, A527, S528, N529, I548, I551, K559, M560, D566, E568, R578, Y588, S594, R595, I596, I598, Y606, Q607, N615, F619, E624
Substitution rules:
glycine (G) and alanine (A) are interchangeable; valine (V), leucine (L), and isoleucine (1) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) and histidine (H) are interchangeable;
W, F, Y, A, I, L, and V are interchangeable;
or wherein 0-20 amino acid positions of each sequence of SEQ ID NO: 2-17 are substituted, wherein amino acids homologous to the fixed amino acids of SEQ ID NO: 1 cannot be substituted, and wherein amino acids homologous to conserved amino acids of SEQ ID NO: 1 can only be substituted according to the substitution rules.

19. The cell according to claim 17, wherein said second amino acid sequence comprises 1 to 8 substitutions with respect to SEQ ID NO: 21, wherein following fixed amino acid positions with respect to SEQ ID NO: 21 are not substituted:
M1, Y2, I4, L5, Y7, D8, K12, R13, R22, Y24, L25, Q29, K30, S31, F33, G35, E39, K41, L45, E48, L49, D54, D58, Y63, L65, S67, K69, Y70, K73, G77, 178, S81, I86
and wherein following conserved amino acid positions with respect to SEQ ID NO: 21 can only be substituted according to substitution rules given below:
V3, V6, I9, V14, K16, A17, L18, I20, C21, K23, I28, V32, I37, T38, K46, I53, Q56, M57, V60, I61, I62, V68, T71, K72, T82, S83, V85
Substitution rules:
glycine (G) and alanine (A) are interchangeable; valine (V), leucine (L), and isoleucine (I) are interchangeable, A and V are interchangeable;
tryptophan (W) and phenylalanine (F) are interchangeable, tyrosine (Y) and F are interchangeable;
serine (S) and threonine (T) are interchangeable;
aspartic acid (D) and glutamic acid (E) are interchangeable
asparagine (N) and glutamine (Q) are interchangeable; N and S are interchangeable; N and D are interchangeable; E and Q are interchangeable;
methionine (M) and Q are interchangeable;
cysteine (C), A and S are interchangeable;
proline (P), G and A are interchangeable;
arginine (R) and lysine (K) and histidine (H) are interchangeable;
W, F, Y, A, I, L, and V are interchangeable;
or wherein 0-8 amino acid positions of each sequence of SEQ ID NO: 22-37 are substituted, wherein amino acids homologous to the fixed amino acids of SEQ ID NO: 21 cannot be substituted, and wherein amino acids homologous to conserved amino acids of SEQ ID NO: 21 can only be substituted according to the substitution rules.


SEQUENCE LISTING (383 sequences)

SEQ ID NO: 1 - protein, 632 aa, Fusicatenibacter saccharivorans
MFTIDEMLSKNNQRLAFEHFATKNDGCGPDGMHVSELEKYWRMNHDQIISDLKNQEYQPGIILIREHMNKTGKRRNIASLNVIDRFITRLLSQKLNRYLAPIFCENSYAYQDSKGVMPAVLKAKEYVELGMRHVIEIDLKNYFDTIPLENLIPEIERYITDEAVLHLIKQYLFCDISFEGKISRKTQGIVQGNAISPILSNLYLNDFDKELDESKLCWIRYADNIYIYMDSYEKALLVYSELTERLERRKLTVNKEKSGVFDVSTRSILGYDILIRNKKVDVRKHIYKSVNQYSNWHDSRLEFINGRYHITSDGILNRQDFGLLFENEQKKHYIPVEVSDQLNIYGNVTLASNVLQSFSNREIKVSFFDKYGRLIGSFLPEKTKKSAEIILVQSKNYLNEDVRMDTARRMEIAGLHNIRANLRYYDKKHKGDFKEKVDAISGYIDALNRAPSVNDMMLLEAKARQLYYTCFNQILETSDFQFEKRTKRPPKDAINACISFGNTLLYNLFVNIIWKKGLDPRFGVVHASNKRNQSLNLDFADIFKPIVIDRIIFTMINKKMLTLLTDFETSNQGVYLSREGKNIFLQMYEEKLKSRITIKGKEMSYYQLLESEVQNYKNFILTGETYKPYKYY

SEQ ID NO: 2 - protein, 880 aa, Candidatus Accumulibacter
MSTPDYCWQRIGLTLRCTFAHPGRKAHPLSVLEAIVKGIGEAAGLTAPTMRSVFRWSGMRSSQMTIESGRILSLEILLFGTDAGAACNWHERAIHYFDPGAPGRNFQVTASEAPVERRWAELLAGRQAPAESNDECCLDFLTPLPFTPAQGRGRTWLDGEGLRRAMQDRLRRLFGAEAELPPIPEVLPAYWYYCQIVHAASSQPGHNKYLNGCLGPLLLRGEHLGEWWPWLVLGEEIGLGGQVSFGQGLFRLHAKSVPILDARLTDPNQIAAIIDQLLLRHDDLAVRLSNTPQAPDLHELAVELAQNLREGAAPLPFQAIRVPRSDGRLRQFETPAARDLVILNHLTRLLSEPFDRLFSVHSIGYRKGHSREDAVERVRAAIAEGCTHVLESDISDFFPSVDLKRLLARLDDVLPRRDVRLRQTLAAYLGAGWRYGEGSVQARNRGLPLGSPLSPLLANLYLDSFDSQLGATVPGVRLIRYADDFIILTESEAAARALLDTARDAAAALGLALNLEKTAIRPLSDGFDFLGIRFSADAAAEQAGDESADSLRKVLYITEPYAFVGSNHGTIEVHAGSKSLGSFPLARTAGVVTLVPCTLSSALIARLADQCIPLAIAGTQGRQIATVAGDTARRFATAATQANRHASLGEAGRCRAAGAFATAKLANYIALIRQRGPAGTAALVARLENGIAAIASATDIDAIRGVEGDCARECFPFIAGWINSPDFPWQGRRRHGEFPDRLNSLLNFGYHLLFTRINALLRVSGLNPYLGFLHAANGRYEALACDVQEAFRPHIDRLVVRLLNLKVIEAADFEESEEGWWLIRPARTRFLQQFAREIERRPMRRRYSLGEAIEGQVRALHAWLIEDRELVLYRWSDSDV

SEQ ID NO: 3 - protein, 660 aa, Candidatus Accumulibacter
MSTLPTPSSTDQDSPPPFWTLARLAEALEHVSARQGGAGADEQTLAEFAADAEAQLGLLALQLTQGSYRPAPARLIPVAKPGGGVRELLLPAVRDRIVQSALARYLADLLEPDFGEASHAYRPGHSVATALHRLQALRDGGLVFVAVCDIHHFFDSVDHRRLFSLLDDLPLERRLREQMKTCVRIEVADVQGQGAWSLARGLAQGSPLSPVLANLFLMAFDAACARAGLALVRYADDCVLACASETEAQSALAFAADALENIGLALNTRKSRLASFAEGFEFLGAFCGAEGMLGGRPGEAACLPPTTGPVHEAAAADDERPPSHGHRPRLRTLYLLENGAVLNKEGERFIVARHGEVLLQVPMMRIDQIMVFGNVQITTPALHECLERGIPVMLLSGRGRFFGVIDPLDARSVPLQRAQFALESDEPARLALARPLIAGKILNCRTFLGRLARARQTNMDAPLAALKSAAQAAGQAADLEILRGIEGAAARTYFAAWQTVLPAKWQFTGRNRQPPTDPVNALLSYGYTIVFYNVLALVRARGLNTHVGVLHDVRPGHPALASDLMEEFRAPVVDAVVMHLVFDGKLQPGDFSWPETPGQPCLMADGSRKHFIHLLEQKLNTTVSHAGQRLDYRRWMDMQVLQYAAALRTPGLPYVPFAIR

SEQ ID NO: 4 - protein, 639 aa, Eubacterium saburreum
MKKLTMEDIFISEQIEEALDHLSTKKDTCGIDGLYLSELRDDWNINGERYLSLLRKGKYKPGIVQIYEIVNYTGKRRSISSFNSIDRLVLRCLATSLEKYYDSIFSSSSFAFRPGLGVDKAVATFANNLNTGLTRVAIIDIKHYFDSIPIDRLEMILKRIIDDNVLLSLFHNLLYCRISEENVIKTKSKGILQGSPISPFLGNLYLSLLDTQLESMHVSFCRYCDDIAMFFASFEEAKETYTKVYDILKNDLEMDINPQKSGIYEGIKQNYLGYSFTKNKKEHQILAIKKKKAPPQIYQHWSTTAIQRVDRNYHIINNGILNRKDFTLLFENDRGKKYLPVEATESLNVYTSVIFSSDFFKYVGNKKICVNIFDKYGELAGTFSPPESLHGGLTMLKQAAIYLDDEKRKLIARKLEIASLHNMRSNLKYYERHHSSENLKDGITSFSEYITAMNEATNIVMLLTIEARARQLYYSLFHEIICDPAFEFTKRTRRPPKDPLNALISFGNVFLYNRIATEIQKTSLDIRIGFVHATNRRNQSLNLDIADLFKPLIVDRAIFTIINRHMIHASEHFEKTEDGGIYLNKEGKQIFINELENKVYQKQTEENKPRTYDTRIREEIHKIFRLVCYDEKYKPFKYN

SEQ ID NO: 5 - protein, 756 aa, Bacteroides fragilis
MPDYYHSITTLHALQNAWRAVRAKNAAGGIDGFTLSHFEKRLNDNLIELQHELISQTWNPEPYLRIEITKNETEKRKLGLLCIKDKIVQQAIKTAIEPQLEKTFLNLSYGYRPNKGPERAIKRVVHDLKKLKSGYVAKLDIDNYFDTINHERLFTRLANWLKDDETLRLIRLCIQTGIVTPQLQWQEINKGVPQGAILSPLLANFYLHPFDQFAANKVPMYIRYADDFLIATSTEKQIKEAVELVKEELESQFYLQLNTPIIHNFHDGIEFLGITISDTGLSITEKKKKTLQERINSIKFIKSSLSSQSKETLQGIKNYYAKLLPESTLKELDCFLMNRLNALIIRNQNSINNKKELVSNLQKIEFYSENSNKNKSQLIQQLCSTYIVHSTKSKTRLTSTHIDNTKLITQKKKEYQKRENEGAELVISIPGSYIGATYKGITVKLQGKIINKPSPALKHITVVGKGISLSSNAITYCMNHKIPIDFFDGRGKQYGTVLNPVFLDGTLWNKQVELPLEQKIKLATQIIIGKLKNQLNLIKYYHKYHKDILGGKLSEKYVEVVLKIDKLIEKAKNYSQRNEKYTAELMAIESQAAIAYWSYIRVLTADDGIDFIRREHQGATDLLNSLLNYGYAILYARVWKNILAAKLNPSIGVLHAKQDGKPTLVFDVVELFRAQMVDRVVISLIQKKVSLKMHDGLLNESSKRVLIRYILERLNRYEKYRGEEITFSQIILRQAQEIALFISGDNLIFKPYVAKW

SEQ ID NO: 6 - protein, 732 aa, Campylobacter fetus
MFEKTLEQILSDKNIQIALNSLKKTALGIDNLSELNEHFIHKLKQSCLNQTYAPEPVLQKLIPKSDGENYRKLAISSLKDKLIQKVLANELTWYFDKHFSDKSYAYRPGKSYKNAIFRLRDFLRVKPYFVIKSDIKDCFESINHSKLVALLAKYIKDKRVLNLVEIWIKNGIFNRQTYIKHSKFGIHQGDVLSPLLANIYLNQMDKFLETNNEIFIRYADDFVILVDDEKFVQAKINSLKTFLSTIDLSLKDTKTAIYSPTQSFEFLGVSFYGSNLSINEAKFDKIQEKIYALSKSNDFKTDFNSYIAHLQTISLNLIKTDQTQLKRFIYALKKCVSLYIKNKTTLKTRLEIFTFLDTLNFALNFKSKGEKDEFYNQIYALTREKQALKAKPQPNPQHVLNKKKKHYLEQFAQNSLLHISTPHCFLGVSNVNFVIRYKGKVILKVRIDQIHQIIIACDISLSTNAIKAATKRNISIDFLGFNNQIYASLFSHTSTITPAYKAQIDFLNSPNSLNLAKEFIKAKATNQINYLKYLDKHYKILASNIDKMHKNLKKALISATTTSELMGYEGAISSLYFDAIASTLEDKEFKRVGKGATDLVNSLLNYGYAILYSTVQSALIKAGLYLNISYFHVSAKFSLSFDFIEEFRVVAVDRVVFTLLHQKTKLSLKDGLLDVPTKKKLTQKVALALLSTHKYKNEELNLEQIIQAQAYLLRKQIFNEAKYKGFLVRFQG

SEQ ID NO: 7 - protein, 1014 aa, Teredinibacter turnerae
MVTRVEEFDPLPLENLEQAALLEQLLPLRSLAVHLQFIDESHPRFFHQAAVSALLRHLLPSADDYSHYLILDAPETARINYQAGASYCFSIVCLAGGESLLATLMLALRQLPYSAPTGNPLASFGANLRWRGFSCNFCDHPVDQVEDLGCYGIEHLAQEAALWAEHHTLFHWQWLTPVRLLKEKSARSTAKGEARYCADAGDLSAALLLARLYDTVNALLTERGDRPLPPRQAAPHMNVEAQHCFWLDAGYQNSSGKEQVMGGLLGEFTYSTPVPLPPPWAHFLVLGQYIGLGQRRSFGWGRYQLVTTENQVSCRRQLAAQPLLERALEPANLRLALQHKTQKTKAKREPLYKWQRDAQECDLSQYECNEETDAEGDQAELPPTLLKRANALAQGRYDVPPLRGVIIPKTDGEWRALAVAPFFDAVLQRAVAQILAPSLDRVMDNRSYGYRRGRSRLDAKEQIQLAYRNGARWVLEADIEDFFDSVAFSLVAQRLRALFHQDPINEAILAWLSAPVDYDGLRLQRKAGLPQGSPLSPVLANLLLDDFDSDMRKAGFNCLRFADDFVVVCQSREEAERAWQRAASSLNEHGLFLAENKTRVISFERGFRFLGYLFVNELALDVGSKALKQHDKLSSPGHAKPQCASGWLADFLAQRPQALLPPNAPHSEQRVVEKTTAMVHSQAVALGNRENDGTFLCVSGAPALISTDHGRVLVQRDDETVMSVPWQGLRSVLLLGRHHLTHPAMIAALSQGVAIHFASRGGQYQGLLDGNQPRLGPRLWLLQEERVADAAACVAVARELTNARIRHQREVLRQRALSGWHTLGDSLSQVDACTDLSALQGIEGAAARTYFAALAQAVHPQWGFHGRNRRPPRAPFNALLSLGFTLLYAHTETLLIIDGLNPRAGFYHKPHGSHSTLASDMMEPFRHLVERCALSFLSSGKVKPADFSVKDNGACELSNAARRLYLERLSERFETSMKGRDGSEGKLIQLLRWQNRSLIELIRAKGPFTAWLQR

SEQ ID NO: 8 - protein, 653 aa, Woodsholea maritima
MMRLEACLTVKSLMTAWKKVYANQGGPGGDGQTLAQFQRTVLLHLHRLGDDVRAGLYMPGPHRVVSIPKRAGGWRSLSIPCVRDRVLQTAVAQRLQPILEPEFEPESYGYRPGRSVAQAIARVATLRRQGFRWTVDADIERFFDCVPHGPLLERLRPFLGDPGLVGLVEMWLAGAGPHGRGLPQGSPISPLLANLYLDDVDEGLKSTHTRLVRFADDFVILTRNEDEALQALERARGLLDKLGLSLNLEKTRIVPFEGGLDFLGRKFVRALVVDDVYEDEGEALAAGEPARTHARAMPGEDALDLASLEAGADTSRAPRLRVLYLLEKGHVLGTHNSSFTVRNAQGDPVSTLPTTRVDRIEVGSQARIADEAIRLALDEDVEMRWINGRGQTEGYLSRPERGHGALHLAQLRLYDNAEARLAAARILVEGRLRNQRALLRRLNRRRKRPFIAERAKQIGGVLKYLPEAQTIEALMGREGQAGALYWPALGACLEHGWTFTQRVRRPPPDPVNLVLSYLASLLCRDIASLAARQGLHVGIGALHAVQDEPRDTLAFDLAEEFRAPLVEGLCIWMLNTRTLGHQMFHTREDGQVYAHGEGIKTILRSWENWLDRPVRSPRSGQDVLWRGLIEEQILCWRDHVSGKSVYQPYRMEY

SEQ ID NO: 9 - protein, 655 aa, Desulfarculus baarsii
MVDMRLYAKIIAKSSLMLAWEKVLANKGAPGGDRQTLDDFAESLERNLEGLHAALRSASYRPGPIRNVSIPKRDGSPRRLSIPSVADRVVQTALCQGLTPILEPEMEDASFAYRPGRSVQMAVERVGRYFRQGYHWVVDGDIDDYFDSIPHHGLMAVLRRYVDDQDVLGLIAQWLAHAHAGGVGVSQGSPLSPLLANIYLDDMDERIGRTGARLVRFADDFLLLCKSEERARESLAAMSALLAEYGLGLNPDKTRIVNFEQGFEFLGRLFVRSMALEREQESDAPQETPPGPTPDDPSPPVEPLHQASEGPGFQDLSPRLRVMYLSRKGCRLDVRGRAFVVRSGPEPDAPELMVVLPSQLDRVELWPGCDISQKAQRFALECRTPVAYVDGWGRTLGVLEPMVADKAALHLAQAAVALDETKRLALARLICAGRVRGQRALLMRLNRRRKNSDIESNLAAFKQLPRRIATATTISELLGLEGEAAKRYWASLALLLDKSWGFSSRQRRPPRDGVNMVISYVASMLYRDLRCLAARHGLHPGFASLHGSLDGKPGCISDLVEEFRAPLCEGLAVYLANNHILKKEMFYKTDKWPCHVTPEGRETIIRAYEAWLDRPVKSPRSGEKVKWRGLLEEQVLAYRDHVMGRSVYAPYDMKY

SEQ ID NO: 10 - protein, 672 aa, Azospirillum lipoferum
  source 1..672: note = strain = 4B
MPTAPPDNADLFEEVTRLDTLERAWGRVLRNAGAAGGDGLTVGRFAEAAPSRLLALHRTLRMGDYRPGPLRRLSIPKPDGALRPLAIPPVTDRVAQTAAALVLTPLLDGEFEDASFGYRPGRSVPQAVARVARWRDQGYDWVVDADIERYFERVPHDRLLIRLERSIGAGPLTELIAVWLESGAENGVGLPQGSPLSPLLSNLYLDDLDEALDGRGLRLVRFADDFVLLCRSRERAERALDHAAAVLEEHGLRLNRDKTRIVPFDQGFRFLGHLFVRSLVLPSPRPDDGEETEGDALLRALAIRDAAAEAEEAAAEERDRQSRAAGLDPALRTLHVQTPRRRLALRNEAFTVVERDDLGGERELIAIHHHHLDRIDLGPEADADAEALRWALATDTELAFVDGHGATHGRLTRPEARRAALHLDQARHALDEGLRLDLARRIVDGRLRNQRALLRRLNRSRKLGVVEDAVLAINALLRRLDTAADVAALLGFEGQGAARYWPALAAQIEGEWEFEGRRRRPPPDPVNAVLSYLSGLLERDVAALIARHGLHPGFGVLHSPQDRHDAGIYDLMEEFRAPLMEGLAVTLFNRRTLRPDHFSRRETGEGEIKGCRIDPDAVGAIIRAYEQWVRRPVASPRDGKRTTWRGLIGFQAQALAAHVQGREPYRAYVMDY

SEQ ID NO: 11 - protein, 617 aa, Cellulomonas bogoriensis
MARVADEGALTRAWSEVLEAAERDGRVPVSVRRFERGVAASLVRLSGELSSGRYQPSRVSEVSLRTGSGSERVLRIGAVVDRVVERSLLNALTPVIDPLLSPFAFGFRRGLGVKDAVAALARARDEGSTHVLRSDIAAAFDSVPRARAVQALSRLVPDRRVCDVVASLLARLDDYGLEGVGIAQGSAVSPLLLNLYLLPFDEALMANGFTPLRYADDIAVPAMSESQAQSAAQDVAHQLECLGLACSAPKTSIRSFDEGVHFLGVTLRARPSSTAPPPARSRPQRISMVVSEGGAVVRTRRGRVRVDRDGETVASMSLSRVARIVVQGRVGLTTPLLHEAAQRGIDVVMLSRSGGYVGRLSRRRPGDPSLRRAQARAYDSGADLERLTTAFVSGKITNMRVAVLRHQRGAGTSEEGARVAAQLAEARARASVMRSVPSLMGVEGAATRAYFGWLGSRVGEEWGFHGRARRPPPDPVNSMLSYGYVLLCAEGVSACEQAGLDPDMGFLHSDRWGRPSLALDLMEEWRPVIVDSTVLRIISNKRLKPSDFTFDAKQGARMTAHARQTFLREYEARMLTLAGSDAGAGRQPYRRLIATQAMRLAEALRTPGGPYRPYVWR

SEQ ID NO: 12 - protein, 558 aa, Micromonospora rosaria
MTRVSIPKPDGGIRSLAIGAIEDRIVERAVLDVLDPVVDPTLSPWSFAYRRGLGVRDAVRALAEARESGLAFVVRCDIDDCFDSIPRWPLLRRLRELVSDAELVALVERLVGRPVTGERASGGRGLHQGGSLSPLLANLYLDTFDRALMRHGHRVVRYGDDIAISVPDRPTGLRVLDLADAEAEALSLRLNTDDRQVIAFDEGVPFCGQVVTASSGPTADLQAKPLQGTVFVTTQGALLRVKGERLRVEDGDRLLANVNLKRVRQIVCFGRVGVTSTLLQRIVERGIELAWLYEDGRHAARVSGLDGTDPEVRLAQYRAADDARQALRIARQLVAGKVTNMRVGLLRAARAQQAPELADRQARLATARQSALIADSTAELMGYEGSATRDYFAGLSQILGPEWGFTTRQRRPPPDPVNAMLSFGYTLLTNEALTACQLAGLDPYLGMLHSPRRNRPSLALDLIEELRPVVVDATVIRLVRTGQVTPKNFTLTDDRGCRLDDHGRRAFLDAYERRMLTLVHHPVEQRRIPWRHVLLAQARTLAAVLSSRRPEYRPVVWR

SEQ ID NO: 13 - protein, 668 aa, Tolypothrix campylonemoides
MFTIEQITSAWLLVRAGSRGAGVDGMTVDLFAAGVNEQLRILLRQLQQESYRASPAKGFFVAKKSGGKRLIGIPTVRDRIVQRLLLEELYFPLEDTFLDCSYAYRPGRNIQQAVQHLYSYYQYQPKWIIKADIAEFFDNLCWALLFTALEDLQLEPILLQLLEQQLKSGIVIAGKPIYPGKGVLQGGVLSGALANLYLTSFERKCLSYGINLVRYGDDFAIACSSWLEANRILDKITTWLGELYLNLQPEKTQIFAPDDEFTFLGYRFAGGEVYAPPPPVMTREGEWVTNESGLPYFRPKSRPVKFVSRPPKACSIASPIKFPTAPISHLWQEFMTTLYVTDQGAYLSVKNQQFQVFYQGELKIKVPATRVNNIVMFGCCNVSHGAVSMALRRRIPIMYLSQKGRYFGHTAVQGDARVEYLMQQVKCCENTQFTRQQAEAIVAAKLHNSRILLMRLNRRRPTEIATQAIDLIEILIDSLPQAESLDALRGYEGKAATVYFQALGSLFTGFFAFDKRTKRPPTDPINSLMSLGYTLLSQQVFSFIQSVGLHTHFGNLHTPRDNHPALVSDLMEEFRAQIVDSFVAYLVNKKILTPEDFTPPDERGGVYLQASALKKYLKHWEEKLQTETTHPHTGYKVAYRRCIELQVREYIACLVGEVEVYRPMVWKL

SEQ ID NO: 14 - protein, 672 aa, Oscillatoriales cyanobacterium
MEFTPEPLHAAWLQVRKGSRSAGIDNITVDLFAGVARYQLQVLLWQLQQENYFPRPAKGFYLRKASGGQRLIGIPTVRDRIVQRFLLDELYWPLEDVFLDCSYAYRPGRGIQMAVKHLYSYYQLGQAWVIKADIEKFFDNLCWPLLLTDLEKLQFEPILRQLIEQHLASGIVVKGQHFHPNQGVLQGGILSGALANLYLNEFDRLCLSHGFNLVRFGDDFAIACADSIQANRCLEQISSWLGSFYLKLQPEKTRIFAPDEEFTFLGYLFRNGEVFAPEKAQPTTQVIRASGTSFSRPATRKVPVFSGKPLACSIDVKPIVLPRANSEHFWREPMSTLYVTNQGSYLSIYNQQFQVFYQRELEIKVPASRVSHIILFGCCNLSHGAVSCALHRRIPILYLSQRGRYFGRLQSDGRAKVEYLTRQIICSLNPEFVRLQAEAIVRAKLHNSRILLMRLNRYRKSKNADEMSVLQAVEMLEILMDSLHKADSMDALRGYEGKAATVYFQALGSLFSGPFAFEIRTKRPPTDPINSLLSLGYTLLSQNVSSFVEAMGLHTHFGNLHVPRDNHPALVSDLMEEFRAQVVDSLVAYLINSQIFIADDFTPPDERGGVFLQPHALKKFLKHWDEKLLSEVTHPHTGYKVAYRRCLELQVREYVSALMGEVEVYRPMMWKI

SEQ ID NO: 15 - protein, 1002 aa, Psychrobacter lutiphocae
MVTASHTLDWQSADCQLIWHSDTTHRKPENIYIRLWGLIQTINTTEDQYLEQLHRCRDEHKKNIIIQHLEAKKQYFNHPNFPLIAFHPTQTSYHKVSLKKGEIIPISLVFGGKYSHLAQAWLDWFAVRLTLENTGFSLANIPSLKPHQIQLPPLTANKLAKCQIANKQAKAPIDAQELETAPVQGSPQEMTILIDTPANFSLNKTQQKQVSNLSTADILKYAFIHNSQKRLCQWFPQQNKVIESWFKQWTPLLLKSPLSTYQLHERHEIKTVSKSSSARSQSKIQFHKGHAGWLTFKGAWLEVDELLTVLSSIHLLGQRMHINGLGYFLTQQQLEHSAPTLWQRHLMDKHKIKHAIEEVLERHELEPVVDDKGRIMQVDALTELIYTQLSTGVYAPKPTRAIFVPKPKGGKRCIEELEQVDMMVHRLVFNSIAKTIESYQSPLSLGYRKGYSRQMARDKVQALIDSGFGWVVEADIESFFDNVPFERLWQRLATILPQRELQTIALIKKLMQVGYTVSNASGTVVKEHLRFKGLMQGSPLSPVLANLYLAMLDEQINAEHFAFVRYADDVLMFCRSEADANTTLAWLDQHLSELGLNLSLSKTAITAVNNGFEFLGYRFDKEGSDDKSIVPVLRQRKPIMITGSRKYLGINGGALEVRQKQARKQPGRSLSTSPLSNQLVQVIPLRRISQLVVMGNHSLSSPLLTACAKHHISVHLVNQWGFQVGTFKPSHAEYYAVSAEQYQRHQQLKPSERMAIAADLVLAKINNYQTWIINSYRKGDAQVNKQLDQIANQASSATTIEALMGYEGQAAKICFQRLQSVMIGDQQAAFSSKRRSRGGPDRLNSMLNFGYYWLFTRISGLLHSHGLNPYLSFLHEPEQNYETLVYDIMELFRVQVDKTVLRLINRKQIQADSFHLDAKKGWKLNNDALHLLSNQLQSTFASKINQTFLEDIILIQVRTLLHWATQQQSLVWFYWYADKDNVQFSLPDESPQTLIIDNIESQ

SEQ ID NO: 16 - protein, 633 aa, Ruminococcus sp.
  source 1..633: note = strain= TF08-4
MFSMEEMLSKKNQREAFSFLKNKKDGIGADGMPLSELEDYWKINSERICSELKKGKYTPGIIKCTEIINNRGKRRVISNLCTVDRFITRLLYQKLRRYISPEFLENSYAYQENKGILNAVQKVQKYISEGSKYTMEIDLKDYFDTIPHEQMMSLIKERITDDRVVDLIYKYMHCSVSQDNEIKEKKKGLVQGNAISTVLSNLYLHSLDQYLQEREYKWVRFADNINVYCRKENDAIRVYNEINIYIKEKLHLTINEKKSGIYPVLDRIFLGYKFYKYNGKYEAKKYKYQKTDCFHNWHESALQRVNHEYHIVQNGVLNKKDYSLIFENEEEKCDIPVGVVDQINIYSEVTIAATALQLISKKKIRLSIIDKYGNLIGNYIPAGYGRSSIEFLKQAVFYESSHRFEIAQKIEVASIHNLRANVKYYSRKNAHALDAVVEQLSEYMNEMRNAPNIEELMIIEAQARQQYYASFNIILKQESFFFEKRTRRPPKDAINALISFGNTLLYNFVLQAIWKTSLDPRIGIIHATTNRNYSLNLDFADLFKPVIVDRVIFSLVNLLQLQSEVHFEEKESGAIYLNKEGKRIFIKAFEEKLADKIKLGEKTYTYKQIIEEEIRKFQRYIVKEEKYKPYKYW

SEQ ID NO: 17 - protein, 936 aa, Vibrio sinaloensis
MTNPILNSIEPIAQLLPLRAMVVTLRFTQDATFQFYHHVAAHAWVRHLCGSPDNFSQQVTVQTLENGKTDYKAGELYRFKLVFTPLGGNLIQPLIDALERLPSSAKALPRGACINNNVELVELKCGVGNEEIHHAIDLYPYDLQALAQDVAHWQQRDDIYMESLTPARLLKEKASQESSRGDARYCRDKVHFSATTITRRFVDTIIGLVESHTGQRYQRDIEASFEIQPELSFWVEHRYGRGEKWQKKPMSGALFSLKVRNVPRLEKWQIALLVLGQWLGIGQSRSMGLGLYWLRDVSETLGGHSNRLIKEYFNQRRLVQLVQESLDSQFSEQERNQYKNKVIGLSHTILAGDYKAPVLTQVEIDKSDGGVRTLSIPPLADRILQKAIARPLAVSLDGLWKTHSYGYRKDLSRHDAKFAINQAIQQGYEWVLESDVDSFFDNVDWRNLQTRLKLLLPNDFLVDVIMAWVKAPVKTPSGQILERTQGLPQGSPLSPLLANLVLDDFDADMLALDYKLIRYADDFVLLFKKQSEAQMALDHVIASLNEHGLNIKAKKTQIVHANKGFRYLGFWFVDGYAIETSRHYRQEEQEYQQAITHHQAQLAESKQREKQQIGEREQLGTLLVIAGEVAMLTCENKRLKITQQDESRYYAWEELETILILGPHNISTPCIRQAMHHQVAIHFASRYGQYQGVACSNMPSQGHQLWQLQIAYLQKVDVALTWSIELVCAKIDGHIHLIRNRERQSPLLEKLRTIKRKARRSDDLQVLLGIEGEAAKLQWEFFKQHLDCEWQFSGRNRRPPKDPINAMLSLGYTYLYHLTDSLIQSNGLCPWAGFYHQPHGAHRTLASDLMEPLRVVVDRTVLALVAKRQIKPDDFLILEDGCEMSREARKVLLTQLLADLTTKRKKSDRVIDQMITQVKEVKIATKLALNPNFWRP

SEQ ID NO: 18 - protein, 632 aa, Blautia sp.
MFTIDEMLSKNNQRLAFEHFATKNDGCGPDGMHVSELEKYWRMNHDQIISDLKNQEYQPGIILIREHMNKTGKRRNIASLNVIDRFITRLLSQKLNRYLAPIFCENSYAYQDSKGVMPAVLKAKEYVELGMRHVIEIDLKNYFDTIPLENLIPEIERYITDEAVLHLIKQYLFCDISFEGKISRKTQGIVQGNAISPILSNLYLNDFDKELDESKLCWIRYADNIYVYMDSYEKALLVYSELTERLERRKLTVNKEKSGVFDVSTRSILGYDILIRNKKVDVRKHIYKSVNQYSNWHDSRLEFINGRYHITSDGILNRQDFGLLFENEQKKHYIPVEVSDQLNIYGNVTLASNVLQSFSNREIKVSFFDKYGRLIGSFLPEKTKKSAEIILVQSKNYLNEDVRMDTARRMEIAGLHNIRANLRYYDKKHKGDFKEKVDAISGYIDALNRAPSVNDMMLLEAKARQLYYTCFNQILETSDFQFEKRTKRPPKDAINACISFGNTLLYNLFVNIIWKKGLDPRFGVVHASNKRNQSLNLDFADIFKPIVIDRIIFTMINKKMLTLLTDFETSNQGVYLSREGKNIFLQMYEEKLKSRITIKGKEMSYYQLLESEVQNYKNFILTGETYKPYKYY

SEQ ID NO: 19 - protein, 374 aa, Rivularia sp.
MGYENIRYLHKPYFAETITEKPPQTEEDDSPFQKNIWNAEMAAIYLIEQGTNIYKDYQRFIIHVSEKPKLEVPIRDVQQIIVFGNIQLSTPVIQACLKEQIPVVFLSQTGTYHGHLWSEKSIHLDNQLVQAERRNDDLFRFSVSRAVVLGKLLNSKQLLMRFNRRRKIGKVEEAIYGINQDIDALNYVDNLDTLRGYEGIAAARYFPAFGNLITNPKFSFSQRFRQPPTDEINSLLSFGYTLLFNNVLSFIITEGLSPYIGHFHYGDKQKTYLAFDLMEEFRSPIVDSLVLKIINKSLFKPQDFDVVASTGGVYLSQTSRRVFLKQFENRMNEEISHPDLISQVTYRHAIQLQVRRYKRCLLSDNIYESFLRAD

SEQ ID NO: 20 - protein, 320 aa, Rivularia sp.
MISSQFIDINNFQRAWEKVADKRGCAGVDGETISSFASNQTVNVYQLMNSVADGSYQPFPCKQVIIPKRNGSQRELKIPTIRDRIVQQALLNVISPLMEEKFSPVSFAYRPNLSYINAVEKIADWRDMGYVWVLDADIVKFFDNIDHHRLLQQVRLHIDHPGILCLIKAWISVGVETREGLILPQKGIPQGAVISPILANIYLHEFDEIISASDLEIVRYADDFLVLSTSQERIAIAKSQVIDLLDSLGLEINTDKTQITSFERGFRFLGHGFLSDAIFPVDTNKAKLKSGIETNREKTRTRKTSKKKLYHNPYRNKKVV

SEQ ID NO: 21 - protein, 86 aa, Fusicatenibacter saccharivorans
MYVILVYDIHQKRVGKALKICRKYLIHIQKSVFEGNITESKLKALKEELGHLIDTQMDSVIIYHLDSVKYTKKEQIGIVQSTSNVI

SEQ ID NO: 22 - protein, 93 aa, Candidatus Accumulibacter
MSDMALYIVAYDITDDRERRQAEHILQGFGFRRQKSVFECRLTRGYLARLQHELATIGMETGFVMIYHLAPNTRPFTIGEVPHFPDDDWAWVT

SEQ ID NO: 23 - protein, 94 aa, Candidatus Accumulibacter
MEQTWLVTYDVSDDGCRRRVERILLGHGEREQYSVFRCRLSSREVRDLRARLAGHLKGSDSIRYYPLCAACLPRQPQRTLVDSAEAGIAWYFAV

SEQ ID NO: 24 - protein, 86 aa, Eubacterium saburreum
MFVIITYDVKAKRDPKVMKTIRKYLTHEQRSVFEGLITPGRLKHLKEELKRIVNVSEDCINIYSLETLRYSKKESIGKQVYHGNII

SEQ ID NO: 25 - protein, 96 aa, Bacteroides fragilis
MVKAKKIFCVVAYDIQDDRSRIQISKILEKYGTRINYSVFECMFTDRQFQKIQINLERWINRRYDTVVYYPMCINCYTRIIYQPIRKKIIKTVEIV

SEQ ID NO: 26 - protein, 90 aa, Campylobacter fetus
MHYIICYDIAQTRRRTRLATLLEAIGTRANKSVFEAKLTAKELELFIAKAKAIIEPKTDSVLIYPLCFDCMIKSLSLGQKGVFEVKDSFV

SEQ ID NO: 27 - protein, 92 aa, Teredinibacter turnerae
MKTYLVCFDITDDKSRNKVGNLLLAYGERVQLSVFEIALNNNRELAALKTQLQTLMEEGDDLRFYYLSKETRRQSEDVYGNAIADFPSVIIV

SEQ ID NO: 28 - protein, 93 aa, Woodsholea maritima
MADPLFVFAYDISQDRVRRRVAGLLEAEAVRVQGSVFEARLSKSRAKALGQRIAIELERGDTLRVYCLSQRDLKSSFQWGGAPMAEAQDFYIL

SEQ ID NO: 29 - protein, 95 aa, Desulfarculus baarsii
MSGAEMLVVFAYDVEDDSRRRRLARVLGNHAVRVQKSVFEAWLDEGAAKIIASRAAAELGPRDSLRVYALDASGVGKTLVFGVTAPPQSHDYYFV

SEQ ID NO: 30 - protein, 95 aa, Azospirillum lipoferum
MSETPMVMVFCYDVANDRRRRRVSAVLEEWGVRVQKSVFEARLTEPQARALLARAGKELGPRDSLRMYALSAHGLAHSAALGGAPVPEQQDFWLL

SEQ ID NO: 31 - protein, 92 aa, Cellulomonas bogoriensis
MIWILACYDIADDDRRSRLSDLLAELGPRVQQSVFECRLPSKKALRRLLGELAALIDPVEDQVRVYELGGQGPRPQIVGTRILEEWRDMWVV

SEQ ID NO: 32 - protein, 95 aa, Micromonospora rosaria
MSRVCYVVAYDIADDDRRADLAMFLSGYGPRVQLSVFEVELPDADTAVAFRERLRSLIDPNDDQVRLYRLTPQALGQRIIYGRRTIEERVDFWIV

SEQ ID NO: 33 - protein, 92 aa, Tolypothrix campylonemoides
MFYLVCYDIVSDNRRNKVSKLLEAYGLRVQKSVFECVLDEKQYEMLSKYLMRLVNRREDQVRFYPMSAHNRCKVAVLGTQPEFVVDDAAFIV

SEQ ID NO: 34 - protein, 92 aa, Oscillatoriales cyanobacterium
MFYLICYDIVDDRRRVKVSKLLETCGCRVQKSVFECVLDEKRQEQLQKRLLKLLNKRQDQIRFYPLSEHCRCKVTVLGVQPKFVIDDEAFIV

SEQ ID NO: 35 - protein, 97 aa, Psychrobacter lutiphocae
MSNVTKQQAPLYVFVYDISNDKERSKVDGVLRDYGFRVQKSVYECHLTRGDKKRLLDKLESLTIETGHIRCYGVNSKVIKIGAPPEDLDEEYIYFIE

SEQ ID NO: 36 - protein, 86 aa, Ruminococcus sp.
MFVIVVYDINRKRVGRALKICRKYLVHVQKSVFEGNITEGQLKQLKNELFQLIKIEEDAVCIYRFESPKYARKEELGTIERKSHII

SEQ ID NO: 37 - protein, 92 aa, Vibrio sinaloensis
MKVYLVCFDIENDKKRRRLSKLLLSYGERVQYSVFEITLKSDASLKTLTKQCKRHVEQGDSLRFYALPFNARQASFDIDGEPIARFPQAEIL

SEQ ID NO: 38 - protein, 86 aa, Blautia sp.; identical to SEQ ID NO: 21

SEQ ID NO: 39 - protein, 91 aa, Rivularia sp.
MLVVVVYDIPNDKRRTKLSNFLEGYGQRVQFSVFECFLNLDEMRQLFEKSKKIVKPSEDNVRFYWISEDAVSRVLTIGSEHPNAPPNYYVI

SEQ ID NO: 40 - DNA, 347 bp, Fusicatenibacter saccharivorans
  source 1..347: mol_type = genomic DNA
agaattaaattggaaaaagtcggtcgatctcatgcctgaaatcatgaattccgcaaaatggcggaaatttaaggaaaatcaggaatctcagaaaaacgatcgaccgacttttgtgataaaatggttgcaaaaaagagaaaaatttgatttaatagaatgtgaaaatagcggaaatgctgatgttgtaccttacctatgaggaattgaaacgctttttcgattgcttcttctacaatctgctgagctttgttgtaccttacctatgaggaattgaaacttgcattcgttacaatatttgctgtcagtcttggtgtgttcgttgtaccttacctatgaggaattgaaac

SEQ ID NO: 41 - DNA, 300 bp, Fusicatenibacter saccharivorans
  source 1..300: mol_type = genomic DNA
gctgaaaggaggaactatatccggataccaggtatgcctgaaatcatgaattccgcaaaatggcggaaatttaaggaaaatcaggaatctcagaaaaacgatcgaccgacttttgtgataaaatggttgcaaaaaagagaaaaatttgatttaatagaatgtgaaaatagcggaaatgctgatgttgtaccttacctatgaggaattgaaacgtcccgcggccgctattcttttgatttataagggattttgcgatcctctggagtgaataccacgacgatttccggcagtttctccaca

SEQ ID NO: 42 - DNA, 300 bp, Fusicatenibacter saccharivorans
  source 1..300: mol_type = genomic DNA
gctgaaaggaggaactatatccggataccaggtttcaattttgattctgtaatatttccctcaaatacagacttttgtatatgaatcaaatatttccggcatattttcaaggctttcccaacgcgtttctgatggatatcgtataccagtattacgtacattcaattcacacctttgttttatgtttcaattcctcataggtaaggtacaacgtcccgcggccgctattcttttgatttataagggattttgcgatcctctggagtgaataccacgacgatttccggcagtttctccaca

SEQ ID NO: 43 - DNA, 354 bp, Fusicatenibacter saccharivorans
  source 1..354: mol_type = genomic DNA
cggggaagcttatgttccatagcaaaaagtcggtcagtctcgtggctgaaatcatgagttccacaaaatggctgaaattcaaggaaaatcaggaatctcagaaaaacgatcgaccgactttttcgataaaatggttgcaaaaatgagaaaaatctgatttaatagaatctgaaaacagcggaaatgctgttgtcgtactttacctaaaaggaattgaaactattatatgagacttcaattgctgcatattccctagcgtcgtactttacctaaaaggaattgaaacacttaccagaatatacgtattacgtaaatcctttgctttgtcgtactttacctaaaaggaattgaaac

SEQ ID NO: 44 - DNA, 300 bp, Fusicatenibacter saccharivorans
  source 1..300: mol_type = genomic DNA
gctgaaaggaggaactatatccggataccaggtgtggctgaaatcatgagttccacaaaatggctgaaattcaaggaaaatcaggaatctcagaaaaacgatcgaccgactttttcgataaaatggttgcaaaaatgagaaaaatctgatttaatagaatctgaaaacagcggaaatgctgttgtcgtactttacctaaaaggaattgaaacgtcccgcggccgctattcttttgatttataagggattttgcgatcctctggagtgaataccacgacgatttccggcagtttctccaca

SEQ ID NO: 45 - DNA, 300 bp, Fusicatenibacter saccharivorans
  source 1..300: mol_type = genomic DNA
gctgaaaggaggaactatatccggataccaggttttgttcggtgttatttttgtactcatttttagagaaaataaagaagaatggttatggcaaagaaaaaggcagcaatatcatcaccgcttccaagctctgacctggttgatctttatattagagtttctgtgccacattggggtctgaccccatatagcacctttaggtaaagtacgacgtcccgcggccgctattcttttgatttataagggattttgcgatcctctggagtgaataccacgacgatttccggcagtttctccaca

SEQ ID NO: 46 - DNA, 275 bp, Candidatus Accumulibacter
  source 1..275: mol_type = genomic DNA
ctcttgccaaaccctgtggataagctgctagacttgccggagtcatcgaggttgggcaagcgacggtacgcaagtctccggcaagtcagccttttttgacacccagtgctgaatagggaatcctgttcgaacaggcttgaaacgaaaagagtcactatataatcttcaagtgagctgaatagggaatcctgttcgaacaggcttgaaaccctctggaagttccacctcatccccaaagtgctgaatagggaatcctgttcgaacaggcttgaaac

SEQ ID NO: 47 - DNA, 307 bp, Candidatus Accumulibacter
  source 1..307: mol_type = genomic DNA
gctgaaaggaggaactatatccggataccaggtggtaccccattttcctgatgacgattgggcatgggtgacctgactcttgccaaaccctgtggataagctgctagacttgccggagtcatcgaggttgggcaagcgacggtacgcaagtctccggcaagtcagccttttttgacacccagtgctgaatagggaatcctgttcgaacaggcttgaaacgtcccgcggccgctattcttttgatttataagggattttgcgatcctctggagtgaataccacgacgatttccggcagtttctccaca

SEQ ID NO: 48 - DNA, 393 bp, Candidatus Accumulibacter
  source 1..393: mol_type = genomic DNA
gaacttcggggcatatggcgctggtgcacattttgccggacatggctttttggacaatggcttgcgaaaatacttcggcatacgggcgccgcgtgttgattttcaagggaaaagaggattttcgtgcgcctggacgggagcgcgggagcgttgtttcgcccgcgccccgaaaagagccgttaaattccttgacgatcatcgggttgtggaagtagagtgaaaactaagccctgccgataaagggattgagacctgtccaacaaatgcttctttggtaccgttttgaggtgaaaactaagccctgccgataaagggattgagactcgaccacagggtcgactaggtcgctattgtacagtgaaaactaagccctgccgataaagggattgagac

SEQ ID NO: 49 - DNA, 307 bp, Candidatus Accumulibacter
  source 1..307: mol_type = genomic DNA
gctgaaaggaggaactatatccggataccaggtaaaatacttcggcatacgggcgccgcgtgttgattttcaagggaaaagaggattttcgtgcgcctggacgggagcgcgggagcgttgtttcgcccgcgccccgaaaagagccgttaaattccttgacgatcatcgggttgtggaagtagagtgaaaactaagccctgccgataaagggattgagacgtcccgcggccgctattcttttgatttataagggattttgcgatcctctggagtgaataccacgacgatttccggcagtttctccaca

SEQ ID NO: 50 - DNA, 307 bp, Candidatus Accumulibacter
  source 1..307: mol_type = genomic DNA
gctgaaaggaggaactatatccggataccaggtcaatgaagaggtttgtgccgggcatttactccagagcatatttattggccaggcttgggaaccgctgattaacacatccttcggagaaacctggcaacgcgaagccgtcctcggtcgtgcccaagtgaatggtgatgactggaggatgaggtctcaatccctttatcggcagggcttagttttcacgtcccgcggccgctattcttttgatttataagggattttgcgatcctctggagtgaataccacgacgatttccggcagtttctccaca

SEQ ID NO: 51 - DNA, 612 bp, Eubacterium saburreum
  source 1..612: mol_type = genomic DNA; strain = DSM 3986
caggggcattgattttctcctattttttatgtacgaatcattatagaataaaaagtctgtcgccctctatttttgcttttaaatatgtaattacatattcatactatagtatgtgtttgtaattacattatttgattaatttttatcatatcttgatatataagttcttgttaagtgtttttaatataagtttactgttttaaacggattttattcgaaatagtgctatatttttgctttaatatacttatttttaaaacaatacttacaattactcttttcatgcaataaagaaaaaaagtctgtcactgattgtatacaatcacgacaccctgaaaagtcccattttaagccattcttaaaaatagattgacagactttttagcctgtaatacaatagttttaggtcaaaacaagagtttttacctatcacaacggcttaaatgcagttgtaagtaccttacctataaggaatggaaacgaataagctgcatagcagtgattccaatctgatttagtaagtaccttacctataaggaatggaaacaattcaagtagacaaagcctgctggatagtacttggtaagtaccttacctataaggaatggaaac

SEQ ID NO: 52 - DNA, 630 bp, Eubacterium saburreum
  source 1..630: mol_type = genomic DNA; strain = DSM 3986
aatgtatgaacttcatatactcatgccatttatatatatttagtgaaaaagtctgtcaatctctacttttactttacggatatgtattacaaacacatattatggtatgggttatgcatgtatattgcatgttcttctttatcttattttttataataatatgatacaaaacctctgtagagctatttcggatgtagatttaatattttaagcatattttacttcaaaaaagtatattatataattttaaatactgcatttattatacaatatttacaaagagcacttttgctcattaagatcaaaaagtctgtcactgattgtatacaatcagtgacagacttaaaagcaccattttaagccattcttaaaaatagattgacagactttttcacctgtactacaataaatttagcacaaagcgagggctttcccatattacaacggcttaaatgcagttgtaagtaccttacctataaggaatggaaactggtcgaccgtcacattctgatgagactgatccgtaagtaccttacctataaggaatggaaacgaaacccatccacccataccttcatcaaaatattggtcagtttctccgtaagtaccttacctataaggaatggaaac

SEQ ID NO: 53 - DNA, 301 bp, Eubacterium saburreum
  source 1..301: mol_type = genomic DNA; strain = DSM 3986
gctgaaaggaggaactatatccggataccaggttctgtcactgattgtatacaatcacgacaccctgaaaagtcccattttaagccattcttaaaaatagattgacagactttttagcctgtaatacaatagttttaggtcaaaacaagagtttttacctatcacaacggcttaaatgcagttgtaagtaccttacctataaggaatggaaacgtcccgcggccgctattcttttgatttataagggattttgcgatcctctggagtgaataccacgacgatttccggcagtttctccaca

SEQ ID NO: 54 - DNA, 301 bp, Eubacterium saburreum
  source 1..301: mol_type = genomic DNA; strain = DSM 3986
gctgaaaggaggaactatatccggataccaggttataatcgtttcaattccttataggtaaggtacttacgcagaggctagaagaaaagatatagaagagcaattgtttcaattccttataggtaaggtacttacgagaactcgataagcataagaatgatagatttgtatcaccagctaatagtttccattccttataggtaaggtacttacgtcccgcggccgctattcttttgatttataagggattttgcgatcctctggagtgaataccacgacgatttccggcagtttctccaca

SEQ ID NO: 55 - DNA, 301 bp, Eubacterium saburreum
  source 1..301: mol_type = genomic DNA; strain = DSM 3986
gctgaaaggaggaactatatccggataccaggtctgtcactgattgtatacaatcagtgacagacttaaaagcaccattttaagccattcttaaaaatagattgacagactttttcacctgtactacaataaatttagcacaaagcgagggctttcccatattacaacggcttaaatgcagttgtaagtaccttacctataaggaatggaaacgtcccgcggccgctattcttttgatttataagggattttgcgatcctctggagtgaataccacgacgatttccggcagtttctccaca

SEQ ID NO: 56 - DNA, 301 bp, Eubacterium saburreum
  source 1..301: mol_type = genomic DNA; strain = DSM 3986
gctgaaaggaggaactatatccggataccaggtgaacactgaacgttgctcatgtgtaaggtattttcggatggttttcatcaccttgggatctcgcttagccttaacatcatatgtgataatgacgaacatctcttcctccttgtattttgattttagtattccaacctggtgaatataggtgtttccattccttataggtaaggtacttacgtcccgcggccgctattcttttgatttataagggattttgcgatcctctggagtgaataccacgacgatttccggcagtttctccaca

SEQ ID NO: 57 - DNA, 620 bp, Bacteroides fragilis
  source 1..620: mol_type = genomic DNA
acctcttgcgaaaatcagaatttgtttaattcattgattttctgcttattcttttaaatgaaaaataatatataatgtataaaagctacgaaagaactagcttttttgtatattagtaaatcatttgcgaaaaaaaacaaataagcaattcgttctttgacataatgcacttattatctgataattataaatataagattaatcttattttcgaaataggctataatataacaaatgaagcaggcatttgcgaataatgaatcgatcatatcaaagtgataatcaattaaatatctctatattctacaaattatttatttaaattacagaaaaaaagagtacatttgcgaaaatcagttcaacacttcatctatctaactgaataataagttattatatatttatacagatagaatacatattttataataatctgattatcaatatatgtagatgtattccagtataataaggattaagacaataggcgtagccgttcataacataactagcctctaatgtagatgtattccagtataataaggattaagacttgaattccaagacctaaagccgtaccgattgatgtagatgtattccagtataataaggattaagac

SEQ ID NO: 58 - DNA, 306 bp, Bacteroides fragilis
  source 1..306: mol_type = genomic DNA
gctgaaaggaggaactatatccggataccaggttatattctacaaattatttatttaaattacagaaaaaaagagtacatttgcgaaaatcagttcaacacttcatctatctaactgaataataagttattatatatttatacagatagaatacatattttataataatctgattatcaatatatgtagatgtattccagtataataaggattaagacgtcccgcggccgctattcttttgatttataagggattttgcgatcctctggagtgaataccacgacgatttccggcagtttctccaca

SEQ ID NO: 59 - DNA, 306 bp, Bacteroides fragilis
  source 1..306: mol_type = genomic DNA
gctgaaaggaggaactatatccggataccaggtaatgttaaaagcaataccgaagtccatgacgatcttatccaagccgttatgtctgaacttctccccttattctatttaatgtagttaaatttttatattggaatacatctacatgctaataaggaaagtttgcagtatttctgggcattggtcttaatccttattatactggaatacatctacatgtcccgcggccgctattcttttgatttataagggattttgcgatcctctggagtgaataccacgacgatttccggcagtttctccaca

SEQ ID NO: 60 - DNA, 756 bp, Campylobacter fetus
  source 1..756: mol_type = genomic DNA
gagctatttggtcgcttttgatttgtaaattttacgagttacatcgtgctatataaatttgattatctataaatttttaaacacggtttttatttttggttccgccataatttcaatatatttttaacaaatttagcactttagagtttctaactttttatattttaaaactcctaaatttagagctttttggttttttgctcttttaaaaaagttagaaactcaataaatttagcatttttaaaaacacactttctaacttttataattatttaaattttaaaagcccatttttgcggtattttaaaacttagatttttcttaaatttggaaactatacgcagtttaaaaatttccccattcgactttctaaatttttaaatctcaaaaaccaccattttaccgatactttaaggacaaatttagcaaaactatcgatttatacattttaaaaatcaatagagtttcgaataaattcgctcgaattcagctctcacaggcttttgcttaaagaatttataatcattaaaaaagtataatcatcttgcaaataactaaatttgcagtgtttagacactttctccgattattaggggattgaaacagaatttggcaatttttttaaattttaattttactagtttagacactttctccgattattaggggattgaaactttaaatctgtagagctaaaactttgtaatctaaagtctatagtttagacactttctccgattattaggggattgaaac

SEQ ID NO: 61 - DNA, 308 bp, Campylobacter fetus
  source 1..308: mol_type = genomic DNA
gctgaaaggaggaactatatccggataccaggtcaaatttagcaaaactatcgatttatacattttaaaaatcaatagagtttcgaataaattcgctcgaattcagctctcacaggcttttgcttaaagaatttataatcattaaaaaagtataatcatcttgcaaataactaaatttgcagtgtttagacactttctccgattattaggggattgaaacgtcccgcggccgctattcttttgatttataagggattttgcgatcctctggagtgaataccacgacgatttccggcagtttctccaca

SEQ ID NO: 62 - DNA, 308 bp, Campylobacter fetus
  source 1..308: mol_type = genomic DNA
gctgaaaggaggaactatatccggataccaggtataatagtaacatagtccatataagttatagcagacataaaaaattcgcccatatcttctccaagttttataatttaaatttaattatacctcttttttatttggctaaagctaaatttttataaaacctgctacctatatacaaattctgtttcaatcccctaataatcggagaaagtgtctaaacgtcccgcggccgctattcttttgatttataagggattttgcgatcctctggagtgaataccacgacgatttccggcagtttctccaca

SEQ ID NO: 63 - DNA, 464 bp, Teredinibacter turnerae
  source 1..464: mol_type = genomic DNA
cgccagtggcgaagcagagttatcggtttcgggcgaagcgaaggacaagccacgccagttcagcacaaaacaacataaaaatatgaaaattcaaaaacatataagaataacttcacgtattacattagctttgtgtcgcgcaggcaaaaaaatcaaacacttagcgttgccgcacaggcaaattagcgcgataatgcgccctaaatcagcccaaactaaccgtttgcaaagagcatcgcccaaacggggttctagcatgctgatttataaagggaaaaatggggagctgtcgcaatcgacattagccgctaaggctgtgaaacaaaagttcaagcatagtagtctccttaatatgcttttgtcgcaatcgacattagccgctaaggctgtgaaacaataagtatgaagatttgttttaagattttataggtcgcaatcgacattagccgctaaggctgtgaaac

SEQ ID NO: 64 - DNA, 306 bp, Teredinibacter turnerae
  source 1..306: mol_type = genomic DNA
gctgaaaggaggaactatatccggataccaggtcgcaggcaaaaaaatcaaacacttagcgttgccgcacaggcaaattagcgcgataatgcgccctaaatcagcccaaactaaccgtttgcaaagagcatcgcccaaacggggttctagcatgctgatttataaagggaaaaatggggagctgtcgcaatcgacattagccgctaaggctgtgaaacgtcccgcggccgctattcttttgatttataagggattttgcgatcctctggagtgaataccacgacgatttccggcagtttctccaca

SEQ ID NO: 65 - DNA, 306 bp, Teredinibacter turnerae
  source 1..306: mol_type = genomic DNA
gctgaaaggaggaactatatccggataccaggtctggttgtcggctcgcacattacattgcgccgcagtgttccagtgcactcgcctcactctaccggtttttaccggcttctaccgccaccttccctctcagccgattacatcaaactagcggcggattgagtccattttaagcacattgtggtttcacagccttagcggctaatgtcgattgcgacgtcccgcggccgctattcttttgatttataagggattttgcgatcctctggagtgaataccacgacgatttccggcagtttctccaca

SEQ ID NO: 66 - DNA, 342 bp, Woodsholea maritima
  source 1..342: mol_type = genomic DNA
ctgagaaaggcctgtagaattaggtcaaaacattgattatatgaatttaaaatcattccagatcgggatttatgtccacctcagccctttggcgcttattatgagcctaagcctgtagatcatggagctgaatgtcacatcttgtcaatgatgtagaagggggtaggctctgctcgaatctttcagcagagctgatcaaaacttctccgctgcttgagattccagcattgtgatggcgctctgctcgaatctttcagcagagctgatcaaaacgctgttgggcgatgttatcggtaaattgcatcagctctgctcgaatctttcagcagagctgatcaaaac

SEQ ID NO: 67 - DNA, 307 bp, Woodsholea maritima
  source 1..307: mol_type = genomic DNA
gctgaaaggaggaactatatccggataccaggtgaattaggtcaaaacattgattatatgaatttaaaatcattccagatcgggatttatgtccacctcagccctttggcgcttattatgagcctaagcctgtagatcatggagctgaatgtcacatcttgtcaatgatgtagaagggggtaggctctgctcgaatctttcagcagagctgatcaaaacgtcccgcggccgctattcttttgatttataagggattttgcgatcctctggagtgaataccacgacgatttccggcagtttctccaca

SEQ ID NO: 68 - DNA, 307 bp, Woodsholea maritima
  source 1..307: mol_type = genomic DNA
gctgaaaggaggaactatatccggataccaggtgcacgcagcgcagcgcctttcgggttggccccgcttatgaacatcggtgtgatgatcttcaatgtcaggttcagttgctgcatcttttccccatcccggcgtgcacgttaaatgtgcattttgcggctttcaagggaaaattaacgcgaagttttgatcagctctgctgaaagattcgagcagagcgtcccgcggccgctattcttttgatttataagggattttgcgatcctctggagtgaataccacgacgatttccggcagtttctccaca

SEQ ID NO: 69 - DNA, 405 bp, Desulfarculus baarsii
  source 1..405: mol_type = genomic DNA
gccaagccgtcggcgcgggggctatttggcttgtttgcaatatatttcgccattttatgtgttttttaaccgtgtggaattgatatggcgccgtattcattatgttttgcgcgcaaacgcccaaaaaatgcgcggaacggccctgttggcgcgccgaatttggcaaaccatgtggaaacagccccaaaaataaatattgcaatcgggcggttggctggggtaccctattgagggatgccgctagaggaagcggattgaaacctagactccacggccccggcgttgccattttttctattgagggatgccgctagaggaagcggattgaaaccaagccgatgctttccgacttcctctctttctcctcgctattgagggatgccgctagaggaagcggattgaaac

SEQ ID NO: 70 - DNA, 308 bp, Desulfarculus baarsii
  source 1..308: mol_type = genomic DNA
gctgaaaggaggaactatatccggataccaggttggaattgatatggcgccgtattcattatgttttgcgcgcaaacgcccaaaaaatgcgcggaacggccctgttggcgcgccgaatttggcaaaccatgtggaaacagccccaaaaataaatattgcaatcgggcggttggctggggtaccctattgagggatgccgctagaggaagcggattgaaacgtcccgcggccgctattcttttgatttataagggattttgcgatcctctggagtgaataccacgacgatttccggcagtttctccaca

SEQ ID NO: 71 - DNA, 308 bp, Desulfarculus baarsii
  source 1..308: mol_type = genomic DNA
gctgaaaggaggaactatatccggataccaggttgcttccgggttggctgctatatttggcgataaataaatggcgtggccggcgtaaaaaacgccgaaaataggcccgccttttcgccagcaacaatcaggcgataatgataaaagccgtggcaaaaacgccacggcttatttttttgccccgtttcaatccgcttcctctagcggcatccctcaataggtcccgcggccgctattcttttgatttataagggattttgcgatcctctggagtgaataccacgacgatttccggcagtttctccaca

SEQ ID NO: 72 - DNA, 308 bp, Desulfarculus baarsii
  source 1..308: mol_type = genomic DNA
gctgaaaggaggaactatatccggataccaggtaaacgatgtttgtcaaatttaatcaaatgattatattgatcatggcccgaaatggcaaggtttttgccaggctgacgaccgataatggcatgtccgtgtggaaacagccccaaaaataaatattgcaatcgggcggttggccagggtaccctattgagggatgccgctagaggaagcggattgaaacgtcccgcggccgctattcttttgatttataagggattttgcgatcctctggagtgaataccacgacgatttccggcagtttctccaca

SEQ ID NO: 73 - DNA, 308 bp, Desulfarculus baarsii
  source 1..308: mol_type = genomic DNA
gctgaaaggaggaactatatccggataccaggtcttgagcggggccgaggccccgaccaagagccggccgctggcggtccgggcgatgatcgggcgtggctggccgcgggcgcgtcgcccggccaaaggccggacaagactagacgttagcatgcgctgcaaggccaggctggcgcaagcaaagtttcaatccgcttcctctagcggcatccctcaataggtcccgcggccgctattcttttgatttataagggattttgcgatcctctggagtgaataccacgacgatttccggcagtttctccaca

SEQ ID NO: 74 - DNA, 412 bp, Azospirillum lipoferum
  source 1..412: mol_type = genomic DNA
ggcgaggccgcgcgcaaggacgttgcgcggaggatcgcggttgtggatgtttgtgtcaagtttggtgtggaaggatgtgtagaacgttgattgctatcgggatttggtgtgtttgatggtggatcttaaccaaaaaatagcgtttccggcttttttatccacaggatgcgtggaaaggcggctgaaaaagcaacttgaatcaaaggactgcgaggggcactgttccgccggtgtccgcgtttgggagcggattgaaacatacgcagcagcttcggttcgttgagttggagttcgacgttccgccggtgtccgcgtttgggagcggattgaaacccttgggcggtgttggacgagccgttgacgccggtgtcctgcgttccgccggtgtccgcgtttgggagcggattgaaac

SEQ ID NO: 75 - DNA, 466 bp, Azospirillum lipoferum
  source 1..466: mol_type = genomic DNA
gcggtggctgtgtcttgcgccggttgtacccctttccgggctatccggcgaggaggaagggcaggcggcggggtaacggaatgatgcgggacgggggtgggattttggcggttgtttcaaatctggtgtggaagagctggtagaccattgaagatattgggatattgggctgttcattgggtggtgttaaccacttttccgggattttggagattttatccacaccatgcgtggaaaccgttgggaattttcccgttgaatcaagcggatgcgagggataccgtcgcgacggcatccgcgttcgggagcggattgaaaccatacaggcttggtcatgtcgggctccgggaaaaagagtcgcgacggcatccgcgttcgggagcggattgaaacgttcagccgccgtggggggagatcagcggggctttcgtcgcgacggcatccgcgttcgggagcggattgaaac

SEQ ID NO: 76 - DNA, 308 bp, Azospirillum lipoferum
  source 1..308: mol_type = genomic DNA
gctgaaaggaggaactatatccggataccaggtagagctggtagaccattgaagatattgggatattgggctgttcattgggtggtgttaaccacttttccgggattttggagattttatccacaccatgcgtggaaaccgttgggaattttcccgttgaatcaagcggatgcgagggataccgtcgcgacggcatccgcgttcgggagcggattgaaacgtcccgcggccgctattcttttgatttataagggattttgcgatcctctggagtgaataccacgacgatttccggcagtttctccaca

SEQ ID NO: 77 - DNA, 308 bp, Azospirillum lipoferum
  source 1..308: mol_type = genomic DNA
gctgaaaggaggaactatatccggataccaggttcggcgcacgggttggcgcacagcgccgcgctgggcggcgccccggtgccggaacagcaggatttctggttgctgtaggggggaggaagccggccgcgacaattggctctcggccggggggatcatcttcctcgacgaaatcacgcgagcgtttcaatccgctcccgaacgcggatgccgtcgcgacgtcccgcggccgctattcttttgatttataagggattttgcgatcctctggagtgaataccacgacgatttccggcagtttctccaca

SEQ ID NO: 78 - DNA, 308 bp, Azospirillum lipoferum
  source 1..308: mol_type = genomic DNA
gctgaaaggaggaactatatccggataccaggtaggatgtgtagaacgttgattgctatcgggatttggtgtgtttgatggtggatcttaaccaaaaaatagcgtttccggcttttttatccacaggatgcgtggaaaggcggctgaaaaagcaacttgaatcaaaggactgcgaggggcactgttccgccggtgtccgcgtttgggagcggattgaaacgtcccgcggccgctattcttttgatttataagggattttgcgatcctctggagtgaataccacgacgatttccggcagtttctccaca

SEQ ID NO: 79 - DNA, 308 bp, Azospirillum lipoferum
  source 1..308: mol_type = genomic DNA
gctgaaaggaggaactatatccggataccaggtcttccgcttcgcaatctggaacgcctccggcctcctcatgacctgttccccaggggctagtcggaagggccatcacgtccaaaggcgcgattgacctgagtttgttgcccggccggatcgcgccgcctgcgcgggacggtcgatccaagagtttcaatccgctcccaaacgcggacaccggcggaacgtcccgcggccgctattcttttgatttataagggattttgcgatcctctggagtgaataccacgacgatttccggcagtttctccaca

SEQ ID NO: 80 - DNA, 329 bp, Cellulomonas bogoriensis
  source 1..329: mol_type = genomic DNA
agcgcggggaccgcgcgcgcgacacatctgcgctggtcacagccatcgatccgccccagtcccccgctggcgcgcacgccgtcccgcgcgcacgccctcgcgcgcgcgttcgcccgcgtcagccctggtcagcccggggtcaaaacgggtagggttccacccatggcccgaaactgagggcattgaaactaaaacatcccctgaggggatgtaaagggtttaaagttccacccatggcccgaaactgagggcattgaaacgggtactccaaacgccaagccttaatcgtgtcagttccacccatggcccgaaactgagggcattgaaac

SEQ ID NO: 81 - DNA, 307 bp, Cellulomonas bogoriensis
  source 1..307: mol_type = genomic DNA
gctgaaaggaggaactatatccggataccaggtgcggggaccgcgcgcgcgacacatctgcgctggtcacagccatcgatccgccccagtcccccgctggcgcgcacgccgtcccgcgcgcacgccctcgcgcgcgcgttcgcccgcgtcagccctggtcagcccggggtcaaaacgggtagggttccacccatggcccgaaactgagggcattgaaacgtcccgcggccgctattcttttgatttataagggattttgcgatcctctggagtgaataccacgacgatttccggcagtttctccaca

SEQ ID NO: 82 - DNA, 307 bp, Cellulomonas bogoriensis
  source 1..307: mol_type = genomic DNA
gctgaaaggaggaactatatccggataccaggtcacacggcacgcgacagcgactcgaccatcgtgagagctcatcagaacgccccgccgtgcacaagggtgaaattgataccgctcgctgacctgaggctggagccgcccgaacgcgcggccgcccgcctctcgttggcgtagccgcccgccgtttcaatgccctcagtttcgggccatgggtggaacgtcccgcggccgctattcttttgatttataagggattttgcgatcctctggagtgaataccacgacgatttccggcagtttctccaca

SEQ ID NO: 83 - DNA, 422 bp, Micromonospora rosaria
  source 1..422: mol_type = genomic DNA
ccgccgcactcaccacttccttatgtagttggacgaaacacctgaacccggactcacccagcgatccccgcaggagcacagatgcgcgcaaacctcttctcgcaagtcaagccagcatctcccaacgcctgcgacctggaccacacccgccacagcacctcttcgccacggtccgcgcgacgatctccatcccagcacgtcagtcccaccgattttcgggtagcatcgccatccacagcccaacccagcgggcatagaaacttcacgaacgcacggaaggcgtccggcgtgaaaccgaggaccggaatcgccatccacagcccaacccagcgggcatagaaacgccatgccgggctgccctgcatgatcttctgccgggtggtgtatcgccatccacagcccaacccagcgggcatagaaac

SEQ ID NO: 84 - DNA, 410 bp, Micromonospora rosaria
  source 1..410: mol_type = genomic DNA
ccgatacaggcatcgaagccctcgaccatcgaacctaaacccaccgaaaccggactacccagaagtgaccgccagaagccgaggtgcgcgccgccctgtttacgcacgtcaaagcccacattcaccaccatcatggcctaggtcacaccgccctcgaaggagaacttcacgaggtgcgcgctacgatctctgttccagcagctcaagacccaccgttttcgggtaggattgccatccacagcccaacccagcgggcatagaaacatggctacgccatagttggcgtcgttgttgaacgcaaaattgccatccacagcccaacccagcgggcatagaaacaggtcaggactggaccggcctggttcttgctgtcattgccatccacagcccaacccagcgggcatagaaac

SEQ ID NO: 85 - DNA, 308 bp, Micromonospora rosaria
  source 1..308: mol_type = genomic DNA
gctgaaaggaggaactatatccggataccaggtagcacagatgcgcgcaaacctcttctcgcaagtcaagccagcatctcccaacgcctgcgacctggaccacacccgccacagcacctcttcgccacggtccgcgcgacgatctccatcccagcacgtcagtcccaccgattttcgggtagcatcgccatccacagcccaacccagcgggcatagaaacgtcccgcggccgctattcttttgatttataagggattttgcgatcctctggagtgaataccacgacgatttccggcagtttctccaca

SEQ ID NO: 86 - DNA, 308 bp, Micromonospora rosaria
  source 1..308: mol_type = genomic DNA
gctgaaaggaggaactatatccggataccaggtcagaatttgcgatccctccagggttgtacctctttctggacctggagccggaggatcgttgcgatcctttcaggggtgatgagcgacaagcaggttgggcggtgggtcgcgatgttatcgagggcgaactccctcccacgcttagcaacagtttctatgcccgctgggttgggctgtggatggcgatgtcccgcggccgctattcttttgatttataagggattttgcgatcctctggagtgaataccacgacgatttccggcagtttctccaca

SEQ ID NO: 87 - DNA, 308 bp, Micromonospora rosaria
  source 1..308: mol_type = genomic DNA
gctgaaaggaggaactatatccggataccaggtgccgaggtgcgcgccgccctgtttacgcacgtcaaagcccacattcaccaccatcatggcctaggtcacaccgccctcgaaggagaacttcacgaggtgcgcgctacgatctctgttccagcagctcaagacccaccgttttcgggtaggattgccatccacagcccaacccagcgggcatagaaacgtcccgcggccgctattcttttgatttataagggattttgcgatcctctggagtgaataccacgacgatttccggcagtttctccaca

SEQ ID NO: 88 - DNA, 308 bp, Micromonospora rosaria
  source 1..308: mol_type = genomic DNA
gctgaaaggaggaactatatccggataccaggttcgaacgctacgactgcgctgcttggctgcccgtcgtcgtaagcgttctcccgtacctcttcccatgcccgcagcagtgcctcatcagtcaccgcccgcgccaacaacgcgcccaatagccaccccttcccaaaatcgatcccaacgggtgtttctatgcccgctgggttgggctgtggatggcaatgtcccgcggccgctattcttttgatttataagggattttgcgatcctctggagtgaataccacgacgatttccggcagtttctccaca

SEQ ID NO: 89 - DNA, 301 bp, Micromonospora rosaria
  source 1..301: mol_type = genomic DNA
gctgaaaggaggaactatatccggataccaggtgccacccccggtgcttgttggccagcttgtccccggggcgatgagtgactggtcggcgagggtgggcagcccagtgtcgtacgagtttcgatcccagggattgtgagcgacccgagcgcggcacgtgggccgcatacgtgacggcgactcgttgcgatccctccaggggtgatgagcgacgtcccgcggccgctattcttttgatttataagggattttgcgatcctctggagtgaataccacgacgatttccggcagtttctccaca

SEQ ID NO: 90 - DNA, 379 bp, Tolypothrix campylonemoides
  source 1..379: mol_type = genomic DNA
cctactataccatttgtgggatagatacaatgcctgaaaccctgatgatttcgttctatcccacaaatcagcttcctcacaaaggtttgaggtactttggctggatagttgttgagagtaattcgcatttatttggacgaaaatttgcatcccacaaaaaagcgctgtagaattatttcagggtaaggcttctaggagctggtctttaaacttcttcggaagttgaattaatggaaacccgaacctccatcccatatcgcatccttacgaaagtctttaaacttcttcggaagttgaattaatggaaactttgtgagccattcctgagtcgtcaccatcatccactttaaacttcttcggaagttgaattaatggaaac

SEQ ID NO: 91 - DNA, 306 bp, Tolypothrix campylonemoides
  source 1..306: mol_type = genomic DNA
gctgaaaggaggaactatatccggataccaggtttctatcccacaaatcagcttcctcacaaaggtttgaggtactttggctggatagttgttgagagtaattcgcatttatttggacgaaaatttgcatcccacaaaaaagcgctgtagaattatttcagggtaaggcttctaggagctggtctttaaacttcttcggaagttgaattaatggaaacgtcccgcggccgctattcttttgatttataagggattttgcgatcctctggagtgaataccacgacgatttccggcagtttctccaca

SEQ ID NO: 92 - DNA, 306 bp, Tolypothrix campylonemoides
  source 1..306: mol_type = genomic DNA
gctgaaaggaggaactatatccggataccaggtgacagtccattttaggcgataacaatttttacattaacagaatgagtgaaaagtagaaaataaaatttcaggatatatttagctggttttgaaaaagaggtgatgacttttccactcaactcacctctttttacttgcctgttaacagccgtttccattaattcaacttccgaagaagtttaaaggtcccgcggccgctattcttttgatttataagggattttgcgatcctctggagtgaataccacgacgatttccggcagtttctccaca

SEQ ID NO: 93 - DNA, 307 bp, Tolypothrix campylonemoides
  source 1..307: mol_type = genomic DNA
gctgaaaggaggaactatatccggataccaggtgttgacttccgcgaacgtcctacagataagggtttgaggcttattgatatctttttattgagaaatgattgcagttattctgacacaatttgacgttccgcgcaaaatggttgtagacttggctcaagggaaagctttgagaaccctgctctgttaaacttctcccgaagttgaattaatggaaacgtcccgcggccgctattcttttgatttataagggattttgcgatcctctggagtgaataccacgacgatttccggcagtttctccaca

SEQ ID NO: 94 - DNA, 307 bp, Tolypothrix campylonemoides
  source 1..307: mol_type = genomic DNA
gctgaaaggaggaactatatccggataccaggtaccaaattctcaacaaggtaatttgagttctgaaaaagagggtttaacactaaccctctttctttttactttttacttctgctaaatgactcttttcggaatctgagttaacccttttactttttactttttacttggctcctgtttatcgtttccattaattcaacttcgggagaagtttaacaggtcccgcggccgctattcttttgatttataagggattttgcgatcctctggagtgaataccacgacgatttccggcagtttctccaca

SEQ ID NO: 95 - DNA, 306 bp, Tolypothrix campylonemoides
  source 1..306: mol_type = genomic DNA
gctgaaaggaggaactatatccggataccaggtttctatcccacaaattagcttcctcacaaaggtttgaggtactttggctggagatttgttgagagtaattcgcatttgtttggacgaaaatttgcatcccacaaaaaagcgctgtagaattatttcagggtaaggcttctaggagctggtctttaaacttcttcggaagttgaattaatggaaacgtcccgcggccgctattcttttgatttataagggattttgcgatcctctggagtgaataccacgacgatttccggcagtttctccaca

SEQ ID NO: 96 - DNA, 306 bp, Tolypothrix campylonemoides
  source 1..306: mol_type = genomic DNA
gctgaaaggaggaactatatccggataccaggtttggaatcattaagttttccaaataaaacacaaaataaatttgctaattaaataaaaaattaaaagatacattatcaatttggtagtttccatttttaaagagagggtttaacactaaccctcttactttttacttggctcctgtttatcgtttccattaattcaacttccgaagaagtttaaaggtcccgcggccgctattcttttgatttataagggattttgcgatcctctggagtgaataccacgacgatttccggcagtttctccaca

SEQ ID NO: 97 - DNA, 440 bp, Oscillatoriales cyanobacterium
  source 1..440: mol_type = genomic DNA
ggtattggtggcttttggcttaatgattggtatttgttatcaattgtcagcctgttgataagagttggggggggtaggcgatctctgaaacccgcattctgtcgttcccccccccaatttgctctctggttatgggtttgagggctgtatttggcagaatttttccagcttattgaaaaaattatgccattattgacgaataaacttgacccccccaaaaaagtgctgtagaatgagcttggggcaaggctcctagagactggccttctcgacttctctgaagtcaaattaatggaaacatgatcttaatcctcaaaaagaaagtgaactgctacttctcgacttctctgaagtcaaattaatggaaactctcctgtgaaattgaaagtcgccgtggtaccggtgcttctcgacttctctgaagtcaaattaatggaaac

SEQ ID NO: 98 - DNA, 306 bp, Oscillatoriales cyanobacterium
  source 1..306: mol_type = genomic DNA
gctgaaaggaggaactatatccggataccaggtcaatttgctctctggttatgggtttgagggctgtatttggcagaatttttccagcttattgaaaaaattatgccattattgacgaataaacttgacccccccaaaaaagtgctgtagaatgagcttggggcaaggctcctagagactggccttctcgacttctctgaagtcaaattaatggaaacgtcccgcggccgctattcttttgatttataagggattttgcgatcctctggagtgaataccacgacgatttccggcagtttctccaca

SEQ ID NO: 99 - DNA, 306 bp, Oscillatoriales cyanobacterium
  source 1..306: mol_type = genomic DNA
gctgaaaggaggaactatatccggataccaggtctcttgaatcgcaaattctgtggctgttacagcgtgtaagcgttctaaaatatctcgctgtaaattctggacgatgatgatactttcatcagggagcaaagccatgattactgtcggtttattagagtgttattgccgataatatcattatttaccattaatttgacttcagagaagtcgagaaggtcccgcggccgctattcttttgatttataagggattttgcgatcctctggagtgaataccacgacgatttccggcagtttctccaca

SEQ ID NO: 100 - DNA, 307 bp, Oscillatoriales cyanobacterium
  source 1..307: mol_type = genomic DNA
gctgaaaggaggaactatatccggataccaggtcgaaatccttttttggtaagggtttcaaagccttattttaccatagatttctaacttattgagaatatcctgcttttattgacaagaaaacttgaccctcacgaaaaagtgctgtagaatgagcttggggcaaggctcctagagactggcctttcgacttcttaggaagtcaaattaatggaaacagtcccgcggccgctattcttttgatttataagggattttgcgatcctctggagtgaataccacgacgatttccggcagtttctccaca

SEQ ID NO: 101 - DNA, 307 bp, Oscillatoriales cyanobacterium
  source 1..307: mol_type = genomic DNA
gctgaaaggaggaactatatccggataccaggtgcgctcttagagttgagattgcatcgacggggctaaacttaagtttattgaacaatttttctcttcaggctttatcaacaaataacgcgatcgcctcataagatttattaactaaaatccctagctttttttaaatggctagggatttattgtttccattaatttgacttcctaagaagtcgaaaggtcccgcggccgctattcttttgatttataagggattttgcgatcctctggagtgaataccacgacgatttccggcagtttctccaca

SEQ ID NO: 102 - DNA, 208 bp, Psychrobacter lutiphocae
  source 1..208: mol_type = genomic DNA
cggcgtatcttgtgacctattttgtataaactattttttaatcaacaatcaaaaaagtgaaaaatccattggcgacctatgatatacttaacctcgaacagccttttgaaattcgacatcgctaattttgtcctctggacgctatatgtggtgtggctttgaaaatggcagggttctgcactatgcagaggtacgtaccgattaagac

SEQ ID NO: 103 - DNA, 279 bp, Ruminococcus sp.
  source 1..279: mol_type = genomic DNA
tgtctacctcgaaagccttgaaaatactggggaaaatggcgattgaagtggctgaaaacaaagggaaaagctataaaaatctcaaaaaaatgaaaaaaagtctgtctaccgagacgaaaaaataaacttttgaaaaaagcttgatttttcaaggatatttgcgataatgaaaagtgaggtcgacagactttttaatgtgattagagggagaaaaaagaatacagagtcggaaaacggcgaaaatactgttgtaataccgtacctataaggaattgaaac

SEQ ID NO: 104 - DNA, 331 bp, Vibrio sinaloensis
  source 1..331: mol_type = genomic DNA
tgtactgctcttttacaatttggactttaagtaccggaatagtttcgctattattgagcacactaataagctgtaacaagtgtcagtttgtggataagtttaggccaaccaaaagtgtattttatcttgttgaatcaaaaggtttaatcgcaggtaggcttcattagtactggtcgttcagaccgttgagacattcctgtttatacaggtgacttttgggtttcgggcttcattagtactggtcgttcagaccgttgagacaacatggttagttttttatgatcacccaagcatcggcttcattagtactggtcgttcagaccgttgagac

SEQ ID NO: 105 - DNA, 197 bp, Blautia sp.
  source 1..197: mol_type = genomic DNA
aaaaagtcggtcgatctcatgcctgaaatcatgaattctgcaaaatggcggaaatttaaggaaaatcaggaatctcagaaaaacgatcgaccgacttttgtgataaaatggttgcaaaaaagagaaaaatttgatttaatagaatgtgaaaatagcggaaatgctgatgttgtaccttacctatgaggaattgaaac

SEQ ID NO: 106 - DNA, 197 bp, Blautia sp.
  source 1..197: mol_type = genomic DNA
aaaaagtcggtcagtctcgtggctgaaatcatgagttccacaaaatggctgaaattcaaggaaaatcaggaatctcagaaaaacgatcgaccgactttttcgataaaatggttgcaaaaatgagaaaaatctgatttaatagaatctgaaaacagcggaaatgctgttgtcgtactttacctaaaaggaattgaaac

SEQ ID NO: 107 - DNA, 398 bp, Rivularia sp.
  source 1..398: mol_type = genomic DNA; strain = PCC 7116
ggttttagttctactacggagataaagtgaacatgaaaatcgacgcatctcgccaaatcgctcaaatgcctattattccgttgagatgcgtcgattgcttgctaggcaagggtttgaggtcggtgttttgcttgattttcgggctcatatataatgtttttcagagatgcgtcgatttggctcctgtaacccttactgggtaagggctgctgagcgaactccccaccgattgggttaattcggattagttggaaacttgggggaagtttgcatattaactcgttacctccccaccgattgggttaattcggattagttggaaacttttaccaatgaagtttccagtgatagcaattatgttaccccaccgattgggttaattcggattagttggaaac

SEQ ID NO: 108 - DNA, 432 bp, Rivularia sp.
  source 1..432: mol_type = genomic DNA; strain = PCC 7116
gtctgctgtttcagctataaagaaactgcggtcaaattaaagtataagcttgccattccggcaaacaatttctttaagtcacatatagctttatccttccaatttgcgtcttaggacaagttatgcttccattgttagaatttgttgacaatcgattaacgcaacaataagggtttcagcccttaaattacttggttatgcttccaggggctgacagcttttgggtatagtgacagaacctgattacgaaggtttccaactaatccgaattaacccaatcggtgggggatgaagaacgttggcatttatccttactgggtaggtttccaactaatccgaattaacccaatcggtgggggatgaatacattcttttggttagacatgatcaaattctgtttccaactaatccgaattaacccaatcggtgggg

SEQ ID NO: 109 - DNA, 307 bp, Rivularia sp.
  source 1..307: mol_type = genomic DNA; strain = PCC 7116
gctgaaaggaggaactatatccggataccaggttattattccgttgagatgcgtcgattgcttgctaggcaagggtttgaggtcggtgttttgcttgattttcgggctcatatataatgtttttcagagatgcgtcgatttggctcctgtaacccttactgggtaagggctgctgagcgaactccccaccgattgggttaattcggattagttggaaacgtcccgcggccgctattcttttgatttataagggattttgcgatcctctggagtgaataccacgacgatttccggcagtttctccaca

SEQ ID NO: 110 - DNA, 307 bp, Rivularia sp.
  source 1..307: mol_type = genomic DNA; strain = PCC 7116
gctgaaaggaggaactatatccggataccaggtatttgcgtcttaggacaagttatgcttccattgttagaatttgttgacaatcgattaacgcaacaataagggtttcagcccttaaattacttggttatgcttccaggggctgacagcttttgggtatagtgacagaacctgattacgaaggtttccaactaatccgaattaacccaatcggtgggggtcccgcggccgctattcttttgatttataagggattttgcgatcctctggagtgaataccacgacgatttccggcagtttctccaca

SEQ ID NO: 111 - DNA, 305 bp, Rivularia sp.
  source 1..305: mol_type = genomic DNA; strain = PCC 7116
gctgaaaggaggaactatatccggataccaggtaaagcagaagtggaagcactttgtggcagtacgcttgtggaagtacttcttggcagttactgccatcttctcgttcaagttacaagatgctcggttgaaaccccaccgattgggttaattcggattagttggaaacgtcccgcggccgctattcttttgatttataagggattttgggatataccaccgttgatatatcccaatggcatcgtaaagaacattttgaggcatttcagtcagttgctcaatgtacctataaccagaccgttcag

SEQ ID NO: 112 - DNA, 305 bp, Rivularia sp.
  source 1..305: mol_type = genomic DNA; strain = PCC 7116
gctgaaaggaggaactatatccggataccaggttaagttaattacagcttgatttttcatccaatttttatattaaaagcctaccgcttaaattggtaggcttttttattgcaagtcattgtaactctttactgtttccaactaatccgaattaacccaatcggtgggggtcccgcggccgctattcttttgatttataagggattttgggatataccaccgttgatatatcccaatggcatcgtaaagaacattttgaggcatttcagtcagttgctcaatgtacctataaccagaccgttcag

SEQ ID NO: 113 - DNA, 1899 bp, Fusicatenibacter saccharivorans
  source 1..1899: mol_type = genomic DNA
atgttcactatagacgagatgctatcaaagaacaaccaaagattggctttcgagcacttcgctactaagaacgacggatgtggccctgatggtatgcacgtatctgagctggagaaatattggcgtatgaatcacgatcagatcatctccgacttgaagaaccaagagtaccaacccggcattattttgattagagaacacatgaacaaaacaggtaagagaaggaacattgcatctctcaacgtgatcgacaggttcataactcgactgctgtcgcaaaagttgaatagatacttagcccccattttctgcgagaactcatacgcttaccaagactcaaaaggggtgatgcccgccgtactaaaagccaaggagtacgtcgagttgggtatgagacacgttatcgagatagacctcaagaattacttcgacacgatcccgttagagaacttaatcccagagatcgagcgttacataacagatgaggcagtgctgcacttgattaaacagtacttattctgtgacatctcgttcgagggtaagatatcacgtaaaactcaagggatcgttcagggtaacgcaatttccccaatactatccaacctctacctgaacgacttcgacaaggagctggacgagtcgaagctgtgctggatcagatacgccgacaacatatacatctatatggacagctacgagaaggccctactggtttattccgagcttaccgagagacttgagagaaggaaattaaccgttaacaaggagaaatcaggggtgtttgatgttagtacccgttctatactaggttacgacatcctgatcagaaacaagaaggttgacgtgcgtaaacacatctacaagtctgttaaccagtactccaactggcacgactctcgattggagttcatcaacggtcgttaccacatcacttccgacggtatcctcaatagacaagacttcggtctactgttcgagaatgagcagaagaagcactacatacctgttgaggtttctgaccagttgaatatttacggaaacgttacgctcgccagcaacgttcttcaatcattctccaaccgagagatcaaagtgtccttcttcgacaagtacgggagacttattggatctttcctgcccgagaaaactaagaaaagcgccgagataatactggttcagtctaagaactaccttaacgaggacgttaggatggacacggcccgacgtatggagatcgccggtttgcataacatcagagccaacctcagatactacgacaagaagcacaaaggtgactttaaggaaaaggtagacgccatttctggctatattgacgccctcaatagagcaccatccgtgaacgacatgatgctcttagaggctaaagctcgtcagctgtactacacatgcttcaaccagatcttagagacatccgactttcagttcgagaagcgtaccaagcgaccaccaaaagacgccatcaacgcctgtatttcattcggcaacacattactgtacaacttgtttgtgaatatcatttggaagaagggacttgatccaaggttcggggttgttcacgccagcaacaagcgtaatcagtcccttaacctggattttgctgacatcttcaagcctatagtcatagataggatcatcttcacaatgatcaacaagaagatgctgaccttgttgacggacttcgagacttctaaccagggagtttacctttcccgagaggggaagaacatcttcttgcagatgtacgaagaaaagctcaaatccagaatcactatcaagggaaaggagatgagttactatcagttgctagaatcagaggtgcagaactacaagaacttcatcctgaccggcgagacatacaagccgtacaagtactactag

SEQ ID NO: 114 - DNA, 2643 bp, Candidatus Accumulibacter
  source 1..2643: mol_type = genomic DNA
atgtctactccagactactgttggcagcgtataggtcttacactaagatgtacatttgctcacccagggagaaaagcccatccactttctgtcctagaggcaatagttaaaggtattggagaagcagctgggttgactgctcctactatgagatcagtatttagatggtccggaatgagatcgtcgcaaatgactattgagagtggtaggatcttgtctctcgaaattttgttatttggtactgacgctggagctgcttgtaattggcacgaaagagccatacactactttgatcctggtgcaccgggtagaaactttcaggttactgcttccgaggcccctgtcgaaagacgatgggcagagctattggctggacgacaggctccagcagaatccaatgatgagtgctgtttggactttcttaccccattaccctttaccccagctcaaggacgtggaagaacatggcttgatggtgaaggtctacgtagagccatgcaagacagattgagaagattatttggtgctgaggctgaacttccccctattcctgaggtcctgcctgcttattggtattattgtcagattgttcacgctgcttcctctcagccaggtcataataaataccttaatggatgtttgggtcctttgttgcttagaggagaacaccttggggaatggtggccttggctggttctaggtgaggagattggacttggtggacaagtttcttttggacaaggtttattcagactccatgctaagtcagttccaattcttgacgccaggttgacggacccaaatcaaattgctgctattattgatcagcttctattaagacatgatgacttagcagtaagactctctaatacccctcaagccccagatcttcatgagttggctgtggagttggctcaaaatctcagagagggagcagctcctctcccattccaagctatcagagttccaagatcggacggtagactccgtcaatttgagacacccgcagcacgagacttagttattttgaatcatttgacgagacttttatcagagccgtttgatcgattgttttctgtgcacagtatcggatacagaaaaggtcactccagagaggatgcagtcgaaagagttagagcagctattgcagagggttgtactcacgttttggagtctgatatttctgacttctttccgtctgttgacttaaaacgactcttggcacgtcttgatgatgttttgccgcgtcgtgatgtaagattgagacaaacactcgctgcatatcttggtgcaggatggcgttacggtgaagggtcggtccaagctagaaatcgtggacttccattaggatcaccattgagcccactcctagcaaacctttacttggattcatttgattcccaactgggagctactgttccaggagtcaggttaattaggtacgctgatgattttattatacttactgaatcagaagcagccgcaagagctttattagacactgctagagatgccgccgccgctttaggtctagctttgaatttagaaaagactgctattagaccactctcggatggttttgacttcttgggaataagatttagtgctgacgctgctgctgaacaagctggagatgaatcagctgatagtttaagaaaagttctttatattactgagccatacgcttttgttggatcaaatcatggtactattgaagtgcacgccggttccaaatccttgggatcattcccactggctagaacagctggtgtcgttactttggtgccatgcactctctcatctgctctaattgctcgtcttgctgatcaatgtattcccttggctattgcaggcacacaaggtagacaaattgctactgtagctggggatacggcaaggagattcgcaactgctgcaactcaagcaaatagacacgcttcattaggtgaggccggtagatgtagggctgctggagccttcgctactgctaaacttgctaattacatagcacttataagacaaagaggaccagcaggaacagcagctctagtggcacgtcttgagaatggtatagctgctattgcttcagcaactgatatagatgcaattagaggtgtagaaggtgactgtgctcgtgagtgtttcccatttattgctggatggattaattctccagacttcccttggcagggcagaagaaggcatggtgagtttcctgacagattaaattctttgctaaattttggatatcatctgttatttacgagaattaatgccctattgcgtgtttccggcctgaacccttatttgggttttcttcacgcagcaaatggaaggtatgaagccctggcttgtgatgtacaagaggcatttagacctcatattgataggttggtcgtaagactactaaatctcaaggttattgaggcagctgactttgaagaatctgaggaaggatggtggctaattcgtccagcacgaacaagatttcttcagcaatttgctagagagatagagcgacgacccatgaggcgacgttattcccttggtgaggctattgagggccaagtcagagccctgcacgcttggttaattgaggatagagagttggtcttgtatagatggtctgattcagacgtgtaa

SEQ ID NO: 115 - DNA, 1983 bp, Candidatus Accumulibacter
  source 1..1983: mol_type = genomic DNA
atgtctactttacccaccccttccagcactgaccaagactcaccaccacctttctggacattagctaggttggcagaagctcttgagcacgtgtcagctcgacaaggcggagctggagctgatgagcaaactttggctgagttcgcagcagacgcagaggcccagttagggcttttggccttgcagctaacacaaggaagttacagaccagcccctgctagacttataccagtcgctaaaccaggaggaggtgttcgtgagcttttactgccagcagtccgagatagaattgtccaaagtgctttggctcgttaccttgctgaccttttggaaccagattttggtgaggctagtcacgcttacagaccgggacactccgtagctactgctttacataggcttcaagccttacgagatggcggattggttttcgtggcagtctgtgatatacatcatttctttgattcggtagatcacagaaggctcttttctcttttagatgaccttccattggaaagacgtttgcgagagcaaatgaaaacttgtgttcgtatagaggtagctgatgtacaaggccagggagcttggtccttagctagaggattggcccaaggttctcctctgtctccagtcttagctaacttgttcttgatggcttttgatgctgcatgtgctcgtgctgggttggctcttgtcagatatgctgatgactgtgtccttgcttgcgcttccgagactgaagcccagtccgccctagcatttgctgctgatgctcttgagaatattgggttggcccttaataccagaaagtcgcgtcttgcctctttcgcagaagggtttgagtttctaggtgcattttgtggagctgagggcatgttgggtggccgaccaggagaagcagcttgtttaccaccaactaccggaccagtacatgaggctgctgctgctgacgatgagaggccaccttcacatggtcatcgtccaaggttacgtacattgtacttgttggagaatggcgcagttctcaataaagaaggggagagatttatagtagctcgacatggtgaggtattacttcaggttcctatgatgcgtattgatcaaattatggtctttggtaatgtccaaattactactccagctttgcatgagtgtttagaaagaggaatccctgttatgcttctaagtggtcgaggacgtttctttggtgttattgatccccttgatgcaagatccgtccctttacaaagggctcaattcgcactagaatctgatgaaccagctagactagccctcgctaggcctttaattgctggaaagattttgaattgtagaaccttcttgggaagattggctagagccagacaaacaaatatggacgctccattagctgctctcaagagcgcagcccaagcagccggacaagctgccgatttggaaattttgagagggattgagggagcagcagctagaacatactttgcagcttggcagactgtcttaccagcaaaatggcagtttactggtagaaataggcaaccgccaactgatcctgtaaatgccctgctgagttacggttatacaatcgtcttttataacgtgctggcattggttagagcaagaggactaaatactcatgttggggtattgcatgacgttagacctggacaccccgccttagcttcggacttaatggaagaatttcgtgcaccagttgttgacgcagttgttatgcacttggttttcgatggaaaattgcagccaggtgatttcagctggccagaaacccccggacaaccctgtctcatggcagacggctcacgaaaacactttattcacttattagaacagaaattgaatacaacagtttctcatgctggtcagcgacttgactataggagatggatggatatgcaagttcttcaatatgcagcagctcttagaactccagggttgccgtatgttccctttgctattagataa

SEQ ID NO: 116 - DNA, 1920 bp, Eubacterium saburreum
  source 1..1920: mol_type = genomic DNA; strain = DSM 3986
atgaagaaactgactatggaagatatcttcatatccgagcaaatcgaggaagccctcgatcacctgtctactaagaaggacacatgcggtattgacggtttgtacttgtccgagcttagggacgactggaacatcaacggtgagaggtacttgtccctcttgcgtaaggggaaatataagccgggtatcgtgcagatttacgagattgttaactacaccgggaagcgtagatccatttcctcttttaattccatagataggctagtcctgagatgccttgctacaagtctggagaagtactacgactcgatcttctctagctcctcgttcgctttccgaccagggctcggggtggacaaagcagtcgccacgttcgccaacaacctgaacaccggactgaccagggtagcaatcatcgacatcaaacactacttcgattctattccaatcgatcgtttggagatgatactgaagcgaatcatagacgacaacgtcttattgagtctattccacaacttactttattgccgtatctctgaggagaacgtaatcaagactaagtctaagggcatactccagggatcaccaatatctccattccttggtaacttgtacttatctttactagatacacagctggagtctatgcacgtatcattttgcagatactgcgacgacatcgctatgttcttcgcttctttcgaggaagctaaggaaacttacactaaggtctacgatatcttgaagaacgacctggagatggacattaacccacagaagtcagggatctacgaaggcatcaagcagaactacttgggttatagtttcactaagaacaagaaggagcaccagatcttagctatcaagaagaagaaggctccacctcagatttaccagcactggtccaccaccgcaatccagagggtcgataggaattatcatatcattaacaacggtattcttaaccgtaaggacttcaccctcttgttcgagaacgacagaggcaagaaatatctccctgttgaagccaccgagtccctaaacgtgtacacaagtgtaattttctcctcagacttcttcaagtacgtaggcaacaagaaaatctgcgtcaacatctttgataagtatggggagttggccggaactttcagtccaccggagtcccttcacggtggcctcaccatgttaaaacaagctgccatttacttggacgacgagaaaaggaagctgatcgccaggaaattggaaattgcatccctgcataacatgaggtctaatctgaagtactatgagcgtcaccactcctctgaaaacctgaaggacggtattacctcgttctctgaatacattaccgctatgaacgaggctacaaacatcgttatgctattaaccattgaggcacgagcacgtcagttgtattattctctgtttcacgagatcatttgcgacccagctttcgagttcacgaagcgtacccgtaggccaccaaaagaccccttgaatgccttaatctcattcggcaacgttttcttgtataacagaatcgccacggaaatccagaaaacatcgttggacataaggatcgggttcgttcacgctacaaacaggcgtaaccaaagcctgaacctagatatagccgacctattcaagcccctgatcgtggatagagctattttcaccatcattaaccgacacatgatccacgcttcagagcacttcgagaaaacagaggacggtggcatttacctcaacaaagagggtaagcagatcttcattaacgagttggagaacaaggtttatcagaaacaaacagaggagaacaaacctcgtacctacgatactcgtatccgagaggagatccacaagattttccgattagtttgttacgacgagaagtataagccattcaagtataactaa

SEQ ID NO: 117 - DNA, 2271 bp, Bacteroides fragilis
  source 1..2271: mol_type = genomic DNA
atgccagactactaccactccatcactactctccatgcacttcagaacgcttggagagcagttagagctaagaacgctgccggtggtatagacggttttacgctttcccacttcgagaaaagacttaatgacaacttaatcgagttgcagcacgagttgatctcacagacttggaaccctgagccctatctgagaatcgagataacgaagaacgaaacggagaagcgtaagctaggtttgctctgtatcaaagataagattgtccagcaggctatcaagaccgctatcgagccacaactggaaaagactttccttaacctgtcctatggatatagacctaataagggcccagagagagccattaagagagttgtgcatgacttgaagaagctgaaatctggatacgtcgctaagttggacatcgataattacttcgacacaataaaccacgagagactatttaccagactagcaaactggctaaaggacgacgagactttgagattaattaggttgtgcattcagaccggcattgtcacgcctcagctacagtggcaggagattaacaagggcgtcccccaaggggccattctctcaccgttgctggccaatttctacttacacccgttcgaccaattcgcagctaacaaggttccaatgtacatcagatatgccgatgacttcctgatagccacttcgactgagaagcagatcaaggaagccgtggagctagtcaaagaggagcttgagtcacagttctacttgcagttaaacactcccatcattcacaactttcacgacggtattgagttcctgggtattaccattagcgacactggactgtccattaccgagaagaagaagaaaaccttgcaggaaaggattaactccattaagttcatcaagtcatccctgagttcgcagtctaaggaaacattacagggcattaagaactattacgctaaattattgcctgagtctaccctgaaagagctagactgttttctgatgaatcgtctgaacgctctcatcataagaaatcagaatagtatcaacaataagaaggagctagtctctaacttgcagaagatcgagttttactctgagaactcaaacaagaacaagagccagctcatccagcagttgtgctcaacttacattgtccacagtactaagtccaaaacgagattgacttccactcacatcgacaacaccaaattaattactcagaagaagaaggagtaccaaaagagagagaacgagggagcagagcttgttattagcattcccggaagttacatcggcgctacatacaagggtataactgttaagctacaaggcaaaatcataaacaagccctcgccggccttgaagcatatcactgtcgttggaaaaggtatctctctttcttcaaacgctataacctactgtatgaatcataagatccctatcgatttctttgacggaaggggtaagcagtacggaacagtcttaaaccccgttttccttgacggcacgctgtggaacaagcaagtcgagcttcccttagagcagaagattaagctggccacccagataatcatcggaaagctgaagaaccagcttaacttgatcaaatactatcacaagtatcacaaggacattctaggcggcaaactttccgagaagtacgtagaggtggtgttgaagattgataaattgatcgaaaaggcaaagaactactcgcaacgaaacgagaagtacaccgctgagttgatggctatcgaatcgcaagccgccattgcttactggtcctatattagagtcctaaccgccgacgatggcatcgacttcattaggcgagaacatcagggagctactgacctgttgaatagtttgctgaattacggttacgccatcttgtacgccagggtgtggaagaacatcttggcagcaaagctcaacccctcaatcggtgttctgcacgccaaacaggacggaaagccaaccttggtgttcgacgtggtggaactgttcagggcacagatggtcgatcgtgtcgttatctctttaatccagaagaaggtgagcttgaagatgcacgatggattattgaatgagtccagtaagagagtgcttataaggtacatcttggaaagattaaacaggtacgagaagtacaggggcgaggagattacttttagtcagatcatcctcaggcaggcacaggagattgctttgttcatatcgggcgataaccttatcttcaagccatacgtagctaagtggtga

SEQ ID NO: 118 - DNA, 2199 bp, Campylobacter fetus
  source 1..2199: mol_type = genomic DNA
atgttcgagaaaaccctagagcagatcctaagtgacaagaacattcagattgctctgaactctctgaagaaaaccgccctgggtatcgacaacttatccgaactgaacgaacacttcatccataaactcaagcagtcctgcctgaatcagacctacgcccctgaaccagtcttacagaagttgattcctaagtccgacggagagaattacaggaaactcgctatttcctcattgaaggataagctaatccagaaggtattggccaatgaactgacttggtacttcgacaaacacttctctgacaagagctacgcatacaggcctggtaagtcatacaagaacgccatcttcaggctgagagacttcttgcgagtgaagccctacttcgtgatcaagtccgacattaaggactgcttcgaatctatcaaccacagtaaattagtggctttactggccaagtacatcaaggacaagcgtgtcttaaatctggttgagatatggattaagaacggcatattcaacagacagacatatatcaagcatagcaagttcggcattcaccaaggggacgtgctgtcaccactgctcgcaaatatctacctcaatcagatggacaagtttctcgaaacaaacaacgagatctttattcgatacgcagacgacttcgtcatccttgtggacgacgagaaattcgtacaggcaaagatcaacagcttaaagaccttcctctcaactattgacttgtccttgaaggacactaagaccgctatttactctccgacccagtctttcgaatttctaggagtctccttctacggatctaacctgagtattaacgaggcaaagttcgataagatccaggagaagatctatgccctgagtaagtctaacgacttcaagaccgacttcaactcctatattgctcatctccagacaatcagcctgaacctgatcaagaccgatcagactcaactaaagaggttcatttacgccttaaagaagtgtgtgtccttgtacatcaagaataagacaactctgaaaacccgattagagatattcacattcctcgacactctgaacttcgcacttaacttcaagtccaaaggtgagaaggacgagttctacaatcagatctacgctctcaccagagagaagcaagcactgaaggccaagccacaacctaacccgcagcatgtcttgaataagaagaagaagcattaccttgagcagttcgctcaaaatagcttgctccatattagtacccctcactgcttcttgggcgtcagcaacgtgaatttcgttatcagatataagggaaaggtgattctgaaggtccgaatagaccagatccaccagattatcatagcctgtgacatctcattgtccactaatgctattaaggccgcaaccaagcgaaacatatctatcgactttcttggattcaataatcagatatacgccagcctgttcagtcacaccagtacaataacaccggcttataaggcccagatcgacttcctaaattccccaaactcattgaatctggctaaggaatttataaaggcaaaggcaaccaatcagatcaactatttgaagtacttggacaagcactacaagatcttggcatccaatatcgacaagatgcacaagaacctgaagaaggcccttatttcagccaccaccacctcggagctgatgggatacgagggagccatcagtagcctgtacttcgatgccattgcctcaactctcgaagataaagagtttaagcgtgttggtaagggtgctaccgaccttgttaactcgcttctgaactacggttacgccatcttgtactccaccgtccagtctgcactgattaaggcaggcttatacctgaacatttcctacttccacgtttcagccaagttcagtttgtctttcgacttcatcgaagaatttagggtggtggccgtggaccgtgtggtattcaccttactccaccagaaaactaagctgtccttgaaggacgggctgctagatgtcccgaccaagaagaagttaacacagaaagtcgccctggcactactcagcactcacaagtacaagaacgaggagctgaacctagagcagatcattcaggcccaggcttacctcttgagaaaacagatcttcaatgaggccaagtataaagggttcctggttcgattccagggttag

SEQ ID NO: 119 - DNA, 3045 bp, Teredinibacter turnerae
  source 1..3045: mol_type = genomic DNA
atggtaactagagtcgaggagtttgatcctctcccccttgagaatttagaacaagcagcactattggagcagctgttacctttgcgttctcttgctgttcatcttcagttcattgacgagtcccatccaaggttctttcatcaagctgctgtttcagctctcctaagacacttgctaccatccgctgatgattactcacactatcttattttggacgctcccgagactgctagaatcaattatcaagctggagcttcttattgcttctctatagtctgcttggctggcggggagtcgcttttagccactctaatgcttgctttgagacagttaccatattccgcacctacagggaaccctttagcttcatttggtgctaatttgcgttggagagggttttcgtgtaacttttgcgaccatcccgtggatcaagtcgaggacttgggttgttatgggatagagcacctagcacaagaggctgctttatgggccgaacaccatactttgttccactggcagtggcttactccagtcaggcttttgaaggagaagtctgctagatctacagctaaaggagaggcaagatactgcgctgacgctggagatttgtcagctgcactcctcttggccagactatatgatactgtgaacgcattgctgactgagagaggggaccgaccactacccccacgacaagcagctccacacatgaatgtagaggctcaacattgtttctggcttgatgctggttaccaaaatagttctggaaaggagcaagtgatgggtggacttttaggggagtttacatattctactcctgttccacttccacccccttgggctcacttcctagttctaggtcagtacattgggttgggacaaagaaggtcattcggttggggaagatatcaattagtcacgaccgagaatcaagtgtcgtgcaggcgtcagttagccgctcaaccactgttagagagagctttggaaccagctaatctaaggcttgctttgcagcacaagactcagaaaactaaggccaagcgagagccactttataaatggcagcgtgacgctcaagagtgtgatttatcccagtatgaatgcaatgaggaaactgatgccgagggtgaccaagccgaactcccaccaactttgcttaaacgagctaacgctttagctcagggacgttatgatgtcccacccttgagaggcgttattattcctaagacagatggagagtggagagctttagctgtcgctcccttcttcgacgcagtattgcagagagcagtcgcccaaattctcgctccctcactagatcgagttatggataataggtcatacggttacaggcgtggcagaagtaggttagatgccaaggagcagattcaactggcctatcgtaatggagctagatgggttctcgaagctgacatcgaagatttcttcgattctgttgccttctctttagttgctcaaaggctaagagcattgtttcatcaagatcccattaacgaagctatcctggcttggctgtctgctcccgtagactatgatggtcttagattacagagaaaggcaggattgcctcaaggttctcctcttagcccagttctagctaatttgttgctagatgattttgactccgacatgaggaaggctggatttaattgtttacgatttgcagatgatttcgtcgttgtttgtcagtcacgagaagaagcagaaagagcatggcagagggcagcttcttctttgaatgagcatggtcttttcctagctgagaataagactagagtcataagtttcgagcgaggctttaggttcctcggttacctatttgttaatgagttagctcttgacgtaggatcgaaggctttaaaacaacatgacaaactttcatcgcctggccacgcaaagccacagtgtgcttccggatggctggccgactttctggcacaaaggccacaggcattgctgccacctaatgcacctcactcagaacaaagagtagtagagaaaactacagctatggtgcactctcaagctgtagctttgggtaatcgtgagaacgatggtactttcctatgcgtgtcgggagccccagcattgatttccactgatcatgggcgagtgctcgtccaaagagatgatgagactgtgatgtctgttccctggcagggattgagatcagttcttcttttgggacgacaccatttgactcatcccgccatgatcgctgcattgtcgcaaggagtagcaatccacttcgcaagtagaggtggacaataccaaggtcttttggacggaaaccagccgagactaggtccgagattatggctgttacaagaggagagagtagctgatgctgctgcatgtgtagctgtagctagagagttgactaatgccaggataagacaccagagagaggttctaagacaaagggcattatctggatggcacactctaggagattcattgtcccaggtagacgcatgtacagacctttcagctctacagggtattgagggtgctgctgctcgaacttatttcgctgctctggctcaagctgttcatcctcagtggggcttccacgggagaaacagacgaccgccacgagccccattcaatgctctgctatctctgggatttacattgttatatgcacataccgagactcttttaatcattgacggccttaatccccgagctggattctatcacaagcctcatgggtcacactcaactttggctagtgatatgatggaaccatttaggcatctggttgagaggtgcgccctatctttcttatcaagtggtaaggttaaacccgctgatttcagtgtaaaggataacggtgcctgtgagttatctaatgctgctcgtagattgtatttggagagattatccgagagattcgagacatctatgaagggtcgtgacggaagtgaggggaagctaatccagttattgaggtggcagaatagaagtcttattgagctgatccgagcaaagggaccctttactgcttggcttcaacgatag

SEQ ID NO: 120 - DNA, 1962 bp, Woodsholea maritima
  source 1..1962: mol_type = genomic DNA
atgatgagattagaggcatgtttaactgttaagtcccttatgacagcttggaagaaagtatacgcaaaccaaggtggtccaggtggagatggacagactctagcacagtttcagagaactgttctcttacaccttcacagattgggtgacgatgttcgagcaggactatacatgcctggacctcacagggttgtttcgatcccaaaacgagccggtggatggagatcactttctattccgtgtgttcgagatcgagttttgcagactgctgttgctcaaaggttacagccgatactcgaacctgaatttgagccagaaagctacggataccgtccaggtagatctgttgctcaagctattgctagggtggctactcttagaaggcaaggcttcagatggactgttgacgctgacatagaacgtttcttcgattgcgtcccgcacggaccactattggaaagacttaggccctttcttggcgacccaggtttagtaggtctagtcgaaatgtggttggctggagctgggccccacggcagaggattgcctcagggtagtccaattagtccactccttgctaacttgtacttggacgacgtagacgaaggtctaaagtcaacacacactagattggttagatttgctgacgactttgttatcttgactcgtaacgaggacgaagctctacaagccctagaacgagctaggggtttgctagacaaactgggtctttcattgaatttggagaaaactaggattgtaccattcgagggtggtcttgatttcttgggtagaaagttcgtaagggctcttgttgtggacgacgtttacgaggacgagggtgaagccctagctgctggcgagccagctcgtacccatgccagagctatgcctggtgaagatgctttagacttggcttcccttgaggcaggcgctgataccagtagagcacctagactaagagttttgtatctgttagagaagggacacgtcttaggtactcacaatagttcatttactgtcagaaacgctcaaggtgacccagtgagtacccttccgacaacgagagtagatagaatcgaagtaggttctcaagctagaattgctgacgaagctatcaggttggcccttgacgaggatgtcgagatgaggtggattaacggtcgaggtcagactgagggatacttgtcacgtcccgagagaggacacggtgctttacatttggctcaacttagactgtacgataatgcagaagctaggcttgcagctgctagaatcttagtcgaaggtagattgcgtaaccaaagagctttactgagaaggctcaatagaagaagaaaacgaccctttatcgctgaaagggctaaacaaatcggtggtgttttaaagtatctccctgaggcccagactatcgaagctctgatgggtagggaaggccaagctggtgccttgtattggccagctttgggtgcttgtttggaacacggatggacttttacccaaagggtaaggcgaccgcccccagaccctgtaaacttggtactgtcgtacttggcttctttgctgtgcagggacatagcttccttggccgccagacaagggctccacgtaggtattggcgctctacatgcagttcaggatgaaccgagggatactttagcattcgacctcgctgaggagttcagagcaccgctcgttgaaggactttgcatttggatgttaaacacacgtacattgggtcaccagatgttccatactagagaggacggacaagtttacgcacacggagaaggaattaaaacaatactgagatcctgggaaaactggttggatagaccagtaagatctcctagatctggccaggatgtcttgtggagagggcttattgaagaacagatcttgtgttggagagatcacgtgagtggaaaatccgtgtaccaaccgtacagaatggagtactaa

SEQ ID NO: 121 - DNA, 1968 bp, Desulfarculus baarsii
  source 1..1968: mol_type = genomic DNA
atggtggatatgaggttgtatgccaaaattattgctaagtctagccttatgcttgcttgggagaaagttttggctaataaaggagcaccgggcggcgatcgtcagaccctagacgacttcgcagagtctttagaaagaaatttagagggattacatgctgcattgaggagcgctagttaccgtccaggaccaattagaaatgtttccatcccaaagagagatggaagccctcgtcgtctgagcattccatccgtcgctgacagagttgtccagacagctctttgtcagggtttaaccccaattttggaacctgaaatggaagatgcttcttttgcatacaggccaggtagatcagtacagatggctgtagaaagggtaggaaggtactttagacaaggttaccattgggtcgtagatggtgatattgatgactacttcgattctattccgcatcatggcctgatggctgtcctgaggagatatgttgatgaccaagatgtcctgggattgatcgcacagtggttagctcatgcacacgctggtggagtcggagtgtctcaaggaagtccattatcacctctcttagctaatatatacctggatgatatggatgagagaattgggagaactggtgctagactcgttagattcgctgacgacttcctattgctctgtaagtcagaagaaagagctagagaatcgttagcagctatgtctgccctgttagctgaatacggattgggtttgaatccggataagactagaatagttaatttcgaacaggggttcgagttcctggggagactgttcgtccgtagcatggcacttgagagagaacaagaatctgatgcaccccaggagactccaccaggacctactcctgatgaccctagtcccccagtagaaccattacaccaagcatccgaaggtcctgggtttcaggatttgtctccgagattgagagtaatgtacctatcaagaaaaggatgtcgtttggacgtaagaggtcgtgctttcgttgttagaagcggtcccgagcccgatgcacctgaacttatggttgtactcccgtcccaactggatagggttgagctatggcctgggtgtgatatctctcagaaggctcaacgatttgctcttgaatgcaggactcctgttgcttatgtagacggatggggtagaactctcggagtccttgagcctatggtagcagataaagctgcccttcatttggctcaagctgccgtcgctttggatgaaacaaagaggttagctcttgctcgtttaatttgtgctggtagagtaaggggtcagagagcacttctaatgagattaaatagaagaagaaagaactcagacatagagagtaacttggctgcttttaaacagcttcctcgtaggattgctaccgctactactatttcagaacttcttggacttgagggcgaagccgctaaaaggtactgggcctctcttgctttgctacttgataagtcgtggggcttcagtagtcgtcagagaaggccaccacgtgacggcgtcaatatggttattagctacgttgcttcaatgttatatagggacttgcgatgtctcgctgcaagacatggactacaccctgggtttgcatcccttcacggatctctggatggaaaacctggatgtatttcagacttagttgaggagttccgagcccctttgtgtgaaggccttgctgtctacctggctaacaaccatattctgaagaaggaaatgttctataagactgacaagtggccctgtcacgttacaccagagggtcgagagacaataatccgtgcctatgaagcatggcttgatagacctgtaaagtctccaagatctggtgagaaggtaaagtggcgagggttgttggaagaacaggttttagcatatagggaccacgttatgggaagatctgtctacgccccttatgatatgaagtactaa

SEQ ID NO: 122 - DNA, 2019 bp, Azospirillum lipoferum
  source 1..2019: mol_type = genomic DNA
atgcctacggcaccccctgataatgcagacctatttgaagaagtgaccagattggacacacttgagagagcttggggtagagtcttgcgaaatgctggtgctgctggtggtgacggtttgactgtgggtcgttttgcagaagctgctccatctaggcttttggcattacatcgtactcttagaatgggtgactataggcctggtccccttagaagattatctattccaaaaccagatggtgctttaagaccactggctatcccaccagtcactgatcgtgtcgctcaaactgctgctgctcttgtattgacgccacttttggacggagaatttgaggacgcttcatttgggtatcgacctggcagatcagttcctcaagcagtagccagggtagctagatggagagatcaaggatacgattgggtcgtggatgcagatattgagagatactttgagcgtgttccacatgatagacttctaatacgattggaaaggagtattggtgctggtccacttactgaattgattgcagtgtggttagagtcgggagccgaaaatggtgttggtttacctcaaggttctccattaagtccattactatctaatttgtatcttgatgacttagatgaagctcttgatggcagaggtctacgtcttgttaggtttgcagatgactttgttctattgtgtagaagcagagaacgtgctgagagagctttagatcacgcagctgctgtattggaagaacacggtttgagactgaatagagataaaaccagaattgttccctttgatcaaggatttaggttcttgggtcacctatttgtaagatccttggttttgccttcaccaagaccagacgatggtgaagaaaccgaaggtgacgcattattgagagcactcgcaattagagatgccgccgctgaagcagaagaagctgccgcagaagaaagagataggcaaagtcgtgctgctgggcttgatcctgcacttagaacgttacacgtacaaacgcctagaaggagactagctttaaggaatgaggcttttactgttgttgagagggatgaccttggtggtgaaagagaactcattgctattcatcatcatcatttggatcgtattgatctaggcccagaagcagatgcagacgctgaagcattgagatgggcccttgctacagatactgagttggcatttgttgatggccacggtgctactcacggtagactaacgaggcctgaagctaggagagctgctttgcatttagaccaagcccgtcatgctcttgatgaaggtttgcgtttagatctagcaagaagaattgtagatggaagattgcgaaatcaaagagcacttcttagaagattgaatcgttccagaaaacttggagttgtcgaggacgctgtcctcgctataaatgcactacttagaagattagatacagctgctgatgttgctgcattgttaggatttgaaggtcagggagctgctcgatactggccagctttagctgcccaaattgaaggagagtgggaatttgaaggaagaaggcgtagaccaccccctgaccctgtaaatgctgtactctcttacctctcgggtttattggagagagatgtagctgctcttattgctaggcatgggctacatccgggatttggagtactacattcaccacaagatcgacatgatgcaggaatttatgatttgatggaagaatttagagcccccttaatggaaggcctggctgttactttgtttaataggagaacgctcaggccagatcactttagtagacgtgaaacgggtgagggagaaattaaaggatgtcgtatcgaccccgatgctgttggagctattattagggcttacgagcaatgggtccgtcgtcctgttgcatctccaagagatggtaaaagaactacttggagaggtcttataggattccaagcacaagcattggctgcacacgtccaaggaagggaaccctatagagcttacgtgatggactattag

SEQ ID NO: 123 - DNA, 1854 bp, Cellulomonas bogoriensis
  source 1..1854: mol_type = genomic DNA
atggctagagtcgcagatgaaggagccttgacgagagcttggtccgaagttcttgaggctgctgaaagagatggcagagtaccagtgagcgtccgaagatttgagagaggtgttgctgctagtctagtgcgactttctggagaattgtcatctggaagatatcagccatcaagagtttcagaagttagcctaagaactggtagtggctcagaaagagttttgagaattggcgcagttgtagatcgtgttgtagaaaggagtttgcttaacgctttgactcccgttattgatcctctactgtccccttttgcttttggttttaggagaggtctaggcgttaaagatgccgtagcagctttggctagagctagagatgaaggttctacacatgtgcttagaagtgatattgccgccgcttttgattccgtgccaagggcacgagctgttcaggcactttcacgtttagtaccagatagacgtgtttgtgatgtcgttgcctctttactggctagactagatgattatggtttagaaggtgtgggtattgctcaaggttcagctgtatctccactcctcttaaatttgtatttacttccctttgatgaggctttgatggctaatggttttacaccacttagatacgctgatgatattgctgttccagctatgtcggaaagtcaagcccaaagtgctgcccaagatgtcgcccatcaattagaatgtctaggtttggcttgtagtgcacccaaaactagcatcaggtcctttgatgaaggtgttcatttcttaggggtaactttgagagctagaccttcttcgacagcaccacccccagctcgatcaaggcctcaaagaatttctatggtcgtttctgagggtggtgctgtagttagaacaagacgtggtcgagttagagtagaccgagatggagaaactgttgcctcaatgtcattgtccagagtagcacgtattgttgttcagggtcgtgtaggtttgacgactccacttcttcatgaagctgctcagagaggtattgatgttgtaatgcttagtagatcgggtgggtatgttggtaggctatcaagaaggcgtccaggtgatccttctttgaggagagcacaagccagagcatacgattctggagctgatttggagaggcttacaactgcttttgtttccggtaagattactaatatgagagtagccgtattgcgacaccaaagaggcgccggaacttccgaagaaggagccagagtcgctgcacaacttgctgaagcccgagcaagggcatctgttatgaggtctgtaccttctttaatgggagtagaaggggccgcaacaagagcctattttggttggctaggctcgcgagttggcgaagaatggggatttcatggacgtgctagaaggccaccccctgaccctgttaatagtatgttatcctatggatatgtattattgtgcgctgaaggagtttctgcttgtgaacaagctggtcttgatcctgatatgggctttctacattctgatcgatggggtcgtccgagtttggcactagatttgatggaagaatggcgtcctgtcattgttgattcgactgtgttgaggattatttcgaataaaagactaaagccatcagactttacatttgatgccaaacaaggagcaaggatgaccgctcatgctagacaaacttttctgagagaatatgaagcaagaatgttgacgcttgctggatcagatgccggggctggtcgtcaaccatatagaagattgattgctactcaagctatgagacttgcagaggctttgagaacacccggtggtccatatcgtccttatgtttggagatag

SEQ ID NO: 124 - DNA, 1677 bp, Micromonospora rosaria
  source 1..1677: mol_type = genomic DNA
atgaccagggtgtctatccctaaacccgatggcggaatacgtagcttggctataggagctattgaagatagaattgtcgaaagagctgttctggatgttcttgatccagttgtggaccccacactttccccgtggagttttgcatacaggagaggcttaggcgtgagggatgcagttcgagctttggcagaagctagggaatctggtctagcttttgttgttcgttgtgatattgatgattgttttgattcgatcccgagatggcctctacttaggagactaagagaattggtttctgacgctgaattggttgccttggtagaaagattggttggcagaccagtcactggagaaagggcttccggtggtaggggattgcatcagggcggatcgctcagtcctttgttggccaacctatatttggatacctttgatcgagcattgatgcgtcatggacaccgtgtagttagatatggagatgatatagctatatctgttcccgataggccaactggacttagagtattggatttggctgatgcagaagcagaagccctgtctttgagactaaatactgatgatagacaagtcatagcttttgatgaaggagtccctttctgtggtcaggttgttactgcatcaagcgggccaacagctgatttacaagctaaacctctgcaaggtacagtctttgttacaacccaaggagcattgctaagagtaaaaggagaacgacttcgtgtcgaagatggagacaggctactggctaatgtaaatttgaagcgtgtgaggcaaatagtctgttttggtagagtaggagtgacttctaccttgttacaaagaattgtagaacgaggaatagaacttgcttggttatatgaggatggtagacatgcagctagagtaagtgggttggatggtactgatccagaggtccgtttagcccaatatagagctgctgatgatgcacgtcaggcactgagaatagctaggcagttagtcgctggtaaagtaacaaatatgagagttggtcttttgcgagctgctagggctcaacaagctccagagcttgctgatcgtcaggcacgattggccacagctcgacagtcagctttgattgcagattctactgctgaattgatgggttatgagggttcagctactagagattattttgctggtctttcacaaattttgggtcctgagtggggctttactactcgtcaaagaaggcctcctcctgaccccgtgaatgccatgcttagttttggatataccttgctgactaatgaggcactcacggcctgtcagctggctggacttgatccatatctcggtatgttacattctcctagaagaaatagaccttctttggcattggatttaatcgaagaactccgacccgttgttgttgatgctaccgttattaggctagtacgaacaggacaagttactcctaagaactttactctgacagatgatagaggatgtagattggatgatcatggtagacgtgcctttctggatgcttatgagcgtagaatgctaacacttgtccatcatcctgtagaacaaagaagaataccgtggagacatgttttgttagcacaagctagaacacttgctgctgtattgtcatcgcgtcgtcccgaatatagacccgtagtatggcgttaa

SEQ ID NO: 125 - DNA, 2007 bp, Tolypothrix campylonemoides
  source 1..2007: mol_type = genomic DNA
atgtttactatagagcagattacttcggcttggttacttgtgagagccggctcaagaggagccggggtcgatggtatgactgtcgatctattcgcagcaggagttaacgagcaacttagaatattactgaggcagttgcagcaggagagttatagagcctctcctgctaagggattcttcgtcgcaaagaagtcgggtggaaagaggctgatcggaatcccaactgttcgagataggatagttcagagattacttttggaagaattgtacttccccctagaggacaccttcctagactgtagttatgcttacagaccgggtagaaacatccaacaggccgtgcagcacctgtacagctattatcaataccagcccaagtggatcattaaggctgatatcgctgagttcttcgataatctatgctgggctcttttatttacagccctggaagatctccaactcgaacctatacttctacagctgttagagcaacagttaaagtccggcatagttattgcaggcaagccaatctatccagggaaaggtgtattgcagggcggggtcttgagtggagcactggctaacttgtatcttacgtcattcgagcgaaagtgcctatcctacggtatcaaccttgttagatatggtgacgacttcgctatagcttgctcgtcctggcttgaggctaacaggatattggataagatcactacatggttaggcgagctgtacctcaacctacaacctgagaaaacccagatcttcgctccggacgacgaatttacttttctaggttatagattcgctggcggtgaggtgtatgccccgcccccacctgtaatgacgagggaaggtgagtgggtcaccaatgagtccggactgccatacttcaggcctaaatctcgtcctgttaagttcgtgagtagaccacccaaagcctgttctattgcttcaccaattaagttcccaacggccccaattagccacttatggcaggagttcatgactaccctatatgttacagatcagggtgcatacctttctgttaagaaccaacaattccaagttttctaccagggcgagctgaaaatcaaggtgcctgctactagagtcaataacattgtaatgttcggatgctgcaacgtcagtcatggcgctgtcagtatggccttgaggagaagaatacctattatgtacttatcacagaagggtagatacttcggccacaccgctgttcagggcgacgcacgagtggagtacctcatgcagcaagttaaatgttgcgaaaatacacaattcacccgacaacaggccgaggctatcgtggctgctaaattacacaactccagaatcttactgatgagactcaataggagaaggccgactgagatagcaacacaggccattgacttgatagaaatccttattgactcactgccacaggccgagtccctcgacgccctaagaggttacgagggaaaggccgctaccgtctactttcaggcactcggctcactattcactggcttcttcgctttcgacaaacgtactaaaagaccacccacagatcccataaattctctgatgtctctggggtatacccttttgtctcaacaagtattcagtttcattcaatcagtaggcttgcatacacatttcggtaacctacacacaccaagggacaaccaccctgccctcgttagcgacctgatggaagaatttcgagcccagatcgtggactctttcgtagcatatctggttaacaagaagatccttacccctgaggacttcacacccccagatgagagaggtggagtgtatctccaggcttcagctctgaagaaatacctgaaacactgggaagaaaagttgcagacagaaactactcacccacatactggttacaaagttgcctacagaagatgtattgagctacaagttagggaatacatcgcttgcctggtcggagaggtcgaagtctatcgacctatggtctggaagttgtag

SEQ ID NO: 126 - DNA, 2019 bp, Oscillatoriales cyanobacterium
  source 1..2019: mol_type = genomic DNA
atggaatttacgccagagcctctccatgccgcttggttacaggttagaaagggtagccgttctgccggcatcgataatatcacagtggaccttttcgctggagtcgcaaggtatcaactgcaagtacttttgtggcagcttcaacaagagaactacttcccaaggccggctaaggggttctatttgaggaaggctagtggtggacaacgacttatcgggattcctactgtaagagacagaatcgtccaacgtttcttactagatgagttatactggccacttgaagatgtgttccttgactgttcctacgcatatcgaccagggagaggcattcagatggccgttaagcacctctactcttactaccagttaggacaggcatgggtgattaaagcagacatcgaaaagttcttcgataacttgtgctggcccttgttgctcaccgacctggagaaattacagttcgaaccgatactgaggcaactgatagaacaacacttagcttcaggcatcgtggtaaagggacaacacttccacccaaaccagggtgtgcttcagggtggaatattatccggggcattagcaaacctctatcttaatgaatttgatcgactgtgcctgtcacatggatttaaccttgttaggttcggtgacgatttcgctattgcttgcgctgactcaatacaggcaaacagatgcttagaacagatctcatcgtggttgggctcattctacttgaagttacaaccagaaaagactcgtatcttcgccccagacgaggagttcacgttcttgggttacttgttcaggaacggtgaggtattcgccccagagaaggcacagccaacaacccaggtaattagagccagcggcacttctttctcaaggcctgcaacacgaaaggttccagtctttagcggcaaaccgttagcatgtagtatcgacgttaaacctatcgtcttaccaagagcaaacagtgagcacttctggagagagccaatgtctacactgtacgtgacaaaccagggatcatacctttctatatataaccaacagtttcaggtattctaccaaagagagttagaaataaaggtccccgcttctagggtcagtcatatcatcctattcggttgttgtaacttgtcccacggtgctgtgtcctgcgcattgcatcgtagaatccctatcctgtacctctctcaaagaggaagatacttcggtagacttcagagtgacgggagagccaaagtcgagtaccttactagacaaataatatgctcactcaacccagagttcgtgcgtctgcaagctgaagccatcgtgcgtgctaaattgcataactctaggatactcttaatgcgtctaaatcgttatagaaaatctaagaacgctgacgagatgtctgtattacaggctgttgagatgctagagatcttaatggattcgcttcacaaagccgactcaatggacgcattgcgtggatacgagggtaaagctgctaccgtctacttccaggcccttggctcacttttcagtggtccattcgcattcgagatcagaaccaaaagaccgccgacggacccaataaactcactcttgtctcttggttacacgctgctctcgcaaaacgtatcttccttcgttgaggctatgggacttcacacccacttcggtaacctacacgtgcctagagacaaccatcctgcacttgtttccgaccttatggaggagttcagggcacaagtcgtagactcgctagttgcttacctgattaactcacaaatcttcatcgccgacgacttcaccccacctgacgaaaggggtggggtgttcttgcagccccacgcattaaagaaattcctgaaacactgggacgagaaattgctttctgaagtcacacacccgcacacaggatataaagttgcttacagaagatgcttggagctacaggtcagggaatacgtatctgctttaatgggtgaagtagaagtctaccgtccaatgatgtggaaaatttag

SEQ ID NO: 127 - DNA, 3009 bp, Psychrobacter lutiphocae
  source 1..3009: mol_type = genomic DNA
atggtaacagcttcacacactctagattggcaatcggcggactgtcagttgatctggcatagcgatactacgcatcgtaaaccggagaacatttatattcgtttatggggtctgatccaaaccatcaacaccaccgaagatcagtatctggaacaactccatcgttgccgtgatgagcacaaaaaaaacatcattattcagcatctggaggctaaaaagcaatacttcaaccacccgaatttcccgctgatcgctttccatccgacccaaacgtcctatcataaggtgagcctgaaaaagggtgagatcatcccgatctcactggtatttggtggcaaatattcccacttggctcaggcgtggctggactggttcgcagtgcggctgactctggagaacaccggttttagcttggcgaatatcccgtctctgaagccgcatcagatccagctgcctccgctgactgcgaacaagctcgcgaagtgccagatcgcgaataaacaagcgaaggcgccaattgacgcccaagagttggaaaccgctccagttcaaggtagccctcaagaaatgaccattctgatcgacaccccggcaaatttcagcctcaacaagacccaacaaaaacaggtgagcaatctgagcactgcggatattcttaagtacgccttcatccacaatagtcagaaaagattatgccagtggttcccgcaacaaaacaaagtgattgagagctggttcaaacagtggaccccgttgctgttgaagagcccgctgtctacgtaccagctgcacgagcgccacgagatcaaaaccgtgagcaaaagcagctccgcgcgtagccagagcaagatccagttccacaaaggtcatgcgggttggctgaccttcaagggcgcatggctggaggttgatgagctgctgactgtcttgagcagcattcatctgttaggccagcgtatgcatatcaacggtctgggctacttcctgacccagcagcaactcgaacactccgcgccgaccctctggcaacgtcatctgatggataaacacaaaattaaacacgccattgaggaagttctggagcgccacgagttggagccggtggttgatgataagggtcgtattatgcaggtagacgccctgaccgagctgatatacacccagctgtccaccggtgtttacgcgccgaaaccaacgcgtgcaatatttgtgccgaaaccgaagggcggcaaacgttgcatcgaagaactggaacaggtcgacatgatggttcatcgtttagtctttaatagtatcgcgaaaaccatcgagtcctatcagagcccgctttccctcggctaccgcaagggctacagccgccagatggcgagagacaaggtgcaagcgctgatcgactctggtttcggttgggttgtggaagctgatattgaatcgttctttgataatgtgccgtttgagcgcctgtggcagcgcctggctaccatactcccgcaacgtgaattgcaaacgatcgcgttgatcaagaaacttatgcaagtgggctacaccgtttctaatgcatctggcaccgttgtcaaagaacacctgcgttttaaaggtctgatgcagggcagcccgttgtcgccggtgcttgctaatctgtatctggcaatgctggacgagcagataaacgcagagcactttgcattcgtgcgttatgccgacgacgtactgatgttttgtagaagcgaagcagatgctaacaccaccttggcgtggctggaccaacatctgagcgaactgggcctgaatctctctctgtcaaagacggccattacagcagtcaacaacggtttcgagtttctgggttaccgcttcgacaaagaaggctcggacgacaagtccattgtgccggttctgcgccagcgtaagccgattatgatcaccggtagccgtaagtatcttggtattaacggcggcgccttggaggttcgccagaaacaagcgcgcaagcagccgggtcgtagcctgtccaccagcccgttgtccaaccaattggttcaggtcatcccgctgcgtcgcatcagccagctggtggttatggggaaccactccctgagcagcccattactgacggcctgcgcgaagcaccatatttctgtgcacttagttaaccagtggggttttcaggtgggtacattcaaaccgagccatgcggagtattacgcggttagcgccgagcagtaccagcgccaccagcagttgaagccgtctgaacgcatggccatcgccgctgacctggtgttggcgaagatcaacaattaccagacctggattattaacagctaccgtaagggcgacgcgcaggtcaataaacaattagaccaaatcgcgaaccaagcgagtagcgccaccaccatcgaagcactaatgggttacgagggtcaggcggcaaagatttgttttcaacgtctccagtctgtgatgattggtgatcagcaagcggcgtttagcagcaagcgtcgtagtcgtggtggccctgatcgtctgaacagcatgctgaacttcggctactactggcttttcacccgtatctctggtttgctgcactcacacggcctgaacccgtatctgtcctttctgcacgaaccggaacagaactatgaaaccttggtgtatgatattatggaattatttcgtgttcaggttgataaaacggtgttgcgtctgattaatcgtaaacaaatccaggctgactcgttccaccttgatgcaaaaaagggctggaaactgaacaatgacgcgctgcacttgctgagcaaccagttgcagagcaccttcgctagcaaaattaaccaaacctttctggaagatatcatcctgatccaagtgcgcacgctgctgcactgggcaacccagcaacaatctttggtttggttctattggtatgctgataaggacaacgtgcaatttagcctgccggacgaaagcccacagactctgatcattgacaacatcgagagccagtaa

SEQ ID NO: 128 - DNA, 1902 bp, Ruminococcus sp.
  source 1..1902: mol_type = genomic DNA
atgtttagtatggaagagatgctatcaaaaaagaaccagcgtgaagcgtttagcttcttgaaaaacaagaaggacggcattggtgcggatggcatgccgctgagcgagttggaagattattggaaaatcaactccgagcgtatctgctctgaactcaaaaagggcaagtacaccccgggtattatcaaatgcaccgaaattattaacaaccgtggtaagcgccgtgtcattagcaacctgtgtaccgtggaccgcttcattacccgtctgctgtaccagaaactgcgccgttatatctctccggagttcctggaaaactcgtatgcatatcaagagaataaaggcatcctgaacgcggtgcagaaggtgcagaaatacatctctgagggctcgaaatacacgatggaaattgatcttaaggactatttcgataccatcccgcatgagcagatgatgtccttaatcaaggagcgcatcaccgatgatcgtgtagttgacctgatctataagtacatgcactgcagcgttagccaggataacgagatcaaggagaagaaaaagggcctggttcagggtaacgcgatctcgaccgttctgtctaatctttatctgcacagcctggatcagtatctgcaagagcgcgaatacaagtgggttcgtttcgctgacaacatcaacgtatactgccgtaaggagaatgacgccatccgtgtgtataacgagatcaatatctacattaaagagaaattgcacctgaccattaacgaaaagaaatccggtatctacccggtcctggaccggattttcttaggttataaattctataagtacaatggcaagtacgaagctaaaaagtacaaataccagaaaaccgactgttttcataattggcacgagagcgcgctgcaacgtgtgaatcatgaatatcatattgttcaaaacggggtgctgaacaaaaaggattatagcctgattttcgagaacgaagaggagaagtgcgatattccggttggtgtcgttgaccagattaacatttactctgaggtcaccatcgcggctaccgcactgcaactgatcagcaaaaaaaagatccgtctctccatcatcgataaatacggcaacctcattggcaactatatcccggcaggttatggccgctctagcattgagtttttaaagcaggctgtgttttacgaatcaagccatcgtttcgaaatcgcccagaaaattgaggttgcatccattcacaacctgcgggcaaatgttaaatactatagccgtaaaaacgcgcacgcgttggacgctgtggtggaacagctgagcgagtatatgaatgaaatgcgtaatgctccgaatattgaagaactgatgattatcgaagcacaagcgcgtcagcagtactacgcgtcttttaatattattctgaaacaagaatcgttcttctttgagaaacgtacgcgtagaccaccgaaagacgccatcaacgcgctgatcagcttcggtaataccctgctgtacaattttgttctgcaagcgatctggaaaaccagcctggacccgcgtatcggcatcatccacgcgactacgaaccgcaactacagcttgaatttggacttcgccgatttgtttaaaccagtgatcgtggaccgcgtcatattcagtttggtgaacttgctacaattacaaagcgaagttcactttgaagaaaaggagtccggtgcgatttacctgaacaaagagggtaaacgcatttttattaaggctttcgaagagaagttggcggacaaaattaagctgggtgaaaaaacctacacgtacaagcaaattatcgaggaggaaatccgcaagttccagcgttatatcgttaaggaggagaagtataagccgtataagtactggtaa

SEQ ID NO: 129 - DNA, 2811 bp, Vibrio sinaloensis
  source 1..2811: mol_type = genomic DNA
atgacaaatcctatccttaactcaatcgaaccaatagctcaattactgccactccgagcaatggttgttactctaagattcactcaggatgccactttccaattctaccaccacgtggctgcccacgcatgggttagacacttgtgcggatcacccgataacttttctcaacaggtaaccgtccagacactagagaacggaaagacggattacaaggcaggagaactgtatagatttaagctagttttcacccctcttggtggtaacctaatacaaccattgatcgatgctctggaaagactgccgtcatcggcaaaggccctaccaaggggagcttgcattaataataatgtagagctggtagaattgaagtgcggtgtggggaacgaggaaatccaccacgccattgatctgtatccttacgatctgcaagcattggctcaggacgttgctcattggcagcaaagagatgatatctatatggaatctctgactcctgctaggttgcttaaagaaaaggcatcacaggaatcctcaagaggtgacgctagatactgccgagataaggtccacttctctgcaacaactataaccagaagattcgtggacacaataattggccttgtcgagagtcatacgggacagagatatcagagggatattgaggcttccttcgaaattcaaccagaactatccttttgggtcgaacaccgatacggccgaggagagaagtggcagaagaaacctatgtcaggtgccttgttctccttgaaggttcgtaatgtaccaagattggagaagtggcagattgccttgttagttttgggtcagtggttgggtatcggtcagagcagaagtatgggtttgggtctttactggcttagggacgtaagtgagactctaggtgggcactctaacagactcattaaagagtacttcaatcagcgtagattggtccaactggtgcaggaatcattagatagtcagttctcagagcaggaaagaaaccagtataagaacaaggtgatcgggctttcccatactatcctggctggtgattacaaggcacccgtgttgactcaggttgagatcgataagagcgacggtggtgtcaggactctgagcatcccacccctggctgatcgtattcttcagaaagccatcgcaagacctctcgctgtatctctggacggtctttggaaaacccattcctacgggtacaggaaggacctatcaagacatgacgctaagtttgctatcaatcaggctatacagcaaggttacgagtgggttttggagtccgatgtcgattcattcttcgataatgttgactggagaaatctacagaccaggcttaaacttcttttgccgaacgacttcttagttgacgttatcatggcctgggtgaaggcccctgtgaaaacccctagcggacagatcctggagcgtactcagggcctaccgcagggttcacctctgtcccctttactcgctaacttggttctggatgatttcgatgcagacatgctggcactggactataagttaatcagatacgcagacgacttcgttctattattcaagaagcagagtgaggctcagatggctttagatcacgttattgcctctttaaatgagcacggattaaacattaaagctaagaaaactcaaattgttcacgctaacaaaggcttcaggtacttgggtttctggttcgtagacggatatgctatagaaacctctcgacattaccgtcaagaggaacaggagtatcagcaggccatcacacaccaccaagcccaactcgccgaaagtaaacagagagagaagcaacaaattggagaaagagagcaactcggtacgcttcttgtcattgccggcgaagtggctatgttaacctgtgagaataagagactgaaaataacacaacaggacgagtctcgatattatgcttgggaagaattggagacaatactgattctgggacctcacaatataagcacaccctgcatccgacaggctatgcaccatcaggtggccattcactttgcttctcgttacggtcagtaccagggtgtcgcatgttcgaacatgccaagccaggggcaccagttgtggcagcttcagattgcttacttgcagaaggtagacgttgctcttacttggagcattgagctggtttgtgccaaaatcgacggtcatattcatttgatcagaaatagagaaaggcagagtcctcttttggagaagttgcgaactatcaagagaaaggcaaggcgatccgacgatttacaagtacttttgggaattgaaggcgaggccgccaaactccagtgggagttcttcaagcaacatctggattgtgaatggcagttctccggaaggaaccgaaggcccccaaaggacccaatcaacgctatgttgtccttaggatatacatatctataccatctgacagattccttgattcaatcaaacgggctttgtccctgggctgggttctaccatcagccacacggagcccacagaactctggccagcgacctcatggaaccactccgagtcgtcgtggaccgaactgttctggcattggtagccaagcgtcagattaagcctgacgatttccttatattggaagatggatgcgaaatgtctagggaagcccgtaaggtgctactgactcagttgttagcagaccttacaacgaaaaggaagaaaagcgatagggttatcgaccaaatgattacccaagtaaaagaggttaagattgctactaagttggctttaaacccaaatttctggcgtccttga

SEQ ID NO: 130 - DNA, 1899 bp, Blautia sp.
  source 1..1899: mol_type = genomic DNA
atgtttacaatagatgaaatgctatcaaaaaacaaccaacgtctggcgtttgagcactttgcgaccaagaacgacggttgtggtccggatggtatgcatgtttccgagctggaaaaatactggcgtatgaatcatgatcaaattatcagcgatttgaagaaccaagaataccaaccgggtattatcctcattcgtgaacacatgaacaagactggtaagcgtagaaacattgcgtctctgaacgtgattgaccgcttcatcacccgtctgcttagtcagaagctgaaccgttacctggctccgatcttctgcgaaaacagctacgcatatcaagatagcaagggcgtgatgccggcggtgttgaaggctaaggagtacgtggagttgggcatgcgtcacgtcatcgagatcgacctgaaaaattattttgataccatcccactggagaatctgatcccggaaatcgaacgctacatcacggatgaagcggttctgcacctgatcaaacagtatttgttttgtgatattagcttcgaaggtaaaatttcgaggaaaacccagggtatcgtgcagggtaacgccatctccccgattctctctaacctgtacttgaatgacttcgacaaagagttagacgaaagcaaattatgctggattcgctatgcggataacatttacgtttacatggattcttacgagaaggcgctgctggtgtacagcgagctgacggaacgtcttgagcgccgcaagctgaccgttaataaagagaagagcggtgtctttgacgtttccacccgttcaattctgggctatgatatcctgatccgcaacaaaaaggtggacgttcgtaaacacatctacaagtccgtcaatcaatatagcaattggcatgatagccgtctggaatttatcaatggtcgttatcatatcacgagcgacggcatcttgaaccgccaggatttcggtctgttgttcgagaacgaacagaagaagcactatatcccggttgaagtgagcgaccagctcaacatctatggcaatgtcaccctggcaagcaacgtgctgcagagcttctccaaccgtgaaattaaagtgtcgtttttcgacaagtacggccgtttgataggcagctttcttccggaaaagaccaaaaaatccgcggaaatcatcctggtccagtccaagaactatctaaacgaggatgttcgtatggacaccgcgcgtcgtatggaaatcgccggtctgcacaacattcgcgcaaatttgcgttattacgacaaaaagcacaaaggtgatttcaaagagaaggtggacgccattagcggttatattgacgctctgaatcgcgctccgagcgttaacgacatgatgctgttggaggcgaaagcgcgccagctgtattacacgtgctttaatcaaattttggaaacctccgactttcagtttgagaaacgtaccaaacgtccgcctaaagatgcaatcaatgcgtgcatttcgttcggcaacaccctgctttacaatctgttcgtgaatatcatctggaaaaaaggcctggacccgcgttttggcgtagttcatgcaagcaataagcggaaccaatctttgaacctggacttcgccgacatcttcaaaccgattgttattgatcgtattatcttcaccatgattaacaagaagatgctcacgctgctgactgatttcgagacatctaaccaaggtgtttatctgagccgtgaaggcaaaaatatattcctgcaaatgtatgaggaaaagctgaaaagtcgcattaccattaagggcaaagagatgagctactaccagctgttggagagcgaggttcagaattacaaaaactttattctgactggtgaaacctataagccgtataaatactactaa

SEQ ID NO: 131 - DNA, 1125 bp, Rivularia sp.
  source 1..1125: mol_type = genomic DNA; strain = PCC 7116
atgggatacgagaacatcagataccttcacaagccgtacttcgcagaaacgattactgagaagccaccacagactgaggaagatgactcaccattccagaagaacatctggaacgctgagatggctgcaatctatcttatcgagcagggtacaaacatctacaaagactaccagcgtttcataatacacgttagtgagaagccaaaacttgaggtccctatcagggacgtacaacagatcatcgtctttggaaatatccagctaagcacgcctgttatccaggcctgcctgaaggagcagatcccagttgttttcctgtctcagaccggaacataccatggccatctgtggtcggagaagtcaatccaccttgataaccagttggttcaggctgagcgaaggaacgacgacttattcagattctcagttagccgagcagtggttctagggaagcttctgaactctaagcaactactgatgagattcaacagaaggagaaagattggtaaggtcgaagaggcaatctacggtatcaaccaggacatagacgcattgaattacgtggacaacctggacacgctgaggggatacgagggtatcgcagccgcccgttacttccccgccttcggtaacttgataacaaacccaaagttcagcttcagtcaaaggttcaggcagccaccaactgacgaaattaactctctactgtccttcggttacacattgttattcaacaacgttttatccttcatcatcactgagggcttgtcgccgtacataggacacttccattacggtgacaagcagaaaacttacctcgcattcgaccttatggaagagttcagatcgcctatcgtagactctcttgtattgaagataattaacaagagcctattcaagccccaagactttgacgttgtcgcctctaccggcggtgtttatttgtctcagaccagccgtagggtgttcctgaagcaattcgagaacagaatgaacgaggaaatctcccacccggacctgatctcccaggttacttaccgtcatgctatccagttgcaggtcagacgatacaaaaggtgcctgctgagtgacaacatctacgagtctttcctaagggctgactaa

SEQ ID NO: 132 - DNA, 963 bp, Rivularia sp.
  source 1..963: mol_type = genomic DNA; strain = PCC 7116
atgatatcatctcagttcatagacataaacaacttccagagagcatgggagaaagtcgcagacaagagagggtgtgctggagtagacggagagacaatctcctcattcgccagtaaccagactgtgaacgtctaccaactcatgaatagtgtggcagacggatcttaccagccgttcccatgtaaacaagtcatcatcccaaagaggaatgggtcccagagggaactaaagataccgaccatcagggataggatagtgcagcaggctctcctaaacgtcatctcacctcttatggaagaaaagttctctccagtctctttcgcatacaggccaaacttgagctacattaacgccgtggagaagatcgcagattggagagatatggggtacgtttgggtcctggacgccgacattgtgaaattcttcgacaacatcgatcaccacagacttttgcagcaagtgcgactacacatcgatcaccctggcatcctgtgcctgataaaggcatggatctctgtcggcgtcgagacacgagagggtttgattcttccacagaaaggaatcccacagggcgctgtcatctcacctatcttagctaacatctacctgcacgagttcgacgagataatctctgcatctgacctagagatagtaagatacgccgacgacttcttagtactatccaccagccaggagagaatagccatagccaagtcccaggttatagacttgctcgactcattgggtctagagattaacacagacaagacacagatcacgtctttcgagagaggattcagattccttggacacgggtttctgtccgacgcaatatttccagtggacaccaacaaggctaaactgaaaagtggaatcgagacaaacagagaaaagactaggacccgaaagacgagcaagaagaagctctaccataatccttacagaaataagaaggtcgtatga

SEQ ID NO: 133 - DNA, 261 bp, Fusicatenibacter saccharivorans
  source 1..261: mol_type = genomic DNA
atgtatgtcattctagtttacgacattcaccagaagcgagtaggtaaggctctaaagatttgtaggaagtacctgatccacatccagaagtcagttttcgaaggcaacataacggagagtaagctaaaggccttaaaagaggagctggggcacctgatcgacactcagatggactcggtaattatctaccacctggactccgtgaagtacactaagaaggagcagatcggcatcgtccaatcgacatccaatgtcatttga

SEQ ID NO: 134 - DNA, 282 bp, Candidatus Accumulibacter
  source 1..282: mol_type = genomic DNA
atgagtgatatggccctgtatatcgtcgcctatgacataactgacgatagagaaaggcgtcaagctgaacacatccttcaaggattcgggttcagaaggcaaaagtctgttttcgagtgcagattgactaggggatatttagcaaggttgcagcatgagttagctacaatcggaatggagacagggttcgttatgatttatcacttagccccaaatacccgaccttttacaataggagaagtgcctcacttcccagacgacgactgggcctgggtaacttaa

SEQ ID NO: 135 - DNA, 285 bp, Candidatus Accumulibacter
  source 1..285: mol_type = genomic DNA
atggagcaaacgtggctggttacgtatgatgtaagtgatgatggttgtagaaggagagtagaaaggatattgttgggtcatggagagcgtgagcaatatagcgtgtttaggtgtagactatcttctagggaagttagggatttacgagctagattggcaggacacttgaagggttctgatagtattcgatactaccccctttgtgctgcatgtttaccaaggcaaccccaacgtaccctagtcgattcagctgaggctggtatcgcttggtatttcgcagtttaa

SEQ ID NO: 136 - DNA, 261 bp, Eubacterium saburreum
  source 1..261: mol_type = genomic DNA
atgtttgtaatcataacctacgacgtaaaggccaagagggacccgaaggtcatgaaaactataaggaagtacttaacccacgagcagaggtctgttttcgaggggctcatcacgcccggtagattgaagcacttgaaggaagaacttaaaaggattgtgaacgtgtcagaggactgtatcaacatttactccctggaaaccctgcgttacagtaagaaggagtccatcgggaagcaagtttaccacggcaacatcatttaa

SEQ ID NO: 137 - DNA, 291 bp, Bacteroides fragilis
  source 1..291: mol_type = genomic DNA
atggttaaggccaagaagatcttctgcgtcgtcgcttatgacatccaggacgacaggtctaggattcagatcagcaagatccttgagaagtacgggaccagaataaactactccgtctttgagtgcatgttcaccgaccgtcagttccagaaaatacagataaacctggagcgatggataaacagacgttacgacacggttgtctactaccctatgtgtattaactgttacacccgtatcatctaccagccaattaggaagaagattatcaagactgtggagatcgtgtga

SEQ ID NO: 138 - DNA, 273 bp, Campylobacter fetus
  source 1..273: mol_type = genomic DNA
atgcactacattatttgctacgatattgctcagactcgacgaagaacaaggctggctaccctactagaggccattggtacacgagctaataagtctgttttcgaggccaaattgactgccaaggaattggaattgttcatcgctaaggctaaggcaataatagaaccaaagacggactctgtcctcatttacccgctttgcttcgattgcatgatcaagtctctgtcgctaggtcagaagggagtgttcgaggtgaaggactctttcgtatga

SEQ ID NO: 139 - DNA, 279 bp, Teredinibacter turnerae
  source 1..279: mol_type = genomic DNA
atgaaaacttacttggtatgctttgatatcaccgacgataagagtagaaacaaagtcggtaaccttcttctggcatacggagagcgtgttcagttgtccgtgttcgagatagcactcaacaacaatcgagaactagcagccttaaagacccagctccagaccctaatggaagaaggggacgaccttaggttctactacttatctaaggaaacccgtcgtcagtcagaggatgtgtatggaaatgctatagccgacttcccaagcgtgatcatagtatga

SEQ ID NO: 140 - DNA, 288 bp, Desulfarculus baarsii
  source 1..288: mol_type = genomic DNA
atgagtggtgccgagatgctagttgtatttgcttatgacgttgaggacgactctcgtaggcgacgactggcacgtgtactaggcaatcatgctgttagagttcagaagtctgttttcgaggcttggttagacgaaggtgctgctaagataattgctagtcgtgccgccgcagaattgggaccgagggactctctgagagtatacgcattagatgcttctggagtcggcaagaccttggtgttcggtgttactgcaccaccacaatcccacgactactatttcgtctaa

SEQ ID NO: 141 - DNA, 288 bp, Azospirillum lipoferum
  source 1..288: mol_type = genomic DNA
atgtctgaaacaccaatggttatggtgttctgctacgatgtcgccaatgatagaagaagaaggagagtctcagcagtacttgaggagtggggagttagggtacaaaagtccgtatttgaagcaagactaactgagcctcaagctagagctttgttagcaagagctggaaaagaactgggtccaagagattctcttagaatgtatgcacttagcgcacatggcctcgctcattctgctgccctcggtggggctccagttcccgagcaacaagacttttggctcttgtaa

SEQ ID NO: 142 - DNA, 279 bp, Cellulomonas bogoriensis
  source 1..279: mol_type = genomic DNA
atgatatggatcttagcatgttatgatatagctgacgatgatcgaagatccagattatctgatcttctagcagagttaggcccaagagtccaacagagcgtctttgaatgtagacttccctctaagaaggctttgagaagattattgggagaacttgctgcacttatcgaccccgttgaagatcaggtgcgtgtttacgagcttggtggccaaggacccagaccgcaaattgttggcactcgtatcctagaagaatggcgagacatgtgggttgtttaa

SEQ ID NO: 143 - DNA, 288 bp, Micromonospora rosaria
  source 1..288: mol_type = genomic DNA
atgtcccgtgtttgttatgtagttgcctatgatatagccgatgacgaccgaagggccgatctcgctatgtttctttcagggtacggtccgcgtgttcagttgtctgtgtttgaagtcgaattgccggatgctgatacagctgttgcttttagggagcgtttgcgtagtttaatagaccctaacgacgatcaagttcgtctgtatcgactcactccacaggcattgggacagagaattatttacggaagaaggaccattgaggagagagttgacttttggatcgtgtaa

SEQ ID NO: 144 - DNA, 279 bp, Tolypothrix campylonemoides
  source 1..279: mol_type = genomic DNA
atgttctacttggtatgctatgacattgtgtcagataatcgaagaaacaaagtatcaaagctgttggaagcatacggacttagagtccagaagtctgtgtttgaatgcgtcctagacgaaaagcaatatgagatgttgtccaagtatttgatgcgtctggttaacaggcgagaggaccaagtcaggttttatccaatgtccgcccacaacagatgcaaggtcgctgtgctgggcacccagcccgagttcgttgttgacgatgctgcattcatcgtgtga

SEQ ID NO: 145 - DNA, 279 bp, Oscillatoriales cyanobacterium
  source 1..279: mol_type = genomic DNA
atgttctacctgatttgttacgatatagttgacgaccgtagacgagtcaaggtaagtaagctcttagagacatgcgggtgtagggtccaaaagtcagtctttgaatgcgtgttagacgagaagcgacaggagcagctgcaaaagcgtcttttgaagttactcaataagcgtcaggatcagattagattctaccccttgagcgagcattgccgatgcaaagttactgtgctcggtgtccagcctaaattcgtcatcgacgacgaagcctttatcgtgtaa

SEQ ID NO: 146 - DNA, 294 bp, Psychrobacter lutiphocae
  source 1..294: mol_type = genomic DNA
atgtcaaatgtaacaaaacagcaagctcccttatatgtttttgtttacgacatcagcaatgataaagagcgctccaaagtggacggcgtgctgcgtgattacggcttccgtgttcagaaatctgtttacgagtgccacttgacgcgtggtgataagaagcgcctgttggacaagctggaaagcctgaccattgaaaccggtcatattcgttgttatggtgtcaacagcaaggtgattaaaatcggcgcgccgccggaagatctggacgaggaatatatctacttcatcgagtaa

SEQ ID NO: 147 - DNA, 261 bp, Ruminococcus sp.
  source 1..261: mol_type = genomic DNA
atgtttgttatagtagtctatgatattaatagaaaacgtgttggtcgtgcactgaagatctgtcgtaagtacctggtgcacgtccaaaaaagcgtttttgagggcaatattaccgaaggccagctcaagcagttgaagaacgaattgttccaactgattaaaatcgaggaggacgcggtgtgcatttatcgcttcgagagcccgaaatacgctcgcaaagaagagctgggtacgatcgaacgtaagagccatattatctaa

SEQ ID NO: 148 - DNA, 279 bp, Vibrio sinaloensis
  source 1..279: mol_type = genomic DNA
atgaaggtgtatctggtgtgtttcgacattgagaacgacaagaaaaggcgtagactgagtaagttgttactatcttatggagaacgagtgcagtattctgtattcgagataactctgaagtctgatgcctcgctgaaaacccttactaagcaatgcaagagacacgttgagcagggagatagtttgagattctacgcacttccatttaacgctcgtcaggccagcttcgacatcgacggcgaacctatagcaaggttcccccaagccgagatcttgtag

SEQ ID NO: 149 - DNA, 261 bp, Blautia sp.
  source 1..261: mol_type = genomic DNA
atgtatgttattctagtatacgatatacaccaaaagcgcgttggtaaggcgctgaagatctgccgtaaatacctgatccacattcagaagagcgtgttcgagggcaacattacggaatccaaactgaaggcgttgaaggaggaactgggtcatttaatcgacacccagatggactcggtcatcatctatcacctggattctgtgaaatacaccaaaaaggagcagattggcattgttcaaagcaccagcaatgttatctaa

SEQ ID NO: 150 - DNA, 276 bp, Rivularia sp.
  source 1..276: mol_type = genomic DNA; strain = PCC 7116
atgcttgtggtggtggtttatgacattcccaatgacaagaggcgaacaaaactgtctaacttcttggaagggtacggacagcgtgtgcagttctcagttttcgagtgcttcttaaacctggacgaaatgagacagctattcgagaaaagcaagaagattgtaaagccgtctgaggataacgtgagattctactggatatcagaggacgcagtgtctcgtgtgcttaccataggctctgagcaccccaatgccccacctaattattacgtcatctaa

SEQ ID NO: 151 - DNA, 282 bp, Woodsholea maritima
  source 1..282: mol_type = genomic DNA
atggcagatccattgtttgttttcgcatacgacatctcacaggaccgtgtgagaaggagagtcgcaggactacttgaagcagaagccgtaagagtacaaggtagcgtctttgaagctagattgtccaaaagtagagctaaggctctgggacaaagaattgctatagaattagaaaggggagacaccttgagagtctactgcttgtctcagagggacctcaagagttctttccagtggggtggcgctccaatggctgaggcacaagacttttatattctgtaa

SEQ ID NO: 152 - DNA, 2220 bp, synthetic construct
  misc_feature 1..2220: note = Codon-optimized Cas1-Cas2
  source 1..2220: mol_type = other DNA
gcggaggagcatgcatgtttaccatcgacgagatgctgagcaagaacaaccaacgtctggcgttcgaacactttgcgaccaaaaacgacggttgcggcccggacggtatgcacgttagcgaactggagaagtactggcgtatgaatcacgaccagatcattagcgatctgaagaaccaggaataccagccgggtattatcctgattcgtgagcacatgaataaaaccggcaaacgccgtaatatcgcgagcctgaacgttattgatcgtttcattacccgtctgctgagccagaaactgaaccgttatctggcgccgattttctgcgagaacagctatgcgtaccaggacagcaaaggcgttatgccggcggttctgaaagcgaaggagtacgttgagctgggtatgcgtcacgtgattgaaatcgatctgaaaaactactttgacaccattccgctggaaaacctgatcccggagatcgaacgctacatcaccgatgaggcggttctgcatctgatcaagcaatacctgttttgcgatattagcttcgagggcaaaatcagccgtaaaacccaaggtatcgttcaaggtaacgcgatcagcccgatcctgagcaacctgtacctgaacgacttcgacaaggaactggacgagagcaagctgtgctggattcgttacgcggacaatatctacatctatatggatagctatgagaaggcgctgctggtgtatagcgagctgaccgagcgtctggagcgtcgcaagctgaccgttaacaaggagaaaagcggcgtttttgatgtgagcacccgtagcattctgggctacgacatcctgattcgtaacaagaaagtggacgttcgtaagcatatttacaaaagcgttaatcagtacagcaactggcacgatagccgcctggagtttatcaacggtcgttaccacatcaccagcgatggcatcctgaatcgccaagacttcggcctgctgttcgagaatgagcagaaaaaacactacatcccggttgaggttagcgatcagctgaacatctacggtaatgttaccctggcgagcaatgtgctgcaaagtttcagcaaccgcgaaatcaaagtgagcttcttcgacaagtatggtcgtctgattggtagcttcctgccggaaaaaaccaagaaaagcgcggagatcattctggtgcagagcaagaactatctgaacgaagatgtgcgtatggataccgcgcgccgtatggaaattgcgggtctgcacaacatccgtgcgaatctgcgctactacgataagaaacacaagggcgactttaaagagaaggttgacgcgattagcggctacattgacgcgctgaaccgtgcgccgagcgttaatgatatgatgctgctggaagcgaaagcgcgccaactgtattatacctgttttaaccaaatcctggaaaccagcgacttccagttcgaaaagcgtaccaagcgtccgccgaaagatgcgatcaatgcgtgcattagcttcggcaataccctgctgtataatctatttgtgaatattatctggaagaagggtctggacccgcgctttggcgttgttcacgcgagcaacaaacgcaatcaaagcctgaacctggacttcgcggacatctttaaaccgatcgtgattgaccgcattatcttcaccatgatcaataagaaaatgctgaccctgctgaccgattttgaaaccagcaaccagggtgtgtatctgagccgtgagggcaagaacatcttcctgcaaatgtacgaggagaagctgaaaagccgcattaccatcaagggtaaagagatgagctattaccagctgctggagagcgaagtgcagaattacaagaatttcattctgaccggcgaaacctataaaccgtataagtattattaacgtacgttaactttaagaaggagaacttaagatgtatgttatcctggtgtacgacatccaccagaagcgtgttggtaaagcgctgaaaatctgccgtaagtacctgatccacatccaaaagagcgtgttcgaaggcaacatcaccgagagcaaactgaaggcgctgaaagaggaactgggtcacctgattgacacccagatggatagcgtgattatctaccacctggacagcgttaagtacaccaaaaaggagcagatcggtattgtgcagagcaccagcaatgtgatctaactcgagatccggctg

SEQ ID NO: 153 - DNA, 858 bp, synthetic construct
  misc_feature 1..858: note = gBlock
  source 1..858: mol_type = other DNA
aggaatggtgcatgcaccggtttgacggctagctcagtcctaggtattgtgctagcataattttgtttaactttaatgaggaggtatacgctggtcttctattcccatggatcatcaccggcaaggaagacaacttgggttaattgaggcctgagtataaggtgacttatacttgtaatctatctaaacggggaacctctctagtagacaatcccgtgctaaattgtaggactgccctttaataaatacttctatatttaaagaggtatttatgaaaagcggaatttatcagattaaaaatactttctctagagaaaatttcgtctggattagttacttatcgtgtaaaatctgataaatggaattggttctacataaatgcctaacgactatccctttggggagtagggtcaagtgactcgaaacgatagacaacttgctttaacaagttggagatatagtctgctctgcatggtgacatgcagctggatataattccggggtaagattaacgaccttatctgaacataatgcttagtcttccatcgacttcaagcttcgatccaacgatcagaagacgcgatctccaggcatcaaataaaacgaaaggctcagtcgaaagactgggcctttcgttttatctgttgtttgtcggtgaacgctctcctgagtaggacaaatccggatcccatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccac

SEQ ID NO: 154 - DNA, 28 bp, synthetic construct
  misc_feature 1..28: note = Primer
  source 1..28: mol_type = other DNA
tcaacacttcatctatctaactgaataa

SEQ ID NO: 155 - DNA, 20 bp, synthetic construct
  misc_feature 1..20: note = Primer
  source 1..20: mol_type = other DNA
tgttatgaacggctacgcct

SEQ ID NO: 156 - DNA, 22 bp, synthetic construct
  misc_feature 1..22: note = Primer
  source 1..22: mol_type = other DNA
cgctcgaattcagctctcacag

SEQ ID NO: 157 - DNA, 24 bp, synthetic construct
  misc_feature 1..24: note = Primer
  source 1..24: mol_type = other DNA
aattgccaaattctgtttcaatcc

SEQ ID NO: 158 - DNA, 19 bp, synthetic construct
  misc_feature 1..19: note = Primer
  source 1..19: mol_type = other DNA
gtcagcccggggtcaaaac

SEQ ID NO: 159 - DNA, 25 bp, synthetic construct
  misc_feature 1..25: note = Primer
  source 1..25: mol_type = other DNA
ggaactttaaaccctttacatcccc

SEQ ID NO: 160 - DNA, 21 bp, synthetic construct
  misc_feature 1..21: note = Primer
  source 1..21: mol_type = other DNA
tcagaaaaacgatcgaccgac

SEQ ID NO: 161 - DNA, 22 bp, synthetic construct
  misc_feature 1..22: note = Primer
  source 1..22: mol_type = other DNA
agaagaagcaatcgaaaaagcg

SEQ ID NO: 162 - DNA, 20 bp, synthetic construct
  misc_feature 1..20: note = Primer
  source 1..20: mol_type = other DNA
agaatctgaaaacagcggaa

SEQ ID NO: 163 - DNA, 21 bp, synthetic construct
  misc_feature 1..21: note = Primer
  source 1..21: mol_type = other DNA
acgctagggaatatgcagcaa

SEQ ID NO: 164 - DNA, 22 bp, synthetic construct
  misc_feature 1..22: note = Primer
  source 1..22: mol_type = other DNA
ccgaaaagagccgttaaattcc

SEQ ID NO: 165 - DNA, 23 bp, synthetic construct
  misc_feature 1..23: note = Primer
  source 1..23: mol_type = other DNA
cctcaaaacggtaccaaagaagc

SEQ ID NO: 166 - DNA, 20 bp, synthetic construct
  misc_feature 1..20: note = Primer
  source 1..20: mol_type = other DNA
cacagcacctcttcgccacg

SEQ ID NO: 167 - DNA, 20 bp, synthetic construct
  misc_feature 1..20: note = Primer
  source 1..20: mol_type = other DNA
cgattccggtcctcggtttc

SEQ ID NO: 168 - DNA, 20 bp, synthetic construct
  misc_feature 1..20: note = Primer
  source 1..20: mol_type = other DNA
ctcaagacccaccgttttcg

SEQ ID NO: 169 - DNA, 22 bp, synthetic construct
  misc_feature 1..22: note = Primer
  source 1..22: mol_type = other DNA
ttcaacaacgacgccaactatg

SEQ ID NO: 170 - DNA, 20 bp, synthetic construct
  misc_feature 1..20: note = Primer
  source 1..20: mol_type = other DNA
gcaagtctccggcaagtcag

SEQ ID NO: 171 - DNA, 31 bp, synthetic construct
  misc_feature 1..31: note = Primer
  source 1..31: mol_type = other DNA
tcacttgaagattatatagtgactcttttcg

SEQ ID NO: 172 - DNA, 21 bp, synthetic construct
  misc_feature 1..21: note = Primer
  source 1..21: mol_type = other DNA
tggcaaaccatgtggaaacag

SEQ ID NO: 173 - DNA, 17 bp, synthetic construct
  misc_feature 1..17: note = Primer
  source 1..17: mol_type = other DNA
aaaatggcaacgccggg

SEQ ID NO: 174 - DNA, 22 bp, synthetic construct
  misc_feature 1..22: note = Primer
  source 1..22: mol_type = other DNA
tggagctgaatgtcacatcttg

SEQ ID NO: 175 - DNA, 21 bp, synthetic construct
  misc_feature 1..21: note = Primer
  source 1..21: mol_type = other DNA
ggaatctcaagcagcggagaa

SEQ ID NO: 176 - DNA, 19 bp, synthetic construct
  misc_feature 1..19: note = Primer
  source 1..19: mol_type = other DNA
cacaggatgcgtggaaagg

SEQ ID NO: 177 - DNA, 19 bp, synthetic construct
  misc_feature 1..19: note = Primer
  source 1..19: mol_type = other DNA
ctcaacgaaccgaagctgc

SEQ ID NO: 178 - DNA, 20 bp, synthetic construct
  misc_feature 1..20: note = Primer
  source 1..20: mol_type = other DNA
ccgttgggaattttcccgtt

SEQ ID NO: 179 - DNA, 20 bp, synthetic construct
  misc_feature 1..20: note = Primer
  source 1..20: mol_type = other DNA
gactctttttcccggagccc

SEQ ID NO: 180 - DNA, 20 bp, synthetic construct
  misc_feature 1..20: note = Primer
  source 1..20: mol_type = other DNA
cccaaacggggttctagcat

SEQ ID NO: 181 - DNA, 25 bp, synthetic construct
  misc_feature 1..25: note = Primer
  source 1..25: mol_type = other DNA
gcgacaaaagcatattaaggagact

SEQ ID NO: 182 - DNA, 23 bp, synthetic construct
  misc_feature 1..23: note = Primer
  source 1..23: mol_type = other DNA
gcgctgtagaattatttcagggt

SEQ ID NO: 183 - DNA, 19 bp, synthetic construct
  misc_feature 1..19: note = Primer
  source 1..19: mol_type = other DNA
atgggatggaggttcgggt

SEQ ID NO: 184 - DNA, 18 bp, synthetic construct
  misc_feature 1..18: note = Primer
  source 1..18: mol_type = other DNA
gagcttggggcaaggctc

SEQ ID NO: 185 - DNA, 25 bp, synthetic construct
  misc_feature 1..25: note = Primer
  source 1..25: mol_type = other DNA
gtcgagaagtagcagttcactttct

SEQ ID NO: 186 - DNA, 22 bp, synthetic construct
  misc_feature 1..22: note = Primer
  source 1..22: mol_type = other DNA
acctatcacaacggcttaaatg

SEQ ID NO: 187 - DNA, 23 bp, synthetic construct
  misc_feature 1..23: note = Primer
  source 1..23: mol_type = other DNA
atcactgctatgcagcttattcg

SEQ ID NO: 188 - DNA, 20 bp, synthetic construct
  misc_feature 1..20: note = Primer
  source 1..20: mol_type = other DNA
aaagcgagggctttcccata

SEQ ID NO: 189 - DNA, 21 bp, synthetic construct
  misc_feature 1..21: note = Primer
  source 1..21: mol_type = other DNA
ctcatcagaatgtgacggtcg

SEQ ID NO: 190 - DNA, 37 bp, synthetic construct
  misc_feature 1..37: note = SENECA adapter oligo
  source 1..37: mol_type = other DNA
ataagatcggaagagcacacgtctgaactccagtcac

SEQ ID NO: 191 - DNA, 37 bp, synthetic construct
  misc_feature 1..37: note = SENECA adapter oligo
  source 1..37: mol_type = other DNA
gaatgatcggaagagcacacgtctgaactccagtcac

SEQ ID NO: 192 - DNA, 37 bp, synthetic construct
  misc_feature 1..37: note = SENECA adapter oligo
  source 1..37: mol_type = other DNA
tagggatcggaagagcacacgtctgaactccagtcac

SEQ ID NO: 193 - DNA, 37 bp, synthetic construct
  misc_feature 1..37: note = SENECA adapter oligo
  source 1..37: mol_type = other DNA
gaaagatcggaagagcacacgtctgaactccagtcac

SEQ ID NO: 194 - DNA, 37 bp, synthetic construct
  misc_feature 1..37: note = SENECA adapter oligo
  source 1..37: mol_type = other DNA
gagggatcggaagagcacacgtctgaactccagtcac

SEQ ID NO: 195 - DNA, 37 bp, synthetic construct
  misc_feature 1..37: note = SENECA adapter oligo
  source 1..37: mol_type = other DNA
gccagatcggaagagcacacgtctgaactccagtcac

SEQ ID NO: 196 - DNA, 37 bp, synthetic construct
  misc_feature 1..37: note = SENECA adapter oligo
  source 1..37: mol_type = other DNA
tgaggatcggaagagcacacgtctgaactccagtcac

SEQ ID NO: 197 - DNA, 37 bp, synthetic construct
  misc_feature 1..37: note = SENECA adapter oligo
  source 1..37: mol_type = other DNA
aggtgatcggaagagcacacgtctgaactccagtcac

SEQ ID NO: 198 - DNA, 37 bp, synthetic construct
  misc_feature 1..37: note = SENECA adapter oligo
  source 1..37: mol_type = other DNA
aaaggatcggaagagcacacgtctgaactccagtcac

SEQ ID NO: 199 - DNA, 37 bp, synthetic construct; identical to SEQ ID NO: 197
  misc_feature 1..37: note = SENECA adapter oligo
  source 1..37: mol_type = other DNA

SEQ ID NO: 200 - DNA, 37 bp, synthetic construct; identical to SEQ ID NO: 198
  misc_feature 1..37: note = SENECA adapter oligo
  source 1..37: mol_type = other DNA

SEQ ID NO: 201 - DNA, 37 bp, synthetic construct
  misc_feature 1..37: note = SENECA adapter oligo
  source 1..37: mol_type = other DNA
ggctgatcggaagagcacacgtctgaactccagtcac

SEQ ID NO: 202 - DNA, 37 bp, synthetic construct
  misc_feature 1..37: note = SENECA adapter oligo
  source 1..37: mol_type = other DNA
gcgggatcggaagagcacacgtctgaactccagtcac

SEQ ID NO: 203 - DNA, 37 bp, synthetic construct
  misc_feature 1..37: note = SENECA adapter oligo
  source 1..37: mol_type = other DNA
ctgtgatcggaagagcacacgtctgaactccagtcac

SEQ ID NO: 204 - DNA, 37 bp, synthetic construct; identical to SEQ ID NO: 202
  misc_feature 1..37: note = SENECA adapter oligo
  source 1..37: mol_type = other DNA

SEQ ID NO: 205 - DNA, 37 bp, synthetic construct; identical to SEQ ID NO: 203
  misc_feature 1..37: note = SENECA adapter oligo
  source 1..37: mol_type = other DNA

SEQ ID NO: 206 - DNA, 37 bp, synthetic construct
  misc_feature 1..37: note = SENECA adapter oligo
  source 1..37: mol_type = other DNA
gggtgatcggaagagcacacgtctgaactccagtcac

SEQ ID NO: 207 - DNA, 37 bp, synthetic construct
  misc_feature 1..37: note = SENECA adapter oligo
  source 1..37: mol_type = other DNA
aacagatcggaagagcacacgtctgaactccagtcac

SEQ ID NO: 208 - DNA, 37 bp, synthetic construct
  misc_feature 1..37: note = SENECA adapter oligo
  source 1..37: mol_type = other DNA
aagcgatcggaagagcacacgtctgaactccagtcac

SEQ ID NO: 209 - DNA, 37 bp, synthetic construct
  misc_feature 1..37: note = SENECA adapter oligo
  source 1..37: mol_type = other DNA
gcatgatcggaagagcacacgtctgaactccagtcac

SEQ ID NO: 210 - DNA, 37 bp, synthetic construct; identical to SEQ ID NO: 208
  misc_feature 1..37: note = SENECA adapter oligo
  source 1..37: mol_type = other DNA

SEQ ID NO: 211 - DNA, 37 bp, synthetic construct; identical to SEQ ID NO: 209
  misc_feature 1..37: note = SENECA adapter oligo
  source 1..37: mol_type = other DNA

SEQ ID NO: 212 - DNA, 37 bp, synthetic construct
  misc_feature 1..37: note = SENECA adapter oligo
  source 1..37: mol_type = other DNA
gagcgatcggaagagcacacgtctgaactccagtcac

SEQ ID NO: 213 - DNA, 37 bp, synthetic construct
  misc_feature 1..37: note = SENECA adapter oligo
  source 1..37: mol_type = other DNA
gattgatcggaagagcacacgtctgaactccagtcac

SEQ ID NO: 214 - DNA, 37 bp, synthetic construct; identical to SEQ ID NO: 212
  misc_feature 1..37: note = SENECA adapter oligo
  source 1..37: mol_type = other DNA

SEQ ID NO: 215 - DNA, 37 bp, synthetic construct
  misc_feature 1..37: note = SENECA adapter oligo
  source 1..37: mol_type = other DNA
gatggatcggaagagcacacgtctgaactccagtcac

SEQ ID NO: 216 - DNA, 37 bp, synthetic construct; identical to SEQ ID NO: 212
  misc_feature 1..37: note = SENECA adapter oligo
  source 1..37: mol_type = other DNA

SEQ ID NO: 217 - DNA, 37 bp, synthetic construct
  misc_feature 1..37: note = SENECA adapter oligo
  source 1..37: mol_type = other DNA
gacagatcggaagagcacacgtctgaactccagtcac

SEQ ID NO: 218 - DNA, 37 bp, synthetic construct
  misc_feature 1..37: note = SENECA adapter oligo
  source 1..37: mol_type = other DNA
taaggatcggaagagcacacgtctgaactccagtcac

SEQ ID NO: 219 - DNA, 37 bp, synthetic construct
  misc_feature 1..37: note = SENECA adapter oligo
  source 1..37: mol_type = other DNA
atgtgatcggaagagcacacgtctgaactccagtcac

SEQ ID NO: 220 - DNA, 37 bp, synthetic construct; identical to SEQ ID NO: 191
  misc_feature 1..37: note = SENECA adapter oligo
  source 1..37: mol_type = other DNA

SEQ ID NO: 221 - DNA, 37 bp, synthetic construct
  misc_feature 1..37: note = SENECA adapter oligo
  source 1..37: mol_type = other DNA
gaaggatcggaagagcacacgtctgaactccagtcac

SEQ ID NO: 222 - DNA, 37 bp, synthetic construct; identical to SEQ ID NO: 191
  misc_feature 1..37: note = SENECA adapter oligo
  source 1..37: mol_type = other DNA

SEQ ID NO: 223 - DNA, 37 bp, synthetic construct
  misc_feature 1..37: note = SENECA adapter oligo
  source 1..37: mol_type = other DNA
gagagatcggaagagcacacgtctgaactccagtcac

SEQ ID NO: 224 - DNA, 37 bp, synthetic construct; identical to SEQ ID NO: 191
  misc_feature 1..37: note = SENECA adapter oligo
  source 1..37: mol_type = other DNA

SEQ ID NO: 225 - DNA, 37 bp, synthetic construct; identical to SEQ ID NO: 221
  misc_feature 1..37: note = SENECA adapter oligo
  source 1..37: mol_type = other DNA

SEQ ID NO: 226 - DNA, 37 bp, synthetic construct
  misc_feature 1..37: note = SENECA adapter oligo
  source 1..37: mol_type = other DNA
aaatgatcggaagagcacacgtctgaactccagtcac

SEQ ID NO: 227 - DNA, 37 bp, synthetic construct; identical to SEQ ID NO: 223
  misc_feature 1..37: note = SENECA adapter oligo
  source 1..37: mol_type = other DNA

SEQ ID NO: 228 - DNA, 37 bp, synthetic construct
  misc_feature 1..37: note = SENECA adapter oligo
  source 1..37: mol_type = other DNA
aattgatcggaagagcacacgtctgaactccagtcac

SEQ ID NO: 229 - DNA, 37 bp, synthetic construct; identical to SEQ ID NO: 218
  misc_feature 1..37: note = SENECA adapter oligo
  source 1..37: mol_type = other DNA

SEQ ID NO: 230 - DNA, 37 bp, synthetic construct; identical to SEQ ID NO: 213
  misc_feature 1..37: note = SENECA adapter oligo
  source 1..37: mol_type = other DNA

SEQ ID NO: 231 - DNA, 37 bp, synthetic construct
  misc_feature 1..37: note = SENECA adapter oligo
  source 1..37: mol_type = other DNA
cccagatcggaagagcacacgtctgaactccagtcac

SEQ ID NO: 232 - DNA, 37 bp, synthetic construct; identical to SEQ ID NO: 213
  misc_feature 1..37: note = SENECA adapter oligo
  source 1..37: mol_type = other DNA

SEQ ID NO: 233 - DNA, 37 bp, synthetic construct; identical to SEQ ID NO: 231
  misc_feature 1..37: note = SENECA adapter oligo
  source 1..37: mol_type = other DNA

SEQ ID NO: 234 - DNA, 37 bp, synthetic construct; identical to SEQ ID NO: 218
  misc_feature 1..37: note = SENECA adapter oligo
  source 1..37: mol_type = other DNA

SEQ ID NO: 235 - DNA, 37 bp, synthetic construct
  misc_feature 1..37: note = SENECA adapter oligo
  source 1..37: mol_type = other DNA
ggtagatcggaagagcacacgtctgaactccagtcac

SEQ ID NO: 236 - DNA, 37 bp, synthetic construct; identical to SEQ ID NO: 218
  misc_feature 1..37: note = SENECA adapter oligo
  source 1..37: mol_type = other DNA

SEQ ID NO: 237 - DNA, 37 bp, synthetic construct; identical to SEQ ID NO: 235
  misc_feature 1..37: note = SENECA adapter oligo
  source 1..37: mol_type = other DNA

SEQ ID NO: 238 - DNA, 37 bp, synthetic construct; identical to SEQ ID NO: 190
  misc_feature 1..37: note = SENECA adapter oligo
  source 1..37: mol_type = other DNA

SEQ ID NO: 239 - DNA, 37 bp, synthetic construct; identical to SEQ ID NO: 191
  misc_feature 1..37: note = SENECA adapter oligo
  source 1..37: mol_type = other DNA

SEQ ID NO: 240 - DNA, 21 bp, synthetic construct
  misc_feature 1..21: note = PCR primer for SENECA acquisition readout
  source 1..21: mol_type = other DNA
cagtataataaggattaagac

SEQ ID NO: 241 - DNA, 18 bp, synthetic construct
  misc_feature 1..18: note = PCR primer for SENECA acquisition readout
  source 1..18: mol_type = other DNA
actggaatacatctacat

SEQ ID NO: 242 - DNA, 16 bp, synthetic construct
  misc_feature 1..16: note = PCR primer for SENECA acquisition readout
  source 1..16: mol_type = other DNA
attaggggattgaaac

SEQ ID NO: 243 - DNA, 17 bp, synthetic construct
  misc_feature 1..17: note = PCR primer for SENECA acquisition readout
  source 1..17: mol_type = other DNA
ggagaaagtgtctaaac

SEQ ID NO: 244 - DNA, 14 bp, synthetic construct
  misc_feature 1..14: note = PCR primer for SENECA acquisition readout
  source 1..14: mol_type = other DNA
gagggcattgaaac

SEQ ID NO: 245 - DNA, 14 bp, synthetic construct
  misc_feature 1..14: note = PCR primer for SENECA acquisition readout
  source 1..14: mol_type = other DNA
gccatgggtggaac

SEQ ID NO: 246 - DNA, 18 bp, synthetic construct
  misc_feature 1..18: note = PCR primer for SENECA acquisition readout
  source 1..18: mol_type = other DNA
cctatgaggaattgaaac

SEQ ID NO: 247 - DNA, 17 bp, synthetic construct
  misc_feature 1..17: note = PCR primer for SENECA acquisition readout
  source 1..17: mol_type = other DNA
cataggtaaggtacaac

SEQ ID NO: 248 - DNA, 18 bp, synthetic construct
  misc_feature 1..18: note = PCR primer for SENECA acquisition readout
  source 1..18: mol_type = other DNA
cctaaaaggaattgaaac

SEQ ID NO: 249 - DNA, 17 bp, synthetic construct
  misc_feature 1..17: note = PCR primer for SENECA acquisition readout
  source 1..17: mol_type = other DNA
tttaggtaaagtacgac

SEQ ID NO: 250 - DNA, 17 bp, synthetic construct
  misc_feature 1..17: note = PCR primer for SENECA acquisition readout
  source 1..17: mol_type = other DNA
gataaagggattgagac

SEQ ID NO: 251 - DNA, 15 bp, synthetic construct
  misc_feature 1..15: note = PCR primer for SENECA acquisition readout
  source 1..15: mol_type = other DNA
gggcttagttttcac

SEQ ID NO: 252 - DNA, 14 bp, synthetic construct
  misc_feature 1..14: note = PCR primer for SENECA acquisition readout
  source 1..14: mol_type = other DNA
gcgggcatagaaac

SEQ ID NO: 253 - DNA, 14 bp, synthetic construct
  misc_feature 1..14: note = PCR primer for SENECA acquisition readout
  source 1..14: mol_type = other DNA
ctgtggatggcgat

SEQ ID NO: 254 - DNA, 14 bp, synthetic construct
  misc_feature 1..14: note = PCR primer for SENECA acquisition readout
  source 1..14: mol_type = other DNA
gcgggcatagaaac

SEQ ID NO: 255 - DNA, 14 bp, synthetic construct
  misc_feature 1..14: note = PCR primer for SENECA acquisition readout
  source 1..14: mol_type = other DNA
ctgtggatggcaat

SEQ ID NO: 256 - DNA, 13 bp, synthetic construct
  misc_feature 1..13: note = PCR primer for SENECA acquisition readout
  source 1..13: mol_type = other DNA
ggtgatgagcgac

SEQ ID NO: 257 - DNA, 15 bp, synthetic construct
  misc_feature 1..15: note = PCR primer for SENECA acquisition readout
  source 1..15: mol_type = other DNA
gaacaggcttgaaac

SEQ ID NO: 258 - DNA, 15 bp, synthetic construct
  misc_feature 1..15: note = PCR primer for SENECA acquisition readout
  source 1..15: mol_type = other DNA
gaagcggattgaaac

SEQ ID NO: 259 - DNA, 15 bp, synthetic construct
  misc_feature 1..15: note = PCR primer for SENECA acquisition readout
  source 1..15: mol_type = other DNA
ggcatccctcaatag

SEQ ID NO: 260 - DNA, 15 bp, synthetic construct
  misc_feature 1..15: note = PCR primer for SENECA acquisition readout
  source 1..15: mol_type = other DNA
gaagcggattgaaac

SEQ ID NO: 261 - DNA, 15 bp, synthetic construct
  misc_feature 1..15: note = PCR primer for SENECA acquisition readout
  source 1..15: mol_type = other DNA
ggcatccctcaatag

SEQ ID NO: 262 - DNA, 16 bp, synthetic construct
  misc_feature 1..16: note = PCR primer for SENECA acquisition readout
  source 1..16: mol_type = other DNA
cagagctgatcaaaac

SEQ ID NO: 263 - DNA, 14 bp, synthetic construct
  misc_feature 1..14: note = PCR primer for SENECA acquisition readout
  source 1..14: mol_type = other DNA
gattcgagcagagc

SEQ ID NO: 264 - DNA, 15 bp, synthetic construct
  misc_feature 1..15: note = PCR primer for SENECA acquisition readout
  source 1..15: mol_type = other DNA
ggagcggattgaaac

SEQ ID NO: 265 - DNA, 14 bp, synthetic construct
  misc_feature 1..14: note = PCR primer for SENECA acquisition readout
  source 1..14: mol_type = other DNA
gatgccgtcgcgac

SEQ ID NO: 266 - DNA, 15 bp, synthetic construct
  misc_feature 1..15: note = PCR primer for SENECA acquisition readout
  source 1..15: mol_type = other DNA
ggagcggattgaaac

SEQ ID NO: 267 - DNA, 14 bp, synthetic construct
  misc_feature 1..14: note = PCR primer for SENECA acquisition readout
  source 1..14: mol_type = other DNA
gacaccggcggaac

SEQ ID NO: 268 - DNA, 16 bp, synthetic construct
  misc_feature 1..16: note = PCR primer for SENECA acquisition readout
  source 1..16: mol_type = other DNA
gctaaggctgtgaaac

SEQ ID NO: 269 - DNA, 17 bp, synthetic construct
  misc_feature 1..17: note = PCR primer for SENECA acquisition readout
  source 1..17: mol_type = other DNA
ctaatgtcgattgcgac

SEQ ID NO: 270 - DNA, 19 bp, synthetic construct
  misc_feature 1..19: note = PCR primer for SENECA acquisition readout
  source 1..19: mol_type = other DNA
aagttgaattaatggaaac

SEQ ID NO: 271 - DNA, 18 bp, synthetic construct
  misc_feature 1..18: note = PCR primer for SENECA acquisition readout
  source 1..18: mol_type = other DNA
ttccgaagaagtttaaag

SEQ ID NO: 272 - DNA, 19 bp, synthetic construct
  misc_feature 1..19: note = PCR primer for SENECA acquisition readout
  source 1..19: mol_type = other DNA
aagttgaattaatggaaac

SEQ ID NO: 273 - DNA, 16 bp, synthetic construct
  misc_feature 1..16: note = PCR primer for SENECA acquisition readout
  source 1..16: mol_type = other DNA
gggagaagtttaacag

SEQ ID NO: 274 - DNA, 19 bp, synthetic construct
  misc_feature 1..19: note = PCR primer for SENECA acquisition readout
  source 1..19: mol_type = other DNA
aagttgaattaatggaaac

SEQ ID NO: 275 - DNA, 18 bp, synthetic construct
  misc_feature 1..18: note = PCR primer for SENECA acquisition readout
  source 1..18: mol_type = other DNA
ttccgaagaagtttaaag

SEQ ID NO: 276 - DNA, 18 bp, synthetic construct
  misc_feature 1..18: note = PCR primer for SENECA acquisition readout
  source 1..18: mol_type = other DNA
agtcaaattaatggaaac

SEQ ID NO: 277 - DNA, 16 bp, synthetic construct
  misc_feature 1..16: note = PCR primer for SENECA acquisition readout
  source 1..16: mol_type = other DNA
cagagaagtcgagaag

SEQ ID NO: 278 - DNA, 18 bp, synthetic construct
  misc_feature 1..18: note = PCR primer for SENECA acquisition readout
  source 1..18: mol_type = other DNA
gtcaaattaatggaaaca

SEQ ID NO: 279 - DNA, 16 bp, synthetic construct
  misc_feature 1..16: note = PCR primer for SENECA acquisition readout
  source 1..16: mol_type = other DNA
cctaagaagtcgaaag

SEQ ID NO: 280 - DNA, 16 bp, synthetic construct
  misc_feature 1..16: note = PCR primer for SENECA acquisition readout
  source 1..16: mol_type = other DNA
cggattagttggaaac

SEQ ID NO: 281 - DNA, 14 bp, synthetic construct
  misc_feature 1..14: note = PCR primer for SENECA acquisition readout
  source 1..14: mol_type = other DNA
cccaatcggtgggg

SEQ ID NO: 282 - DNA, 16 bp, synthetic construct
  misc_feature 1..16: note = PCR primer for SENECA acquisition readout
  source 1..16: mol_type = other DNA
cggattagttggaaac

SEQ ID NO: 283 - DNA, 14 bp, synthetic construct
  misc_feature 1..14: note = PCR primer for SENECA acquisition readout
  source 1..14: mol_type = other DNA
cccaatcggtgggg

SEQ ID NO: 284 - DNA, 18 bp, synthetic construct
  misc_feature 1..18: note = PCR primer for SENECA acquisition readout
  source 1..18: mol_type = other DNA
cctataaggaatggaaac

SEQ ID NO: 285 - DNA, 19 bp, synthetic construct
  misc_feature 1..19: note = PCR primer for SENECA acquisition readout
  source 1..19: mol_type = other DNA
ttataggtaaggtacttac

SEQ ID NO: 286 - DNA, 18 bp, synthetic construct
  misc_feature 1..18: note = PCR primer for SENECA acquisition readout
  source 1..18: mol_type = other DNA
cctataaggaatggaaac

SEQ ID NO: 287 - DNA, 19 bp, synthetic construct
  misc_feature 1..19: note = PCR primer for SENECA acquisition readout
  source 1..19: mol_type = other DNA
ttataggtaaggtacttac

SEQ ID NO: 288 - DNA, 21 bp, synthetic construct
  misc_feature 1..21: note = PCR primer for SENECA acquisition readout
  source 1..21: mol_type = other DNA
cagtataataaggattaagac

SEQ ID NO: 289 - DNA, 18 bp, synthetic construct
  misc_feature 1..18: note = PCR primer for SENECA acquisition readout
  source 1..18: mol_type = other DNA
actggaatacatctacat

SEQ ID NO: 290 - DNA, 97 bp, synthetic construct
  misc_feature 1..97: note = Primer
  source 1..97: mol_type = other DNA
atgcttcatgtcaccaggtagtcttccatcgacttcaaaactcgatccaacatcctgaagacgcggccgctattcttttgatttataagggattttg

SEQ ID NO: 291 - DNA, 35 bp, synthetic construct
  misc_feature 1..35: note = Primer
  source 1..35: mol_type = other DNA
caacaacatgaatgatcttcggtttccgtgtttcg

SEQ ID NO: 292 - DNA, 38 bp, synthetic construct
  misc_feature 1..38: note = Primer
  source 1..38: mol_type = other DNA
cacggaaaccgaagatcattcatgttgttgctcaggtc

SEQ ID NO: 293 - DNA, 38 bp, synthetic construct
  misc_feature 1..38: note = Primer
  source 1..38: mol_type = other DNA
cgccgcacttatgactatcttctttatcatgcaactcg

SEQ ID NO: 294 - DNA, 31 bp, synthetic construct
  misc_feature 1..31: note = Primer
  source 1..31: mol_type = other DNA
gataaagaagatagtcataagtgcggcgacg

SEQ ID NO: 295 - DNA, 33 bp, synthetic construct
  misc_feature 1..33: note = Primer
  source 1..33: mol_type = other DNA
gataccgaagatagctcatgttatatcccgccg

SEQ ID NO: 296 - DNA, 36 bp, synthetic construct
  misc_feature 1..36: note = Primer
  source 1..36: mol_type = other DNA
gatataacatgagctatcttcggtatcgtcgtatcc

SEQ ID NO: 297 - DNA, 28 bp, synthetic construct
  misc_feature 1..28: note = Primer
  source 1..28: mol_type = other DNA
ctcccatgaagatggtacgcgactgggc

SEQ ID NO: 298 - DNA, 38 bp, synthetic construct
  misc_feature 1..38: note = Primer
  source 1..38: mol_type = other DNA
gtcgcgtaccatcttcatgggagaaaataatactgttg

SEQ ID NO: 299 - DNA, 114 bp, synthetic construct
  misc_feature 1..114: note = Primer
  source 1..114: mol_type = other DNA
gaagactacctggtgacatgaagcatctcgagggtcttccttgccggtggtgcagatgttgaacagaagaccacatatgtatatctccttcttaaagttaaacaaaattatttc

SEQ ID NO: 300 - DNA, 141 bp, synthetic construct
  misc_feature 1..141: note = Primer
  source 1..141: mol_type = other DNA
tcgagatccggctgctaacaaagcccgaaaggaagctgagttggctgctgccaccgctgagcaataactagcataaccccttggggcctctaaacgggtcttgaggggttttttgctgaaaggaggaactatatccggata

SEQ ID NO: 301 - DNA, 142 bp, synthetic construct
  misc_feature 1..142: note = Primer
  source 1..142: mol_type = other DNA
cctggtatccggatatagttcctcctttcagcaaaaaacccctcaagacccgtttagaggccccaaggggttatgctagttattgctcagcggtggcagcagccaactcagcttcctttcgggctttgttagcagccggatc

SEQ ID NO: 302 - DNA, 36 bp, synthetic construct
  misc_feature 1..36: note = Primer
  source 1..36: mol_type = other DNA
gctcagcatatggacatcctgatcagaaacaagaag

SEQ ID NO: 303 - DNA, 35 bp, synthetic construct
  misc_feature 1..35: note = Primer
  source 1..35: mol_type = other DNA
gctcagcatatgcagtactccaactggcacgactc

SEQ ID NO: 304 - DNA, 36 bp, synthetic construct
  misc_feature 1..36: note = Primer
  source 1..36: mol_type = other DNA
gctcagcatatgttcatcaacggtcgttaccacatc

SEQ ID NO: 305 - DNA, 23 bp, synthetic construct
  misc_feature 1..23: note = Primer
  source 1..23: mol_type = other DNA
cctactcgcttctggtgaatgtc

SEQ ID NO: 306 - DNA, 26 bp, synthetic construct
  misc_feature 1..26: note = Primer
  source 1..26: mol_type = other DNA
ccggataccaggtgagaattaaattg

SEQ ID NO: 307 - DNA, 70 bp, synthetic construct
  misc_feature 1..70: note = Primer
  source 1..70: mol_type = other DNA
gtttagcggccgcgggacgtttcaattcctcataggtaaggtacaacatcagcatttccgctattttcac

SEQ ID NO: 308 - DNA, 18 bp, synthetic construct
  misc_feature 1..18: note = Primer
  source 1..18: mol_type = other DNA
gtgactggagttcagacg

SEQ ID NO: 309 - DNA, 33 bp, synthetic construct
  misc_feature 1..33: note = Primer
  source 1..33: mol_type = other DNA
gtgactggagttcagacgtgtgctcttccgatc

SEQ ID NO: 310 - DNA, 37 bp, synthetic construct; identical to SEQ ID NO: 198
  misc_feature 1..37: note = Primer
  source 1..37: mol_type = other DNA

SEQ ID NO: 311 - DNA, 30 bp, synthetic construct
  misc_feature 1..30: note = Primer
  source 1..30: mol_type = other DNA
gatatacatatgttcactatagacgagatg

SEQ ID NO: 312 - DNA, 23 bp, synthetic construct
  misc_feature 1..23: note = Primer
  source 1..23: mol_type = other DNA
atatagctgcggcgtatctgatc

SEQ ID NO: 313 - DNA, 45 bp, synthetic construct
  misc_feature 1..45: note = Primer
  source 1..45: mol_type = other DNA
agatacgccgcagctatatacatctatatggacagctacgagaag

SEQ ID NO: 314 - DNA, 22 bp, synthetic construct
  misc_feature 1..22: note = Primer
  source 1..22: mol_type = other DNA
gtcggatgtctctaagatctgg

SEQ ID NO: 315 - DNA, 26 bp, synthetic construct
  misc_feature 1..26: note = Primer
  source 1..26: mol_type = other DNA
gcgaaattaatacgactcactatagg

SEQ ID NO: 316 - DNA, 21 bp, synthetic construct
  misc_feature 1..21: note = Primer
  source 1..21: mol_type = other DNA
tactcgcttctggtgaatgtc

SEQ ID NO: 317 - DNA, 26 bp, synthetic construct
  misc_feature 1..26: note = Primer
  source 1..26: mol_type = other DNA
gagctttagccgctaagagcatcatg

SEQ ID NO: 318 - DNA, 26 bp, synthetic construct
  misc_feature 1..26: note = Primer
  source 1..26: mol_type = other DNA
catgatgctcttagcggctaaagctc

SEQ ID NO: 319 - DNA, 21 bp, synthetic construct
  misc_feature 1..21: note = Primer
  source 1..21: mol_type = other DNA
gttgctggcggcaacaacccc

SEQ ID NO: 320 - DNA, 21 bp, synthetic construct
  misc_feature 1..21: note = Primer
  source 1..21: mol_type = other DNA
ggggttgttgccgccagcaac

SEQ ID NO: 321 - DNA, 25 bp, synthetic construct
  misc_feature 1..25: note = Primer
  source 1..25: mol_type = other DNA
gatgtcagcaaaagccaggttaagg

SEQ ID NO: 322 - DNA, 25 bp, synthetic construct
  misc_feature 1..25: note = Primer
  source 1..25: mol_type = other DNA
ccttaacctggcttttgctgacatc

SEQ ID NO: 323 - DNA, 23 bp, synthetic construct
  misc_feature 1..23: note = Primer
  source 1..23: mol_type = other DNA
gcttgaagatggcagcaaaatcc

SEQ ID NO: 324 - DNA, 23 bp, synthetic construct
  misc_feature 1..23: note = Primer
  source 1..23: mol_type = other DNA
ggattttgctgccatcttcaagc

SEQ ID NO: 325 - DNA, 28 bp, synthetic construct
  misc_feature 1..28: note = Primer
  source 1..28: mol_type = other DNA
ctatgactataggcgcgaagatgtcagc

SEQ ID NO: 326 - DNA, 28 bp, synthetic construct
  misc_feature 1..28: note = Primer
  source 1..28: mol_type = other DNA
gctgacatcttcgcgcctatagtcatag

SEQ ID NO: 327 - DNA, 20 bp, synthetic construct
  misc_feature 1..20: note = Primer
  source 1..20: mol_type = other DNA
acgcatgtccggtaaaatga

SEQ ID NO: 328 - DNA, 20 bp, synthetic construct
  misc_feature 1..20: note = Primer
  source 1..20: mol_type = other DNA
caagtcattttaccggacat

SEQ ID NO: 329 - DNA, 42 bp, synthetic construct
  misc_feature 1..42: note = Primer
  source 1..42: mol_type = other DNA
gctcaggaagactttgcttaaaatggttcaacgctgacaaag

SEQ ID NO: 330 - DNA, 40 bp, synthetic construct
  misc_feature 1..40: note = Primer
  source 1..40: mol_type = other DNA
gtttagaagacttgatcttacaggctggttacgttaccag

SEQ ID NO: 331 - DNA, 28 bp, synthetic construct
  misc_feature 1..28: note = Primer
  source 1..28: mol_type = other DNA
acgcatgagtcagaatacgctgaaagtt

SEQ ID NO: 332 - DNA, 28 bp, synthetic construct
  misc_feature 1..28: note = Primer
  source 1..28: mol_type = other DNA
caagaactttcagcgtattctgactcat

SEQ ID NO: 333 - DNA, 40 bp, synthetic construct
  misc_feature 1..40: note = Primer
  source 1..40: mol_type = other DNA
gctcaggaagactttgctaatgaagatgcggaatttgatg

SEQ ID NO: 334 - DNA, 35 bp, synthetic construct
  misc_feature 1..35: note = Primer
  source 1..35: mol_type = other DNA
gtttagaagacttgatcttactcgcggaacagcgc

SEQ ID NO: 335 - DNA, 34 bp, synthetic construct
  misc_feature 1..34: note = Primer
  source 1..34: mol_type = other DNA
acgcatgcgaagctcggctaagcaagaagaacta

SEQ ID NO: 336 - DNA, 34 bp, synthetic construct
  misc_feature 1..34: note = Primer
  source 1..34: mol_type = other DNA
caagtagttcttcttgcttagccgagcttcgcat

SEQ ID NO: 337 - DNA, 50 bp, synthetic construct
  misc_feature 1..50: note = Primer
  source 1..50: mol_type = other DNA
gtttagaagactttgcttttaaagcattacttaaagaagagaaatttagc

SEQ ID NO: 338 - DNA, 38 bp, synthetic construct
  misc_feature 1..38: note = Primer
  source 1..38: mol_type = other DNA
gtttagaagacttgatcttaaagctcctggtcgaacag

SEQ ID NO: 339 - DNA, 39 bp, synthetic construct
  misc_feature 1..39: note = Primer
  source 1..39: mol_type = other DNA
gctcaggaagactaccggtggcacgtaagaggttccaac

SEQ ID NO: 340 - DNA, 89 bp, synthetic construct
  misc_feature 1..89: note = Primer
  source 1..89: mol_type = other DNA
gtttaggatccgatcgcgtcttctgatcgttggaatcgccatgggaagtcgaatggaagactactctagtagtgctcagtatctctatc

SEQ ID NO: 341 - DNA, 62 bp, synthetic construct
  misc_feature 1..62: note = Primer
  source 1..62: mol_type = other DNA
gctcaggaagacttagagaagcttgcggaggagcatgcatgagcaaaggagaagaacttttc

SEQ ID NO: 342 - DNA, 44 bp, synthetic construct
  misc_feature 1..44: note = Primer
  source 1..44: mol_type = other DNA
gtttagaagacttgatcctatcatttgtagagttcatccatgcc

SEQ ID NO: 343 - DNA, 57 bp, synthetic construct
  misc_feature 1..57: note = Primer
  source 1..57: mol_type = other DNA
gctcaggaagacttagagaagcttgcggaggagcatgcatggcttccaaggtgtacg

SEQ ID NO: 344 - DNA, 41 bp, synthetic construct
  misc_feature 1..41: note = Primer
  source 1..41: mol_type = other DNA
gtttagaagacttgatctcattactgctcgttcttcagcac

SEQ ID NO: 345 - DNA, 55 bp, synthetic construct
  misc_feature 1..55: note = Primer
  misc_feature 34..35: note = n is a, c, g, or t
  source 1..55: mol_type = other DNA
acactctttccctacacgacgctcttccgatctnnagctcggctaagcaagaaga

SEQ ID NO: 346 - DNA, 56 bp, synthetic construct
  misc_feature 1..56: note = Primer
  misc_feature 34..36: note = n is a, c, g, or t
  source 1..56: mol_type = other DNA
acactctttccctacacgacgctcttccgatctnnnagctcggctaagcaagaaga

SEQ ID NO: 347 - DNA, 57 bp, synthetic construct
  misc_feature 1..57: note = Primer
  misc_feature 34..37: note = n is a, c, g, or t
  source 1..57: mol_type = other DNA
acactctttccctacacgacgctcttccgatctnnnnagctcggctaagcaagaaga

SEQ ID NO: 348 - DNA, 58 bp, synthetic construct
  misc_feature 1..58: note = Primer
  misc_feature 34..38: note = n is a, c, g, or t
  source 1..58: mol_type = other DNA
acactctttccctacacgacgctcttccgatctnnnnnagctcggctaagcaagaaga

SEQ ID NO: 349 - DNA, 55 bp, synthetic construct
  misc_feature 1..55: note = Primer
  misc_feature 34..35: note = n is a, c, g, or t
  source 1..55: mol_type = other DNA
gtgactggagttcagacgtgtgctcttccgatcnnggtcaacatccgcgagactt

SEQ ID NO: 350 - DNA, 56 bp, synthetic construct
  misc_feature 1..56: note = Primer
  misc_feature 34..36: note = n is a, c, g, or t
  source 1..56: mol_type = other DNA
gtgactggagttcagacgtgtgctcttccgatcnnnggtcaacatccgcgagactt

SEQ ID NO: 351 - DNA, 57 bp, synthetic construct
  misc_feature 1..57: note = Primer
  misc_feature 34..37: note = n is a, c, g, or t
  source 1..57: mol_type = other DNA
gtgactggagttcagacgtgtgctcttccgatcnnnnggtcaacatccgcgagactt

SEQ ID NO: 352 - DNA, 58 bp, synthetic construct
  misc_feature 1..58: note = Primer
  misc_feature 34..38: note = n is a, c, g, or t
  source 1..58: mol_type = other DNA
gtgactggagttcagacgtgtgctcttccgatcnnnnnggtcaacatccgcgagactt

SEQ ID NO: 353 - DNA, 23 bp, synthetic construct
  misc_feature 1..23: note = Primer
  source 1..23: mol_type = other DNA
gctgaaaggaggaactatatccg

SEQ ID NO: 354 - DNA, 29 bp, synthetic construct
  misc_feature 1..29: note = Primer
  source 1..29: mol_type = other DNA
caaaatcccttataaatcaaaagaatagc

SEQ ID NO: 355 - DNA, 121 bp, synthetic construct
  misc_feature 1..121: note = Primer
  source 1..121: mol_type = other DNA
cgccgcaaggaatggtgcatgcaactagtatacagtgactcttggcgcgccttgacggctagctcagtcctaggtacagtgctagctactagagaaagaggagaaatactagatgaaaaac

SEQ ID NO: 356 - DNA, 45 bp, synthetic construct
  misc_feature 1..45: note = Primer
  source 1..45: mol_type = other DNA
cgatcctacaggtgaattcatgcctttaattataaacgcagaaag

SEQ ID NO: 357 - DNA, 144 bp, synthetic construct
  misc_feature 1..144: note = Primer
  source 1..144: mol_type = other DNA
ggcatgaattcacctgtaggatcgtacaggtttacgcaagaaaatggtttgttatagtcgaataaatactgagtcttcaccacgacgatttccggcagtttctccacagaagacaacgattaaaggcatcaaataaaacgaaag

SEQ ID NO: 358 - DNA, 48 bp, synthetic construct
  misc_feature 1..48: note = Primer
  source 1..48: mol_type = other DNA
gaaagttggaacctcttacgtgccagtcgaccccagctgtctagggcg

SEQ ID NO: 359 - DNA, 36 bp, synthetic construct
  misc_feature 1..36: note = Primer
  source 1..36: mol_type = other DNA
tcgaccattcgacttcccacgattccaacgatcagg

SEQ ID NO: 360 - DNA, 36 bp, synthetic construct
  misc_feature 1..36: note = Primer
  source 1..36: mol_type = other DNA
gatccctgatcgttggaatcgtgggaagtcgaatgg

SEQ ID NO: 361 - DNA, 63 bp, synthetic construct
  misc_feature 1..63: note = Primer
  source 1..63: mol_type = other DNA
gctcagggtctcatactagagaaagaggagaaatactagatggaagatgccaaaaacataaag

SEQ ID NO: 362 - DNA, 38 bp, synthetic construct
  misc_feature 1..38: note = Primer
  source 1..38: mol_type = other DNA
gtttaggtctcaatcgtcattacacggcgatctttccg

SEQ ID NO: 363 - DNA, 37 bp, synthetic construct
  misc_feature 1..37: note = Primer
  source 1..37: mol_type = other DNA
gctcaagaagacaaagagatggcttccaaggtgtacg

SEQ ID NO: 364 - DNA, 41 bp, synthetic construct
  misc_feature 1..41: note = Primer
  source 1..41: mol_type = other DNA
gctcagggtctcatactatggaagatgccaaaaacataaag

SEQ ID NO: 365 - DNA, 37 bp, synthetic construct
  misc_feature 1..37: note = Primer
  source 1..37: mol_type = other DNA
gctcaggccatgccggcggcacgtaagaggttccaac

SEQ ID NO: 366 - DNA, 19 bp, synthetic construct
  misc_feature 1..19: note = Primer
  source 1..19: mol_type = other DNA
ctcctttgctcatgcatgc

SEQ ID NO: 367 - DNA, 38 bp, synthetic construct
  misc_feature 1..38: note = Primer
  source 1..38: mol_type = other DNA
gctcaggcatgcatgttcactatagacgagatgctatc

SEQ ID NO: 368 - DNA, 23 bp, synthetic construct
  misc_feature 1..23: note = Primer
  source 1..23: mol_type = other DNA
aagtcggatgtctctaagatctg

SEQ ID NO: 369 - DNA, 35 bp, synthetic construct
  misc_feature 1..35: note = Primer
  source 1..35: mol_type = other DNA
gcggaggagcatgcatgtttaccatcgacgagatg

SEQ ID NO: 370 - DNA, 19 bp, synthetic construct
  misc_feature 1..19: note = Primer
  source 1..19: mol_type = other DNA
cagccggatctcgagttag

SEQ ID NO: 371 - DNA, 20 bp, synthetic construct
  misc_feature 1..20: note = TaqMan Primer
  source 1..20: mol_type = other DNA
tggcgcatacaaagagaagc

SEQ ID NO: 372 - DNA, 20 bp, synthetic construct
  misc_feature 1..20: note = TaqMan Primer
  source 1..20: mol_type = other DNA
actccaatccggactacgac

SEQ ID NO: 373 - DNA, 22 bp, synthetic construct
  misc_feature 1..22: note = TaqMan Probe
  source 1..22: mol_type = other DNA
acctcgcgagagcaagcggacc

SEQ ID NO: 374 - DNA, 20 bp, synthetic construct
  misc_feature 1..20: note = TaqMan Primer
  source 1..20: mol_type = other DNA
cggatcacatgaaacggcat

SEQ ID NO: 375 - DNA, 20 bp, synthetic construct
  misc_feature 1..20: note = TaqMan Primer
  source 1..20: mol_type = other DNA
cgtcttgtaggtcccgtcat

SEQ ID NO: 376 - DNA, 22 bp, synthetic construct
  misc_feature 1..22: note = TaqMan Probe
  source 1..22: mol_type = other DNA
accttcgggcatggcactcttg

SEQ ID NO: 377 - DNA, 20 bp, synthetic construct
  misc_feature 1..20: note = TaqMan Primer
  source 1..20: mol_type = other DNA
aatgggtaagtccggcaaga

SEQ ID NO: 378 - DNA, 20 bp, synthetic construct
  misc_feature 1..20: note = TaqMan Primer
  source 1..20: mol_type = other DNA
cgtggcccacaaagatgatt

SEQ ID NO: 379 - DNA, 24 bp, synthetic construct
  misc_feature 1..24: note = TaqMan Probe
  source 1..24: mol_type = other DNA
acctcaccgcttggttcgagctgc

SEQ ID NO: 380 - DNA, 22 bp, synthetic construct
  misc_feature 1..22: note = TaqMan Primer
  source 1..22: mol_type = other DNA
gctccaacaccccaacatcttc

SEQ ID NO: 381 - DNA, 20 bp, synthetic construct
  misc_feature 1..20: note = TaqMan Primer
  source 1..20: mol_type = other DNA
gctccaaaacaacaacggcg

SEQ ID NO: 382 - DNA, 25 bp, synthetic construct
  misc_feature 1..25: note = TaqMan Probe
  source 1..25: mol_type = other DNA
caggtgtcgcaggtcttcccgacga

SEQ ID NO: 383 - DNA, 6350 bp, synthetic construct
  misc_feature 1..6350: note = Plasmid with RT-Cas1/Cas2
  source 1..6350: mol_type = other DNA
ggccgctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagatcattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcggcacgtaagaggttccaactttcaccataatgaaacatactagagaaagaggagaaatactagatgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcctaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagtccctatcagtgatagagattgacatccctatcagtgatagagatactgagcactactagagaagcttgcggaggagcatgcatgtttaccatcgacgagatgctgagcaagaacaaccaacgtctggcgttcgaacactttgcgaccaaaaacgacggttgcggcccggacggtatgcacgttagcgaactggagaagtactggcgtatgaatcacgaccagatcattagcgatctgaagaaccaggaataccagccgggtattatcctgattcgtgagcacatgaataaaaccggcaaacgccgtaatatcgcgagcctgaacgttattgatcgtttcattacccgtctgctgagccagaaactgaaccgttatctggcgccgattttctgcgagaacagctatgcgtaccaggacagcaaaggcgttatgccggcggttctgaaagcgaaggagtacgttgagctgggtatgcgtcacgtgattgaaatcgatctgaaaaactactttgacaccattccgctggaaaacctgatcccggagatcgaacgctacatcaccgatgaggcggttctgcatctgatcaagcaatacctgttttgcgatattagcttcgagggcaaaatcagccgtaaaacccaaggtatcgttcaaggtaacgcgatcagcccgatcctgagcaacctgtacctgaacgacttcgacaaggaactggacgagagcaagctgtgctggattcgttacgcggacaatatctacatctatatggatagctatgagaaggcgctgctggtgtatagcgagctgaccgagcgtctggagcgtcgcaagctgaccgttaacaaggagaaaagcggcgtttttgatgtgagcacccgtagcattctgggctacgacatcctgattcgtaacaagaaagtggacgttcgtaagcatatttacaaaagcgttaatcagtacagcaactggcacgatagccgcctggagtttatcaacggtcgttaccacatcaccagcgatggcatcctgaatcgccaagacttcggcctgctgttcgagaatgagcagaaaaaacactacatcccggttgaggttagcgatcagctgaacatctacggtaatgttaccctggcgagcaatgtgctgcaaagtttcagcaaccgcgaaatcaaagtgagcttcttcgacaagtatggtcgtctgattggtagcttcctgccggaaaaaaccaagaaaagcgcggagatcattctggtgcagagcaagaactatctgaacgaagatgtgcgtatggataccgcgcgccgtatggaaattgcgggtctgcacaacatccgtgcgaatctgcgctactacgataagaaacacaagggcgactttaaagagaaggttgacgcgattagcggctacattgacgcgctgaaccgtgcgccgagcgttaatgatatgatgctgctggaagcgaaagcgcgccaactgtattatacctgttttaaccaaatcctggaaaccagcgacttccagttcgaaaagcgtaccaagcgtccgccgaaagatgcgatcaatgcgtgcattagcttcggcaataccctgctgtataatctatttgtgaatattatctggaagaagggtctggacccgcgctttggcgttgttcacgcgagcaacaaacgcaatcaaagcctgaacctggacttcgcggacatctttaaaccgatcgtgattgaccgcattatcttcaccatgatcaataagaaaatgctgaccctgctgaccgattttgaaaccagcaaccagggtgtgtatctgagccgtgagggcaagaacatcttcctgcaaatgtacgaggagaagctgaaaagccgcattaccatcaagggtaaagagatgagctattaccagctgctggagagcgaagtgcagaattacaagaatttcattctgaccggcgaaacctataaaccgtataagtattattaacgtacgttaactttaagaaggagaacttaagatgtatgttatcctggtgtacgacatccaccagaagcgtgttggtaaagcgctgaaaatctgccgtaagtacctgatccacatccaaaagagcgtgttcgaaggcaacatcaccgagagcaaactgaaggcgctgaaagaggaactgggtcacctgattgacacccagatggatagcgtgattatctaccacctggacagcgttaagtacaccaaaaaggagcagatcggtattgtgcagagcaccagcaatgtgatctaactcgagatccggctgctaacaaagcccgaaaggaagctgagttggctgctgccaccgctgagcaataactagcataaccccttggggcctctaaacgggtcttgaggggttttttgctgaaaggaggaactatatccggataccaggtgcggggaagcttatgttccatagcaaaaagtcggtcagtctcgtggctgaaatcatgagttccacaaaatggctgaaattcaaggaaaatcaggaatctcagaaaaacgatcgaccgactttttcgataaaatggttgcaaaaatgagaaaaatctgatttaatagaatctgaaaacagcggaaatgctgttgtcgtactttacctaaaaggaattgaaacgtcccgc